(19) United States
(12) Patent Application Publication
(10) Pub. No.: US 2025/0144227 A1
(43) Pub. Date: May 8, 2025

(54) C-16 MODIFIED TRIOXACARCINS, ANTIBODY DRUG CONJUGATES, AND USES THEREOF

(71) Applicant: President and Fellows of Harvard College, Cambridge, MA (US)

(72) Inventors: Andrew G. Myers, Cambridge, MA (US); Jason Anesini, Cambridge, MA (US); Lucas Valdes, Cambridge, MA (US); Ethan L. Magno, Cambridge, MA (US); Wojtek Gruchot, Cambridge, MA (US)

(73) Assignee: President and Fellows of Harvard College, Cambridge, MA (US)

(21) Appl. No.: 18/712,928

(22) PCT Filed: Nov. 22, 2022
(86) PCT No.: PCT/US2022/050726

§ 371 Date: May 23, 2024

Related U.S. Application Data
(60) Provisional application No. 63/354,967, filed on Jun. 23, 2022.
(60) Provisional application No. 63/306,347, filed on Feb. 3, 2022.
(60) Provisional application No. 63/283,160, filed on Nov. 24, 2021.

(51) Int. Cl. A61K 47/68; A61K 31/357; C07D 493/22

(52) U.S. Cl. CPC A61K 47/6803; A61K 31/357; A61K 47/6849; A61K 47/6889; C07D 493/22

(57) ABSTRACT

Provided herein are compound of Formula (I) and (II), and pharmaceutically acceptable salts thereof. Also provided are methods of preparing the compounds, pharmaceutically acceptable compositions thereof, and methods of their use and treatment.

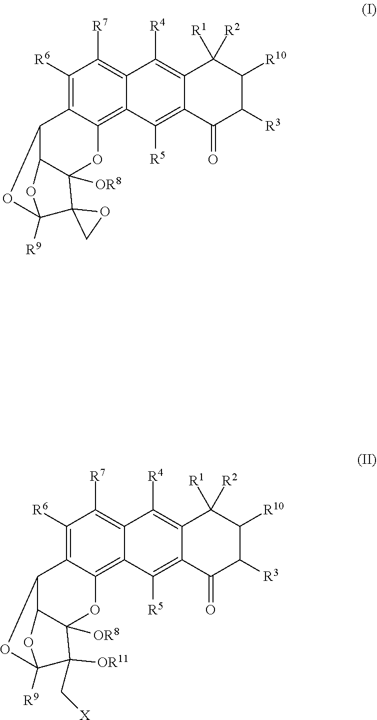

### RELATED APPLICATIONS

This application claims priority under 35 U.S.C. § 119 (e) to U.S. Provisional Applications, U.S. Ser. No. 63/283,160, filed Nov. 24, 2021; U.S. Ser. No. 63/306,347, filed Feb. 3, 2022; and U.S. Ser. No. 63/354,967, filed Jun. 23, 2022. The entire content of each is incorporated herein by reference.

### GOVERNMENT SUPPORT

This invention was made with Government support under CA047148 awarded by the National Institutes of Health. The Government has certain rights in the invention.

### BACKGROUND

Antibody-drug conjugates (ADCs) have emerged as an effective, targeted therapeutic tool wherein a small molecule warhead is coupled to a cancer cell-selective antibody. This simple design strategy belies the careful coordination of toxic payload, linker chemistry, and antibody engineering that has proved to be a challenge in the discovery of effective ADCs. Advances are enabled through the expansion of synthetically accessible payloads differentiated with respect to their mechanisms of action, as well as innovation in the means by which these warheads are coupled to the antibody.

Unlike their traditional chemotherapeutic counterparts which rely on marginal differences between the growth rates of healthy and cancerous cells to affect ‘selective’ cytotoxicity, ADCs are designed to act on specific molecular targets associated with cancer progression thereby specifically inhibiting the growth of cancerous cells. To date, the Food and Drug Administration (FDA) has approved eleven ADCs to treat metastatic and relapsed cancers. In addition, more than 100 ADCs are currently in phase 1/2 trials, a testament to the broad commercial interest in this technology.

Despite the numerous advances in ADC technology since its inception, a number of drawbacks still exist, demanding the development of new strategies to combat these shortcomings. FDA-approved ADCs (and a large majority of those in development) draw from just four classes of natural products. This limited spectrum of cytotoxic agents not only leaves the treatment susceptible to resistance, but also fails to address the diversity of cancer cell sensitivities across tumor types that has been established over half a century of clinical experience.

### SUMMARY

In order to further diversify the arsenal of ADC toxins, the inventors have built upon their modular synthetic platform of the trioxacarcin natural product class to enable incorporation of the trioxacarcins into ADC constructs. Unlike existing payloads, which are cytotoxic by DNA strand scission, tubulin polymerization inhibition, or topoisomerase I inhibition, the trioxacarcins' trimodal binding pattern-intercalation, alkylation, and base flip-out-is a unique mechanism for DNA complexation among known antitumor antibiotics, making it an attractive candidate warhead for novel ADCs. Beyond their distinct mechanism of action, the trioxacarcins are attractive candidates for novel ADCs because they demonstrate a favorable safety profile relative to many other ADC toxins, such as calicheamicin. A recent clinical trial of LL-D49194a1, a distinct member of the trioxacarcin natural product class, was dosed in patients with metastatic cancer refractory to conventional treatment found that pancarditis was the only significant dose-limiting toxicity, and that this was only problematic at doses beyond those which affected tumor volume decrease.

Prior to this work, the inventors developed a fully synthetic method to access known trioxacarcins and novel trioxacarcin analogs. See, e.g., PCT Application Publication No. WO 2011/119549 and PCT Application Publication No. WO 2014/082065, both of which are incorporated herein by reference. A wide variety of fully synthetic natural and non-natural trioxacarcin compounds have been prepared by a process that is amenable to scaling. The inventors also created ADCs of novel trioxacarcin-antibody drug conjugates. See, e.g., PCT Application Publication No. WO 2019/032961 and International Application No. PCT/US2021/036718, which are incorporated herein by reference. However, there still exists a need for controlled administration of therapeutic compounds, such as the trioxacarcins via ADC technology.

The present disclosure provides trioxacarcin analogs, antibody drug conjugates, and antibody drug conjugate precursor compounds. In particular, the present disclosure provides access to novel trioxacarcin-antibody drug conjugates and trioxacarcin-antibody drug conjugate precursor compounds with advantageous properties (e.g., stability, release kinetics). The trioxacarcins are highly toxic to a variety of cell types. Linking a trioxacarcin to an antibody preserves the trioxacarcin's potency against a particular cell type while increasing specificity for the target cell, and potentially increasing endocytosis of the trioxacarcin. These effects enable lowering the overall amount of trioxacarcin to be delivered, thereby reducing the associated toxicity and any undesired side effects.

In one aspect, provided is a compound of Formula (I):

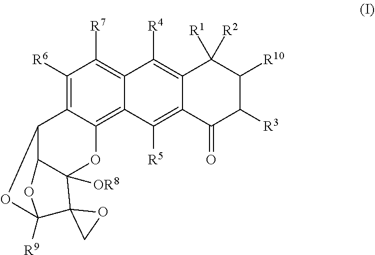

or a pharmaceutically acceptable salt, solvate, hydrate, polymorph, co-crystal, tautomer, stereoisomer, isotopically labeled derivative, or prodrug thereof;
wherein:
R¹ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{A1}; —C(═O)R^{A2}; —CO₂R^{A2}; —CN; —SCN; —SR^{A1}; —SOR^{A1}; —SO₂R^{A1}; —NO₂; —N₃; —N(R^{A2})₂; —NR^{A2}C(═O)R^{A2}; —NR^{A2}C(═O)N(R^{A2})₂; —OC(═O)OR^{A1}; —OC(═O)R^{A2}; —OC(═O)N(R^{A2})₂; —NR^{A}C(═O)OR^{A1}; or —C(R^{A2})₃; wherein each occurrence of R^{A1} is independently hydrogen; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{A1} groups are optionally joined to form a substituted or unsubstituted heterocyclyl or substituted or unsubstituted heteroaryl ring; and wherein each occurrence of R^{A2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{A1}; —SR^{A1}; or —N(R^{A1})₂, or two R^{A2} groups are optionally joined to form a substituted or unsubstituted heterocyclyl or substituted or unsubstituted heteroaryl ring; with the proviso that if R¹ is —OR^{A1}, R^{A1} is not 4-methoxybenzyl or carbohydrate.
R² is hydrogen; or R¹ and R² are joined to form ═O; ═N(R^{A2}); or ═S;
R³ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{C1}; —C(═O)R^{C2}; —CO₂R^{C1}; —CN; —SCN; —SR^{C1}; —SOR^{C1}; —SO₂R^{C2}; —NO₂; —N₃; —N(R^{C2})₂; —NHC(═O)R^{C2}; —NR^{C2}C(═O)N(R^{C2})₂; —OC(═O)OR^{C1}; —OC(═O)R^{C2}; —OC(═O)N(R^{C2})₂; —NR^{C2}C(═O)OR^{C1}; or —C(R^{C2})₃; wherein each occurrence of R^{C1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{C1} groups are optionally joined to form a substituted or unsubstituted heterocyclyl or substituted or unsubstituted heteroaryl ring; and wherein each occurrence of R^{C2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{C1}; —SR^{C1}; or —N(R^{C1})₂; or two R^{C2} groups are optionally joined to form a substituted or unsubstituted heterocyclyl or substituted or unsubstituted heteroaryl ring;
R⁴ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{D1}; —C(═O)R^{D2}; —CO₂R^{D2}; —CN; —SCN; —SR^{D1}; —SOR^{D1}; —SO₂R^{D2}; —NO₂; —N₃; —N(R^{D2})₂; —NR^{D2}C(═O)R^{D2}; —NR^{D2}C(═O)N(R^{D2})₂; —OC(═O)OR^{D1}; —OC(═O)R^{D2}; —OC(═O)N(R^{D2})₂; —NR^{D2}C(═O)OR^{D1}; or —C(R^{D2})₃; wherein each occurrence of R^{D1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{D1} groups are optionally joined to form a substituted or unsubstituted heterocyclyl or substituted or unsubstituted heteroaryl ring; and wherein each occurrence of R^{D2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{D1}; —SR^{D1}; or —N(R^{D1})₂; or two R^{D2} groups are optionally joined to form a substituted or unsubstituted heterocyclyl or substituted or unsubstituted heteroaryl ring;
R⁵ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{E1}; —C(═O)R^{E2}; —CO₂R^{E1}; —CN; —SCN; —SR^{E1}; —SOR^{E1}; —SO₂R^{E2}; —NO₂; —N₃; —N(R^{E2})₂; —NR^{E2}C(═O)R^{E2}; —NR^{E2}C(═O)N(R^{E2})₂; —OC(═O)OR^{E1}; —OC(═O)R^{E2}; —OC(═O)N(R^{E2})₂; —NR^{E2}C(═O)OR^{E1}; or —C(R^{E2})₃; wherein each occurrence of R^{E1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{E1} groups are optionally joined to form a substituted or unsubstituted heterocyclyl or substituted or unsubstituted heteroaryl ring; and wherein each occurrence of R^{E2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{E1}; —SR^{E1}; or —N(R^{E1})₂; or two R^{E2} groups are optionally joined to form a substituted or unsubstituted heterocyclyl or substituted or unsubstituted heteroaryl ring;
R⁶ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{F1}; —C(═O)R^{F2}; —CO₂R^{F1}; —CN; —SCN; —SR^{F1}; —SOR^{F1}; —SO₂R^{F2}; —NO₂; —N₃; —N(R^{F2})₂; —NR^{F2}C(═O)R^{F2}; —NR^{F2}C(═O)N(R^{F2})₂; —OC(═O)OR^{F1}; —OC(═O)R^{F2}; —OC(═O)N(R^{F2})₂; —NR^{F2}C(═O)OR^{F1}; or —C(R^{F2})₃; wherein each occurrence of R^{F1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{F1} groups are optionally joined to form a substituted or unsubstituted heterocyclyl or substituted or unsubstituted heteroaryl ring; and wherein each occurrence of R^{F2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{F1}; —SR^{F1}; or —N(R^{F1})₂; or two R^{F2} groups are optionally joined to form a substituted or unsubstituted heterocyclyl or substituted or unsubstituted heteroaryl ring;
R⁷ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{G1}; —C(═O)R^{G2}; —CO₂R^{G1}; —CN; —SCN; —SR^{G1}; —SOR^{G1}; —SO₂R^{G2}; —NO₂; —N₃; —N(RG)₂; —NR^{G2}C(═O)R^{G2}; —NR^{G2}C(═O)N(R^{G2})₂; —OC(═O)OR^{G1}; —OC(═O)R^{G2}; —OC(═O)N(R^{G2})₂; —NR^{G2}C(═O)OR^{G1}; or —C(R^{G2})₃; wherein each occurrence of R^{G1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{G1} groups are optionally joined to form a substituted or unsubstituted heterocyclyl or substituted or unsubstituted heteroaryl ring; and wherein each occurrence of R^{G2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{G1}; —SR^{G1}; or —N(R^{G1})₂; or two R^{G2} groups are optionally joined to form a substituted or unsubstituted heterocyclyl or substituted or unsubstituted heteroaryl ring;
R⁸ is hydrogen; an oxygen protecting group; a carbohydrate; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; substituted or unsubstituted heteroaliphatic; or acyl; —OR^{H9}; —OC(═O)R^{H9}; —N(R^{H9})₂; or —NHC(═O)R^{H9}; wherein each occurrence of R^{H9} is independently hydrogen; substituted or unsubstituted alkyl; an oxygen protecting group when attached to an oxygen atom; or a nitrogen protecting group when attached to a nitrogen atom; or two R^{H9} groups are joined to form a substituted or unsubstituted heterocyclic or substituted or unsubstituted heteroaryl ring;
R⁹ is hydrogen or —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring;
R¹⁰ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{B1}; —C(═O)R^{B2}; —CO₂R^{B2}; —CN; —SCN; —SR^{B1}; —SOR^{B1}; —SO₂R^{B2}; —NO₂; —N₃; —N(R^{B2})₂; —NR^{B2}C(═O)R^{B2}; —NR^{B2}C(═O)N(R^{B2})₂; —OC(═O)OR^{B1}; —OC(═O)R^{B2}; —OC(═O)N(R^{B2})₂; —NR^{B2}C(═O)OR^{B1}; or —C(R^{B2})₃; wherein each occurrence of R^{B1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{B1} groups are optionally joined to form a substituted or unsubstituted heterocyclyl or substituted or unsubstituted heteroaryl ring; and wherein each occurrence of R^{B2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{B1}; —SR^{B1}; or —N(R^{B1})₂; or two R^{B2} groups are optionally joined to form a substituted or unsubstituted heterocyclyl or substituted or unsubstituted heteroaryl ring;
or R¹ and R¹⁰ are optionally taken together with the intervening carbon atoms to form an optionally substituted cyclic moiety;
or R¹⁰ and R³ are optionally taken together with the intervening carbon atoms to form an optionally substituted cyclic moiety;
or R¹ and R⁴ are optionally taken together with the intervening carbon atoms to form an optionally substituted cyclic moiety;
or R⁴ and R⁷ are optionally taken together with the intervening carbon atoms to form an optionally substituted cyclic moiety;
or R⁶ and R⁷ are optionally taken together with the intervening carbon atoms to form an optionally substituted cyclic moiety;
L is a linker;
A is a bond or a group of formula:

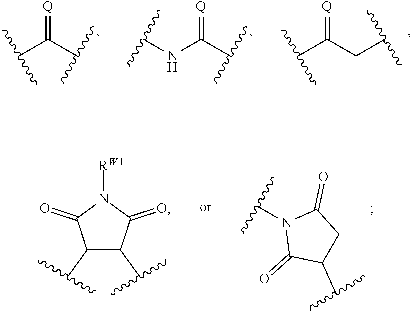

Q is —S— or —O—;
R^{W1} is independently hydrogen, substituted or unsubstituted alkyl; or a nitrogen protecting group;
B is a targeting moiety; and

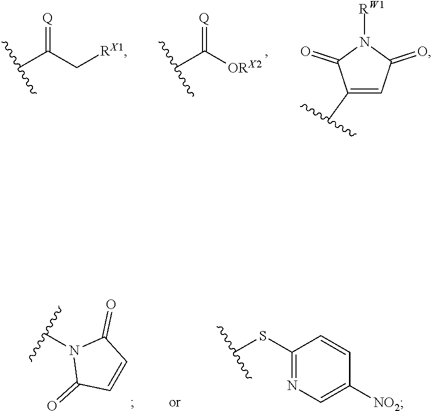

T is hydrogen, —N═C═S,
R^{X1} is a leaving group; and
R^{X2} is hydrogen; substituted or unsubstituted alkyl; substituted or unsubstituted heterocyclyl; substituted or unsubstituted aryl; substituted or unsubstituted heteroaryl; or an oxygen protecting group

In certain embodiments, the compound of Formula (I) is of formula:

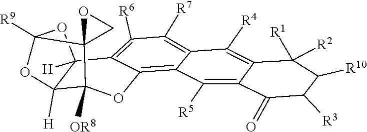

or a pharmaceutically acceptable salt thereof.

In certain embodiments, the compound of Formula (I) is of formula:

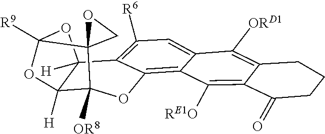

or a pharmaceutically acceptable salt thereof.

In certain embodiments, the compound of Formula (I) is of formula:

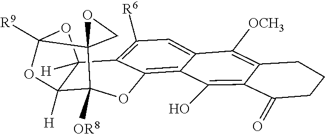

or a pharmaceutically acceptable salt thereof.

In certain embodiments, the compound of Formula (I) is of formula:

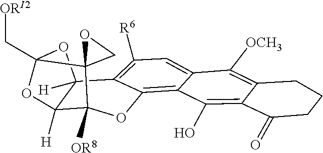

or a pharmaceutically acceptable salt thereof.

In certain embodiments, the compound of Formula (I) is of formula:

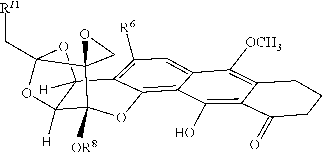

or a pharmaceutically acceptable salt thereof.

In another aspect, provided is a compound of Formula (II):

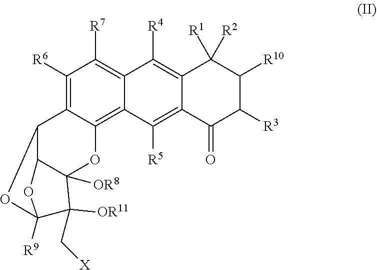

or a pharmaceutically acceptable salt, solvate, hydrate, polymorph, co-crystal, tautomer, stereoisomer, isotopically labeled derivative, or prodrug thereof;
wherein:
R¹ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{A1}; —C(═O)R^{A2}; —CO₂R^{A2}; —CN; —SCN; —SR^{A1}; —SOR^{A1}; —SO₂R^{A1}; —NO₂; —N₃; —N(R^{A2})₂; —NR^{A2}C(═O)R^{A2}; —NR^{A2}C(═O)N(R^{A2})₂; —OC(═O)OR^{A1}; —OC(═O)R^{A2}; —OC(═O)N(R^{A2})₂; —NRAC(═O)OR^{A1}; or —C(R^{A2})₃; wherein each occurrence of R^{A1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{A1} groups are optionally joined to form a substituted or unsubstituted heterocyclyl or substituted or unsubstituted heteroaryl ring; and wherein each occurrence of R^{A2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{A1}; —SR^{A1}; or —N(R^{A1})₂; or two R^{A2} groups are optionally joined to form a substituted or unsubstituted heterocyclyl or substituted or unsubstituted heteroaryl ring; with the proviso that if R¹ is —OR^{A1}, R^{A1} is not 4-methoxybenzyl or carbohydrate.
R² is hydrogen; or R¹ and R² are joined to form ═O; ═N(R^{A2}); or ═S;
R³ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{C1}; —C(═O)R^{C2}; —CO₂R^{C1}; —CN; —SCN; —SR^{C1}; —SOR^{C1}; —SO₂R^{C2}; —NO₂; —N₃; —N(R^{C2})₂; —NHC(═O)R^{C2}; —NR^{C2}C(═O)N(R^{C2})₂; —OC(═O)OR^{C1}; —OC(═O)R^{C2}; —OC(═O)N(R^{C2})₂; —NR^{C2}C(═O)OR^{C1}; or —C(R^{C2})₃; wherein each occurrence of R^{C1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{C1} groups are optionally joined to form a substituted or unsubstituted heterocyclyl or substituted or unsubstituted heteroaryl ring; and wherein each occurrence of R^{C2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{C1}; —SR^{C1}; or —N(R^{C1})₂; or two R^{C2} groups are optionally joined to form a substituted or unsubstituted heterocyclyl or substituted or unsubstituted heteroaryl ring;
R⁴ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{D1}; —C(═O)R^{D2}; —CO₂R^{D2}; —CN; —SCN; —SR^{D1}; —SOR^{D1}; —SO₂R^{D2}; —NO₂; —N₃; —N(R^{D2})₂; —NR^{D2}C(═O)R^{D2}; —NR^{D2}C(═O)N(R^{D2})₂; —OC(═O)OR^{D1}; —OC(═O)R^{D2}; —OC(═O)N(R^{D2})₂; —NR^{D2}C(═O)OR^{D1}; or —C(R^{D2})₃; wherein each occurrence of R^{D1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{D1} groups are optionally joined to form a substituted or unsubstituted heterocyclyl or substituted or unsubstituted heteroaryl ring; and wherein each occurrence of R^{D2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{D1}; —SR^{D1}; or —N(R^{D1})₂; or two R^{D2} groups are optionally joined to form a substituted or unsubstituted heterocyclyl or substituted or unsubstituted heteroaryl ring;
R⁵ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{E1}; —C(═O)R^{E2}; —CO₂R^{E1}; —CN; —SCN; —SR^{E1}; —SOR^{E1}; —SO₂R^{E2}; —NO₂; —N₃; —N(R^{E2})₂; —NR^{E2}C(═O)R^{E2}; —NR^{E2}C(═O)N(R^{E2})₂; —OC(═O)OR^{E1}; —OC(═O)R^{E2}; —OC(═O)N(R^{E2})₂; —NR^{E2}C(═O)OR^{E1}; or —C(R^{E2})₃; wherein each occurrence of R^{E1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{E1} groups are optionally joined to form a substituted or unsubstituted heterocyclyl or substituted or unsubstituted heteroaryl ring; and wherein each occurrence of R^{E2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{E1}; —SR^{E1}; or —N(R^{E1})₂; or two R^{E2} groups are optionally joined to form a substituted or unsubstituted heterocyclyl or substituted or unsubstituted heteroaryl ring;
R⁶ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{F1}; —C(═O)R^{F2}; —CO₂R^{F1}; —CN; —SCN; —SR^{F1}; —SOR^{F1}; —SO₂R^{F2}; —NO₂; —N₃; —N(R^{F2})₂; —NR^{F2}C(═O)R^{F2}; —NR^{F2}C(═O)N(R^{F2})₂; —OC(═O)OR^{F1}; —OC(═O)R^{F2}; —OC(═O)N(R^{F2})₂; —NR^{F2}C(═O)OR^{F1}; or —C(R^{F2})₃; wherein each occurrence of R^{F1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{F1} groups are optionally joined to form a substituted or unsubstituted heterocyclyl or substituted or unsubstituted heteroaryl ring; and wherein each occurrence of R^{F2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{F1}; —SR^{F1}; or —N(R^{F1})₂; or two R^{F2} groups are optionally joined to form a substituted or unsubstituted heterocyclyl or substituted or unsubstituted heteroaryl ring;
R⁷ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{G1}; —C(═O)R^{G2}; —CO₂R^{G1}; —CN; —SCN; —SR^{G1}; —SOR^{G1}; —SO₂R^{G2}; —NO₂; —N₃; —N(R^{G})₂; —NR^{G2}C(═O)R^{G2}; —NR^{G2}C(═O)N(R^{G2})₂; —OC(═O)OR^{G1}; —OC(═O)R^{G2}; —OC(═O)N(R^{G2})₂; —NR^{G2}C(═O)OR^{G1}; or —C(R^{G2})₃; wherein each occurrence of R^{G1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{G1} groups are optionally joined to form a substituted or unsubstituted heterocyclyl or substituted or unsubstituted heteroaryl ring; and wherein each occurrence of R^{G2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{G1}; —SR^{G1}; or —N(R^{G1})₂; or two R^{G2} groups are optionally joined to form a substituted or unsubstituted heterocyclyl or substituted or unsubstituted heteroaryl ring;
R⁸ is hydrogen; an oxygen protecting group; a carbohydrate; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; substituted or unsubstituted heteroaliphatic; or acyl; —OR^{H9}; —OC(═O)R^{H9}; —N(R^{H9})₂; or —NHC(═O)R^{H9}; wherein each occurrence of R^{H9} is independently hydrogen; substituted or unsubstituted alkyl; an oxygen protecting group when attached to an oxygen atom; or a nitrogen protecting group when attached to a nitrogen atom; or two R^{H9} groups are joined to form a substituted or unsubstituted heterocyclic or substituted or unsubstituted heteroaryl ring;
R⁹ is hydrogen or —C(R^{I1})₃; wherein each occurrence of R^{I1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{I2}; —SR^{I2}; —N(R^{I2})₂; azido; or halogen; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring;
R¹⁰ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{B1}; —C(═O)R^{B2}; —CO₂R^{B2}; —CN; —SCN; —SR^{B1}; —SOR^{B1}; —SO₂R^{B2}; —NO₂; —N₃; —N(R^{B2})₂; —NR^{B2}C(═O)R^{B2}; —NR^{B2}C(═O)N(R^{B2})₂; —OC(═O)OR^{B1}; —OC(═O)R^{B2}; —OC(═O)N(R^{B2})₂; —NR^{B2}C(═O)OR^{B1}; or —C(R^{B2})₃; wherein each occurrence of R^{B1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{B1} groups are optionally joined to form a substituted or unsubstituted heterocyclyl or substituted or unsubstituted heteroaryl ring; and wherein each occurrence of R^{B2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{B1}; —SR^{B1}; or —N(R^{B1})₂; or two R^{B2} groups are optionally joined to form a substituted or unsubstituted heterocyclyl or substituted or unsubstituted heteroaryl ring;
R^{I1} is hydrogen; substituted or unsubstituted alkyl; acyl; -L-A-B; or -L-T;
X is a halogen;
or R¹ and R¹⁰ are optionally taken together with the intervening carbon atoms to form an optionally substituted cyclic moiety;
or R¹⁰ and R³ are optionally taken together with the intervening carbon atoms to form an optionally substituted cyclic moiety;
or R¹ and R⁴ are optionally taken together with the intervening carbon atoms to form an optionally substituted cyclic moiety;
or R⁴ and R⁷ are optionally taken together with the intervening carbon atoms to form an optionally substituted cyclic moiety;
or R⁶ and R⁷ are optionally taken together with the intervening carbon atoms to form an optionally substituted cyclic moiety;
L is a linker;
A is a bond or a group of formula:

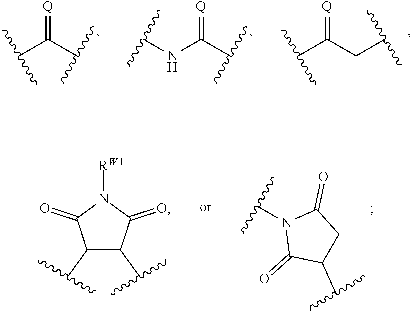

Q is —S— or —O—;
R^{W1} is independently hydrogen, substituted or unsubstituted alkyl; or a nitrogen protecting group;
B is a targeting moiety; and
T is hydrogen, —N═C═S,

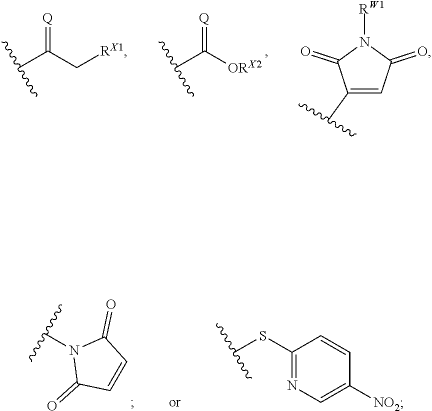

R^{X1} is a leaving group; and
R^{X2} is hydrogen; substituted or unsubstituted alkyl; substituted or unsubstituted heterocyclyl; substituted or unsubstituted aryl; substituted or unsubstituted heteroaryl; or an oxygen protecting group.

In certain embodiments, the compound of Formula (II) is of formula:

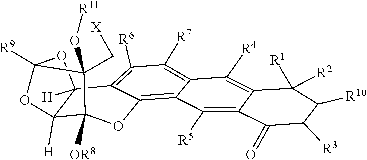

or a pharmaceutically acceptable salt thereof.

In certain embodiments, the compound of Formula (II) is of formula:

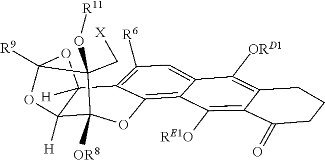

or a pharmaceutically acceptable salt thereof.

In certain embodiments, the compound of Formula (II) is of formula:

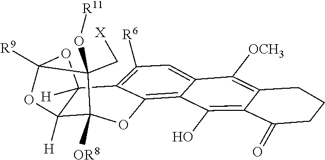

or a pharmaceutically acceptable salt thereof.

In certain embodiments, the compound of Formula (II) is of formula:

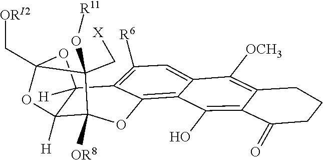

or a pharmaceutically acceptable salt thereof.

In certain embodiments, the compound of Formula (II) is of formula:

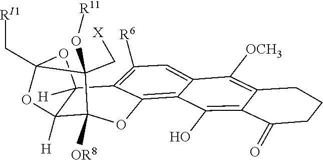

or a pharmaceutically acceptable salt thereof.

In another aspect, provided are intermediate compounds, such as compounds of formula:

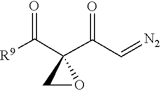

or a salt thereof; wherein:
R⁹ is hydrogen or —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; C(═O)R^{I1}; -L-A-B; or -L-T; or two R^{I2} groups are optionally joined to form a substituted or unsubstituted heterocyclyl or substituted or unsubstituted heteroaryl ring.

In certain embodiments, the compound is:

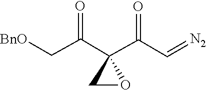

or a salt thereof.

In another aspect, provided are pharmaceutical compositions comprising any of the compounds of Formula (I) or (II), or pharmaceutically acceptable salts thereof, and optionally a pharmaceutically acceptable carrier.

In another aspect, provided are methods of treating cardiovascular disease, proliferative disease (e.g., cancer), diabetic retinopathy, inflammatory disease, autoimmune disease, or infectious disease in a subject in need thereof, the method comprising administering to the subject an effective amount of any of the compounds of Formula (I) or (II) or pharmaceutically acceptable salts thereof, or a pharmaceutical composition comprising such to the subject.

In another aspect, provided are kits comprising compounds of Formula (I) or (II), or pharmaceutically acceptable salts thereof, or pharmaceutical compositions comprising such. In certain embodiments, the kits further comprise instructions for administration (e.g., human administration).

Also within the scope of the present disclosure are pharmaceutical compositions comprising any of the compounds of Formula (I) or (II), or pharmaceutically acceptable salts thereof, and a pharmaceutically acceptable carrier for use in treating cardiovascular disease, proliferative disease (e.g., cancer), diabetic retinopathy, inflammatory disease, autoimmune disease, or infectious disease, as well as uses of any of the compounds of Formula (I) or (II), or pharmaceutically acceptable salts thereof, for manufacturing a medicament for use in treating any of the target diseases.

Also provided are methods of preparing compounds of Formula (I) or (II).

The details of one or more embodiments of the invention are set forth in the Detailed Description below. Other features, objects, and advantages of the invention will be apparent from the Examples and the Claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1B show the results of drug-linker stability assays for compound 42 (FIG. 1A) and compound 39 (FIG. 1B), determined by LC-MS at various time points. LC-MS peaks were integrated using Agilent OpenLab ChemStation and the areas of each peak were normalized to 1-naphthalene acetic acid at 280 nM. Percent drug-linker remaining is defined as the normalized peak area at a given time point divided by the normalized peak area at 0 hours×100%.

FIGS. 2A-2B show the results of drug-linker release assays using a specified enzyme, determined by LC-MS at various time points. FIG. 2A shows the results of release assays for compound 42 using cathepsin B, and without cathepsin B as a control (i.e., showing both stability and release profiles). FIG. 2B shows the results of release assays for compound 39 using glucuronidase, and without glucuronidase as a control (i.e., showing both stability and release profiles). LC-MS peaks were integrated using Agilent OpenLab ChemStation and the areas of each peak were normalized to 1-napthalene acetic acid at 280 nM. Percent drug-linker remaining is defined as the normalized peak area at a given time point divided by the normalized peak area at 0 hours×100%.

FIG. 3 shows an alternate synthetic strategy for compound 45.

FIG. 4 shows the product of conjugation of drug-linker compound 42 to the monoclonal antibody Rituximab, and associated mass spectrometry data.

FIG. 5 shows the structures of a matched glucuronide/glucuronide prodrug/drug-linker system and a mismatched glucuronide/dipeptide prodrug/drug-linker system.

FIG. 6 shows the synthesis of the matched glucuronide/glucuronide prodrug/drug-linker system.

FIGS. 7A-7B show two synthetic schemes for the mismatched glucuronide/dipeptide prodrug/drug-linker system.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein may comprise one or more asymmetric centers, and thus may exist as stereoisomers, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). Compounds may exist as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

As used herein, “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

As used herein, “heteroalkyl” refers to an alkyl group as described herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

As used herein, “alkenyl” refers to a radical of a straight-chain or branched hydrocarbon group having from 2 to 10 carbon atoms and one or more double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl.

As used herein, “heteroalkenyl” refers to an alkenyl group as described herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

As used herein, “alkynyl” refers to a radical of a straight-chain or branched hydrocarbon group having from 2 to 10 carbon atoms and one or more triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

As used herein, “heteroalkynyl” refers to an alkynyl group as described herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro [2,3-b]pyridinyl, 4,5,6,7-tetrahydro-1H-pyrrolo-[2,3-b]pyridinyl, 4,5,6,7-tetrahydrofuro [3,2-c]pyridinyl, 4,5,6,7-tetrahydrothieno[3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group, as described herein, substituted by an aryl group, as described herein, wherein the point of attachment is on the alkyl moiety.

As used herein, “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group, as described herein, substituted by a heteroaryl group, as described herein, wherein the point of attachment is on the alkyl moiety.

As used herein, the term “partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a ring moiety that does not contain a double or triple bond, i.e., the ring contains all single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

As understood from the above, alkyl, alkenyl, alkynyl, heteroalkyl, heteroalkenyl, heteroalkynyl, carbocyclyl, heterocyclyl, aryl, and heteroaryl groups, as described herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa},—NR^{bb}CO₂R^{aa}, —NR^{bb}C(═O)N(R^{bb})₂, —C(═NR^{bb})R^{aa}, —C(═NR^{bb}) OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O) NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR)₂, —OP(═O)(R^{aa})₂, —OP(═O)(OR^{cc})₂, —P(═O)(N(R^{bb})₂)₂, —OP(═O)(N(R^{bb})₂)₂,—NR^{bb}P(═O)(R^{aa})₂, —NR^{bb}P(═O)(OR)₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR)₄, —OP (R^{cc})₂, —OP(R^{cc})₃⁺X⁻, —OP(OR)₂, —OP(OR^{cc})₃⁺X⁻, —OP(R^{cc})₄, —OP(OR^{cc})₄, —B(R^{aa})₂, —B(OR)₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀alkynyl, C₃₋₁₀ carbocyclyl, C₃₋₁₄ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, C(═NR^{cc})N(R^{cc})₂, —SO₂N(R^{cc})₂, —SO₂R^{cc}, —SO₂OR^{cc}, —SOR^{aa}, C(═S)N(R^{cc})₂, —C(═O)SR^{cc}, —C(═S)SR^{cc}, —P(═O)(R^{aa})₂, —P(═O)(OR)₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff}); “X”, —N(OR)R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂,—NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂,—NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆ alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl) (C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂ (C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH (C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆ alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

As used herein, the term “halo” or “halogen” refers to fluorine (fluoro, —F), chlorine (chloro, —Cl), bromine (bromo, —Br), or iodine (iodo, —I).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S) R^{X1}, —C(═S)N(R^{X1})₂, —C(═S)O(R^{X1}), —C(═S)S(R^{X1}), —C(═NR^{X1}) R^{X1}, —C(═NR^{X1}) OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH, H₂PO₄⁻, HCO₃⁻. HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻. B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “leaving group” is given its ordinary meaning in the art of synthetic organic chemistry and refers to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O)-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂ (CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties. Further exemplary leaving groups include, but are not limited to, halo (e.g., chloro, bromo, iodo) and activated substituted hydroxyl groups (e.g., —OC(═O) SR^{aa}, —OC(═O)R^{aa}, —OCO₂R^{aa}, —OC(═O)N(R^{bb})₂, —OC(═NR^{bb}) R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein).

As used herein, the term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR)₂, —OP(OR)₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as described herein.

As used herein, the term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as described herein.

As used herein, the term, “amino” refers to the group —NH₂. The term “substituted amino,” by extension, refers to a monosubstituted amino, a disubstituted amino, or a trisubstituted amino, as described herein. In certain embodiments, the “substituted amino” is a monosubstituted amino or a disubstituted amino group.

As used herein, the term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as described herein, and wherein R^{bb} of the group —NH(R^{bb}) is not hydrogen.

As used herein, the term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa},—NR^{bb}C(═O)N(R^{bb})₂,—NR^{bb}C(═NR^{bb})N(R^{bb})₂,—NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as described herein, with the proviso that the nitrogen atom directly attached to the parent molecule is not substituted with hydrogen.

As used herein, the term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as described herein.

As used herein, the term “oxo” refers to the group ═O, and the term “thiooxo” refers to the group ═S.

The term “phosphono” refers to the group —(P═O)(OR^{cc})₂, wherein R^{cc} is as defined herein.

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃, ClO₄, OH, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc}) OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O) SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as described herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o nitrobenzamide and o (benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-1-butyl-[9-(10,10-dioxo-10,10,10, 10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-1-BOC), 1,1-dimethyl-2,2,2-trichloroethyl carbamate (TCBOC), 1-methyl-1-(4-biphenylyl)ethyl carbamate (Bpoc), 1-(3,5-di-1-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, 1-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o)-nitrophenyl)methyl carbamate, 1-amyl carbamate, S-benzyl thiocarbamate, p cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido) benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo) benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo) benzyl carbamate, 2,4,6-tri-1-butylphenyl carbamate, 4-(trimethylammonium) benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5, 7,8-pentamethylchroman-6-sulfonamide (Pmc), methanesulfonamide (Ms), β-trimethylsilylethanesulfonamide (SES), 9-anthracenesulfonamide, 4 (4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1, 1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N (1-isopropyl-4-nitro-2-oxo-3-pyroolin-3-yl) amine, quaternary ammonium salts, N-benzylamine, N-di (4-methoxyphenyl)methylamine, N-5-dibenzosuberylamine, N-triphenylmethylamine (Tr), N-[(4-methoxyphenyl) diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N p-methoxybenzylideneamine, N-diphenylmethyleneamine, N-[(2-pyridyl) mesityl]methyleneamine, N—(N′,N′-dimethylaminomethylene) amine, N,N′-isopropylidenediamine, N p-nitrobenzylideneamine, N-salicylideneamine, N 5-chlorosalicylideneamine, N-(5-chloro-2-hydroxyphenyl)phenylmethyleneamine, N-cyclohexylideneamine, N-(5,5-dimethyl-3-oxo-1-cyclohexenyl) amine, N-borane derivative, N-diphenylborinic acid derivative, N-[phenyl (pentaacylchromium- or tungsten)acyl]amine, N-copper chelate, N-zinc chelate, N-nitroamine, N-nitrosoamine, amine N oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O) SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P (OR)₃⁺X⁻, —P(═O) (R^{aa})₂, —P(═O)(OR^{cc})₂, and —P(═O)(N(R^{bb})₂)₂, wherein X⁻, R^{aa}, R^{bb}, and R^{cc} are as described herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), 1-butylthiomethyl, (phenyldimethylsilyl)methoxymethyl (SMOM), benzyloxymethyl (BOM), p-methoxybenzyloxymethyl (PMBM), (4-methoxyphenoxy) methyl (p-AOM), guaiacolmethyl (GUM), 1-butoxymethyl, 4-pentenyloxymethyl (POM), siloxymethyl, 2-methoxyethoxymethyl (MEM), 2,2,2-trichloroethoxymethyl, bis(2-chloroethoxy) methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, 1-butyl, allyl, p-chlorophenyl, p-methoxyphenyl, 2,4-dinitrophenyl, benzyl (Bn), p-methoxybenzyl, 3,4-dimethoxybenzyl, o nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl) diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, 1-butyldimethylsilyl (TBDMS), 1-butyldiphenylsilyl (TBDPS), tribenzylsilyl, tri-p-xylylsilyl, triphenylsilyl, diphenylmethylsilyl (DPMS), 1-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), alkyl isobutyl carbonate, alkyl vinyl carbonate alkyl allyl carbonate, alkyl p nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4 (methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisoproylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb}) R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O) (R^{aa})₂, —P(═O)(OR^{cc})₂, and —P(═O)(N(R^{bb})₂)₂, wherein X⁻, R^{aa}, R^{bb}, and R^{cc} are as described herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

As used herein, a “peptidyl group” refers to a divalent amino acid moiety. A “dipeptidyl” group refers to a divalent moiety comprising two amino acid residues linked together by peptide bonds, i.e., —C(═O)—N—(amide) bonds. A “polypeptidyl” group refers to a divalent moiety comprising three or more consecutively linked amino acid residues (e.g., 3, 4, 5, 6, 7, 8, 9, 10, 11, 12, 13, 14, 15, 16, 17, 18 19, 20, or more linked amino acid residues). Peptidyl, dipeptidyl, and polypeptidyl moieties may contain only natural amino acids, although non-natural amino acids (i.e., compounds that do not occur in nature but that can be incorporated into a polypeptide chain) and/or amino acid analogs as are known in the art may alternatively be employed. Furthermore, one or more of the amino acids in a peptidyl, dipeptidyl, or polypeptidyl moiety may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification. For example, a cysteine (—CH₂SH) side chain may be modified to a formyl (—CHO) side chain.

Exemplary amino acids contemplated useful in providing the peptidyl, dipeptidyl, and polypeptidyl moieties of interest include, without limitation, natural alpha-amino acids such as D- and L-isomers of the 20 common naturally occurring alpha-amino acids found in peptides (e.g., A, R, N, C, D, Q, E, G, H, I, L, K, M, F, P, S, T, W, Y, V), natural beta-amino acids (e.g., beta-alanine), and unnnatural amino acids. There are many known unnatural amino acids any of which may be included in the peptides of the present invention. See for example, S. Hunt, The Non Protein Amino Acids: In Chemistry and Biochemistry of the Amino Acids, edited by G. C. Barrett, Chapman and Hall, 1985. Additional examples of amino acids contemplated useful in providing the peptidyl, dipeptidyl, and polypeptidyl moieties of interest include without limitation, ornithine, citrulline (Cit), α-methyl-Alanine (Aib), 4-hydroxyproline, desmosine, gamma-aminobutyric acid, beta-cyanoalanine, norvaline, 4-(E)-butenyl-4 (R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; CH₃), disubstituted phenylalanines, substituted tyrosines (e.g., further substituted with —C(═O)C₆H₅; —CF₃; —CN; -halo; —NO₂; CH₃), and statine.

The term “carbohydrate” or “saccharide” refers to an aldehydic or ketonic derivative of polyhydric alcohols. Carbohydrates include compounds with relatively small molecules (e.g., sugars) as well as macromolecular or polymeric substances (e.g., starch, glycogen, and cellulose polysaccharides). The term “sugar” refers to monosaccharides, disaccharides, or polysaccharides. Monosaccharides are the simplest carbohydrates in that they cannot be hydrolyzed to smaller carbohydrates. Most monosaccharides can be represented by the general formula C_{y}H_{2y}O_{y} (e.g., C₆H₁₂O₆ (a hexose such as glucose)), wherein y is an integer equal to or greater than 3. Certain polyhydric alcohols not represented by the general formula described above may also be considered monosaccharides. For example, deoxyribose is of the formula C₅H₁₀O₄ and is a monosaccharide. Monosaccharides usually consist of five or six carbon atoms and are referred to as pentoses and hexoses, receptively. If the monosaccharide contains an aldehyde it is referred to as an aldose; and if it contains a ketone, it is referred to as a ketose. Monosaccharides may also consist of three, four, or seven carbon atoms in an aldose or ketose form and are referred to as trioses, tetroses, and heptoses, respectively. Glyceraldehyde and dihydroxyacetone are considered to be aldotriose and ketotriose sugars, respectively. Examples of aldotetrose sugars include erythrose and threose; and ketotetrose sugars include erythrulose. Aldopentose sugars include ribose, arabinose, xylose, and lyxose; and ketopentose sugars include ribulose, arabulose, xylulose, and lyxulose. Examples of aldohexose sugars include glucose (for example, dextrose), mannose, galactose, allose, altrose, talose, gulose, and idose; and ketohexose sugars include fructose, psicose, sorbose, and tagatose. Ketoheptose sugars include sedoheptulose. Each carbon atom of a monosaccharide bearing a hydroxyl group (—OH), with the exception of the first and last carbons, is asymmetric, making the carbon atom a stereocenter with two possible configurations (R or S). Because of this asymmetry, a number of isomers may exist for any given monosaccharide formula. The aldohexose D-glucose, for example, has the formula C₆H₁₂O₆, of which all but two of its six carbons atoms are stereogenic, making D-glucose one of the 16 (i.e., 24) possible stereoisomers. The assignment of D or L is made according to the orientation of the asymmetric carbon furthest from the carbonyl group: in a standard Fischer projection if the hydroxyl group is on the right the molecule is a D sugar, otherwise it is an L sugar. The aldehyde or ketone group of a straight-chain monosaccharide will react reversibly with a hydroxyl group on a different carbon atom to form a hemiacetal or hemiketal, forming a heterocyclic ring with an oxygen bridge between two carbon atoms. Rings with five and six atoms are called furanose and pyranose forms, respectively, and exist in equilibrium with the straight-chain form. During the conversion from the straight-chain form to the cyclic form, the carbon atom containing the carbonyl oxygen, called the anomeric carbon, becomes a stereogenic center with two possible configurations: the oxygen atom may take a position either above or below the plane of the ring. The resulting possible pair of stereoisomers is called anomers. In an a anomer, the —OH substituent on the anomeric carbon rests on the opposite side (trans) of the ring from the —CH₂OH side branch. The alternative form, in which the —CH₂OH substituent and the anomeric hydroxyl are on the same side (cis) of the plane of the ring, is called a β anomer. A carbohydrate including two or more joined monosaccharide units is called a disaccharide or polysaccharide (e.g., a trisaccharide), respectively. The two or more monosaccharide units bound together by a covalent bond known as a glycosidic linkage formed via a dehydration reaction, resulting in the loss of a hydrogen atom from one monosaccharide and a hydroxyl group from another. Exemplary disaccharides include sucrose, lactulose, lactose, maltose, isomaltose, trehalose, cellobiose, xylobiose, laminaribiose, gentiobiose, mannobiose, melibiose, nigerose, or rutinose. Exemplary trisaccharides include, but are not limited to, isomaltotriose, nigerotriose, maltotriose, melezitose, maltotriulose, raffinose, and kestose. The term carbohydrate also includes other natural or synthetic stereoisomers of the carbohydrates described herein.

These and other exemplary substituents are described in more detail in the Detailed Description, Examples, and Claims. The invention is not intended to be limited in any manner by the above exemplary listing of substituents.

### Other Definitions

The term “salt” and “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g. infant, child, adolescent) or adult subject (e.g., young adult, middle-aged adult or senior adult)) and/or other non-human animals, for example mammals (e.g., primates (e.g., cynomolgus monkeys, rhesus monkeys); commercially relevant mammals such as rodents (e.g., mice, rats), guinea pigs, cattle, pigs, horses, sheep, goats, cats, and/or dogs. The non-human animal may be male or female and at any stage of development. A non-human animal may be a transgenic animal.

“Disease,” “disorder,” and “condition” are used interchangeably herein.

As used herein, and unless otherwise specified, the terms “treat,” “treating” and “treatment” contemplate an action that occurs while a subject is suffering from the specified disease, disorder, or condition, which reduces the severity of the disease, disorder, or condition, or retards or slows the progression of the disease, disorder, or condition (“therapeutic treatment” or “therapeutically treating”), and also contemplates an action that occurs before a subject begins to suffer from the specified disease, disorder, or condition, and which inhibits or reduces the severity of the disease, disorder, or condition (“prophylactic treatment” or “prophylactically treating”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. For example, the effective amount of a compound with anti-proliferative activity is the amount that results in a sufficient concentration to inhibit the proliferation of cells. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder, or condition, or to delay or minimize one or more symptoms associated with the disease, disorder, or condition. A therapeutically effective amount of a compound means an amount of therapeutic agent, alone or in combination with other therapies, which provides a therapeutic benefit in the treatment of the disease, disorder, or condition. The term “therapeutically effective amount” can encompass an amount that improves overall therapy, reduces or avoids symptoms or causes of the disease, disorder, or condition, or enhances the therapeutic efficacy of another therapeutic agent.

As used herein, and unless otherwise specified, a “prophylactically effective amount” of a compound is an amount sufficient to prevent a disease, disorder, or condition, or one or more symptoms associated with the disease, disorder, or condition, or prevent its recurrence. A prophylactically effective amount of a compound means an amount of a therapeutic agent, alone or in combination with other agents, which provides a prophylactic benefit in the prevention of the disease, disorder, or condition. The term “prophylactically effective amount” can encompass an amount that improves overall prophylaxis or enhances the prophylactic efficacy of another prophylactic agent.

As used herein, use of the phrase “at least one instance” refers to 1, 2, 3, 4, or more instances, but also encompasses a range, for example, from 1 to 4, from 1 to 3, from 1 to 2, from 2 to 4, from 2 to 3, or from 3 to 4 instances, inclusive.

As used herein, “small molecule” refers to molecules, whether naturally occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins of the disclosure preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

Exemplary amino acids contemplated useful in providing the proteins of interest include, without limitation, natural alpha-amino acids such as D- and L-isomers of the 20 common naturally occurring alpha-amino acids found in peptides (e.g., A, R, N, C, D, Q, E, G, H, I, L, K, M, F, P, S, T, W, Y, V), natural beta-amino acids (e.g., beta-alanine), and unnnatural amino acids. There are many known unnatural amino acids any of which may be included in the proteins of the present disclosure. See for example, S. Hunt, The Non-Protein Amino Acids: In Chemistry and Biochemistry of the Amino Acids, edited by G. C. Barrett, Chapman and Hall, 1985. Additional examples of amino acids contemplated useful in providing the proteins of interest include without limitation, ornithine, citrulline (Cit), α-methyl-Alanine (Aib), 4-hydroxyproline, desmosine, gamma-aminobutyric acid, beta-cyanoalanine, norvaline, 4-(E)-butenyl-4 (R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; CN; -halo; —NO₂; CH₃), disubstituted phenylalanines, substituted tyrosines (e.g., further substituted with —C(═O) C₆H₅; —CF₃; —CN; -halo; —NO₂; CH₃), and statine.

The term “targeting moiety” or “targeting agent” refers to a member of a specific binding pair, i.e., a member of a pair of molecules, wherein one of the pair of molecules has an area on its surface, or a cavity that specifically binds to, and is, therefore, defined as complementary with a particular spatial and polar organization of the other molecule, so that the pair have the property of binding specifically to each other. Examples of types of specific binding pairs are antigen-antibody, biotin-avidin, hormone-hormone receptor, receptor-ligand, enzyme-substrate, and IgG-protein A. In one example, a targeting moiety is an antibody. In another example, a targeting moiety is an antibody fragment.

### Detailed Description of Certain Embodiments of the Invention

Trioxacarcins are highly toxic to a variety of cell types. Linking a trioxacarcin to an antibody preserves the trioxacarcin's potency against the cell type while increasing specificity for the target cell, and optionally increasing endocytosis of the trioxacarcin. These effects enable lowering the overall amount of trioxacarcin to be delivered, thereby reducing the associated toxicity. By taking advantage of established synthetic methods, complex and therapeutically relevant trioxacarcins are accessible. In turn, conjugating these trioxacarcins to antibodies through linking groups provide trioxacarcin-antibody drug conjugates.

Accordingly, provided herein are novel trioxacarcin analogs, antibody drug conjugates, and antibody drug conjugate precursor compounds, including trioxacarcin-antibody drug conjugate precursors comprising a trioxacarcin and a linking group, and novel trioxacarcin analogs (e.g., compounds of Formula (I) and (II)). The compounds may be provided for use in any composition, kit, or method described herein as a pharmaceutically acceptable salt.

Provided are compounds of Formula (I):

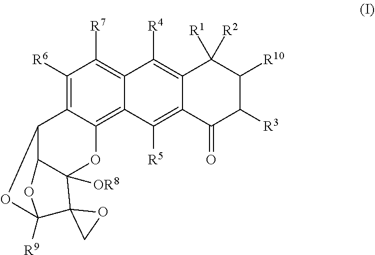

or a pharmaceutically acceptable salt, solvate, hydrate, polymorph, co-crystal, tautomer, stereoisomer, isotopically labeled derivative, or prodrug thereof;
wherein:
R¹ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{A1}; —C(═O)R^{A2}; —CO₂R^{A2}; —CN; —SCN; —SR^{A1}; —SOR^{A1}; —SO₂R^{A1}; —NO₂; —N₃; —N(R^{A2})₂; —NR^{A2}C(═O)R^{A2}; —NR^{A2}C(═O)N(R^{A2})₂; —OC(═O)OR^{A1}; —OC(═O)R^{A2}; —OC(═O)N(R^{A2})₂; —NRAC(═O)OR^{A1}; or —C(R^{A2})₃; wherein each occurrence of R^{A1} is independently hydrogen; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{A1} groups are optionally joined to form a substituted or unsubstituted heterocyclyl or substituted or unsubstituted heteroaryl ring; and wherein each occurrence of R^{A2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{A1}; —SR^{A1}; or —N(R^{A1})₂, or two R^{A2} groups are optionally joined to form a substituted or unsubstituted heterocyclyl or substituted or unsubstituted heteroaryl ring; with the proviso that if R¹ is —OR^{A1}, R^{A1} is not 4-methoxybenzyl or carbohydrate.
R² is hydrogen; or R¹ and R² are joined to form ═O; ═N(R^{A2}); or ═S;
R³ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{C1}. —C(═O)R^{C2}; —CO₂R^{C1}; —CN; —SCN; —SR^{C1}; —SOR^{C1}; —SO₂R^{C2}; —NO₂; —N₃; —N(R^{C2})₂; —NHC(═O)R^{C2}; —NR^{C2}C(═O)N(R^{C2})₂; —OC(═O)OR^{C1}; —OC(═O)R^{C2}; —OC(═O)N(R^{C2})₂; —NR^{C2}C(═O)OR^{C1}; or —C(R^{C2})₃; wherein each occurrence of R^{C1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{C1} groups are optionally joined to form a substituted or unsubstituted heterocyclyl or substituted or unsubstituted heteroaryl ring; and wherein each occurrence of R^{C2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{C1}; —SR^{C1}; or —N(R^{C1})₂; or two R^{C2} groups are optionally joined to form a substituted or unsubstituted heterocyclyl or substituted or unsubstituted heteroaryl ring;
R⁴ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{D1}; —C(═O)R^{D2}; —CO₂R^{D2}; —CN; —SCN; —SR^{D1}; —SOR^{D1}; —SO₂R^{D2}; —NO₂; —N₃; —N(R^{D2})₂; —NR^{D2}C(═O)R^{D2}; —NR^{D2}C(═O)N(R^{D2})₂; —OC(═O)OR^{D1}; —OC(═O)R^{D2}; —OC(═O)N(R^{D2})₂; —NR^{D2}C(═O)OR^{D1}; or —C(R^{D2})₃; wherein each occurrence of R^{D1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{D1} groups are optionally joined to form a substituted or unsubstituted heterocyclyl or substituted or unsubstituted heteroaryl ring; and wherein each occurrence of R^{D2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{D1}; —SR^{D1}; or —N(R^{D1})₂; or two R^{D2} groups are optionally joined to form a substituted or unsubstituted heterocyclyl or substituted or unsubstituted heteroaryl ring;
R⁵ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{E1};
—C(═O)R^{E2}; —CO₂R^{E1}; —CN; —SCN; —SR^{E1}; —SOR^{E1}; —SO₂R^{E2}; —NO₂; —N₃; —N(R^{E2})₂; —NR^{E2}C(═O)R^{E2}; —NR^{E2}C(═O)N(R^{E2})₂; —OC(═O)OR^{E1}; —OC(═O)R^{E2}; —OC(═O)N(R^{E2})₂; —NR^{E2}C(═O)OR^{E1}; or —C(R^{E2})₃; wherein each occurrence of R^{E1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{E1} groups are optionally joined to form a substituted or unsubstituted heterocyclyl or substituted or unsubstituted heteroaryl ring; and wherein each occurrence of R^{E2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{E1}; —SR^{E1}; or —N(R^{E1})₂; or two R^{E2} groups are optionally joined to form a substituted or unsubstituted heterocyclyl or substituted or unsubstituted heteroaryl ring;
R⁶ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{F1}; —C(═O)R^{F2}; —CO₂R^{F1}; —CN; —SCN; —SR^{F1}; —SOR^{F1}; —SO₂R^{F2}; —NO₂; —N₃; —N(R^{F2})₂; —NR^{F2}C(═O)R^{F2}; —NR^{F2}C(═O)N(R^{F2})₂; —OC(═O)OR^{F1}; —OC(═O)R^{F2}; —OC(═O)N(R^{F2})₂; —NR^{F2}C(═O)OR^{F1}; or —C(R^{F2})₃; wherein each occurrence of R^{F1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{F1} groups are optionally joined to form a substituted or unsubstituted heterocyclyl or substituted or unsubstituted heteroaryl ring; and wherein each occurrence of R^{F2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{F1}; —SR^{F1}; or —N(R^{F1})₂; or two R^{F2} groups are optionally joined to form a substituted or unsubstituted heterocyclyl or substituted or unsubstituted heteroaryl ring;
R⁷ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{G1}; —C(═O)R^{G2}; —CO₂R^{G1}; —CN; —SCN; —SR^{G1}; —SOR^{G1}; —SO₂R^{G2}; —NO₂; —N₃; —N(RG)₂; —NR^{G2}C(═O)R^{G2}; —NR^{G2}C(═O)N(R^{G2})₂; —OC(═O)OR^{G1}; —OC(═O)R^{G2}; —OC(═O)N(R^{G2})₂; —NR^{G2}C(═O)OR^{G1}; or —C(R^{G2})₃; wherein each occurrence of R^{G1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{G1} groups are optionally joined to form a substituted or unsubstituted heterocyclyl or substituted or unsubstituted heteroaryl ring; and wherein each occurrence of R^{G2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{G1}; —SR^{G1}; or —N(R^{G1})₂; or two R^{G2} groups are optionally joined to form a substituted or unsubstituted heterocyclyl or substituted or unsubstituted heteroaryl ring;
R⁸ is hydrogen; an oxygen protecting group; a carbohydrate; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; substituted or unsubstituted heteroaliphatic; or acyl; —OR^{H9}; —OC(═O)R^{H9}; —N(R^{H9})₂; or —NHC(═O)R^{H9}; wherein each occurrence of R^{H9} is independently hydrogen; substituted or unsubstituted alkyl; an oxygen protecting group when attached to an oxygen atom; or a nitrogen protecting group when attached to a nitrogen atom; or two R^{H9} groups are joined to form a substituted or unsubstituted heterocyclic or substituted or unsubstituted heteroaryl ring;
R⁹ is hydrogen or —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring;
R¹⁰ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{B1}; —C(═O)R^{B2}; —CO₂R^{B2}; —CN; —SCN; —SR^{B1}; —SOR^{B1}; —SO₂R^{B2}; —NO₂; —N₃; —N(R^{B2})₂; —NR^{B2}C(═O)R^{B2}; —NR^{B2}C(═O)N(R^{B2})₂; —OC(═O)OR^{B1}; —OC(═O)R^{B2}; —OC(═O)N(R^{B2})₂; —NR^{B2}C(═O)OR^{B1}; or —C(R^{B2})₃; wherein each occurrence of R^{B1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{B1} groups are optionally joined to form a substituted or unsubstituted heterocyclyl or substituted or unsubstituted heteroaryl ring; and wherein each occurrence of R^{B2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{B1}; —SR^{B1}; or —N(R^{B1})₂; or two R^{B2} groups are optionally joined to form a substituted or unsubstituted heterocyclyl or substituted or unsubstituted heteroaryl ring;
or R¹ and R¹⁰ are optionally taken together with the intervening carbon atoms to form an optionally substituted cyclic moiety;
or R¹⁰ and R³ are optionally taken together with the intervening carbon atoms to form an optionally substituted cyclic moiety;
or R¹ and R⁴ are optionally taken together with the intervening carbon atoms to form an optionally substituted cyclic moiety;
or R⁴ and R⁷ are optionally taken together with the intervening carbon atoms to form an optionally substituted cyclic moiety;
or R⁶ and R⁷ are optionally taken together with the intervening carbon atoms to form an optionally substituted cyclic moiety;
L is a linker;
A is a bond or a group of formula:

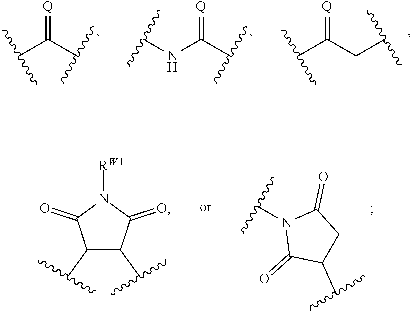

Q is —S— or —O—;
R^{W1} is independently hydrogen, substituted or unsubstituted alkyl; or a nitrogen protecting group;
B is a targeting moiety; and
T is hydrogen, —N═C═S,

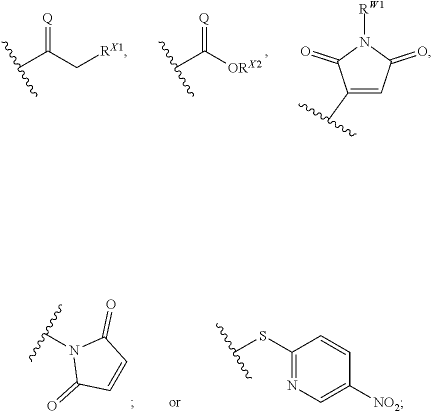

R^{X1} is a leaving group; and
R^{X2} is hydrogen; substituted or unsubstituted alkyl; substituted or unsubstituted heterocyclyl; substituted or unsubstituted aryl; substituted or unsubstituted heteroaryl; or an oxygen protecting group.

In certain embodiments of Formula (I):
R¹ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{A1}; —C(═O)R^{A2}; —CO₂R^{A2}; —CN; —SCN; —SR^{A1}; —SOR^{A1}; —SO₂R^{A1}; —NO₂; —N₃; —N(R^{A2})₂; —NR^{A2}C(═O)R^{A2}; —NR^{A2}C(═O)N(R^{A2})₂; —OC(═O)OR^{A1}; —OC(═O)R^{A2}; —OC(═O)N(R^{A2})₂; —NRAC(═O)OR^{A1}; or —C(R^{A2})₃; wherein each occurrence of R^{A1} is independently hydrogen; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{A1} groups are optionally joined to form a substituted or unsubstituted heterocyclyl or substituted or unsubstituted heteroaryl ring; and wherein each occurrence of R^{A2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{A1}; —SR^{A1}; or —N(R^{A1})₂, or two RAZ groups are optionally joined to form a substituted or unsubstituted heterocyclyl or substituted or unsubstituted heteroaryl ring; with the proviso that if R¹ is —OR^{A1}, R^{A1} is not 4-methoxybenzyl or carbohydrate.
R² is hydrogen; or R¹ and R² are joined to form ═O; ═N(R^{A2}); or ═S;
R³ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{C1}; —C(═O)R^{C2}; —CO₂R^{C1}; —CN; —SCN; —SR^{C1}; —SOR^{C1}; —SO₂R^{C2}; —NO₂; —N₃; —N(R^{C2})₂; —NHC(═O)R^{C2}; —NR^{C2}C(═O)N(R^{C2})₂; —OC(═O)OR^{C1}; —OC(═O)R^{C2}; —OC(═O)N(R^{C2})₂; —NR^{C2}C(═O)OR^{C1}; or —C(R^{C2})₃; wherein each occurrence of R^{C1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{C1} groups are optionally joined to form a substituted or unsubstituted heterocyclyl or substituted or unsubstituted heteroaryl ring; and wherein each occurrence of R^{C2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{C1}; —SR^{C1}; or —N(R^{C1})₂; or two R^{C2} groups are optionally joined to form a substituted or unsubstituted heterocyclyl or substituted or unsubstituted heteroaryl ring;
R⁴ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{D1}; —C(═O)R^{D2}; —CO₂R^{D2}; —CN; —SCN; —SR^{D1}; —SOR^{D1}; —SO₂R^{D2}; —NO₂; —N₃; —N(R^{D2})₂; —NR^{D2}C(═O)R^{D2}; —NR^{D2}C(═O)N(R^{D2})₂; —OC(═O)OR^{D1}; —OC(═O)R^{D2}; —OC(═O)N(R^{D2})₂; —NR^{D2}C(═O)OR^{D1}; or —C(R^{D2})₃; wherein each occurrence of R^{D1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{D1} groups are optionally joined to form a substituted or unsubstituted heterocyclyl or substituted or unsubstituted heteroaryl ring; and wherein each occurrence of R^{D2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{D1}; —SR^{D1}; or —N(R^{D1})₂; or two R^{D2} groups are optionally joined to form a substituted or unsubstituted heterocyclyl or substituted or unsubstituted heteroaryl ring;
R⁵ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{E1}; —C(═O)R^{E2}; —CO₂R^{E1}; —CN; —SCN; —SR^{E1}; —SOREL; —SO₂R^{E2}; —NO₂; —N₃; —N(R^{E2})₂; —NR^{E2}C(═O)R^{E2}; —NR^{E2}C(═O)N(R^{E2})₂; —OC(═O)OR^{E1}; —OC(═O)R^{E2}; —OC(═O)N(R^{E2})₂; —NR^{E2}C(═O)OR^{E1}; or —C(R^{E2})₃; wherein each occurrence of R^{E1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{E1} groups are optionally joined to form a substituted or unsubstituted heterocyclyl or substituted or unsubstituted heteroaryl ring; and wherein each occurrence of R^{E2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{E1}; —SR^{E1}; or —N(R^{E1})₂; or two R^{E2} groups are optionally joined to form a substituted or unsubstituted heterocyclyl or substituted or unsubstituted heteroaryl ring;
R⁶ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{F1}; —C(═O)R^{F2}; —CO₂R^{F1}; —CN; —SCN; —SR^{F1}; —SOR^{F1}; —SO₂R^{F2}; —NO₂; —N₃; —N(R^{F2})₂; —NR^{F2}C(═O)R^{F2}; —NR^{F2}C(═O)N(R^{F2})₂; —OC(═O)OR^{F1}; —OC(═O)R^{F2}; —OC(═O)N(R^{F2})₂; —NR^{F2}C(═O)OR^{F1}; or —C(R^{F2})₃; wherein each occurrence of R^{F1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{F1} groups are optionally joined to form a substituted or unsubstituted heterocyclyl or substituted or unsubstituted heteroaryl ring; and wherein each occurrence of R^{F2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{F1}; —SR^{F1}; or —N(R^{F1})₂; or two R^{F2} groups are optionally joined to form a substituted or unsubstituted heterocyclyl or substituted or unsubstituted heteroaryl ring;
R⁷ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{G1}; —C(═O)R^{G2}; —CO₂R^{G1}; —CN; —SCN; —SR^{G1}; —SOR^{G1}; —SO₂R^{G2}; —NO₂; —N₃; —N(RG)₂; —NR^{G2}C(═O)R^{G2}; —NR^{G2}C(═O)N(R^{G2})₂; —OC(═O)OR^{G1}; —OC(═O)R^{G2}; —OC(═O)N(R^{G2})₂; —NR^{G2}C(═O)OR^{G1}; or —C(R^{G2})₃; wherein each occurrence of R^{G1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{G1} groups are optionally joined to form a substituted or unsubstituted heterocyclyl or substituted or unsubstituted heteroaryl ring; and wherein each occurrence of R^{G2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{G1}; —SR^{G1}; or —N(R^{G1})₂; or two R^{G2} groups are optionally joined to form a substituted or unsubstituted heterocyclyl or substituted or unsubstituted heteroaryl ring;
R⁸ is hydrogen; an oxygen protecting group; a carbohydrate; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; substituted or unsubstituted heteroaliphatic; or acyl; —OR^{H9}; —OC(═O)R^{H9}; —N(R^{H9})₂; or —NHC(═O)R^{H9}; wherein each occurrence of R^{H9} is independently hydrogen; substituted or unsubstituted alkyl; an oxygen protecting group when attached to an oxygen atom; or a nitrogen protecting group when attached to a nitrogen atom; or two R^{H9} groups are joined to form a substituted or unsubstituted heterocyclic or substituted or unsubstituted heteroaryl ring;
R⁹ is —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂; with the proviso that not more than one occurrence of R^{I1} is —OR^{I2}, wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring;
R¹⁰ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{B1}; —C(═O)R^{B2}; —CO₂R^{B2}; —CN; —SCN; —SR^{B1}; —SOR^{B1}; —SO₂R^{B2}; —NO₂; —N₃; —N(R^{B2})₂; —NR^{B2}C(═O)R^{B2}; —NR^{B2}C(═O)N(R^{B2})₂; —OC(═O)OR^{B1}; —OC(═O)R^{B2}; —OC(═O)N(R^{B2})₂; —NR^{B2}C(═O)OR^{B1}; or —C(R^{B2})₃; wherein each occurrence of R^{B1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{B1} groups are optionally joined to form a substituted or unsubstituted heterocyclyl or substituted or unsubstituted heteroaryl ring; and wherein each occurrence of R^{B2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{B1}; —SR^{B1}; or —N(R^{B1})₂; or two R^{B2} groups are optionally joined to form a substituted or unsubstituted heterocyclyl or substituted or unsubstituted heteroaryl ring;
or R¹ and R¹⁰ are optionally taken together with the intervening carbon atoms to form an optionally substituted cyclic moiety;
or R¹⁰ and R³ are optionally taken together with the intervening carbon atoms to form an optionally substituted cyclic moiety;
or R¹ and R⁴ are optionally taken together with the intervening carbon atoms to form an optionally substituted cyclic moiety;
or R⁴ and R⁷ are optionally taken together with the intervening carbon atoms to form an optionally substituted cyclic moiety;
or R⁶ and R⁷ are optionally taken together with the intervening carbon atoms to form an optionally substituted cyclic moiety;
L is a linker;
A is a bond or a group of formula:

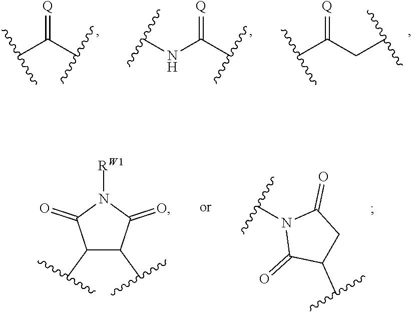

Q is —S— or —O—;
R^{W1} is independently hydrogen, substituted or unsubstituted alkyl; or a nitrogen protecting group;
B is a targeting moiety; and
T is hydrogen, —N═C═S,

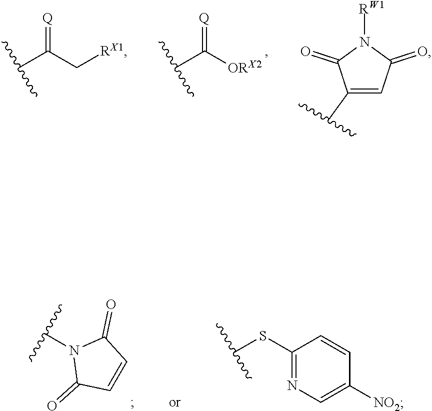

R^{X1} is a leaving group; and
R^{X2} is hydrogen; substituted or unsubstituted alkyl; substituted or unsubstituted heterocyclyl; substituted or unsubstituted aryl; substituted or unsubstituted heteroaryl; or an oxygen protecting group.

In certain embodiments, the compound of Formula (I) is not

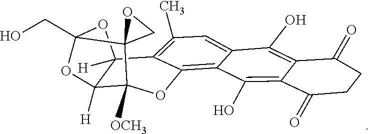

In certain embodiments, the compound of Formula (I) is of Formula (I-a):

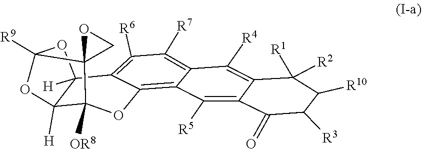

or a pharmaceutically acceptable salt thereof.

In certain embodiments, the compound of Formula (I) is of Formula (I-b):

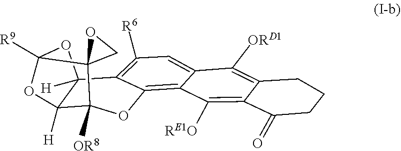

or a pharmaceutically acceptable salt thereof.

In certain embodiments, the compound of Formula (I) is of Formula (I-c):

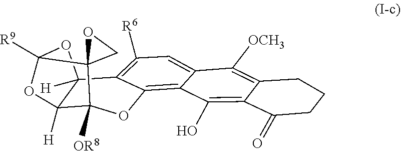

or a pharmaceutically acceptable salt thereof.

In certain embodiments, the compound of Formula (I) is of Formula (I-d):

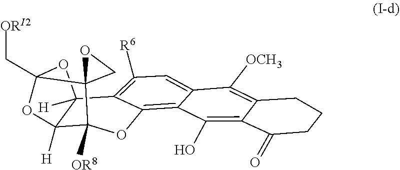

or a pharmaceutically acceptable salt thereof.

In certain embodiments, the compound of Formula (I-d) is of Formula (I-d-i):

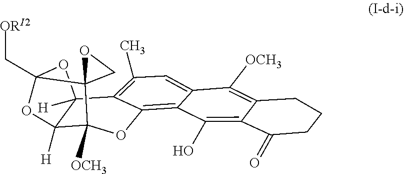

or a pharmaceutically acceptable salt thereof.

In certain embodiments, the compound of Formula (I) is of Formula (I-e):

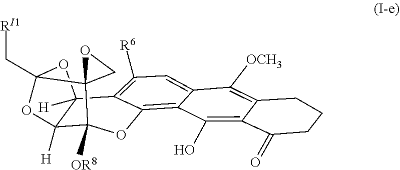

or a pharmaceutically acceptable salt thereof.

In certain embodiments, the compound of Formula (I-e) is of Formula (I-e-i):

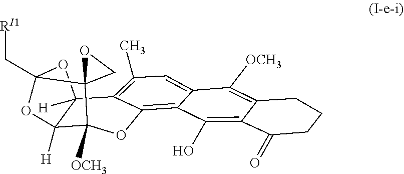

or a pharmaceutically acceptable salt thereof.

Also provided are compounds of Formula (II):

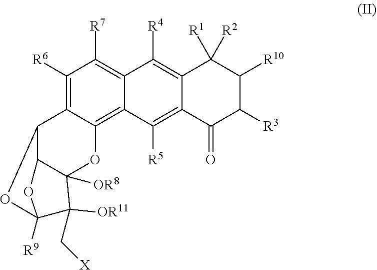

or a pharmaceutically acceptable salt, solvate, hydrate, polymorph, co-crystal, tautomer, stereoisomer, isotopically labeled derivative, or prodrug thereof;
wherein:
R¹ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{A1}; —C(═O)R^{A2}; —CO₂R^{A2}; —CN; —SCN; —SR^{A1}; —SOR^{A1}; —SO₂R^{A1}; —NO₂; —N₃; —N(R^{A2})₂; —NR^{A2}C(═O)R^{A2}; —NRAZC(═O)N(R^{A2})₂; —OC(═O)OR^{A1}; —OC(═O)R^{A2}; —OC(═O)N(R^{A2})₂; —NRAC(═O)OR^{A1}; or —C(R^{A2})₃; wherein each occurrence of R^{A1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{A1} groups are optionally joined to form a substituted or unsubstituted heterocyclyl or substituted or unsubstituted heteroaryl ring; and wherein each occurrence of R^{A2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{A1}; —SR^{A1}; or —N(R^{A1})₂; or two R^{A2} groups are optionally joined to form a substituted or unsubstituted heterocyclyl or substituted or unsubstituted heteroaryl ring; with the proviso that if R¹ is —OR^{A1}, R^{A1} is not 4-methoxybenzyl or carbohydrate.
R² is hydrogen; or R¹ and R² are joined to form ═O; ═N(R^{A2}); or ═S;
R³ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{C1}; —C(═O)R^{C2}; —CO₂R^{C1}; —CN; —SCN; —SR^{C1}; —SOR^{C1}; —SO₂R^{C2}; —NO₂; —N₃; —N(R^{C2})₂; —NHC(═O)R^{C2}; —NR^{C2}C(═O)N(R^{C2})₂; —OC(═O)OR^{C1}; —OC(═O)R^{C2}; —OC(═O)N(R^{C2})₂; —NR^{C2}C(═O)OR^{C1}; or —C(R^{C2})₃; wherein each occurrence of R^{C1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{C1} groups are optionally joined to form a substituted or unsubstituted heterocyclyl or substituted or unsubstituted heteroaryl ring; and wherein each occurrence of R^{C2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{C1}; —SR^{C1}; or —N(R^{C1})₂; or two R^{C2} groups are optionally joined to form a substituted or unsubstituted heterocyclyl or substituted or unsubstituted heteroaryl ring;
R⁴ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{D1}; —C(═O)R^{D2}; —CO₂R^{D2}; —CN; —SCN; —SR^{D1}; —SOR^{D1}; —SO₂R^{D2}; —NO₂; —N₃; —N(R^{D2})₂; —NR^{D2}C(═O)R^{D2}; —NR^{D2}C(═O)N(R^{D2})₂; —OC(═O)OR^{D1}; —OC(═O)R^{D2}; —OC(═O)N(R^{D2})₂; —NR^{D2}C(═O)OR^{D1}; or —C(R^{D2})₃; wherein each occurrence of R^{D1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{D1} groups are optionally joined to form a substituted or unsubstituted heterocyclyl or substituted or unsubstituted heteroaryl ring; and wherein each occurrence of R^{D2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{D1}; —SR^{D1}; or —N(R^{D1})₂; or two R^{D2} groups are optionally joined to form a substituted or unsubstituted heterocyclyl or substituted or unsubstituted heteroaryl ring;
R⁵ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{E1}; —C(═O)R^{E2}; —CO₂R^{E1}; —CN; —SCN; —SR^{E1}; —SOR^{E1}; —SO₂R^{E2}; —NO₂; —N₃; —N(R^{E2})₂; —NR^{E2}C(═O)R^{E2}; —NR^{E2}C(═O)N(R^{E2})₂; —OC(═O)OR^{E1}; —OC(═O)R^{E2}; —OC(═O)N(R^{E2})₂; —NR^{E2}C(═O)OR^{E1}; or —C(R^{E2})₃; wherein each occurrence of R^{E1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{E1} groups are optionally joined to form a substituted or unsubstituted heterocyclyl or substituted or unsubstituted heteroaryl ring; and wherein each occurrence of R^{E2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{E1}; —SR^{E1}; or —N(R^{E1})₂; or two R^{E2} groups are optionally joined to form a substituted or unsubstituted heterocyclyl or substituted or unsubstituted heteroaryl ring;
R⁶ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{F1}; —C(═O)R^{F2}; —CO₂R^{F1}; —CN; —SCN; —SR^{F1}; —SOR^{F1}; —SO₂R^{F2}; —NO₂; —N₃; —N(R^{F2})₂; —NR^{F2}C(═O)R^{F2}; —NR^{F2}C(═O)N(R^{F2})₂; —OC(═O)OR^{F1}; —OC(═O)R^{F2}; —OC(═O)N(R^{F2})₂; —NR^{F2}C(═O)OR^{F1}; or —C(R^{F2})₃; wherein each occurrence of R^{F1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{F1} groups are optionally joined to form a substituted or unsubstituted heterocyclyl or substituted or unsubstituted heteroaryl ring; and wherein each occurrence of R^{F2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{F1}; —SR^{F1}; or —N(R^{F1})₂; or two R^{F2} groups are optionally joined to form a substituted or unsubstituted heterocyclyl or substituted or unsubstituted heteroaryl ring;
R⁷ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{G1}; —C(═O)R^{G2}; —CO₂R^{G1}; —CN; —SCN; —SR^{G1}; —SOR^{G1}; —SO₂R^{G2}; —NO₂; —N₃; —N(R^{G})₂; —NR^{G2}C(═O)R^{G2}; —NR^{G2}C(═O)N(R^{G2})₂; —OC(═O)OR^{G1}; —OC(═O)R^{G2}; —OC(═O)N(R^{G2})₂; —NR^{G2}C(═O)OR^{G1}; or —C(R^{G2})₃; wherein each occurrence of R^{G1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{G1} groups are optionally joined to form a substituted or unsubstituted heterocyclyl or substituted or unsubstituted heteroaryl ring; and wherein each occurrence of R^{G2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{G1}; —SR^{G1}; or —N(R^{G1})₂; or two R^{G2} groups are optionally joined to form a substituted or unsubstituted heterocyclyl or substituted or unsubstituted heteroaryl ring;
R⁸ is hydrogen; an oxygen protecting group; a carbohydrate; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; substituted or unsubstituted heteroaliphatic; acyl; —OR^{H9}; —OC(═O)R^{H9}; —N(R^{H9})₂; or —NHC(═O)R^{H9}; wherein each occurrence of R^{H9} is independently hydrogen; substituted or unsubstituted alkyl; an oxygen protecting group when attached to an oxygen atom; or a nitrogen protecting group when attached to a nitrogen atom; or two R^{H9} groups are joined to form a substituted or unsubstituted heterocyclic or substituted or unsubstituted heteroaryl ring;
R⁹ is hydrogen or —C(R^{I1})₃; wherein each occurrence of R^{I1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{I2}; —SR^{I2}; —N(R^{I2})₂; azido; or halogen; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring;
R¹⁰ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{B1}; —C(═O)R^{B2}; —CO₂R^{B2}; —CN; —SCN; —SR^{B1}; —SOR^{B1}; —SO₂R^{B2}; —NO₂; —N₃; —N(R^{B2})₂; —NR^{B2}C(═O)R^{B2}; —NR^{B2}C(═O)N(R^{B2})₂; —OC(═O)OR^{B1}; —OC(═O)R^{B2}; —OC(═O)N(R^{B2})₂; —NR^{B2}C(═O)OR^{B1}; or —C(R^{B2})₃; wherein each occurrence of R^{B1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{B1} groups are optionally joined to form a substituted or unsubstituted heterocyclyl or substituted or unsubstituted heteroaryl ring; and wherein each occurrence of R^{B2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{B1}; —SR^{B1}; or —N(R^{B1})₂; or two R^{B2} groups are optionally joined to form a substituted or unsubstituted heterocyclyl or substituted or unsubstituted heteroaryl ring;
R^{I1} is hydrogen; substituted or unsubstituted alkyl; acyl; -L-A-B; or -L-T;
X is a halogen;
or R¹ and R¹⁰ are optionally taken together with the intervening carbon atoms to form an optionally substituted cyclic moiety;
or R¹⁰ and R³ are optionally taken together with the intervening carbon atoms to form an optionally substituted cyclic moiety;
or R¹ and R⁴ are optionally taken together with the intervening carbon atoms to form an optionally substituted cyclic moiety;
or R⁴ and R⁷ are optionally taken together with the intervening carbon atoms to form an optionally substituted cyclic moiety;
or R⁶ and R⁷ are optionally taken together with the intervening carbon atoms to form an optionally substituted cyclic moiety;
L is a linker;
A is a bond or a group of formula:

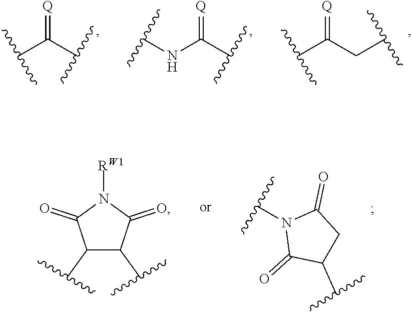

Q is —S— or —O—;
R^{W1} is independently hydrogen, substituted or unsubstituted alkyl; or a nitrogen protecting group;
B is a targeting moiety; and
T is hydrogen, —N═C═S,

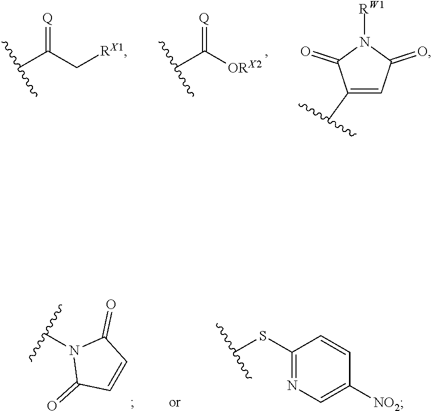

R^{X1} is a leaving group; and
R^{X2} is hydrogen; substituted or unsubstituted alkyl; substituted or unsubstituted heterocyclyl; substituted or unsubstituted aryl; substituted or unsubstituted heteroaryl; or an oxygen protecting group.

In certain embodiments of Formula (II):
R¹ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{A1}; —C(═O)R^{A2}; —CO₂R^{A2}; —CN; —SCN; —SR^{A1}; —SOR^{A1}; —SO₂R^{A1}; —NO₂; —N₃; —N(R^{A2})₂; —NRAZC(═O)R^{A2}; —NR^{A2}C(═O)N(R^{A2})₂; —OC(═O)OR^{A1}; —OC(═O)R^{A2}; —OC(═O)N(R^{A2})₂; —NRAC(═O)OR^{A1}; or —C(R^{A2})₃; wherein each occurrence of R^{A1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{A1} groups are optionally joined to form a substituted or unsubstituted heterocyclyl or substituted or unsubstituted heteroaryl ring; and wherein each occurrence of R^{A2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{A1}; —SR^{A1}; or —N(R^{A1})₂; or two R^{A2} groups are optionally joined to form a substituted or unsubstituted heterocyclyl or substituted or unsubstituted heteroaryl ring; with the proviso that if R¹ is —OR^{A1}, R^{A1} is not 4-methoxybenzyl or carbohydrate.
R² is hydrogen; or R¹ and R² are joined to form ═O; ═N(R^{A2}); or ═S;
R³ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{C1}; —C(═O)R^{C2}; —CO₂R^{C1}; —CN; —SCN; —SR^{C1}; —SOR^{C1}; —SO₂R^{C2}; —NO₂; —N₃; —N(R^{C2})₂; —NHC(═O)R^{C2}; —NR^{C2}C(═O)N(R^{C2})₂; —OC(═O)OR^{C1}; —OC(═O)R^{C2}; —OC(═O)N(R^{C2})₂; —NR^{C2}C(═O)OR^{C1}; or —C(R^{C2})₃; wherein each occurrence of RCl is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{C1} groups are optionally joined to form a substituted or unsubstituted heterocyclyl or substituted or unsubstituted heteroaryl ring; and wherein each occurrence of R^{C2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{C1}; —SR^{C1}; or —N(R^{C1})₂; or two R^{C2} groups are optionally joined to form a substituted or unsubstituted heterocyclyl or substituted or unsubstituted heteroaryl ring;
R⁴ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{D1}; —C(═O)R^{D2}; —CO₂R^{D2}; —CN; —SCN; —SR^{D1}; —SOR^{D1}; —SO₂R^{D2}; —NO₂; —N₃; —N(R^{D2})₂; —NR^{D2}C(═O)R^{D2}; —NR^{D2}C(═O)N(R^{D2})₂; —OC(═O)OR^{D1}; —OC(═O)R^{D2}; —OC(═O)N(R^{D2})₂; —NR^{D2}C(═O)OR^{D1}; or —C(R^{D2})₃; wherein each occurrence of R^{D1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{D1} groups are optionally joined to form a substituted or unsubstituted heterocyclyl or substituted or unsubstituted heteroaryl ring; and wherein each occurrence of R^{D2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{D1}; —SR^{D1}; or —N(R^{D1})₂; or two R^{D2} groups are optionally joined to form a substituted or unsubstituted heterocyclyl or substituted or unsubstituted heteroaryl ring;
R⁵ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{E1}; —C(═O)R^{E2}; —CO₂R^{E1}; —CN; —SCN; —SR^{E1}; —SOR^{E1}; —SO₂R^{E2}; —NO₂; —N₃; —N(R^{E2})₂; —NR^{F2}C(═O)R^{E2}; —NR=2C(═O)N(R^{E2})₂; —OC(═O)OR^{E1}; —OC(═O)R^{E2}; —OC(═O)N(R^{E2})₂; —NR^{F2}C(═O)OR^{E1}; or —C(R^{E2})₃; wherein each occurrence of R^{E1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{E1} groups are optionally joined to form a substituted or unsubstituted heterocyclyl or substituted or unsubstituted heteroaryl ring; and wherein each occurrence of R^{E2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{E1}; —SR^{E1}; or —N(R^{E1})₂; or two R^{E2} groups are optionally joined to form a substituted or unsubstituted heterocyclyl or substituted or unsubstituted heteroaryl ring;
R⁶ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{F1}; —C(═O)R^{F2}; —CO₂R^{F1}; —CN; —SCN; —SR^{F1}; —SOR^{F1}; —SO₂R^{F2}; —NO₂; —N₃; —N(R^{F2})₂; —NR^{F2}C(═O)R^{F2}; —NR^{F2}C(═O)N(R^{F2})₂; —OC(═O)OR^{F1}; —OC(═O)R^{F2}; —OC(═O)N(R^{F2})₂; —NR^{F2}C(═O)OR^{F1}; or —C(R^{F2})₃; wherein each occurrence of R^{F1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{F1} groups are optionally joined to form a substituted or unsubstituted heterocyclyl or substituted or unsubstituted heteroaryl ring; and wherein each occurrence of R^{F2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{F1}; —SR^{F1}; or —N(R^{F1})₂; or two R^{F2} groups are optionally joined to form a substituted or unsubstituted heterocyclyl or substituted or unsubstituted heteroaryl ring;
R⁷ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{G1}; —C(═O)R^{G2}; —CO₂R^{G1}; —CN; —SCN; —SR^{G1}; —SOR^{G1}; —SO₂R^{G2}; —NO₂; —N₃; —N(R^{G})₂; —NR^{G2}C(═O)R^{G2}; —NR^{G2}C(═O)N(R^{G2})₂; —OC(═O)OR^{G1}; —OC(═O)R^{G2}; —OC(═O)N(R^{G2})₂; —NR^{G2}C(═O)OR^{G1}; or —C(R^{G2})₃; wherein each occurrence of R^{G1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{G1} groups are optionally joined to form a substituted or unsubstituted heterocyclyl or substituted or unsubstituted heteroaryl ring; and wherein each occurrence of R^{G2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{G1}; —SR^{G1}; or —N(R^{G1})₂; or two R^{G2} groups are optionally joined to form a substituted or unsubstituted heterocyclyl or substituted or unsubstituted heteroaryl ring;
R⁸ is hydrogen; an oxygen protecting group; a carbohydrate; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; substituted or unsubstituted heteroaliphatic; acyl; OR^{H9}; —OC(═O)R^{H9}; —N(R^{H9})₂; or —NHC(═O)R^{H9}; wherein each occurrence of R^{H9} is independently hydrogen; substituted or unsubstituted alkyl; an oxygen protecting group when attached to an oxygen atom; or a nitrogen protecting group when attached to a nitrogen atom; or two R^{H9} groups are joined to form a substituted or unsubstituted heterocyclic or substituted or unsubstituted heteroaryl ring;
R⁹ is —C(R^{I1})₃; wherein each occurrence of R^{I1} is independently hydrogen;
carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{I2}; —SR^{I2}; —N(R^{I2})₂; azido; or halogen; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring;
R¹⁰ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{B1}; —C(═O)R^{B2}; —CO₂R^{B2}; —CN; —SCN; —SR^{B1}; —SOR^{B1}; —SO₂R^{B2}; —NO₂; —N₃; —N(R^{B2})₂; —NR^{B2}C(═O)R^{B2}; —NR^{B2}C(═O)N(R^{B2})₂; —OC(═O)OR^{B1}; —OC(═O)R^{B2}; —OC(═O)N(R^{B2})₂; —NR^{B2}C(═O)OR^{B1}; or —C(R^{B2})₃; wherein each occurrence of R^{B1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{B1} groups are optionally joined to form a substituted or unsubstituted heterocyclyl or substituted or unsubstituted heteroaryl ring; and wherein each occurrence of R^{B2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{B1}; —SR^{B1}; or —N(R^{B1})₂; or two R^{B2} groups are optionally joined to form a substituted or unsubstituted heterocyclyl or substituted or unsubstituted heteroaryl ring;
R^{I1} is hydrogen; substituted or unsubstituted alkyl; acyl; -L-A-B; or -L-T;
X is a halogen;
or R¹ and R¹⁰ are optionally taken together with the intervening carbon atoms to form an optionally substituted cyclic moiety;
or R¹⁰ and R³ are optionally taken together with the intervening carbon atoms to form an optionally substituted cyclic moiety;
or R¹ and R⁴ are optionally taken together with the intervening carbon atoms to form an optionally substituted cyclic moiety;
or R⁴ and R⁷ are optionally taken together with the intervening carbon atoms to form an optionally substituted cyclic moiety;
or R⁶ and R⁷ are optionally taken together with the intervening carbon atoms to form an optionally substituted cyclic moiety;
L is a linker;
A is a bond or a group of formula:

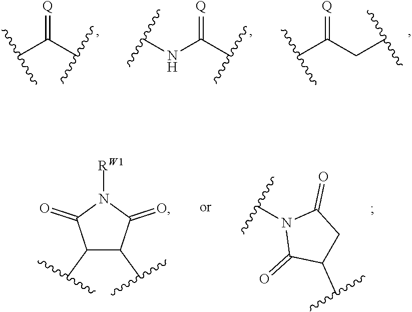

Q is —S— or —O—;
R^{W1} is independently hydrogen, substituted or unsubstituted alkyl; or a nitrogen protecting group;
B is a targeting moiety; and
T is hydrogen, —N═C═S,

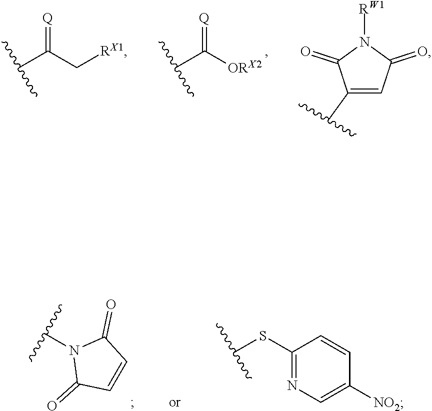

R^{X1} is a leaving group; and
R^{X2} is hydrogen; substituted or unsubstituted alkyl; substituted or unsubstituted heterocyclyl; substituted or unsubstituted aryl; substituted or unsubstituted heteroaryl; or an oxygen protecting group.

In certain embodiments, the compound of Formula (II) is of Formula (II-a):

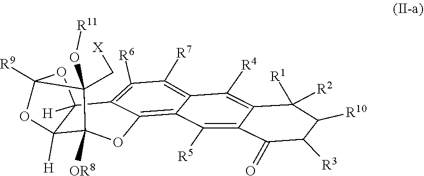

or a pharmaceutically acceptable salt thereof.

In certain embodiments, the compound of Formula (II) is of Formula (II-b):

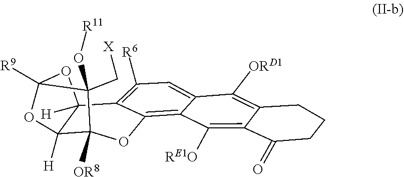

or a pharmaceutically acceptable salt thereof.

In certain embodiments, the compound of Formula (II) is of Formula (II-c):

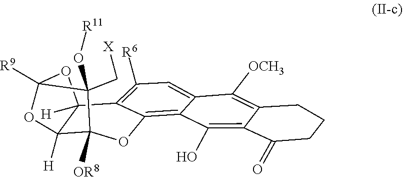

or a pharmaceutically acceptable salt thereof.

In certain embodiments, the compound of Formula (II) is of Formula (II-d):

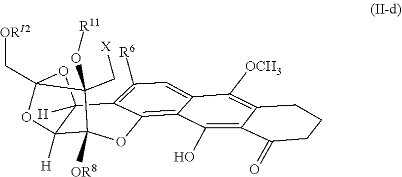

or a pharmaceutically acceptable salt thereof.

In certain embodiments, the compound of Formula (II-d) is of Formula (II-d-i):

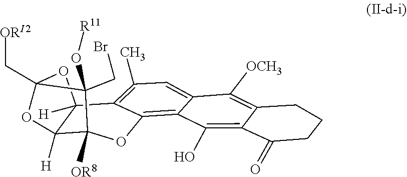

or a pharmaceutically acceptable salt thereof.

In certain embodiments, the compound of Formula (II-d) is of Formula (II-d-ii):

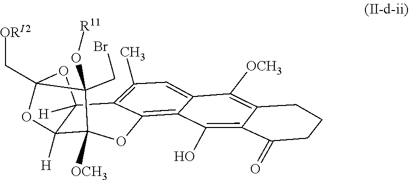

or a pharmaceutically acceptable salt thereof.

In certain embodiments, the compound of Formula (II) is of Formula (II-e):

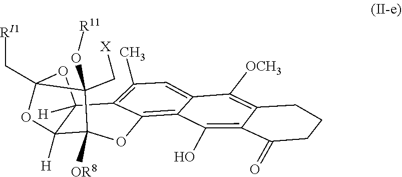

or a pharmaceutically acceptable salt thereof.

In certain embodiments, the compound of Formula (II-e) is of Formula (II-e-i):

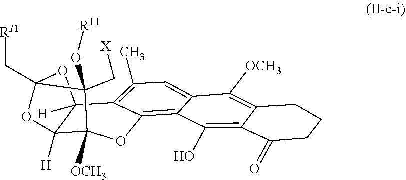

or a pharmaceutically acceptable salt thereof.

In certain embodiments, the compound of Formula (II-e) is of Formula (II-e-ii):

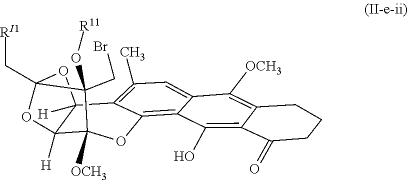

or a pharmaceutically acceptable salt thereof.

Also provided are intermediate compounds of Formula:

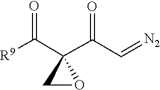

or a salt thereof; wherein:
R⁹ is hydrogen or —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; C(═O)R^{I1}; -L-A-B; or -L-T; or two R^{I2} groups are optionally joined to form a substituted or unsubstituted heterocyclyl or substituted or unsubstituted heteroaryl ring.

In certain embodiments, R⁹ is —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; C(═O)R^{I1}; -L-A-B; or -L-T; or two R^{I2} groups are optionally joined to form a substituted or unsubstituted heterocyclyl or substituted or unsubstituted heteroaryl ring.

In certain embodiments, the intermediate compound is:

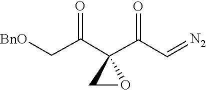

or a salt thereof.
Groups R¹ and R²

As generally defined above for compounds of Formula (I) and (II), R¹ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{A1}; —C(═O)R^{A2}; —CO₂R^{A2}; —CN; —SCN; —SR^{A1}; —SOR^{A1}; —SO₂R^{A1}; —NO₂; —N₃; —N(R^{A2})₂; —NR^{A2}C(═O)R^{A2}; —NR^{A2}C(═O)N(R^{A2})₂; —OC(═O)OR^{A1}; —OC(═O)R^{A2}; —OC(═O)N(R^{A2})₂; —NRAC(═O)OR^{A1}; or —C(R^{A2})₃; and R² is hydrogen; or R¹ and R² together form ═O; ═N(R^{A2}); or ═S.

In certain embodiments, R¹ is hydrogen; and R² is hydrogen.

In certain embodiments, R¹ is halogen (e.g., —F, —Cl, Br, or —I); and R² is hydrogen.

In certain embodiments, R¹ is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl, and R² is hydrogen. Exemplary R¹ C₁₋₆ alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆).

In certain embodiments, R¹ is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl, and R² is hydrogen.

In certain embodiments, R¹ is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl, and R² is hydrogen.

In certain embodiments, R¹ is substituted or unsubstituted carbocyclyl, e.g., substituted or unsubstituted C₃₋₆carbocyclyl, substituted or unsubstituted C₃₋₄ carbocyclyl, substituted or unsubstituted C₄₋₅ carbocyclyl, or substituted or unsubstituted C₅₋₆ carbocyclyl, and R² is hydrogen.

In certain embodiments, R¹ is substituted or unsubstituted heterocyclyl, e.g., substituted or unsubstituted 3-6 membered heterocyclyl, substituted or unsubstituted 3-4 membered heterocyclyl, substituted or unsubstituted 4-5 membered heterocyclyl, or substituted or unsubstituted 5-6 membered heterocyclyl, and R² is hydrogen.

In certain embodiments, R¹ is substituted or unsubstituted aryl, e.g., substituted or unsubstituted phenyl, and R² is hydrogen.

In certain embodiments, R¹ is substituted or unsubstituted heteroaryl, e.g., substituted or unsubstituted 5-6 membered heteroaryl, and R² is hydrogen.

In certain embodiments, R¹ is —OR^{A1}, and R² is hydrogen. In certain embodiments, R¹ is —C(═O)R^{A2} and R² is hydrogen. In certain embodiments, R¹ is —CO₂R^{A2} and R² is hydrogen. In certain embodiments, R¹ is —CN and R² is hydrogen. In certain embodiments, R¹ is —SCN and R² is hydrogen. In certain embodiments, R¹ is —SR^{A1} and R² is hydrogen. In certain embodiments, R¹ is —SOR^{A1} and R² is hydrogen. In certain embodiments, R¹ is —SO₂R^{A2} and R² is hydrogen. In certain embodiments, R¹ is —NO₂ and R² is hydrogen. In certain embodiments, R¹ is —N₃ and R² is hydrogen. In certain embodiments, R¹ is —N(R^{A2})₂ and R² is hydrogen. In certain embodiments, R¹ is —NR^{A2}C(═O)R^{A2} and R² is hydrogen. In certain embodiments, R¹ is —NR^{A2}C(═O)N(R^{A2})₂ and R² is hydrogen. In certain embodiments, R¹ is —OC(═O)OR^{A1} and R² is hydrogen. In certain embodiments, R¹ is —OC(═O)R^{A2} and R² is hydrogen. In certain embodiments, R¹ is —OC(═O)N(R^{A2})₂ and R² is hydrogen. In certain embodiments, R¹ is —NR^{A2}C(═O)OR^{A1} and R² is hydrogen. In certain embodiments, R¹ is —C(R^{A2})₃ and R² is hydrogen.

In certain embodiments, R¹ and R² are joined to form ═O.

As generally defined above for compounds of Formula (I) and (II), each occurrence of R^{A1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{A1} groups are optionally joined to form a substituted or unsubstituted heterocyclyl or substituted or unsubstituted heteroaryl ring; and wherein each occurrence of R^{A2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{A1}; —SR^{A1}; or —N(R^{A1})₂; or two R^{A2} groups are optionally joined to form a substituted or unsubstituted heterocyclyl or substituted or unsubstituted heteroaryl ring; with the proviso that if R¹ is —OR^{A1}, R^{A1} is not 4-methoxybenzyl or carbohydrate.

In certain embodiments, R^{A1} or R^{A2} represent a group of Formula (i):

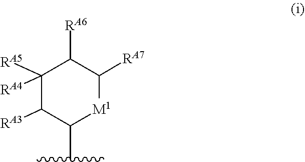

each occurrence of R^{A3}, R^{A4}, R^{A5}, R^{A6}, and R^{A7} is independently hydrogen, substituted or unsubstituted alkyl; —OR^{A9}; —OC(═O)R^{A9}; —N(R^{A9})₂; or —NHC(═O)R^{A9}; wherein each occurrence of R^{A9} is independently hydrogen; substituted or unsubstituted alkyl; an oxygen protecting group when attached to an oxygen atom; or a nitrogen protecting group when attached to a nitrogen atom; or two R^{A9} groups are joined to form a substituted or unsubstituted heterocyclic or substituted or unsubstituted heteroaryl ring; and
M¹ is —O—,—NR^{A8}—, or —CHR^{A8}— wherein R^{A8} is hydrogen; substituted or unsubstituted alkyl; or a nitrogen protecting group if attached to nitrogen; or —OR^{A9}; wherein R^{A9} is independently hydrogen; substituted or unsubstituted alkyl; acyl; or an oxygen protecting group.

In certain embodiments, R^{A1} or R^{A2} is hydrogen.

In certain embodiments, R^{A3} is hydrogen, substituted or unsubstituted alkyl; —OR^{A9}; —OC(═O)R^{A9}; or —NHC(═O)R^{A9}; wherein each occurrence of R^{A9} is independently hydrogen; substituted or unsubstituted alkyl; or an oxygen protecting group. In certain embodiments, R^{A3} is hydrogen. In certain embodiments, R^{A3} is substituted or unsubstituted alkyl, e.g., methyl. In certain embodiments, R^{A3} is —OR^{A9}, e.g., —OH or —O-alkyl.

In certain embodiments, R^{A4} is hydrogen; substituted or unsubstituted alkyl; —OR^{A9}; —OC(═O)R^{A9}; or —NHC(═O)R^{A9}; wherein each occurrence of R^{A9} is independently hydrogen; substituted or unsubstituted alkyl; or an oxygen protecting group. In certain embodiments, R^{A4} is hydrogen. In certain embodiments, R^{A4} is substituted or unsubstituted alkyl, e.g., methyl. In certain embodiments, R^{A4} is —OR^{A9}, e.g., —OH or —O-alkyl.

In certain embodiments, R^{A5} is hydrogen, substituted or unsubstituted alkyl; —OR^{A9}; —OC(═O)R^{A9}; or —NHC(═O)R^{A9}; wherein each occurrence of R^{A9} is independently hydrogen; substituted or unsubstituted alkyl; or an oxygen protecting group. In certain embodiments, R^{A5} is hydrogen. In certain embodiments, R^{A5} is substituted or unsubstituted alkyl, e.g., methyl. In certain embodiments, R^{A5} is —OR^{A9}, e.g., —OH or —O-alkyl.

In certain embodiments, R^{A6} is hydrogen, substituted or unsubstituted alkyl; —OR^{A9}; —OC(═O)R^{A9}; or —NHC(═O)R^{A9}; wherein each occurrence of R^{A9} is independently hydrogen; substituted or unsubstituted alkyl; or an oxygen protecting group. In certain embodiments, R^{A6} is hydrogen. In certain embodiments, R^{A6} is substituted or unsubstituted alkyl, e.g., methyl. In certain embodiments, R^{A6} is —OR^{A9}, e.g., —OH or —O-alkyl.

In certain embodiments, R^{A7} is hydrogen; substituted or unsubstituted alkyl; —OR^{A9}; —OC(═O)R^{A9}; or —NHC(═O)R^{A9}; wherein each occurrence of R^{A9} is independently hydrogen; substituted or unsubstituted alkyl; or an oxygen protecting group. In certain embodiments, R^{A7} is hydrogen. In certain embodiments, R^{A7} is substituted or unsubstituted alkyl, e.g., methyl. In certain embodiments, R^{A7} is —OR^{A9}, e.g., —OH or —O-alkyl.

In certain embodiments, M¹ is —O—,—NR^{A8}—, or —CHR^{A8}— wherein R^{A8} is hydrogen; substituted or unsubstituted alkyl; a nitrogen protecting group if attached to nitrogen; or —OR^{A9}; wherein R^{A9} is independently hydrogen; substituted or unsubstituted alkyl; acyl; or an oxygen protecting group.

In certain embodiments, M¹ is —O—. In certain embodiments, M¹ is —NR^{A8}—, e.g., —NH—. In certain embodiments, M¹ is —CHR^{A8}—, e.g., —CH₂—.

In certain embodiments, R^{A3} is hydrogen; R^{A4} is hydrogen or —OR^{A9}; R^{A5} is methyl, —NHC(═O)R^{A9}; R^{A6} is hydrogen, —OR^{A9}, —OC(═O)R^{A9}, or —NHC(═O)R^{A9}; R^{A7} is methyl; and M¹ is —O—.

In certain embodiments, R^{A3} is hydrogen; R^{A4} is hydrogen or —OR^{A9}; R^{A5} is methyl, —NHC(═O)R^{A9}; R^{A6} is hydrogen, —OR^{A9}, —OC(═O)R^{A9}, or —NHC(═O)R^{A9}; and R^{A7} is methyl. In certain embodiments, R^{A3} is a non-hydrogen equatorial group.

Groups R³, R⁴, R⁵, R⁶ and R⁷

As generally defined above for compounds of Formula (I) and (II), R³ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{C1}; —C(═O)R^{C2}; —CO₂R^{C1}; —CN; —SCN; —SR^{C1}; —SOR^{C1}; —SO₂R^{C2}; —NO₂; —N₃; —N(R^{C2})₂; —NHC(═O)R^{C2}; —NR^{C2}C(═O)N(R^{C2})₂; —OC(═O)OR^{C1}; —OC(═O)R^{C2}; —OC(═O)N(R^{C2})₂; —NR^{C2}C(═O)OR^{C1}; or —C(R^{C2})₃; wherein each occurrence of R^{C1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{C1} groups are optionally joined to form a substituted or unsubstituted heterocyclyl or substituted or unsubstituted heteroaryl ring; and wherein each occurrence of R^{C2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{C1}; —SR^{C1}; or —N(R^{C1})₂; or two R^{C2} groups are optionally joined to form a substituted or unsubstituted heterocyclyl or substituted or unsubstituted heteroaryl ring.

In certain embodiments, R³ is hydrogen or —OR^{C1}, wherein R^{C1} is hydrogen; an oxygen protecting group; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; or acyl. In certain embodiments, R³ is hydrogen. In certain embodiments, R³ is —OR^{C1}, e.g., —OH or —OCH3.

As generally defined above for compounds of Formula (I) and (II), R⁴ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{D1}; —C(═O)R^{D2}; —CO₂R^{D2}; —CN; —SCN; —SR^{D1}; —SOR^{D1}; —SO₂R^{D2}; —NO₂; —N₃; —N(R^{D2})₂; —NR^{D2}C(═O)R^{D2}; —NR^{D2}C(═O)N(R^{D2})₂; —OC(═O)OR^{D1}; —OC(═O)R^{D2}; —OC(═O)N(R^{D2})₂; —NR^{D2}C(═O)OR^{D1}; or —C(R^{D2})₃; wherein each occurrence of R^{D1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{D1} groups are optionally joined to form a substituted or unsubstituted heterocyclyl or substituted or unsubstituted heteroaryl ring; and wherein each occurrence of R^{D2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{D1}; —SR^{D1}; or —N(R^{D1})₂; or two R^{D2} groups are optionally joined to form a substituted or unsubstituted heterocyclyl or substituted or unsubstituted heteroaryl ring.

In certain embodiments, R⁴ is hydrogen or —OR^{D1}, wherein R^{D1} is hydrogen; an oxygen protecting group; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; or acyl. In certain embodiments, R⁴ is hydrogen. In certain embodiments, R⁴ is —OR^{D1}, e.g., —OH or —OCH₃. In certain embodiments, R^{D1} is C₁₋₆ alkyl.

As generally defined above for compounds of Formula (I) and (II), R⁵ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{E1}; —C(═O)R^{E2}; —CO₂R^{E1}; —CN; —SCN; —SR^{E1}; —SOR^{E1}; —SO₂R^{E2}; —NO₂; —N₃; —N(R^{E2})₂; —NR^{E2}C(═O)R^{E2}; —NR^{E2}C(═O)N(R^{E2})₂; —OC(═O)OR^{E1}; —OC(═O)R^{E2}; —OC(═O)N(R^{E2})₂; —NR^{F2}C(═O)OR^{E1}; or —C(R^{E2})₃; wherein each occurrence of R^{E1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{E1} groups are optionally joined to form a substituted or unsubstituted heterocyclyl or substituted or unsubstituted heteroaryl ring; and wherein each occurrence of R^{E2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{E1}; —SR^{E1}; or —N(R^{E1})₂; or two R^{E2} groups are optionally joined to form a substituted or unsubstituted heterocyclyl or substituted or unsubstituted heteroaryl ring.

In certain embodiments, R⁵ is hydrogen or —OR^{E1}, wherein R^{E1} is hydrogen; an oxygen protecting group; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; or acyl. In certain embodiments, R⁵ is hydrogen. In certain embodiments, R⁵ is —OR^{E1}, e.g., —OH or —OCH₃. In certain embodiments, R⁵ is —OR^{E1}, wherein R^{E1} is a protecting group. In certain embodiments, R⁵ is —OR^{E1}, wherein R^{E1} is a Boc protecting group.

As generally defined above for compounds of Formula (I) and (II), R⁶ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{F1}; —C(═O)R^{F2}; —CO₂R^{F1}; —CN; —SCN; —SR^{F1}; —SOR^{F1}; —SO₂R^{F2}; —NO₂; —N₃; —N(R^{F2})₂; —NR^{F2}C(═O)R^{F2}; —NR^{F2}C(═O)N(R^{F2})₂; —OC(═O)OR^{F1}; —OC(═O)R^{F2}; —OC(═O)N(R^{F2})₂; —NR^{F2}C(═O)OR^{F1}; or —C(R^{F2})₃; wherein each occurrence of R^{F1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{F1} groups are optionally joined to form a substituted or unsubstituted heterocyclyl or substituted or unsubstituted heteroaryl ring; and wherein each occurrence of R^{F2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{F1}; —SR^{F1}; or —N(R^{F1})₂; or two R^{F2} groups are optionally joined to form a substituted or unsubstituted heterocyclyl or substituted or unsubstituted heteroaryl ring.

In certain embodiments, R⁶ is hydrogen; halogen; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; —OR^{F1}; —C(═O)R^{F2}; —CO₂R^{F1}; —CN; —SCN; —SR^{F1}; —SOR^{F1}; —SO₂R^{F2}; —NO₂; —N₃; —N(R^{F2})₂; —NR^{F2}C(═O)R^{F2}; —NR^{F2}C(═O)N(R^{F2})₂; —OC(═O)OR^{F1}; —OC(═O)R^{F2}; —OC(═O)N(R^{F2})₂; —NR^{F2}C(═O)OR^{F1}; or —C(R^{F2})₃.

In certain embodiments, R⁶ is hydrogen.

In certain embodiments, R⁶ is halogen; e.g., —F, —Cl, Br, or —I.

In certain embodiments, R⁶ is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R⁶ C₁₋₆ alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆).

In certain embodiments, R⁶ is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, R⁶ is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, R⁶ is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl. In certain embodiments, R⁶ is substituted or unsubstituted cyclopropyl.

In certain embodiments, R⁶ is substituted or unsubstituted heterocyclyl, e.g., substituted or unsubstituted 3-6 membered heterocyclyl, substituted or unsubstituted 3-4 membered heterocyclyl, substituted or unsubstituted 4-5 membered heterocyclyl, or substituted or unsubstituted 5-6 membered heterocyclyl.

In certain embodiments, R⁶ is substituted or unsubstituted aryl, e.g., substituted or unsubstituted phenyl.

In certain embodiments, R⁶ is substituted or unsubstituted heteroaryl, e.g., substituted or unsubstituted5-6 membered heteroaryl.

In certain embodiments, R⁶ is —OR^{F1}. In certain embodiments, R⁶ is —C(═O)R^{F2}. In certain embodiments, R⁶ is —CO₂R^{F1}. In certain embodiments, R⁶ is —CN. In certain embodiments, R⁶ is —SCN. In certain embodiments, R⁶ is —SR^{F1}. In certain embodiments, R⁶ is —SOR^{F1}. In certain embodiments, R⁶ is —SO₂R^{F2}. In certain embodiments, R⁶ is —NO₂. In certain embodiments, R⁶ is —N₃. In certain embodiments, R⁶ is —N(R^{F2})₂. In certain embodiments, R⁶ is —NR^{F2}C(═O)R^{F2}. In certain embodiments, R⁶ is —NR^{F2}C(═O)N(R^{F2})₂. In certain embodiments, R⁶ is —OC(═O)OR^{F1}. In certain embodiments, R⁶ is —OC(═O)R^{F2}. In certain embodiments, R^{o} is —OC(═O)N(R^{F2})₂. In certain embodiments, R⁶ is —NR^{F2}C(═O)OR^{F1}. In certain embodiments, R⁶ is —C(R^{F2})₃.

In certain embodiments, each occurrence of R^{F1} is independently hydrogen; an oxygen protecting group if attached to oxygen; a sulfur protecting group if attached to sulfur; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; or acyl; and each occurrence of R^{F2} is independently hydrogen; a nitrogen protecting group if attached to nitrogen; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; acyl; hydroxyl; substituted hydroxyl; thiol; substituted thiol; amino; or substituted amino; or two R^{F2} groups are optionally joined to form a heterocyclyl or heteroaryl ring.

In certain embodiments, R^{F1} or R^{F2} is hydrogen.

In certain embodiments, R³ is hydrogen or —OH, R⁴ is —OCH₃, and R⁵ is —OH.

In certain embodiments, R³ is hydrogen or —OH, R₄ is —OCH₃, R⁵ is —OH, and R⁶ substituted or unsubstituted alkyl or —OR^{F1}

As generally defined above for compounds of Formula (I) and (II), R⁷ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{G1}; —C(═O)R^{G2}; —CO₂R^{G1}; —CN; —SCN; —SR^{G1}; —SOR^{G1}; —SO₂R^{G2}; —NO₂; —N₃; —N(R^{G})₂; —NR^{G2}C(═O)R^{G2}; —NR^{G2}C(═O)N(R^{G2})₂; —OC(═O)OR^{G1}; —OC(═O)R^{G2}; —OC(═O)N(R^{G2})₂; —NR^{G2}C(═O)OR^{G1}; or —C(R^{G2})₃; wherein each occurrence of R^{G1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{G1} groups are optionally joined to form a substituted or unsubstituted heterocyclyl or substituted or unsubstituted heteroaryl ring; and wherein each occurrence of R^{G2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{G1}; —SR^{G1}; or —N(R^{G1})₂; or two R^{G2} groups are optionally joined to form a substituted or unsubstituted heterocyclyl or substituted or unsubstituted heteroaryl ring.

In certain embodiments, R⁷ is hydrogen; halogen; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; —OR^{G1}; —C(═O)R^{G2}; —CO₂R^{G1}; —CN; —SCN; —SR^{G1}; —SOR^{G1}; —SO₂R^{G2}; —NO₂; —N₃; —N(R^{G2})₂; —NR^{G2}C(═O)R^{G2}; —NR^{G2}C(═O)N(R^{G2})₂; —OC(═O)OR^{G1}; —OC(═O)R^{G2}; —OC(═O)N(R^{G2})₂; —NR^{G2}C(═O)OR^{G1}; or —C(R^{G2})₃.

In certain embodiments, R⁷ is hydrogen.

In certain embodiments, R⁷ is halogen; e.g., —F, —Cl, Br, or —I.

In certain embodiments, R⁷ is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. Exemplary R⁷ C₁₋₆ alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆).

In certain embodiments, R⁷ is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, R⁷ is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, R⁷ is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl. In certain embodiments, R⁷ is substituted or unsubstituted cyclopropyl.

In certain embodiments, R⁷ is substituted or unsubstituted heterocyclyl, e.g., substituted or unsubstituted 3-6 membered heterocyclyl, substituted or unsubstituted 3-4 membered heterocyclyl, substituted or unsubstituted 4-5 membered heterocyclyl, or substituted or unsubstituted 5-6 membered heterocyclyl.

In certain embodiments, R⁷ is substituted or unsubstituted aryl, e.g., substituted or unsubstituted phenyl.

In certain embodiments, R⁷ is substituted or unsubstituted heteroaryl, e.g., substituted or unsubstituted, 5-6 membered heteroaryl.

In certain embodiments, R⁷ is —OR^{G1}. In certain embodiments, R⁷ is —C(═O)R^{G2}. In certain embodiments, R⁷ is —CO₂R^{G1}. In certain embodiments, R⁷ is —CN. In certain embodiments, R⁷ is —SCN. In certain embodiments, R⁷ is —SR^{G1}. In certain embodiments, R⁷ is —SOR^{G1}. In certain embodiments, R⁷ is —SO₂R^{G2}. In certain embodiments, R⁷ is —NO₂. In certain embodiments, R⁷ is —N₃. In certain embodiments, R⁷ is —N(R^{G2})₂. In certain embodiments, R⁷ is —NR^{G2}C(═O)R^{G2}. In certain embodiments, R⁷ is —NR^{G2}C(═O)N(R^{G2})₂. In certain embodiments, R⁷ is —OC(═O)OR^{G1}. In certain embodiments, R⁷ is —OC(═O)R^{G2}. In certain embodiments, R⁷ is —OC(═O)N(R^{G2})₂. In certain embodiments, R⁷ is —NR^{G2}C(═O)OR^{G1}. In certain embodiments, R⁷ is —C(R^{G2})₃.

In certain embodiments, each occurrence of R^{G1} is independently hydrogen; an oxygen protecting group if attached to oxygen; a sulfur protecting group if attached to sulfur; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; or acyl; and each occurrence of R^{G2} is independently hydrogen; a nitrogen protecting group if attached to nitrogen; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; acyl; hydroxyl; substituted hydroxyl; thiol; substituted thiol; amino; or substituted amino; or two R^{G2} groups are optionally joined to form a heterocyclyl or heteroaryl ring.

In certain embodiments, R^{G1} or R^{G2} is hydrogen.

### Group RS

As generally defined above for compounds of Formula (I) and (II), R⁸ is hydrogen; an oxygen protecting group; a carbohydrate; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; substituted or unsubstituted heteroaliphatic; or acyl; —OR^{H9}; —OC(═O)R^{H9}; —N(R^{H9})₂; or —NHC(═O)R^{H9}; wherein each occurrence of R^{H9} is independently hydrogen; substituted or unsubstituted alkyl; an oxygen protecting group when attached to an oxygen atom; or a nitrogen protecting group when attached to a nitrogen atom; or two R^{H9} groups are joined to form a substituted or unsubstituted heterocyclic or substituted or unsubstituted heteroaryl ring.

In certain embodiments, R⁸ is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl.

In certain embodiments, R⁸ is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, R⁸ is substituted or unsubstituted alkynyl, e.g., substituted or unsubstituted C₂₋₆ alkynyl, substituted or unsubstituted C₂₋₃alkynyl, substituted or unsubstituted C₃₋₄ alkynyl, substituted or unsubstituted C₄₋₅ alkynyl, or substituted or unsubstituted C₅₋₆ alkynyl.

In certain embodiments, R⁸ is substituted or unsubstituted carbocyclyl, e.g., substituted or unsubstituted C₃₋₆ carbocyclyl, substituted or unsubstituted C₃₋₄ carbocyclyl, substituted or unsubstituted C₄₋₅ carbocyclyl, or substituted or unsubstituted C₅₋₆ carbocyclyl.

In certain embodiments, R⁸ is substituted or unsubstituted heterocyclyl, e.g., substituted or unsubstituted 3-6 membered heterocyclyl, substituted or unsubstituted 3-4 membered heterocyclyl, or substituted or unsubstituted 4-5 membered heterocyclyl.

In certain embodiments, R⁸ is substituted or unsubstituted aryl, e.g., substituted or unsubstituted phenyl.

In certain embodiments, R⁸ is substituted or unsubstituted heteroaryl, e.g., substituted or unsubstituted 5-6 membered heteroaryl.

In certain embodiments, R⁸ is hydrogen; an oxygen protecting group; or substituted or unsubstituted alkyl. In certain embodiments, R⁸ is —CH₃.

In certain embodiments, R⁸ is a carbohydrate. In certain embodiments, the carbohydrate of R⁸ is:

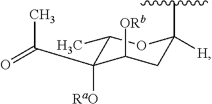

wherein R^{a} and R^{b} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl.

### Group R⁹

As generally defined above for compounds of Formula (I) and (II), R⁹ is hydrogen or —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{I2}; —SR^{I2}; —N(R^{I2})₂; azido; or halogen; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring.

In certain embodiments, R⁹ is —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{I2}; —SR^{I2}; —N(R^{I2})₂; azido; or halogen; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring.

In certain embodiments, R⁹ is hydrogen. In certain embodiments, R⁹ is —C(R^{I1})₃.

In certain embodiments, each occurrence of R^{I1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{I2}; —SR^{I2}; —N(R^{I2})₂; azido; or halogen; with the proviso that not more than one occurrence of R^{I1} is —OR^{I2}.

In certain embodiments, at least one instance of R^{I1} is independently hydrogen. In certain embodiments, two of R^{I1} are each hydrogen.

In certain embodiments, at least once instance of R^{I1} is carbohydrate.

In certain embodiments, at least one instance of R^{I1} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl.

In certain embodiments, at least one instance of R^{I1} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, at least one instance of R^{I1} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, at least one instance of R^{I1} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, at least one instance of R^{I1} is substituted or unsubstituted heterocyclyl, e.g., substituted or unsubstituted 3-6 membered heterocyclyl, substituted or unsubstituted 3-4 membered heterocyclyl, substituted or unsubstituted 4-5 membered heterocyclyl, or substituted or unsubstituted 5-6 membered heterocyclyl.

In certain embodiments, at least one instance of R^{I1} is substituted or unsubstituted aryl, e.g., substituted or unsubstituted phenyl.

In certain embodiments, at least one instance of R^{I1} is substituted or unsubstituted heteroaryl, e.g., substituted or unsubstituted 5-6 membered heteroaryl.

In certain embodiments, at least one instance of R^{I1} is acyl.

In certain embodiments, one occurrence of R^{I1} is —OR^{I2}; —SR^{I2}; or —N(R^{I2})₂. In certain embodiments, one occurrence of R^{I1} is —OR^{I2}; —SR^{I2}; or —N(R^{I2})₂; and the remaining two are hydrogen. In certain embodiments, one occurrence of R^{I1} is —OR^{I2}; —SR^{I2}; or —NHR^{I2}. In certain embodiments, one occurrence of R^{I1} is —OR^{I2}; —SR^{I2}; or —NHR^{I2}; and the remaining two are hydrogen. In certain embodiments, one occurrence of R^{I1} is —OR^{I2}; and the remaining two are hydrogen. In certain embodiments, one occurrence of R^{I1} is —SR^{I2}; and the remaining two are hydrogen. In certain embodiments, one occurrence of R^{I1} is —N(R^{I2})₂; and the remaining two are hydrogen. In certain embodiments, one occurrence of R^{I1} is —NHR^{I2}; and the remaining two are hydrogen.

In certain embodiments, each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring.

In certain embodiments, R^{I2} is hydrogen.

In certain embodiments, at least one instance of R^{I2} is a nitrogen protecting group if attached to nitrogen. In certain embodiments, R^{I2} is an oxygen protecting group if attached to oxygen or a sulfur protecting group if attached to sulfur.

In certain embodiments, R^{I2} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl.

In certain embodiments, R^{I2} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, R^{I2} is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl.

In certain embodiments, R^{I2} is substituted or unsubstituted carbocyclyl, e.g., substituted or unsubstituted C₃₋₆carbocyclyl, substituted or unsubstituted C₃₋₄carbocyclyl, substituted or unsubstituted C₄₋₅ carbocyclyl, or substituted or unsubstituted C₅₋₆ carbocyclyl.

In certain embodiments, R^{I2} is substituted or unsubstituted heterocyclyl, e.g., substituted or unsubstituted 3-6 membered heterocyclyl, substituted or unsubstituted 3-4 membered heterocyclyl, substituted or unsubstituted 4-5 membered heterocyclyl, or substituted or unsubstituted 5-6 membered heterocyclyl.

In certain embodiments, R^{I2} is substituted or unsubstituted aryl, e.g., substituted or unsubstituted phenyl.

In certain embodiments, R^{I2} is substituted or unsubstituted heteroaryl, e.g., substituted or unsubstituted 5-6 membered heteroaryl.

In certain embodiments, R^{I2} is substituted or unsubstituted heteroarylalkyl.

In certain embodiments, R^{I2} is acyl.

In certain embodiments, two R^{I2} groups are optionally joined to form a heterocyclyl or heteroaryl ring.

In certain embodiments, R⁹ is —CH₂R^{I1} and R^{I1} is hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{I2}; —SR^{I2}; —N(R^{I2})₂; azido; or halogen.

In certain embodiments, R⁹ is —CH₂R^{I1} and R^{I1} is hydrogen. In certain embodiments, R⁹ is methyl.

In certain embodiments, R⁹ is —CH₂R^{I1} and R^{I1} is carbohydrate.

In certain embodiments, R⁹ is —CH₂R^{I1} and R^{I1} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl.

In certain embodiments, R⁹ is —CH₂R^{I1} and R^{I1} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, R⁹ is —CH₂R^{I1} and R^{I1} is substituted or unsubstituted alkynyl, e.g., substituted or unsubstituted C₂₋₆ alkynyl, substituted or unsubstituted C₂₋₃alkynyl, substituted or unsubstituted C₃₋₄ alkynyl, substituted or unsubstituted C₄₋₅ alkynyl, or substituted or unsubstituted C₅₋₆ alkynyl.

In certain embodiments, R⁹ is —CH₂R^{I1} and R^{I1} is substituted or unsubstituted carbocyclyl, e.g., substituted or unsubstituted C₃₋₆ carbocyclyl, substituted or unsubstituted C₃₋₄ carbocyclyl, substituted or unsubstituted C₄₋₅ carbocyclyl, or substituted or unsubstituted C₅₋₆ carbocyclyl.

In certain embodiments, R⁹ is —CH₂R^{I1} and R^{I1} is substituted or unsubstituted heterocyclyl, e.g., substituted or unsubstituted 3-6 membered heterocyclyl, substituted or unsubstituted 3-4 membered heterocyclyl, or substituted or unsubstituted 4-5 membered heterocyclyl.

In certain embodiments, R⁹ is —CH₂R^{I1} and R^{I1} is substituted or unsubstituted aryl, e.g., substituted or unsubstituted phenyl.

In certain embodiments, R⁹ is —CH₂R^{I1} and R^{I1} is substituted or unsubstituted heteroaryl, e.g., substituted or unsubstituted 5-6 membered heteroaryl.

In certain embodiments, R⁹ is —CH₂R^{I1} and R^{I1} is acyl.

In certain embodiments, R⁹ is —CH₂R^{I1} and R^{I1} is substituted or unsubstituted heteroaliphatic.

In certain embodiments, R⁹ is —CH₂R^{I1} and R^{I1} is halogen, e.g., —F, —Cl, —Br, or —I.

In certain embodiments, R⁹ is —CH₂R^{I1} and R^{I1} is azide, e.g., —N₃.

In certain embodiments, R⁹ is —CH₂SR^{I2} and R^{I2} is hydrogen. In certain embodiments, R⁹ is —CH₂SR^{I2} and R^{I2} is acyl.

In certain embodiments, R⁹ is —CH₂OR^{I2} and R^{I2} is hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T.

In certain embodiments, R⁹ is —CH₂OR^{I2} and R^{I2} is hydrogen.

In certain embodiments, R⁹ is —CH₂OR^{I2} and R^{I2} is a carbohydrate.

In certain embodiments, R⁹ is —CH₂OR^{I2} and R^{I2} is

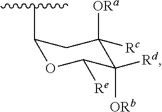

wherein R^{a} and R^{b} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or acyl; and
R^{c}, R^{d}, and R^{e} are independently hydrogen; cyclic or acyclic, substituted or unsubstituted, branched or unbranched aliphatic; cyclic or acyclic, substituted or unsubstituted, branched or unbranched heteroaliphatic; or acyl.

In certain embodiments, R⁹ is —CH₂OR^{I2} and R^{I2} is

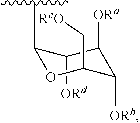

wherein R^{a}, R^{b}, R^{c}, and R^{d} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl.

In certain embodiments, R⁹ is —CH₂OR^{I2} and R^{I2} is an oxygen protecting group. In certain embodiments, R⁹ is —CH₂OBn.

In certain embodiments, R⁹ is —CH₂OR^{I2} and R^{I2} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. In certain embodiments, R⁹ is —CH₂OCH₃. In certain embodiments, R⁹ is —CH₂OCH₂CH₂CH₃.

In certain embodiments, R⁹ is —CH₂OR^{I2} and R^{I2} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl. In certain embodiments, R⁹ is —CH₂OCH₂CHCH₂.

In certain embodiments, R⁹ is —CH₂OR^{I2} and R^{I2} is substituted or unsubstituted alkynyl, e.g., substituted or unsubstituted C₂₋₆ alkynyl, substituted or unsubstituted C₂₋₃alkynyl, substituted or unsubstituted C₃₋₄ alkynyl, substituted or unsubstituted C₄₋₅ alkynyl, or substituted or unsubstituted C₅₋₆ alkynyl. In certain embodiments, R⁹ is —CH₂OCH₂CCH.

In certain embodiments, R⁹ is —CH₂OR^{I2} and R^{I2} is substituted or unsubstituted carbocyclyl, e.g., substituted or unsubstituted C₃₋₆ carbocyclyl, substituted or unsubstituted C₃₋₄ carbocyclyl, substituted or unsubstituted C₄₋₅ carbocyclyl, or substituted or unsubstituted C₅₋₆ carbocyclyl.

In certain embodiments, R⁹ is —CH₂OR^{I2} and R^{I2} is substituted or unsubstituted aryl, e.g., substituted or unsubstituted phenyl.

In certain embodiments, R⁹ is —CH₂OR^{I2} and R^{I2} is substituted or unsubstituted heteroaryl, e.g., substituted or unsubstituted 5-6 membered heteroaryl.

In certain embodiments, R⁹ is —CH₂OR^{I2} and R^{I2} is acyl. In certain embodiments, R⁹ is —CH₂OC(O)O(4-nitrophenyl).

In certain embodiments, R⁹ is —CH₂OR^{I2} and R^{I2} is substituted or unsubstituted heteroaliphatic.

In certain embodiments, R⁹ is —CH₂OR^{I2} and R^{I2} is substituted or unsubstituted heteroarylalkyl. In certain embodiments, R⁹ is substituted or unsubstituted triazolylmethyl.

In certain embodiments, R⁹ is —CH₂OR^{I2} and R^{I2} is substituted or unsubstituted phosphono. In certain embodiments R⁹ is —CH₂OP(O)(OH)₂.

In certain embodiments, R⁹ is —CH₂OR^{I2} and R^{I2} is -L-A-B.

In certain embodiments, R⁹ is —CH₂OR^{I2} and R^{I2} is -L-T.

In certain embodiments, R⁹ is —CH₂SR^{I2}. In certain embodiments, R⁹ is —CH₂SR^{I2} and R^{I2} is hydrogen. In certain embodiments, R⁹ is —CH₂SR^{I2} and R^{I2} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. In certain embodiments, R⁹ is —CH₂SR^{I2} and R^{I2} is acyl. In certain embodiments, R⁹ is —CH₂SC(O)CH₃.

In certain embodiments, R⁹ is —CH₂N(R^{I2})₂ and each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T.

In certain embodiments, R⁹ is —CH₂N(R^{I2})₂ and at least one instance of R^{I2} is hydrogen.

In certain embodiments, R⁹ is —CH₂N(R^{I2})₂ and at least one instance of R^{I2} is an oxygen protecting group.

In certain embodiments, R⁹ is —CH₂N(R^{I2})₂ and at least one instance of R^{I2} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl.

In certain embodiments, R⁹ is —CH₂N(R^{I2})₂ and at least one instance of R^{I2} is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl.

In certain embodiments, R⁹ is —CH₂N(R^{I2})₂ and at least one instance of R^{I2} is substituted or unsubstituted alkynyl, e.g., substituted or unsubstituted C₂₋₆ alkynyl, substituted or unsubstituted C₂₋₃alkynyl, substituted or unsubstituted C₃₋₄ alkynyl, substituted or unsubstituted C₄₋₅ alkynyl, or substituted or unsubstituted C₅₋₆ alkynyl.

In certain embodiments, R⁹ is —CH₂N(R^{I2})₂ and at least one instance of R^{I2} is substituted or unsubstituted carbocyclyl, e.g., substituted or unsubstituted C₃₋₆ carbocyclyl, substituted or unsubstituted C₃₋₄ carbocyclyl, substituted or unsubstituted C₄₋₅ carbocyclyl, or substituted or unsubstituted C₅₋₆ carbocyclyl.

In certain embodiments, R⁹ is —CH₂N(R^{I2})₂ and at least one instance of R^{I2} is substituted or unsubstituted aryl, e.g., substituted or unsubstituted phenyl.

In certain embodiments, R⁹ is —CH₂N(R^{I2})₂ and at least one instance of R^{I2} is substituted or unsubstituted heteroaryl, e.g., substituted or unsubstituted 5-6 membered heteroaryl.

In certain embodiments, R⁹ is —CH₂N(R^{I2})₂ and at least one instance of R^{I2} is acyl.

In certain embodiments, R⁹ is —CH₂N(R^{I2})₂ and at least one instance of R^{I2} is substituted or unsubstituted heteroaliphatic.

In certain embodiments, R⁹ is —CH₂N(R^{I2})₂ and at least one instance of R^{I2} is substituted or unsubstituted heteroarylalkyl.

In certain embodiments, R⁹ is —CH₂N(R^{I2})₂ and at least one instance of R^{I2} is substituted or unsubstituted phosphono.

In certain embodiments, R⁹ is —CH₂N(R^{I2})₂ and at least one instance of R^{I2} is or -L-T. In certain embodiments, R⁹ is —CH₂N(R^{I2})₂ and one instance of R^{I2} is -L-A-B.

In certain embodiments, R⁹ is —CH₂N(R^{I2})₂ and at least one instance of R^{I2} is -L-T. In certain embodiments, R⁹ is —CH₂N(R^{I2})₂ and one instance of R^{I2} is -L-T.

In certain embodiments, one instance of R^{I2} is -L-A-B. In certain embodiments, one instance of R^{I2} is -L-T.

In certain embodiments of Formula (I-d) and (II-d), R^{I2} is hydrogen, carbohydrate, protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted phosphono; substituted or unsubstituted aryl; or substituted or unsubstituted heteroaryl.

In certain embodiments of Formula (I-d) and (II-d), R^{I2} is hydrogen; oxygen protecting group; carbohydrate; substituted or unsubstituted alkyl; substituted or unsubstituted alkenyl; substituted or unsubstituted alkynyl; substituted or unsubstituted phosphono; or substituted or unsubstituted heteroarylalkyl.

In certain embodiments of Formula (I-d) and (II-d), R^{I2} is hydrogen, oxygen protecting group, substituted or unsubstituted alkyl, substituted or unsubstituted alkenyl; substituted or unsubstituted alkynyl; or substituted or unsubstituted phosphono.

In certain embodiments of Formula (I-d) and (II-d), R^{I2} is hydrogen, protecting group, substituted or unsubstituted C₁₋₆ alkyl, substituted or unsubstituted C₁₋₆ alkenyl, substituted or unsubstituted C₁₋₆ alkynyl, or substituted or unsubstituted phosphono.

In certain embodiments of Formula (I-d) and (II-d), R^{I2} is hydrogen; benzyl; n-propyl; 2-propenyl; 2-propynyl; or —P(═O)(OH)₂.

In certain embodiments of Formula (I-d) and (II-d), R^{I2} is substituted or unsubstituted heteroarylalkyl.

In certain embodiments of Formula (I-d) and (II-d), R^{I2} is a carbohydrate. In certain embodiments of Formula (I-d and (II-d)), R^{I2} is:

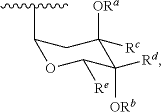

wherein R^{a} and R^{b} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; or acyl; and R^{c}, R^{d}, and R^{e} are independently hydrogen; cyclic or acyclic, substituted or unsubstituted, branched or unbranched aliphatic; cyclic or acyclic, substituted or unsubstituted, branched or unbranched heteroaliphatic. In certain embodiments, R^{a} and R^{b} are independently hydrogen; carbohydrate; oxygen protecting group; or acyl. In certain embodiments, R^{c}, R^{d}, and R^{e} are independently hydrogen; substituted or unsubstituted alkyl; or acyl. In certain embodiments, R^{c}, R^{d}, and R^{e} are independently hydrogen; substituted or unsubstituted C₁₋₆ alkyl; or acyl.

In certain embodiments of Formula (I-d) and (II-d), R^{I2} is:

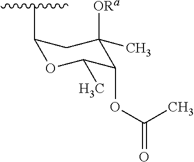

wherein R^{a} is hydrogen; carbohydrate; or oxygen protecting group.

In certain embodiments of Formula (I-d) and (II-d), R^{I2} is:

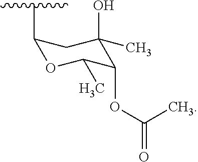 [SMILES: *C1CC(C)(O)C(OC(C)=O)C(C)O1]

In certain embodiments of Formula (I-d) and (II-d), R^{I2} is

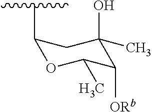

wherein R^{b} is hydrogen; carbohydrate; or oxygen protecting group.

In certain embodiments of Formula (I-d) and (II-d), R^{I2} is

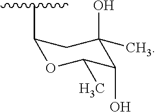 [SMILES: *C1CC(C)(O)C(O)C(C)O1]

In certain embodiments of Formula (I-d) and (II-d), R^{I2} is

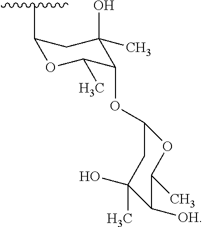 [SMILES: *C1CC(C)(O)C(OC2CC(C)(O)C(O)C(C)O2)C(C)O1]

In certain embodiments of Formula (I-d) and (II-d), R^{I2} is

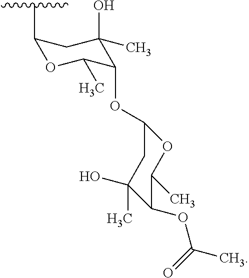 [SMILES: *C1CC(C)(O)C(OC2CC(C)(O)C(OC(C)=O)C(C)O2)C(C)O1]

In certain embodiments of Formula (I-d) and (II-d), R^{I2} is:

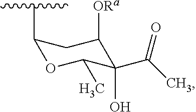

wherein R^{a} is hydrogen; carbohydrate; or oxygen protecting group.

In certain embodiments of Formula (I-d) and (II-d), R^{I2} is:

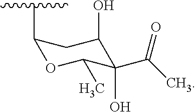 [SMILES: *C1CC(O)C(O)(C(C)=O)C(C)O1]

In certain embodiments of Formula (I-d) and (II-d), R^{I2} is:

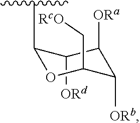

wherein R^{a}, R^{b}, R^{e}, and R^{d} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl. In certain embodiments R^{a}, R^{b}, R^{c}, and R^{d} are independently hydrogen; carbohydrate; or oxygen protecting group.

In certain embodiments of Formula (I-d) and (II-d), R^{I2} is:

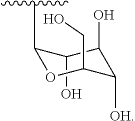 [SMILES: *C1OC(CO)C(O)C(O)C1O]

In certain embodiments of Formula (I-e) and (II-e), R^{I1} is hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂.

In certain embodiments of Formula (I-e) and (II-e), R^{I1} is azido; —N(R^{I2})₂; —SR^{I2}; or halogen.

In certain embodiments of Formula (I-e) and (II-e), each occurrence of R^{I2} is independently hydrogen; protecting group; cyclic or acyclic, substituted or unsubstituted, branched or unbranched aliphatic; or acyl; or two R^{I2} groups are optionally joined to form a substituted or unsubstituted heterocyclyl or substituted or unsubstituted heteroaryl ring.

In certain embodiments of Formula (I-e) and (II-e), R^{I1} is azido; —NH₂; —SH; S(C═O)CH₃; chloro; bromo; or iodo.

### Groups L, A, and T

As generally defined above for compounds of Formula (I) and (II), L is a linker.

In certain embodiments, L is a bond; substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted heteroalkynylene; substituted or unsubstituted heterocyclylene; substituted or unsubstituted carbocyclylene; substituted or unsubstituted arylene; substituted or unsubstituted heteroarylene; peptidyl groups; dipeptidyl groups; polypeptidyl groups; or combination thereof.

In certain embodiments, L is of formula:

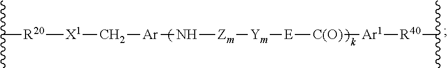

R²⁰ is substituted or unsubstituted alkylene; or substituted or unsubstituted heteroalkylene;
X¹ is

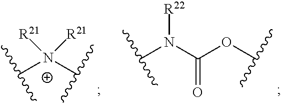

heterocyclylene; or heteroarylene;
R²¹ is independently substituted or unsubstituted alkyl; or substituted or unsubstituted carbocyclyl; or two R²¹ groups are joined to form an optionally substituted heterocyclyl ring;
R²² is hydrogen; substituted or unsubstituted alkyl; substituted or unsubstituted carbocyclyl; or

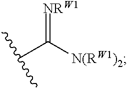

Ar is substituted or unsubstituted arylene;
each occurrence of Z is independently an amino acid;
each occurrence of Y is independently an amino acid;
E is a bond or an amino acid;
m is independently 1, 2, or 3;
k is 0 or 1;
Ar¹ is a bond or substituted or unsubstituted heteroarylene; and
R⁴⁰ is substituted or unsubstituted alkylene; or substituted or unsubstituted heteroalkylene.

In certain embodiments, R²⁰ is substituted or unsubstituted alkylene. In certain embodiments, R²⁰ is unsubstituted alkylene. In certain embodiments, R²⁰ is unsubstituted C₁₆ alkylene. In certain embodiments, R²⁰ is unsubstituted C₁₋₅ alkylene. In certain embodiments, R²⁰ is unsubstituted C₁₋₄ alkylene. In certain embodiments, R²⁰ is unsubstituted C₂₋₆ alkylene. In certain embodiments, R²⁰ is unsubstituted C₂₋₅ alkylene. In certain embodiments, R²⁰ is unsubstituted C₂₋₄ alkylene. In certain embodiments, R²⁰ is unsubstituted C₂₋₃ alkylene. In certain embodiments, R²⁰ is unsubstituted methylene. In certain embodiments, R²⁰ is unsubstituted ethylene. In certain embodiments, R²⁰ is unsubstituted propylene. In certain embodiments, R²⁰ is unsubstituted butylene. In certain embodiments, R²⁰ is unsubstituted pentylene. In certain embodiments, R²⁰ is unsubstituted hexylene.

In certain embodiments, X¹ is

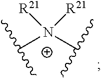

and R²¹ is independently substituted or unsubstituted alkyl; or substituted or unsubstituted carbocyclyl; or two R²¹ groups are joined to form an optionally substituted heterocyclyl ring. In certain embodiments, X¹ is

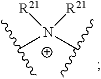

and R²¹ is independently substituted or unsubstituted alkyl; or substituted or unsubstituted carbocyclyl. In certain embodiments, X¹ is

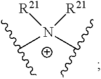

and R²¹ is independently substituted or unsubstituted alkyl. In certain embodiments, X¹ is

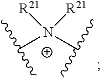

and R²¹ is independently unsubstituted alkyl. In certain embodiments, X¹ is

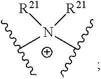

and R²¹ is independently unsubstituted C₁₋₆ alkyl. In certain embodiments, X¹ is

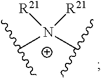

and R²¹ is independently unsubstituted C₁₋₄ alkyl. In certain embodiments, X¹ is

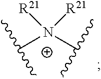

and R²¹ is independently unsubstituted C₁₋₃ alkyl. In certain embodiments, X¹ is

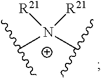

and R²¹ is independently unsubstituted C₁₋₂ alkyl. In certain embodiments, X¹ is

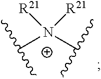

and two R²¹ groups are joined to form an optionally substituted heterocyclyl ring. In certain embodiments, X¹ is

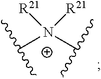

and two R²¹ groups are optionally joined to form an optionally substituted 5-6 membered heterocyclyl ring. In certain embodiments, X¹ is

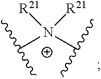

and two R²¹ groups are joined to form an optionally substituted 6 membered heterocyclyl ring. In certain embodiments, X¹ is

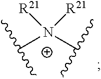

and two R²¹ groups are joined to form an unsubstituted 6 membered heterocyclyl ring. In certain embodiments, X¹ is

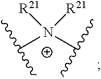

and two R²¹ groups are joined to form an optionally substituted pyrrolidine, piperidine, piperazine, thiomorpholine 1,1-dioxide, or morpholine. In certain embodiments, X¹ is

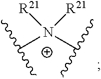

and two R²¹ groups are joined to form an optionally substituted morpholine. In certain embodiments, X¹ is

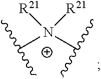

and two R²¹ groups are joined to form an unsubstituted morpholine.

In certain embodiments, X¹ is

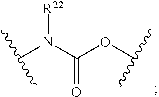

and R²² is hydrogen; substituted or unsubstituted carbocyclyl; substituted or unsubstituted alkyl; or

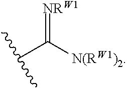

In certain embodiments, X¹ is

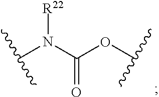

and R²² is hydrogen; substituted or unsubstituted carbocyclyl, or substituted or unsubstituted alkyl. In certain embodiments, X¹ is

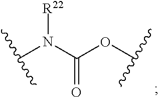

and R²² is hydrogen; or substituted or unsubstituted alkyl. In certain embodiments, X¹ is

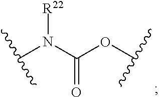

and R²² is hydrogen; or substituted or unsubstituted C₁₋₆ alkyl. In certain embodiments, X¹ is

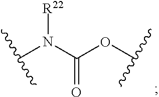

and R²² is hydrogen; or substituted or unsubstituted C₁₋₂ alkyl. In certain embodiments, X¹ is

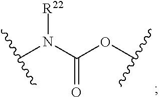

and R²² is hydrogen; or substituted or unsubstituted C₁₋₂ alkyl. In certain embodiments, X¹ is

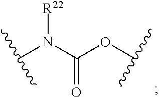

and R²² is hydrogen; or substituted C₁₋₂ alkyl. In certain embodiments, X¹ is

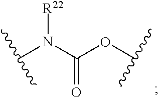

and R²² is hydrogen; or unsubstituted C₁₋₂ alkyl. In certain embodiments, X¹ is

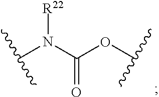

and R²² is hydrogen. In certain embodiments, X¹ is

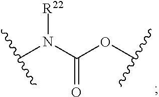

and R²² is substituted or unsubstituted C₁₋₂ alkyl. In certain embodiments, X¹ is

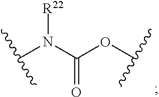

and R²² is substituted C₁₋₂ alkyl (e.g., haloalkyl). In certain embodiments, X¹ is

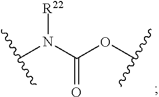

and R²² is unsubstituted C₁₋₂ alkyl. In certain embodiments, X¹ is

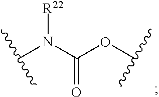

and R²² is unsubstituted carbocyclyl (e.g., C₃₋₆ cycloalkyl). In certain embodiments, X¹ is

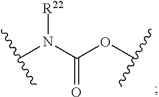

### and R²² is

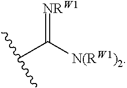

In certain embodiments, X¹ is

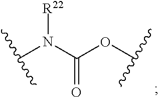

### and R²² is

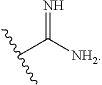 [SMILES: *C(=N)N]

In certain embodiments, X¹ is

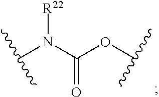

### and R²² is

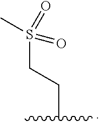 [SMILES: *CCS(C)(=O)=O]

In certain embodiments, X¹ is

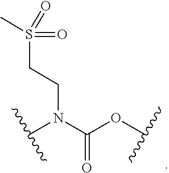 [SMILES: *OC(=O)N(*)CCS(C)(=O)=O]

In certain embodiments, X¹ is heterocyclylene or heteroarylene. In certain embodiments, X¹ is heterocyclylene. In certain embodiments, X¹ is a 6-membered heterocyclylene. In certain embodiments, X¹ is a piperazinylene. In certain embodiments, X¹ is heteroarylene. In certain embodiments, X¹ is a 5-membered heteroarylene. In certain embodiments, X¹ is an imidazolylene.

In certain embodiments, Ar is substituted or unsubstituted arylene. In certain embodiments, Ar is substituted or unsubstituted phenylene. In certain embodiments, Ar is unsubstituted phenylene. In certain embodiments, Ar is substituted phenylene. In certain embodiments, Ar is phenylene substituted with —OR^{a}, wherein R^{a} is a substituted or unsubstituted heterocycle. In certain embodiments, Ar is phenylene substituted with —OR^{a}, wherein R^{a} is a substituted heterocycle. In certain embodiments, R^{a} is a sugar moiety. In certain embodiments, Ar is

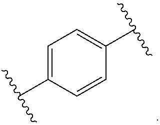 [SMILES: *c1ccc(*)cc1]

In certain embodiments, Ar is

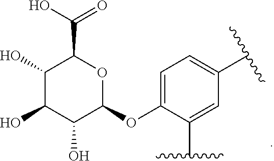 [SMILES: *c1ccc(O[C@@H]2O[C@H](C(=O)O)[C@@H](O)[C@H](O)[C@H]2O)c(*)c1]

In certain embodiments, each occurrence of Z is independently an amino acid. In certain embodiments, Z is independently a naturally occurring amino acid. In certain embodiments, Z is independently a non-natural amino acid. In certain embodiments, Z is independently alanine, lysine, arginine, histidine, ornithine, or citrulline. In certain embodiments, Z is alanine, lysine, or citrulline. In certain embodiments, Z is alanine or citrulline. In certain embodiments, Z is citrulline. In certain embodiments, Z is alanine.

In certain embodiments, each occurrence of Y is independently an amino acid. In certain embodiments, Y is independently a naturally occurring amino acid. In certain embodiments, Y is independently a non-natural amino acid. In certain embodiments, Y is alanine, valine, leucine, isoleucine, methionine, phenylalanine, or tryptophan. In certain embodiments, Y is valine or phenylalanine. In certain embodiments, Y is valine.

In certain embodiments, each occurrence of m is 1, 2, or 3. In certain embodiments, each occurrence of m is 1 or 2. In certain embodiments, each occurrence of m is 1.

In certain embodiments, —Zₘ—Yₘ— is -citrulline-valine-. In certain embodiments, —Zₘ—Yₘ— is -alanine-valine-.

In certain embodiments, E is a bond or an amino acid. In certain embodiments, E is a bond or a naturally occurring amino acid. In certain embodiments, Z is independently a non-natural amino acid. In certain embodiments, E is a bond or a substituted naturally occurring amino acid. In certain embodiments, E is a bond. In certain embodiments, E is a naturally occurring amino acid. In certain embodiments, E is a substituted naturally occurring amino acid. In certain embodiments, E is a substituted lysine.

In certain embodiments, E is of the formula:

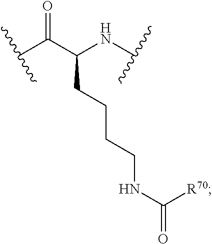

wherein R⁷⁰ is substituted or unsubstituted alkyl, or substituted or unsubstituted heteroalkyl.

In certain embodiments, E is of the formula:

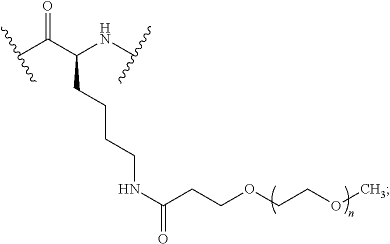

wherein n is 2-30.

In certain embodiments, E is of the formula:

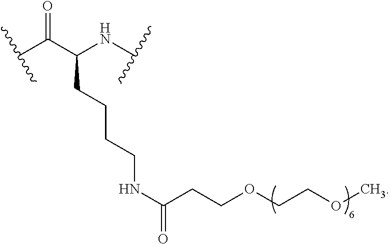

In certain embodiments, k is 0 or 1. In certain embodiments, k is 0. In certain embodiments, k is 1.

In certain embodiments, Ar¹ is a bond or substituted or unsubstituted heteroarylene. In certain embodiments, Ar¹ is a bond. In certain embodiments, Ar¹ is substituted or unsubstituted heteroarylene. In certain embodiments, Ar¹ is unsubstituted heteroarylene. In certain embodiments, Ar¹ is unsubstituted 5-6-membered heteroarylene. In certain embodiments, Ar¹ is unsubstituted 5-membered heteroarylene. In certain embodiments, Ar¹ is tetrazolene, triazolene, or imidazolene. In certain embodiments, Ar¹ is triazolene. In certain embodiments, Ar¹ is

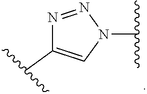 [SMILES: *c1cn(*)nn1]

In certain embodiments, k is 1; and Ar¹ is a bond. In certain embodiments, k is 1; and Ar¹ is

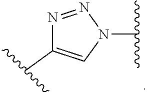 [SMILES: *c1cn(*)nn1]

In certain embodiments, R⁴⁰ is substituted or unsubstituted alkylene; or substituted or unsubstituted heteroalkylene. In certain embodiments, R⁴⁰ is substituted or unsubstituted C₁₋₆ alkylene; or substituted or unsubstituted C₁₋₄₀ heteroalkylene. In certain embodiments, R⁴⁰ is substituted or unsubstituted alkylene. In certain embodiments, R⁴⁰ is substituted or unsubstituted C₁₋₆ alkylene. In certain embodiments, R⁴⁰ is substituted C₁₋₆ alkylene. In certain embodiments, R⁴⁰ is unsubstituted C₁₋₆ alkylene. In certain embodiments, R⁴⁰ is substituted or unsubstituted heteroalkylene. In certain embodiments, R⁴⁰ is substituted or unsubstituted C₁₋₄₀ heteroalkylene. In certain embodiments, R⁴⁰ is substituted C₁₋₄₀ heteroalkylene. In certain embodiments, R⁴⁰ is unsubstituted C₁₋₄₀ heteroalkylene. In certain embodiments, R⁴⁰ is

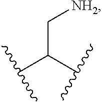 [SMILES: *C(*)CN]

C₁₋₆ unsubstituted alkylene,

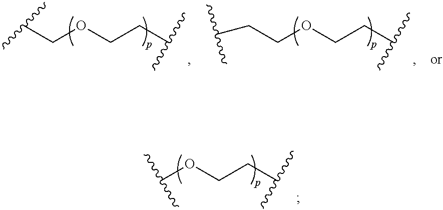

wherein p is 1-8. In certain embodiments, R⁴⁰ is

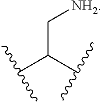 [SMILES: *C(*)CN]

In certain embodiments, R⁴⁰ is

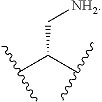

In certain embodiments, R⁴⁰ is

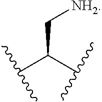

In certain embodiments, R⁴⁰ is C₁₋₆ unsubstituted alkylene. In certain embodiments, R⁴⁰ is

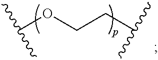

wherein p is 1-8. In certain embodiments, R⁴⁰ is

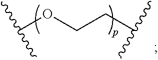

wherein p is 2-8. In certain embodiments, R⁴⁰ is

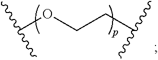

wherein p is 2, 4, or 8. In certain embodiments, R⁴⁰ is

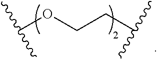

In certain embodiments, R⁴⁰ is

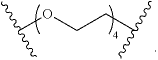

In certain embodiments, R⁴⁰ is

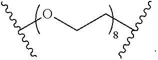

In certain embodiments, R⁴⁰ is

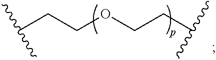

wherein p is 1-8. In certain embodiments, R⁴⁰ is

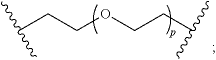

wherein p is 2-8. In certain embodiments, R⁴⁰ is

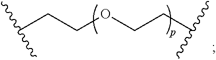

wherein p is 2, 4, or 8. In certain embodiments, R⁴⁰ is

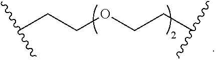

In certain embodiments, R⁴⁰ is

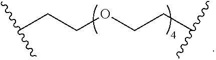

In certain embodiments, R⁴⁰ is

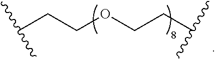

In certain embodiments, R⁴⁰ is

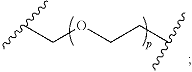

wherein p is 1-8. In certain embodiments, R⁴⁰ is

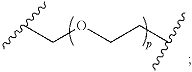

wherein p is 2-8. In certain embodiments, R⁴⁰ is

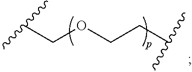

wherein p is 2, 4, or 8. In certain embodiments, R⁴⁰ is

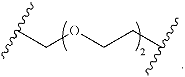

In certain embodiments, R⁴⁰ is

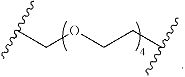

In certain embodiments, R⁴⁰ is

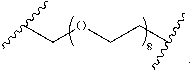

In certain embodiments, A is a group of the formula:

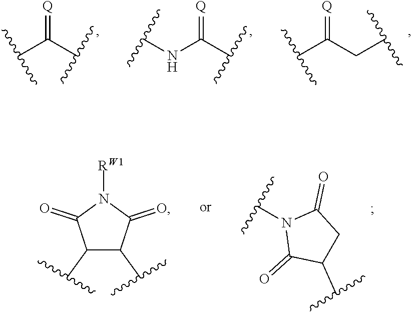

and
Q is —S—, or —O—; and R^{W1} is hydrogen, substituted or unsubstituted alkyl; or a nitrogen protecting group.

In certain embodiments, A is a group of the formula:

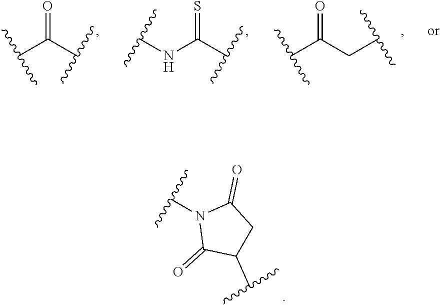

In certain embodiments, A is a group of the formula:

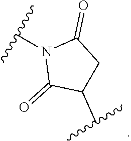 [SMILES: *C1CC(=O)N(*)C1=O]

In certain embodiments, L is a group of Formula (L-1):

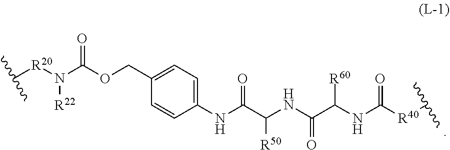

In certain embodiments of Formula (L-1), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; R⁵⁰ is the sidechain of alanine, lysine, arginine, histidine, ornithine, or citrulline; and R⁶⁰ is the sidechain of alanine, valine, leucine, isoleucine, methionine, phenylalanine, or tryptophan.

In certain embodiments, L is a group of Formula (L-1-a):

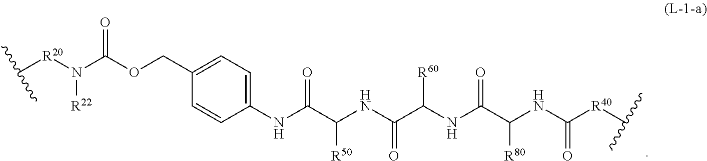

In certain embodiments of Formula (L-1-a), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; R⁵⁰ is the sidechain of alanine, lysine, arginine, histidine, ornithine, or citrulline; R⁶⁰ is the sidechain of alanine, valine, leucine, isoleucine, methionine, phenylalanine, or tryptophan; and R⁸⁰ is a substituted sidechain of a lysine, arginine, histidine, ornithine, or citrulline.

In certain embodiments, L is a group of Formula (L-1-b):

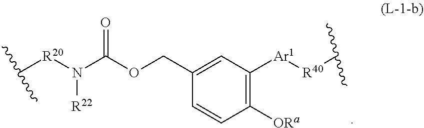

In certain embodiments of Formula (L-1-b), R²⁰ is substituted or unsubstituted C₁₋₆ alkylene; R²² is hydrogen, or substituted or unsubstituted C₁₋₆ alkyl; R⁴⁰ is substituted or unsubstituted heterocycle; Ar¹ is substituted or unsubstituted heteroarylene; R^{a} is a substituted heterocycle; and R⁴⁰ is substituted or unsubstituted C₁₋₆ alkylene, or substituted or unsubstituted C₁₋₄₀ heteroalkylene.

In certain embodiments, -L-A- is a group of Formula (L-2):

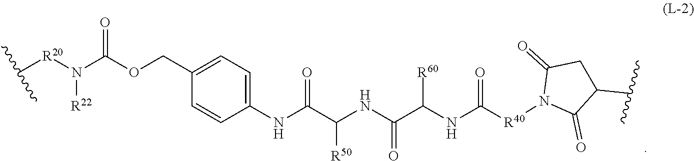

In certain embodiments of Formula (L-2), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; R⁵⁰ is the sidechain of lysine, arginine, histidine, ornithine, or citrulline; and R⁶⁰ is the sidechain of alanine, valine, leucine, isoleucine, methionine, phenylalanine, or tryptophan.

In certain embodiments of Formula (L-2), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; R⁵⁰ is the sidechain of alanine, lysine, arginine, histidine, ornithine, or citrulline; and R⁶⁰ is the sidechain of alanine, valine, leucine, isoleucine, methionine, phenylalanine, or tryptophan.

In certain embodiments, -L-A- is a group of Formula (L-2-a):

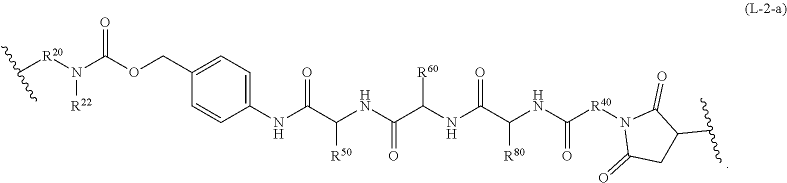

In certain embodiments of Formula (L-2-a), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; R⁵⁰ is the sidechain of alanine, lysine, arginine, histidine, ornithine, or citrulline; R⁶⁰ is the sidechain of alanine, valine, leucine, isoleucine, methionine, phenylalanine, or tryptophan; and R⁸⁰ is a substituted sidechain of a lysine, arginine, histidine, ornithine, or citrulline.

In certain embodiments, -L-A- is a group of Formula (L-2-b):

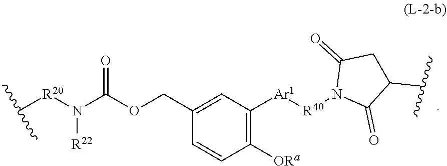

In certain embodiments of Formula (L-2-b), R²⁰ is substituted or unsubstituted C₁₋₆ alkylene; R²² is hydrogen, or substituted or unsubstituted C₁₋₆ alkyl; R⁴⁰ is substituted or unsubstituted heterocycle; R^{a} is a substituted heterocycle; Ar¹ is substituted or unsubstituted heteroarylene; and R⁴⁰ is substituted or unsubstituted C₁₋₆ alkylene, or substituted or unsubstituted C₁₋₄₀ heteroalkylene.

In certain embodiments, -L-A- is a group of Formula (L-3):

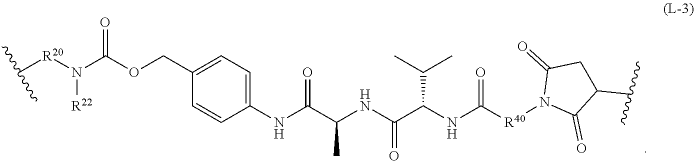

In certain embodiments of Formula (L-3), R²⁰ is substituted or unsubstituted C₁₋₆ alkylene; R²² is hydrogen, or substituted or unsubstituted C₁₋₆ alkyl; and R⁴⁰ is substituted or unsubstituted C₁₋₆ alkylene, or substituted or unsubstituted C₁₋₄₀ heteroalkylene.

In certain embodiments, -L-A- is a group of Formula (L-3-a):

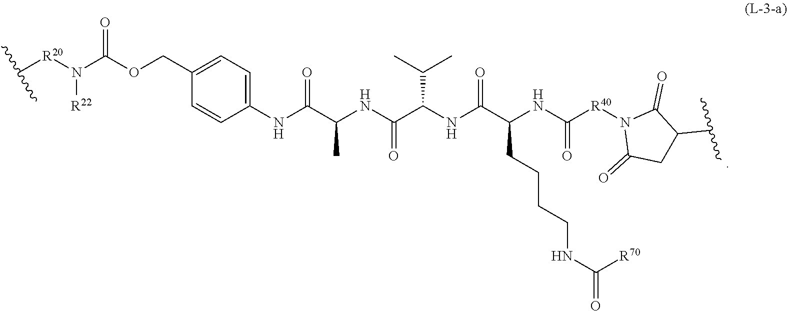

In certain embodiments of Formula (L-3-a), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; and R⁷⁰ is substituted or unsubstituted heteroalkyl.

In certain embodiments, -L-A- is a group of Formula (L-3-b):

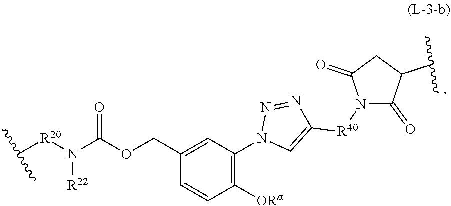

In certain embodiments of Formula (L-3-b), R²⁰ is substituted or unsubstituted C₁₋₆ alkylene; R²² is hydrogen, or substituted or unsubstituted C₁₋₆ alkyl; R^{a} is substituted or unsubstituted heterocycle; and R⁴⁰ is substituted or unsubstituted C₁₋₆ alkylene, or substituted or unsubstituted C₁₋₄₀ heteroalkylene.

In certain embodiments, -L-A- is a group of Formula (L-4):

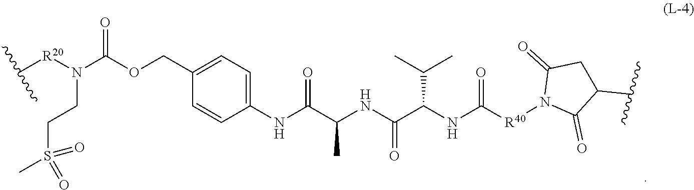

In certain embodiments of Formula (L-4), R²⁰ is substituted or unsubstituted C₁₋₆ alkylene; and R⁴⁰ is substituted or unsubstituted C₁₋₆ alkylene, or substituted or unsubstituted C₁₋₄₀ heteroalkylene.

In certain embodiments, -L-A- is a group of Formula (L-4-a):

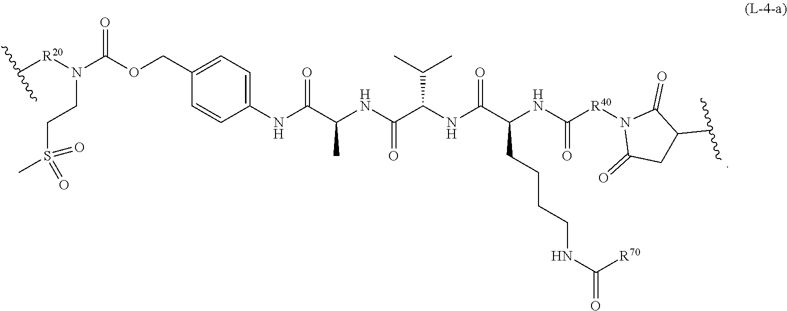

In certain embodiments of Formula (L-4-a), R²⁰ is substituted or unsubstituted C₁₋₆ alkylene; R⁴⁰ is substituted or unsubstituted C₁₋₆ alkylene, or substituted or unsubstituted C₁₋₄₀ heteroalkylene; and R⁷⁰ is substituted or unsubstituted heteroalkyl.

In certain embodiments, -L-A- is a group of Formula (L-4-b):

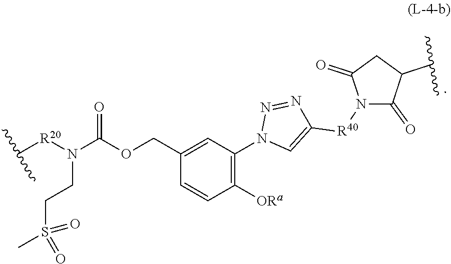

In certain embodiments of Formula (L-4-b), R²⁰ is substituted or unsubstituted C₁₋₆ alkylene; R^{a} is substituted or unsubstituted heterocycle; and R⁴⁰ is substituted or unsubstituted C₁₋₆ alkylene, or substituted or unsubstituted C₁₋₄₀ heteroalkylene.

In certain embodiments, -L-A- is a group of Formula (L-5):

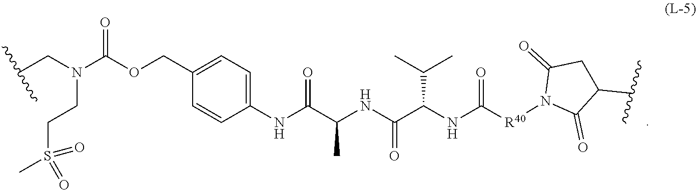

In certain embodiments of Formula (L-5), R⁴⁰ is substituted or unsubstituted C₁₋₆ alkylene, or substituted or unsubstituted C₁₋₄₀ heteroalkylene. In certain embodiments of Formula (L-5), R⁴⁰ is substituted or unsubstituted C₁₋₆ alkylene. In certain embodiments of Formula (L-5), R⁴⁰ is unsubstituted C₁₋₆ alkylene.

In certain embodiments, -L-A- is a group of Formula (L-5-a):

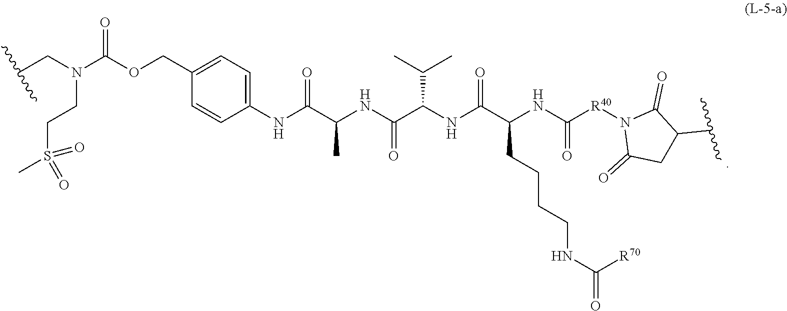

In certain embodiments of Formula (L-5-a), R⁴⁰ is substituted or unsubstituted C₁₋₆ alkylene, or substituted or unsubstituted C₁₋₄₀ heteroalkylene; and R⁷⁰ is substituted or unsubstituted heteroalkyl.

In certain embodiments, -L-A- is a group of Formula (L-5-a1):

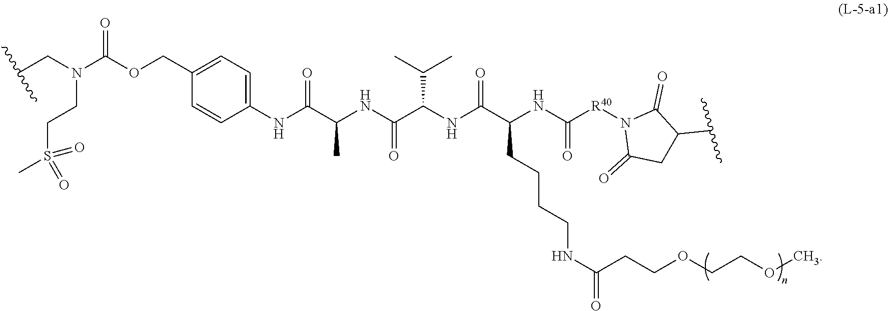

In certain embodiments of Formula (L-5-a1), R⁴⁰ is substituted or unsubstituted C₁₋₆ alkylene, or substituted or unsubstituted C₁₋₄₀ heteroalkylene; and n is 2-8.

In certain embodiments, -L-A- is a group of Formula (L-5-b):

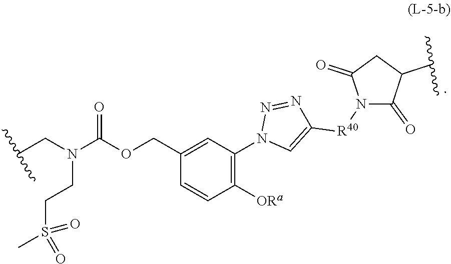

In certain embodiments of Formula (L-5-b), R^{a} is substituted or unsubstituted heterocycle; and R⁴⁰ is substituted or unsubstituted C₁₋₆ alkylene, or substituted or unsubstituted C₁₋₄₀ heteroalkylene.

In certain embodiments, -L-A- is a group of Formula (L-5-b1):

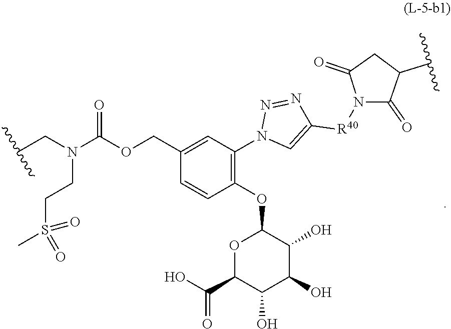

In certain embodiments of Formula (L-5-b), R⁴⁰ is substituted or unsubstituted C₁₋₆ alkylene, or substituted or unsubstituted C₁₋₄₀ heteroalkylene.

In certain embodiments, -L-A- is a group of Formula (L-6):

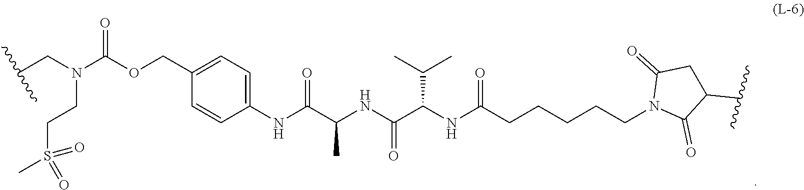 [SMILES: *CN(CCS(C)(=O)=O)C(=O)OCc1ccc(NC(=O)[C@H](C)NC(=O)[C@@H](NC(=O)CCCCCN2C(=O)CC(*)C2=O)C(C)C)cc1]

In certain embodiments, -L-A- is a group of Formula (L-6-a):

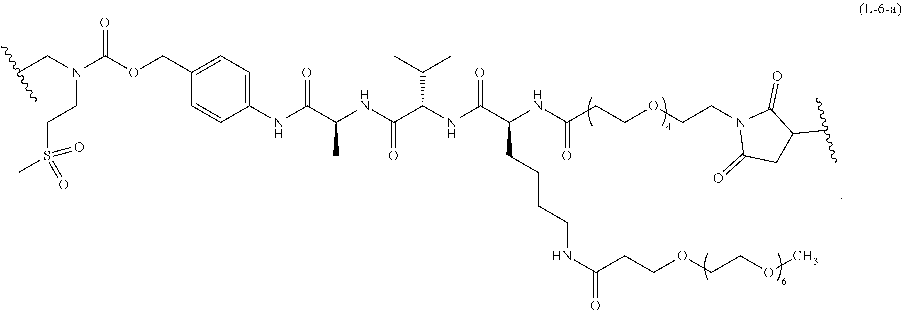

In certain embodiments, -L-A- is a group of Formula (L-6-b):

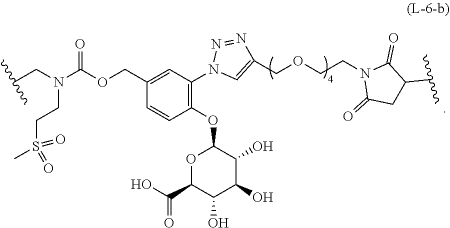

In certain embodiments, -L-A- is a group of Formula (L-6-c):

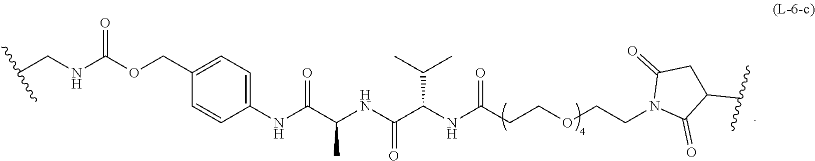

In certain embodiments, T is a group of the formula:
—N═C═S,

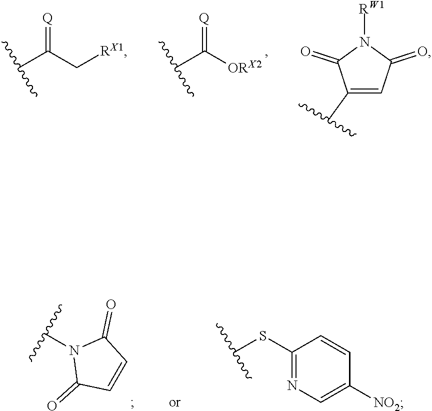

Q is —S—, or —O—; R^{X1} is halogen; R^{X2} is substituted or unsubstituted heterocyclyl; and R^{W1} is hydrogen, substituted or unsubstituted alkyl; or a nitrogen protecting group.

In certain embodiments, T is a group of the formula:
—N═C═S,

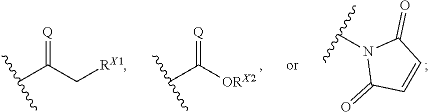

R^{X1} is halogen; and
R^{X2} is substituted or unsubstituted heterocyclyl.

In certain embodiments, T is a group of the formula:

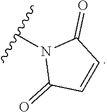 [SMILES: *N1C(=O)C=CC1=O]

In certain embodiments, T is a group of the formula:

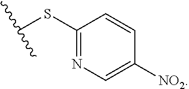 [SMILES: *Sc1ccc([N+](=O)[O-])cn1]

In certain embodiments, -L-T is a group of Formula (L^{T}-1):

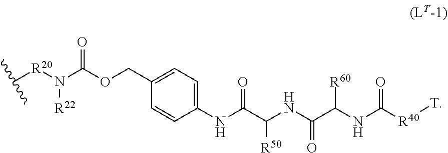

In certain embodiments of Formula (L^{T}-1), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; R⁵⁰ is the sidechain of alanine, lysine, arginine, histidine, ornithine, or citrulline; and R⁶⁰ is the sidechain of alanine, valine, leucine, isoleucine, methionine, phenylalanine, or tryptophan.

In certain embodiments, -L-T is a group of Formula (L^{T}-1-a):

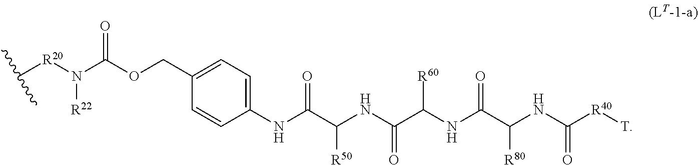

In certain embodiments of Formula (L^{T}-1-a), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; R⁵⁰ is the sidechain of alanine, lysine, arginine, histidine, ornithine, or citrulline; R⁶⁰ is the sidechain of alanine, valine, leucine, isoleucine, methionine, phenylalanine, or tryptophan; and R⁸⁰ is a substituted sidechain of a lysine, arginine, histidine, ornithine, or citrulline.

In certain embodiments, -L-T is a group of Formula (L^{T}-1-b):

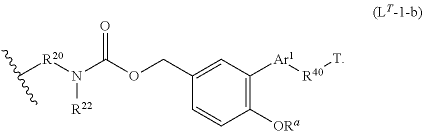

In certain embodiments of Formula (L^{T}-1-b), R²⁰ is substituted or unsubstituted C₁₋₆ alkylene; R²² is hydrogen, or substituted or unsubstituted C₁₋₆ alkyl; R⁴⁰ is substituted or unsubstituted heterocycle; Ar¹ is substituted or unsubstituted heteroarylene; R^{a} is a substituted heterocycle; and R⁴⁰ is substituted or unsubstituted C₁₋₆ alkylene, or substituted or unsubstituted C₁₋₄₀ heteroalkylene.

In certain embodiments, -L-T is a group of Formula (L^{T}-2):

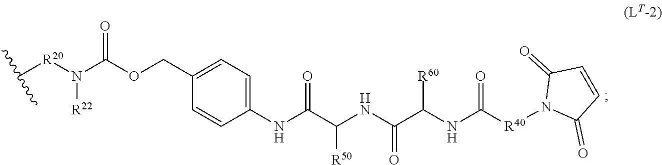

In certain embodiments of Formula (L^{T}-2), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; R⁵⁰ is the sidechain of alanine, lysine, arginine, histidine, ornithine, or citrulline; and R⁶⁰ is the sidechain of alanine, valine, leucine, isoleucine, methionine, phenylalanine, or tryptophan.

In certain embodiments, -L-T is a group of Formula (L^{T}-2-a):

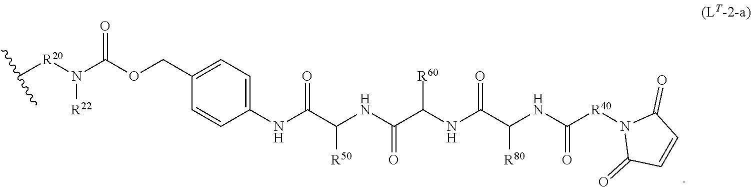

In certain embodiments of Formula (L^{T}-2-a), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; R⁵⁰ is the sidechain of alanine, lysine, arginine, histidine, ornithine, or citrulline; R⁶⁰ is the sidechain of alanine, valine, leucine, isoleucine, methionine, phenylalanine, or tryptophan; and R⁸⁰ is a substituted sidechain of a lysine, arginine, histidine, ornithine, or citrulline.

In certain embodiments, -L-T is a group of Formula (L-2-b):

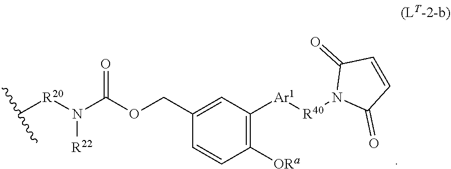

In certain embodiments of Formula (L^{T}-2-b), R²⁰ is substituted or unsubstituted C₁₋₆ alkylene; R²² is hydrogen, or substituted or unsubstituted C₁₋₆ alkyl; R⁴⁰ is substituted or unsubstituted heterocycle; R^{a} is a substituted heterocycle; Ar¹ is substituted or unsubstituted heteroarylene; and R⁴⁰ is substituted or unsubstituted C₁₋₆ alkylene, or substituted or unsubstituted C₁₋₄₀ heteroalkylene.

In certain embodiments, -L-T is a group of Formula (L^{T}-3):

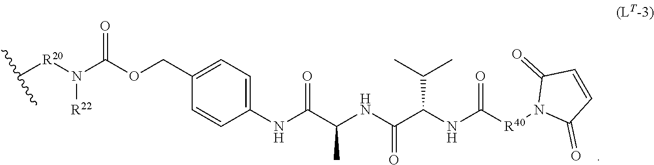

In certain embodiments of Formula (L^{T}-3), R²⁰ is substituted or unsubstituted C₁₋₆ alkylene; R²² is hydrogen, or substituted or unsubstituted C₁₋₆ alkyl; and R⁴⁰ is substituted or unsubstituted C₁₋₆ alkylene, or substituted or unsubstituted C₁₋₄₀ heteroalkylene.

In certain embodiments, -L-T is a group of Formula (L^{T}-3-a):

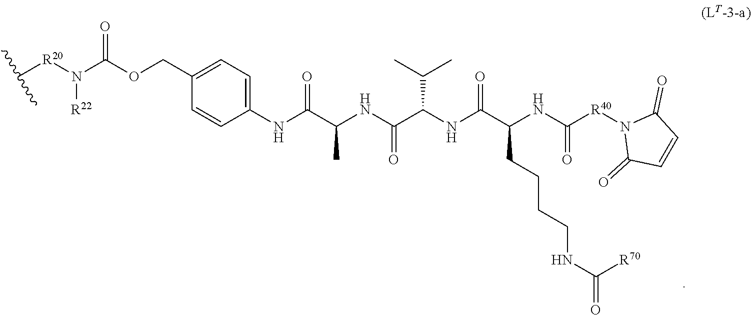

In certain embodiments of Formula (L^{T}-3-a), R²⁰ is substituted or unsubstituted C₁₋₆ alkylene; R²² is hydrogen, or substituted or unsubstituted C₁₋₆ alkyl; R⁴⁰ is substituted or unsubstituted C₁₋₆ alkylene, or substituted or unsubstituted C₁₋₄₀ heteroalkylene; and R⁷⁰ is substituted or unsubstituted heteroalkyl.

In certain embodiments, -L-T is a group of Formula (L^{T}-3-b):

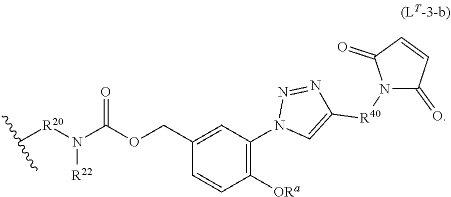

In certain embodiments of Formula (L^{T}-3-b), R²⁰ is substituted or unsubstituted C₁₋₆ alkylene; R²² is hydrogen, or substituted or unsubstituted C₁₋₆ alkyl; R^{a} is substituted or unsubstituted heterocycle; and R⁴⁰ is substituted or unsubstituted C₁₋₆ alkylene, or substituted or unsubstituted C₁₋₄₀ heteroalkylene.

In certain embodiments, -L¹-T is a group of Formula (L^{T}-4):

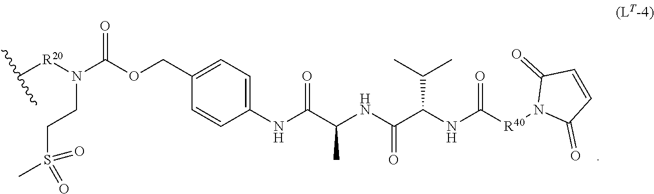

In certain embodiments of Formula (L^{T}-4), R²⁰ is substituted or unsubstituted C₁₋₆ alkylene; and R⁴⁰ is substituted or unsubstituted C₁₋₆ alkylene, or substituted or unsubstituted C₁₋₄₀ heteroalkylene. In certain embodiments of Formula (L^{T}-4), R²⁰ is substituted or unsubstituted C₁₋₆ alkylene; and R⁴⁰ is substituted or unsubstituted C₁₋₆ alkylene.

In certain embodiments, -L-T is a group of Formula (L^{T}-4-a):

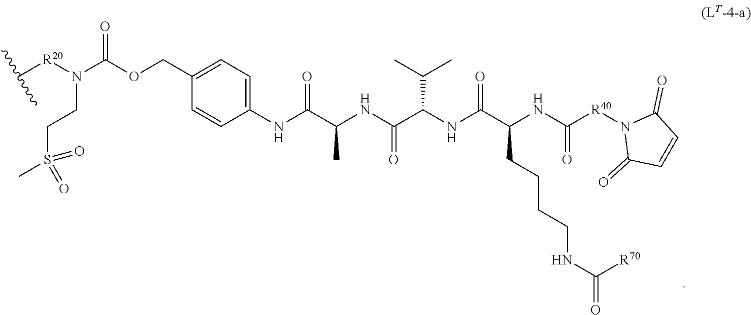

In certain embodiments of Formula (L^{T}-4-a), R²⁰ is substituted or unsubstituted C₁₋₆ alkylene; R⁴⁰ is substituted or unsubstituted C₁₋₆ alkylene, or substituted or unsubstituted C₁₋₄₀ heteroalkylene; and R⁷⁰ is substituted or unsubstituted heteroalkyl.

In certain embodiments, -L-T is a group of Formula (L^{T}-4-b):

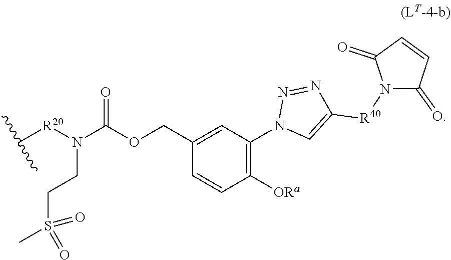

In certain embodiments of Formula (L^{T}-4-b), R²⁰ is substituted or unsubstituted C₁₋₆ alkylene; R^{a} is substituted or unsubstituted heterocycle; and R⁴⁰ is substituted or unsubstituted C₁₋₆ alkylene, or substituted or unsubstituted C₁₋₄₀ heteroalkylene.

In certain embodiments, -L-T is a group of Formula (L^{T}-5):

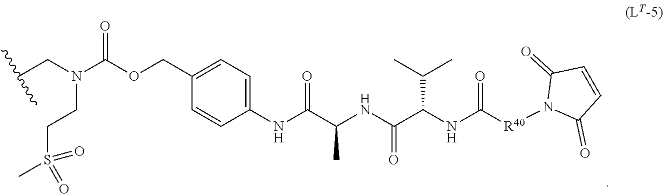

In certain embodiments of Formula (L^{T}-5), R⁴⁰ is substituted or unsubstituted C₁₋₆ alkylene. In certain embodiments of Formula (L^{T}-5), R⁴⁰ is unsubstituted C₁₋₆ alkylene.

In certain embodiments, -L-T is a group of Formula (L^{T}-5-a):

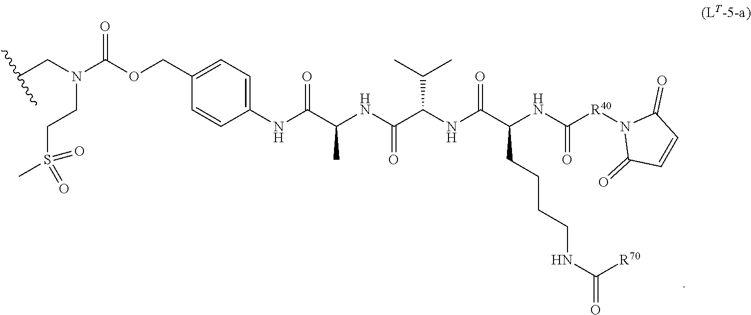

In certain embodiments of Formula (L^{T}-5-a), R⁴⁰ is substituted or unsubstituted C₁₋₆ alkylene, or substituted or unsubstituted C₁₋₄₀ heteroalkylene; and R⁷⁰ is substituted or unsubstituted heteroalkyl.

In certain embodiments, -L-T is a group of Formula (L^{T}-5-a1):

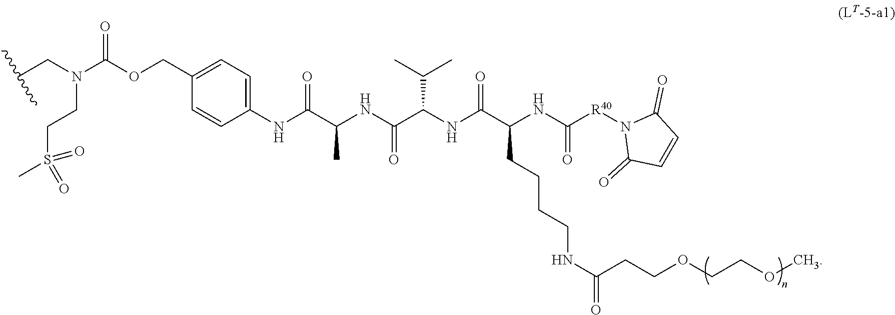

In certain embodiments of Formula (L^{T}-5-a1), R⁴⁰ is substituted or unsubstituted C₁₋₆ alkylene, or substituted or unsubstituted C₁₋₄₀ heteroalkylene; and n is 2-8.

In certain embodiments, -L-T is a group of Formula (L^{T}-5-b):

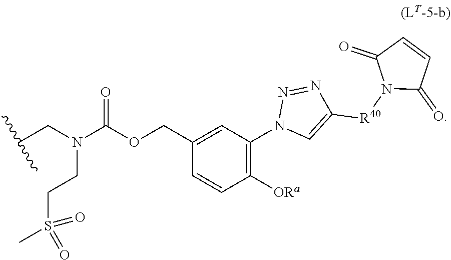

In certain embodiments of Formula (L^{T}-5-b), R^{a} is substituted or unsubstituted heterocycle; and R⁴⁰ is substituted or unsubstituted C₁₋₆ alkylene, or substituted or unsubstituted C₁₋₄₀ heteroalkylene.

In certain embodiments, -L-T is a group of Formula (L^{T}-5-b1):

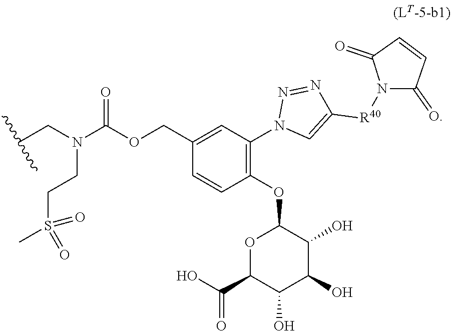

In certain embodiments of Formula (L^{T}-5-b), R⁴⁰ is substituted or unsubstituted C₁₋₆ alkylene, or substituted or unsubstituted C₁₋₄₀ heteroalkylene.

In certain embodiments, -L1-T is a group of Formula (L^{T}-6):

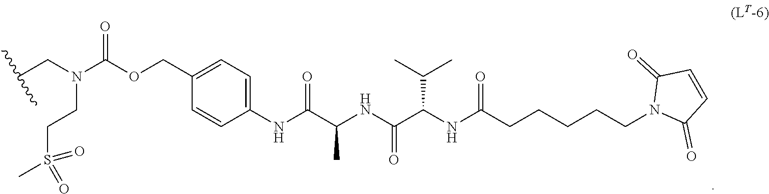 [SMILES: *CN(CCS(C)(=O)=O)C(=O)OCc1ccc(NC(=O)[C@H](C)NC(=O)[C@@H](NC(=O)CCCCCN2C(=O)C=CC2=O)C(C)C)cc1]

In certain embodiments, -L-T is a group of Formula (L^{T}-6-a):

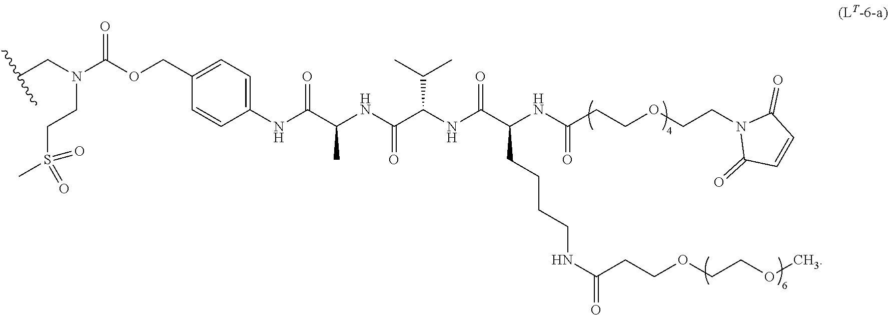

In certain embodiments, -L-T is a group of Formula (L^{T}-6-b):

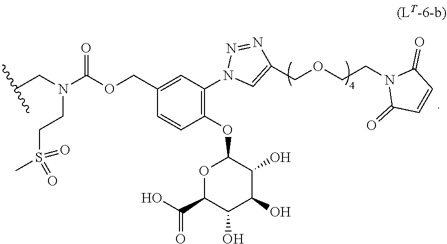

In certain embodiments, -L-T is a group of Formula (L^{T}-6-c):

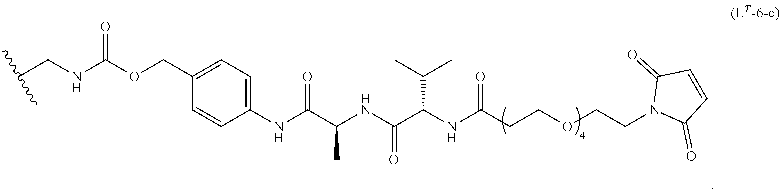

### Group R¹⁰

As generally defined herein, R¹⁰ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{B1}; —C(═O)R^{B2}; —CO₂R^{B2}; —CN; —SCN; —SR^{B1}; —SOR^{B1}; —SO₂R^{B2}; —NO₂; —N₃; —N(R^{B2})₂; —NR^{B2}C(═O)R^{B2}; —NR^{B2}C(═O)N(R^{B2})₂; —C(═O)OR^{B1}; —OC(═O)R^{B2}; —OC(═O)N(R^{B2})₂; —NR^{B2}C(═O)OR^{B1}; or —C(R^{B2})₃; wherein each occurrence of R^{B1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{B1} groups are optionally joined to form a substituted or unsubstituted heterocyclyl or substituted or unsubstituted heteroaryl ring; and wherein each occurrence of R^{B2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{B1}; —SR^{B1}; or —N(R^{B1})₂; or two R^{B2} groups are optionally joined to form a substituted or unsubstituted heterocyclyl or substituted or unsubstituted heteroaryl ring.

In certain embodiments, R¹⁰ is hydrogen.

### Group R^{I1}

As generally defined above for compounds of Formula (I) and (II), R^{I1} is hydrogen; substituted or unsubstituted alkyl; acyl; -L-A-B; or -L-T.

In certain embodiments, R^{I1} is L-A-B, wherein L, A, and B are as defined herein.

In certain embodiments, R^{I1} is -L-T, wherein L and T are as defined herein.

In certain embodiments, R^{I1} is of formula:

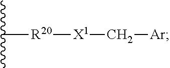

R²⁰ is substituted or unsubstituted alkylene; or substituted or unsubstituted heteroalkylene;
X¹ is

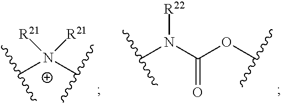

heterocyclylene; or heteroarylene;
R²¹ is independently substituted or unsubstituted alkyl; or substituted or unsubstituted carbocyclyl; or two R²¹ groups are joined to form an optionally substituted heterocyclyl ring;
R²² is hydrogen; substituted or unsubstituted alkyl; substituted or unsubstituted carbocyclyl; or

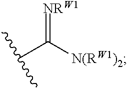

Ar is substituted or unsubstituted aryl.

### Group X

As generally defined above, X is a halogen. In certain embodiments, X is —Cl, —Br, or —I. In certain embodiments, X is —Br or —I. In certain embodiments, X is —Cl. In certain embodiments, X is —Br. In certain embodiments, X is —I.

### Additional Embodiments of Formula (I) and (II)

Various combinations of the above embodiments are further contemplated herein. One of skill in the art would appreciate that the various embodiments described herein may be combined in various ways and are contemplated by the inventors.

In certain embodiments of the compound of Formula (I) and (II), R¹ and R² are each hydrogen or together form ═O; and R¹⁰ and R³ are hydrogen; or R¹ and R¹⁰ are optionally taken together with the intervening carbon atoms to form an optionally substituted cyclic moiety; or R¹⁰ and R³ are optionally taken together with the intervening carbon atoms to form an optionally substituted cyclic moiety; or R¹ and R⁴ are optionally taken together with the intervening carbon atoms to form an optionally substituted cyclic moiety; or R⁴ and R⁷ are optionally taken together with the intervening carbon atoms to form an optionally substituted cyclic moiety; or R⁶ and R⁷ are optionally taken together with the intervening carbon atoms to form an optionally substituted cyclic moiety.

In certain embodiments, the compound of Formula (I) is:

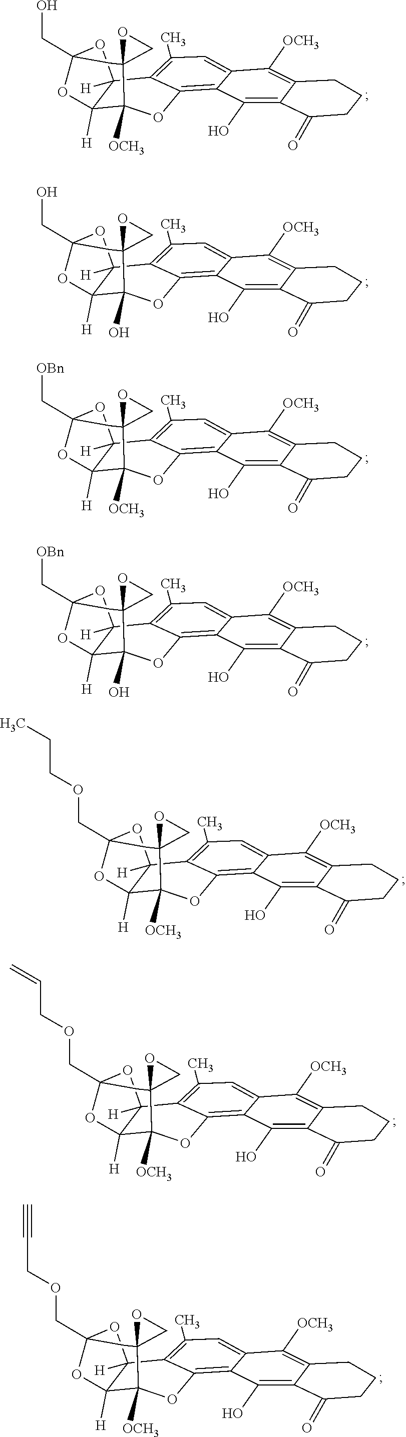

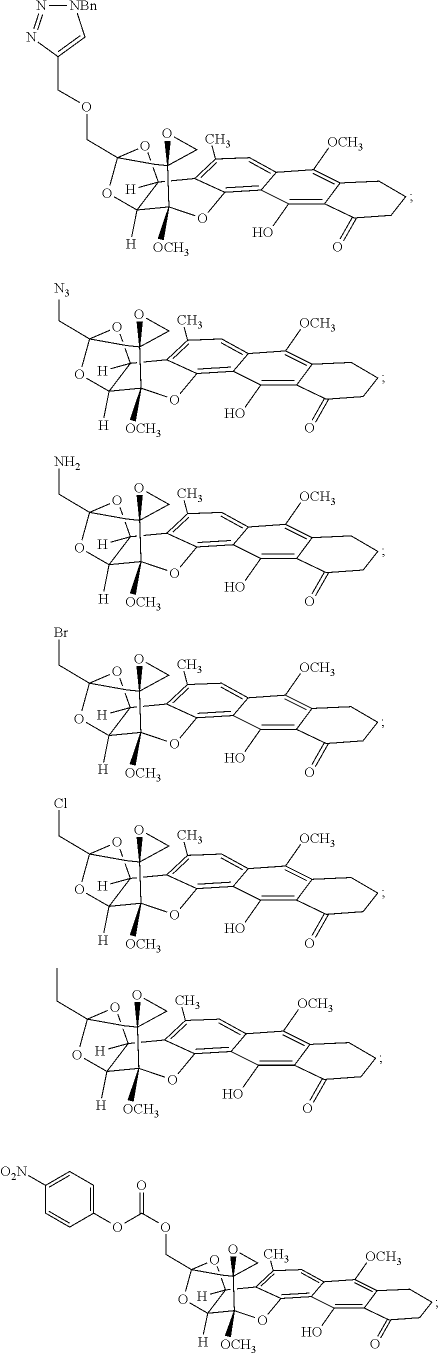

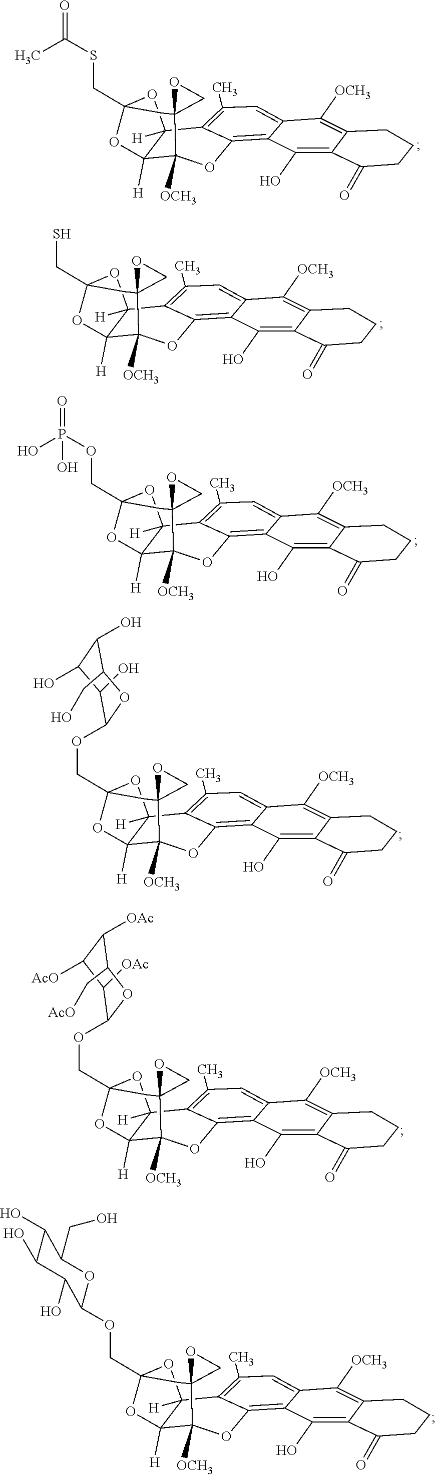

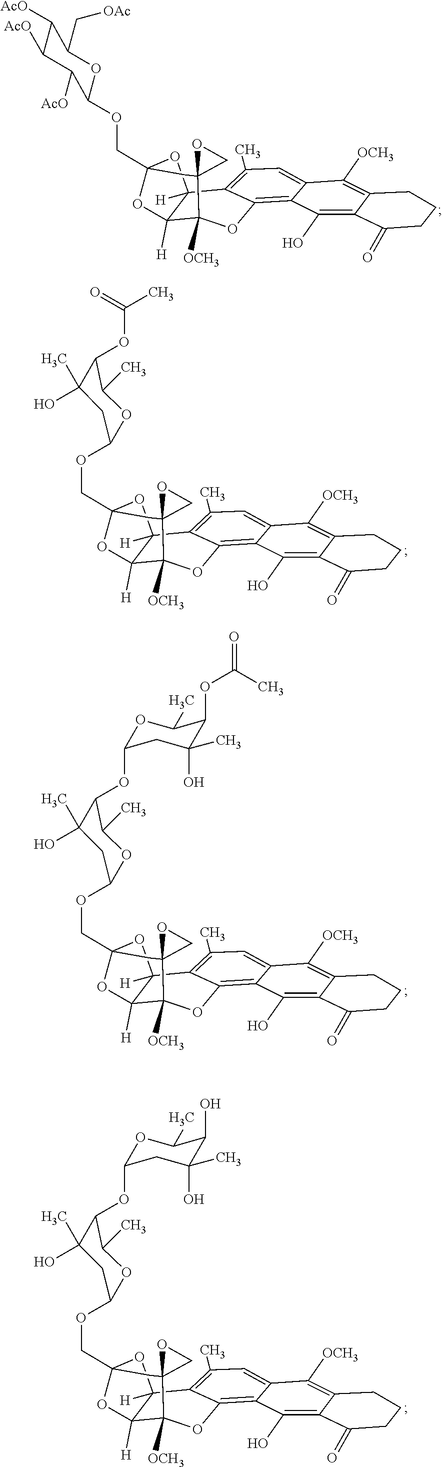

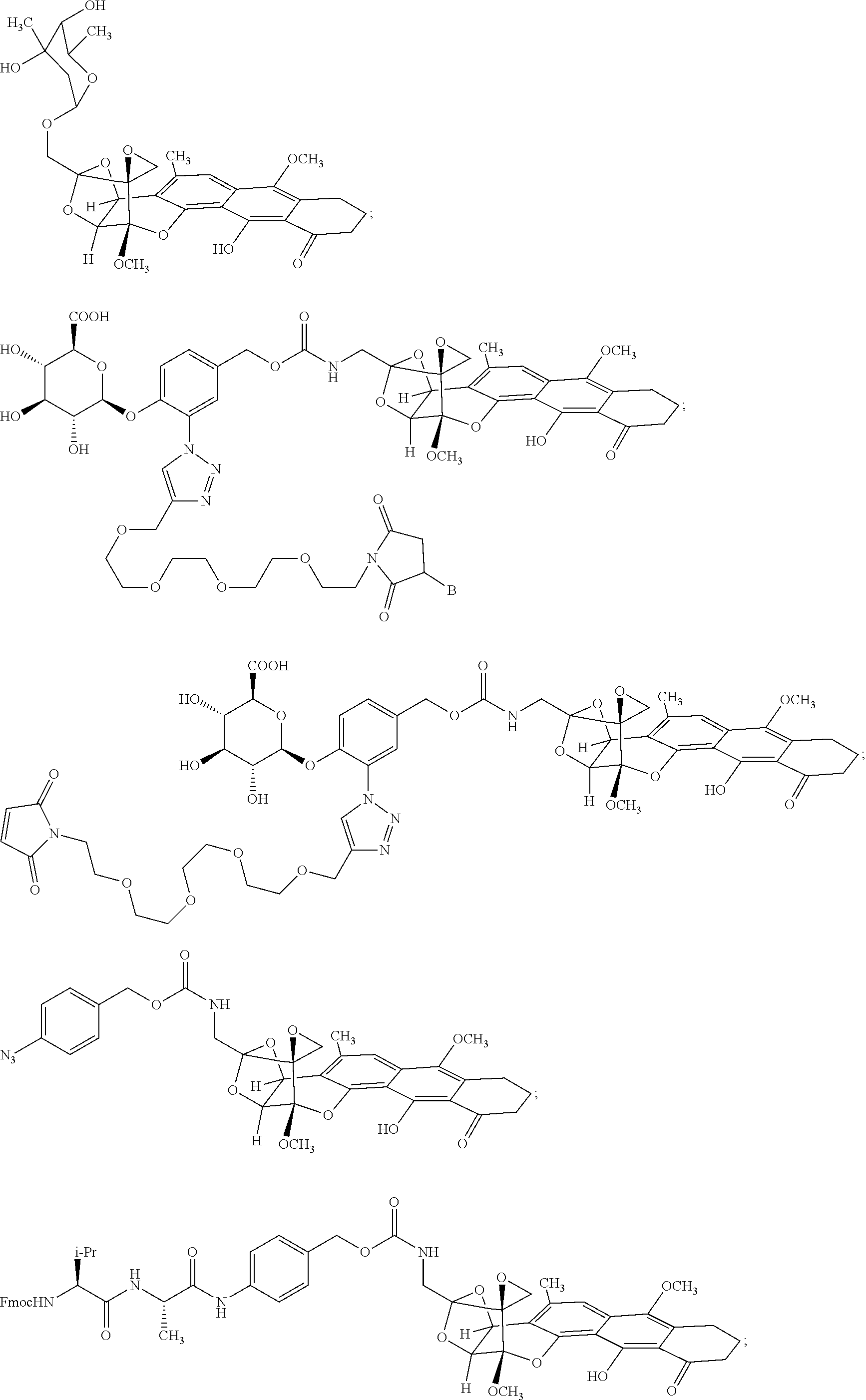

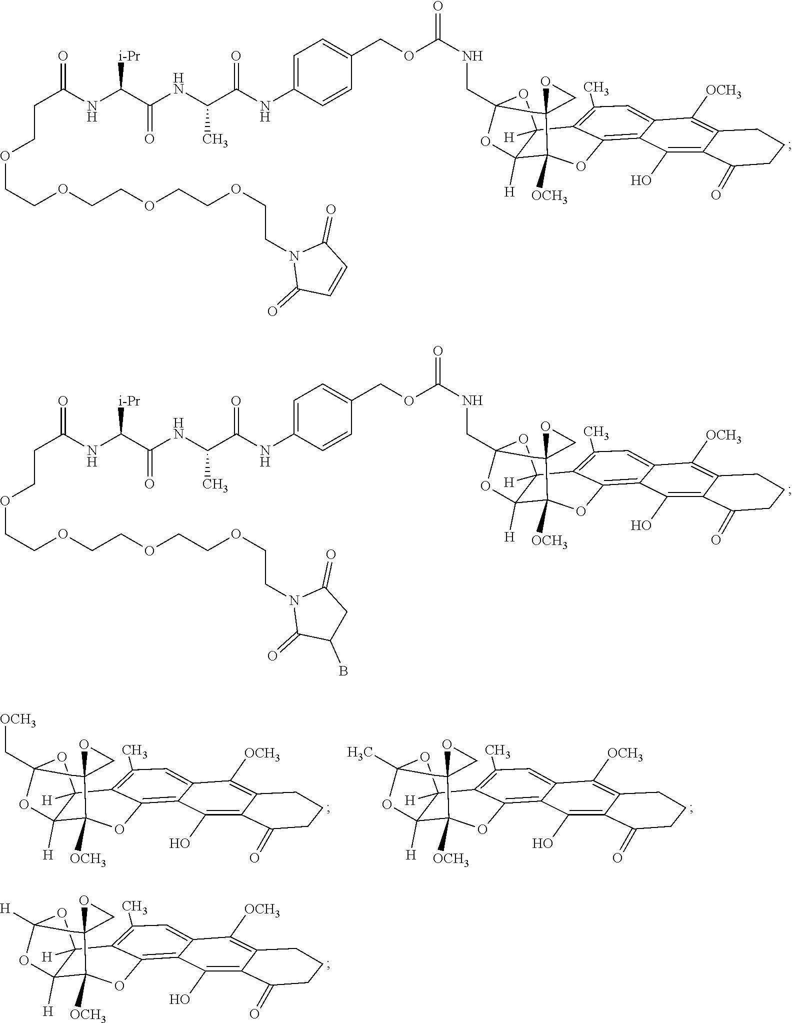

or a pharmaceutically acceptable salt thereof.

In certain embodiments, the compound of Formula (I) is

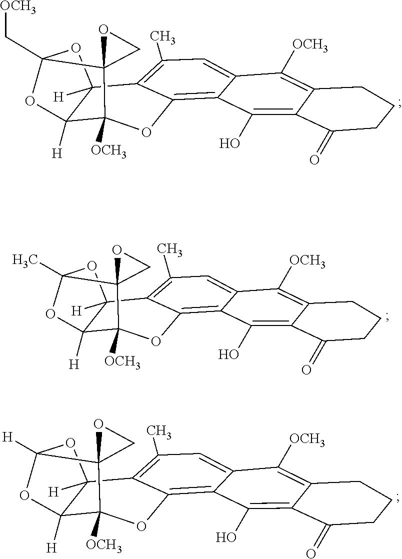

or a pharmaceutically acceptable salt thereof.

In certain embodiments, the compound of Formula (I) is

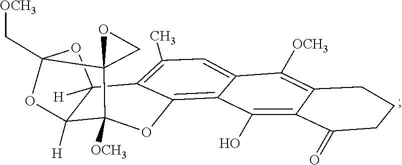

or a pharmaceutically acceptable salt thereof.

In certain embodiments, the compound of Formula (I) is

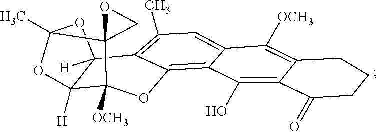

or a pharmaceutically acceptable salt thereof.

In certain embodiments, the compound of Formula (I) is

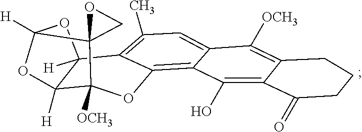

or a pharmaceutically acceptable salt thereof.

In certain embodiments, the compound of Formula (I) is not:

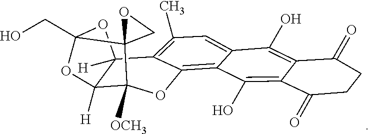

In certain embodiments, the compound of Formula (II) is:

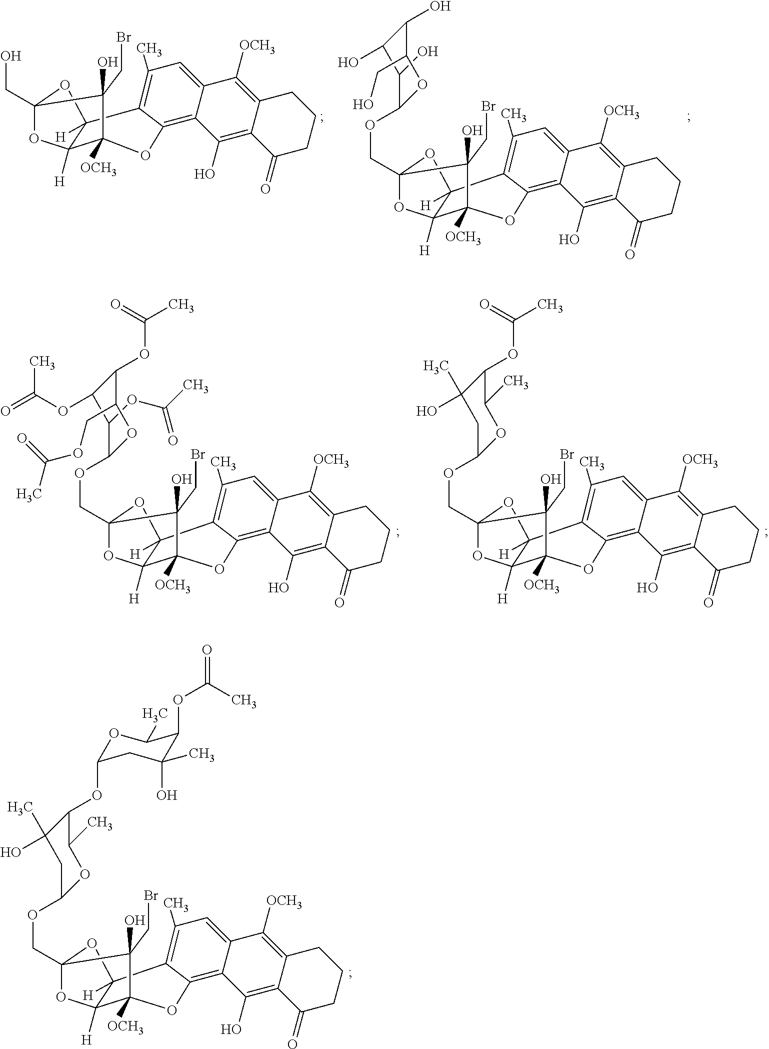

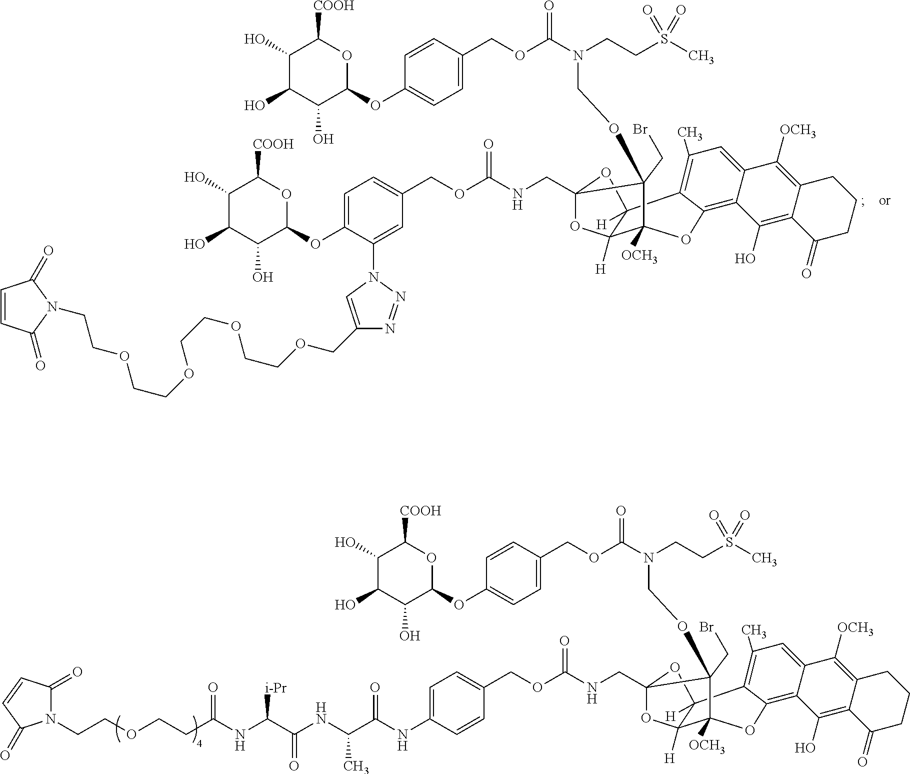

or a pharmaceutically acceptable salt thereof.

In certain embodiments, the compound of Formula (II) is:

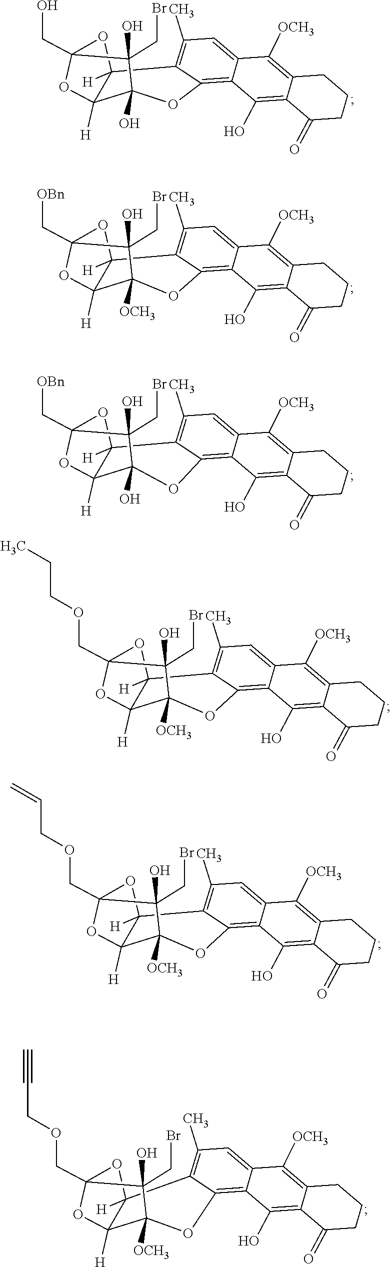

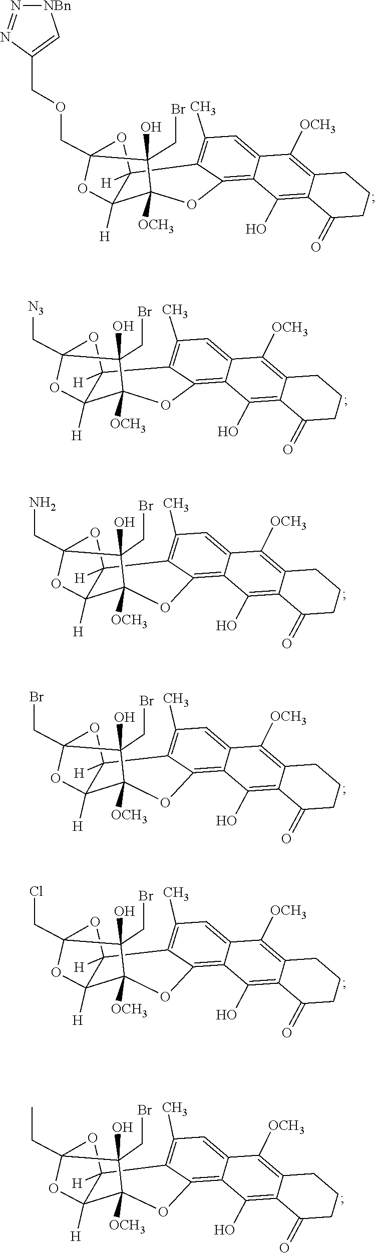

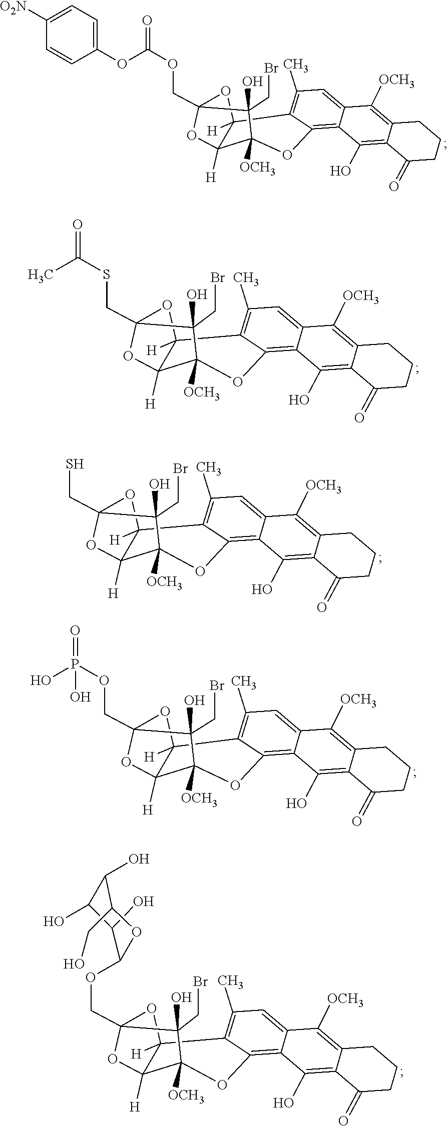

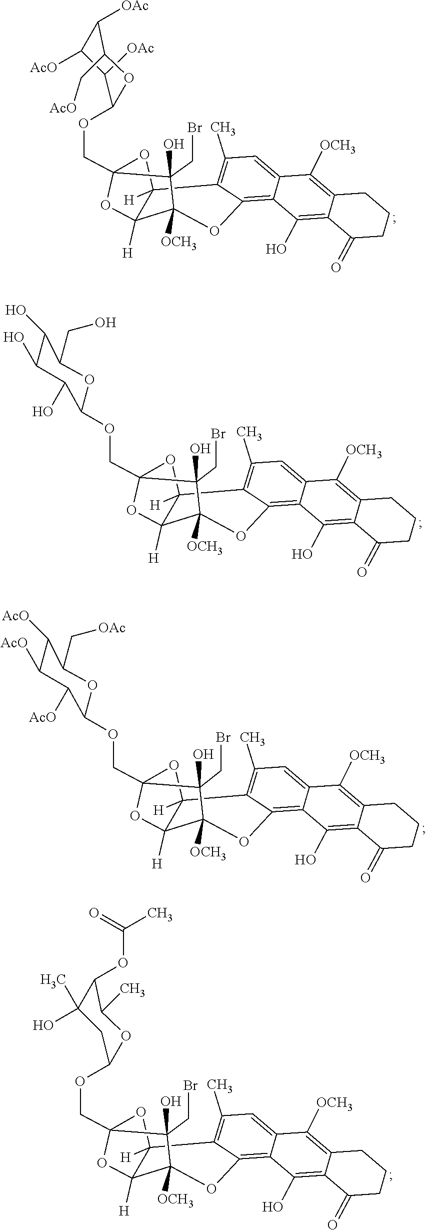

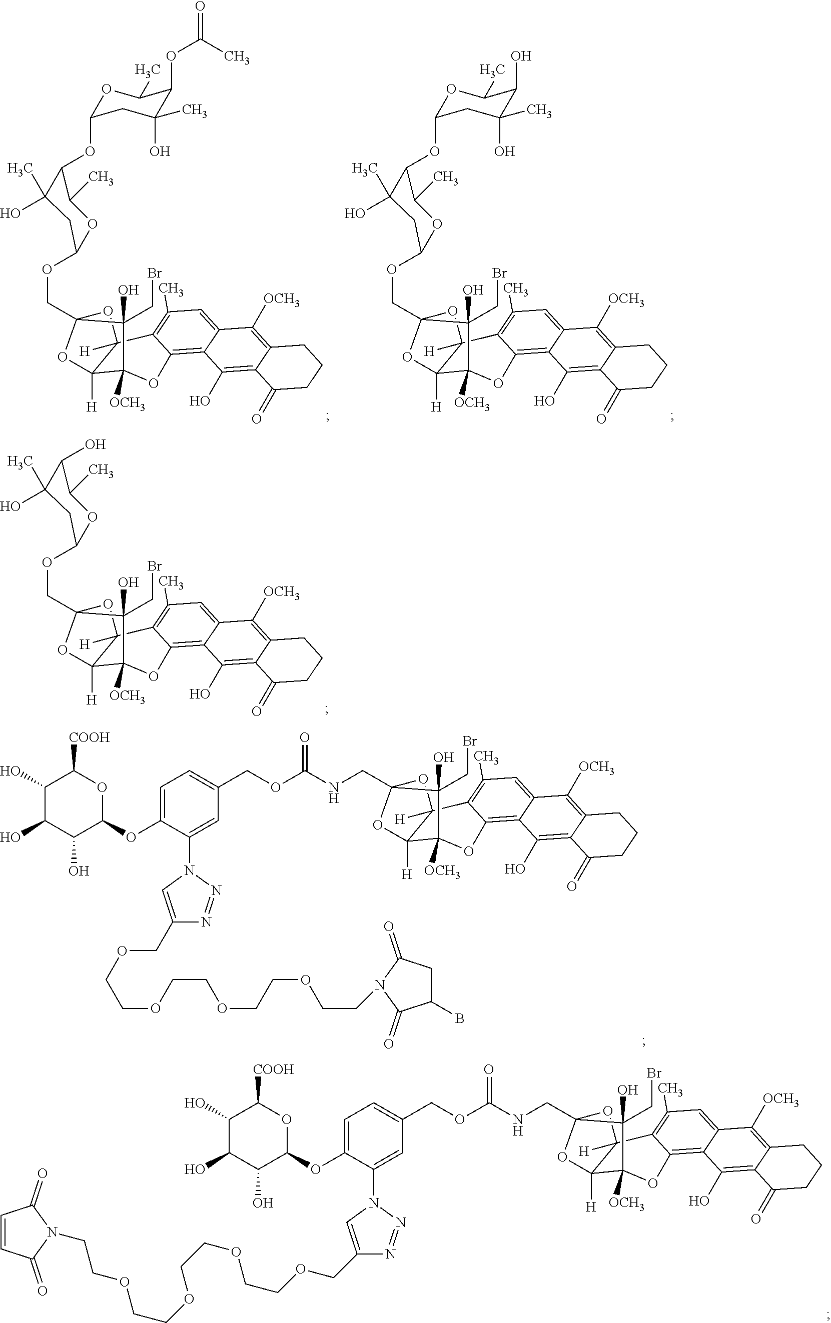

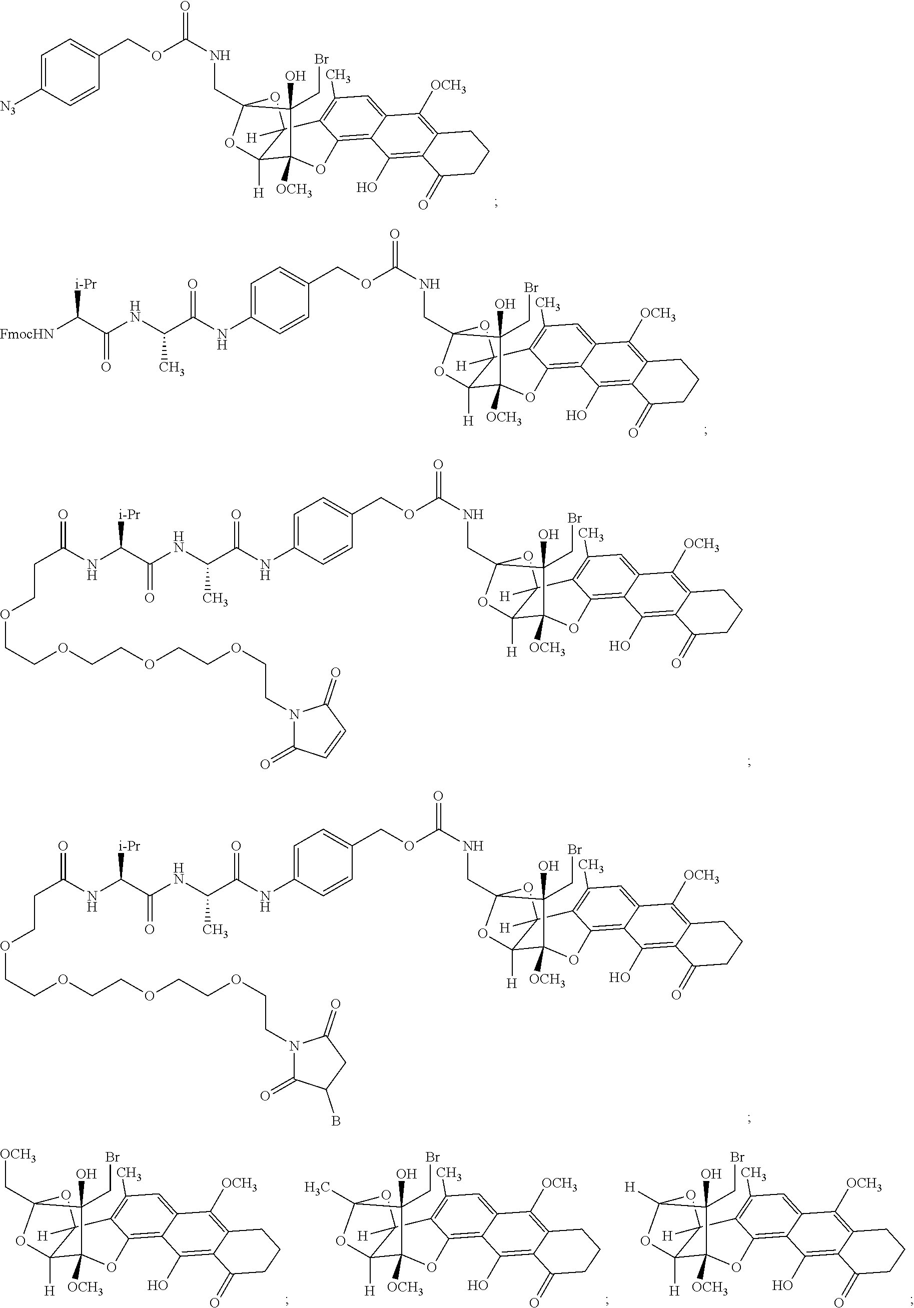

or a pharmaceutically acceptable salt thereof.

In certain embodiments, the compound of Formula (II) is:

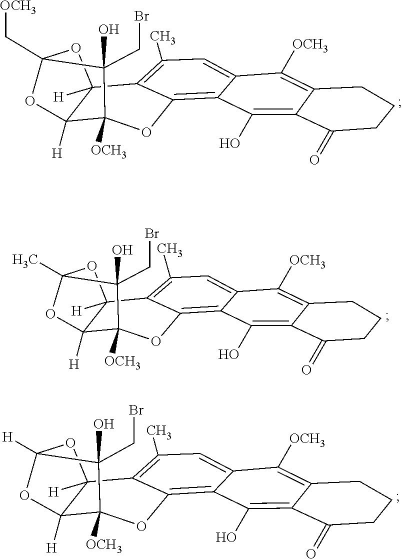

or a pharmaceutically acceptable salt thereof.

In certain embodiments, the compound of Formula (II) is:

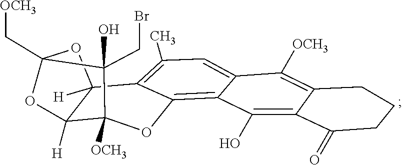

or a pharmaceutically acceptable salt thereof.

In certain embodiments, the compound of Formula (III) is:

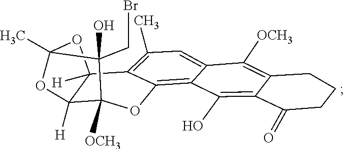

or a pharmaceutically acceptable salt thereof.

In certain embodiments, the compound of Formula (II) is:

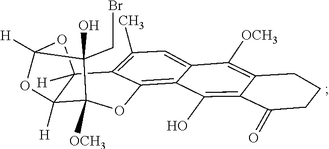

or a pharmaceutically acceptable salt thereof.

In certain embodiments, the compound of Formula (II) is:

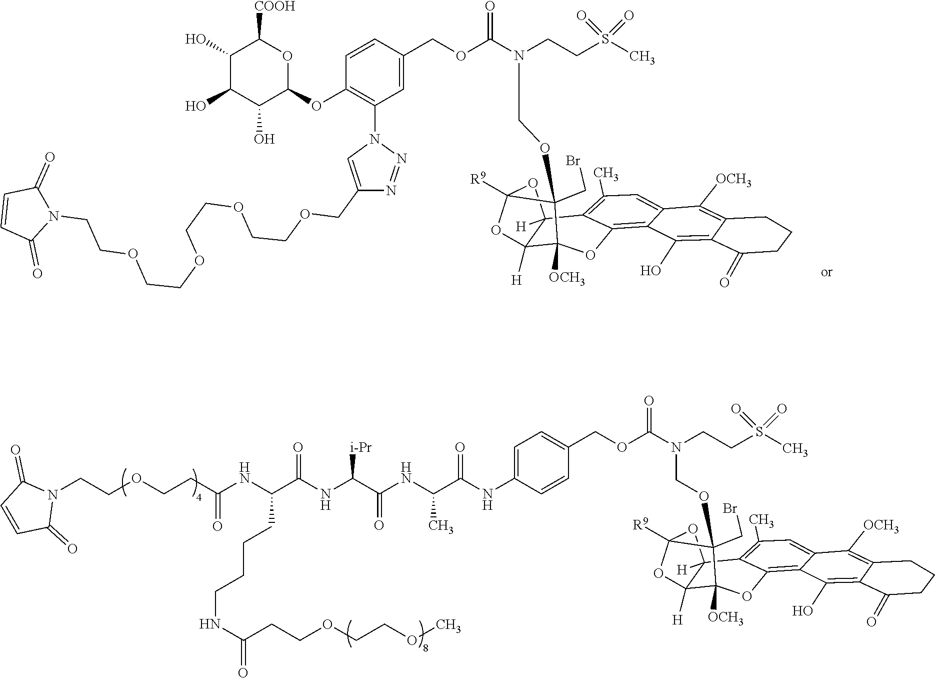

or a pharmaceutically acceptable salt thereof.

In certain embodiments, the compound of Formula (II) is:

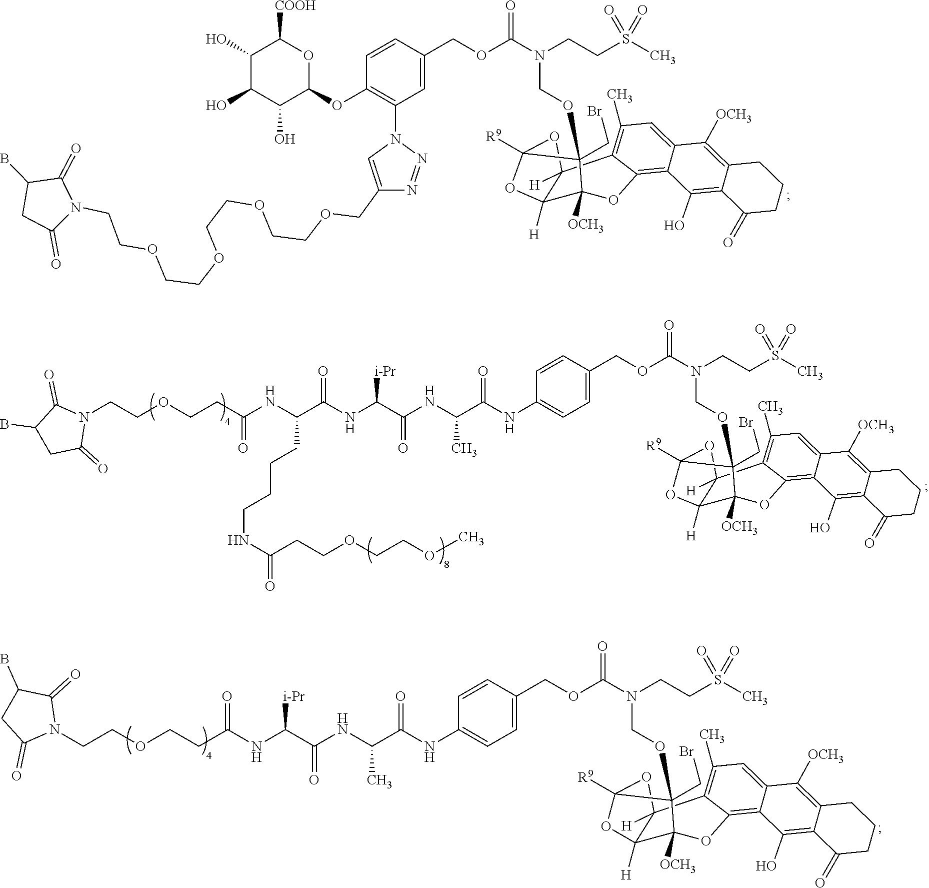

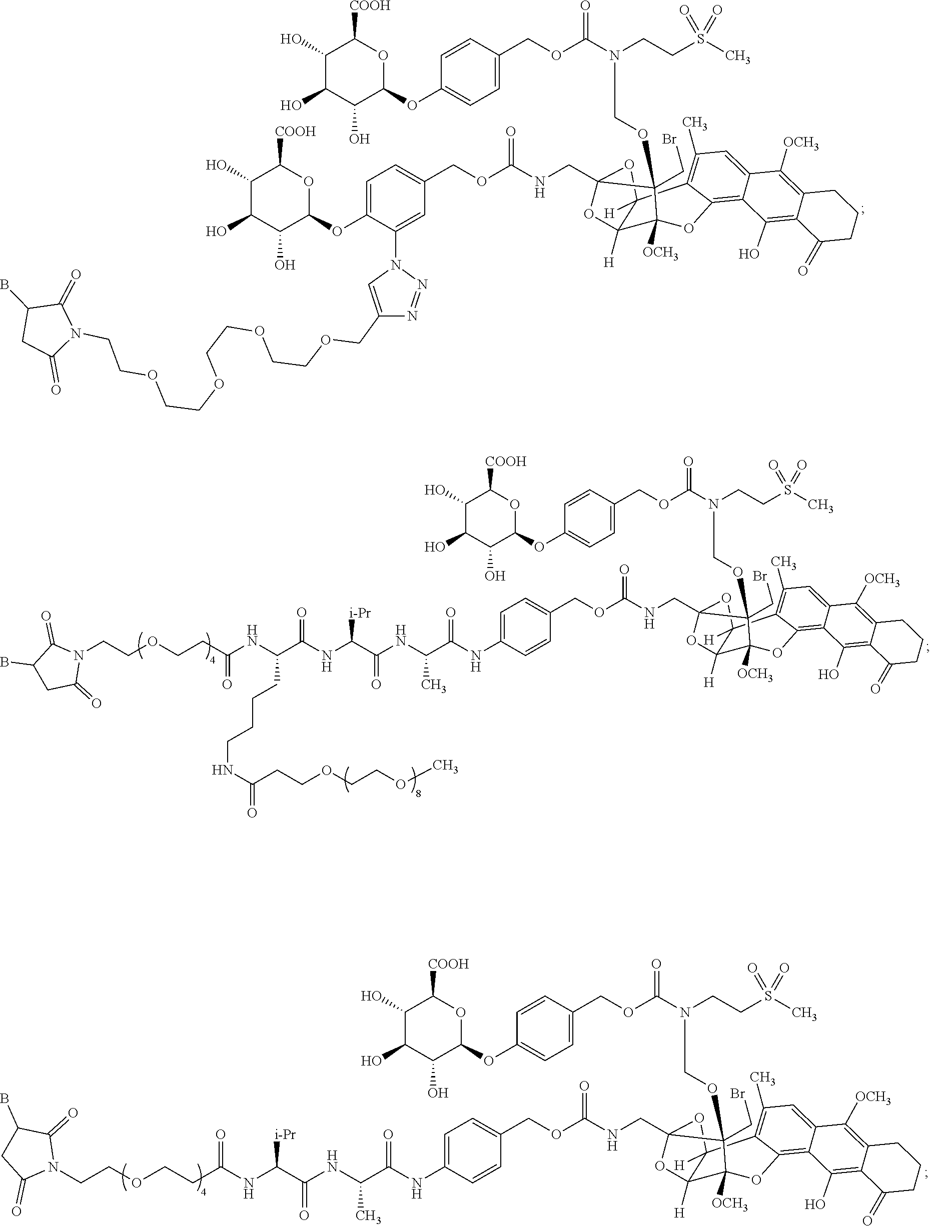

or a pharmaceutically acceptable salt thereof.

In certain embodiments, the compound of Formula (II) is:

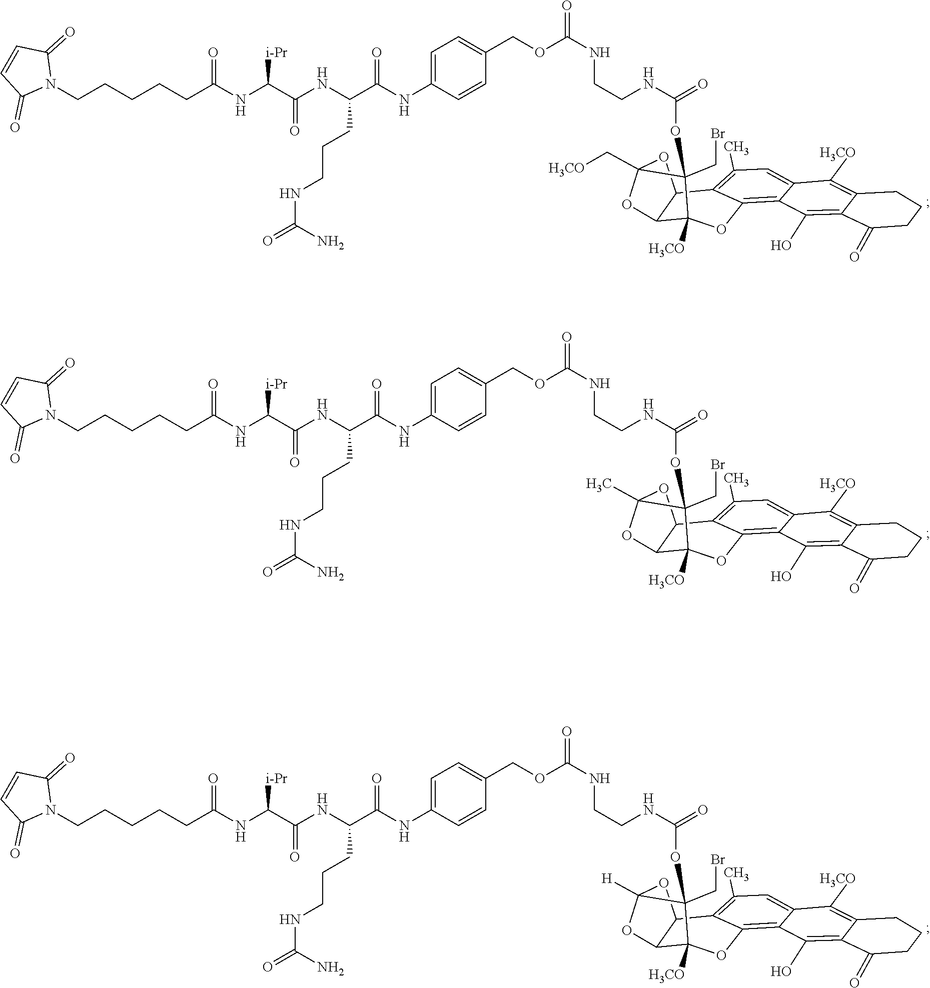

or a pharmaceutically acceptable salt thereof.

In certain embodiments, the compound of Formula (II) is:

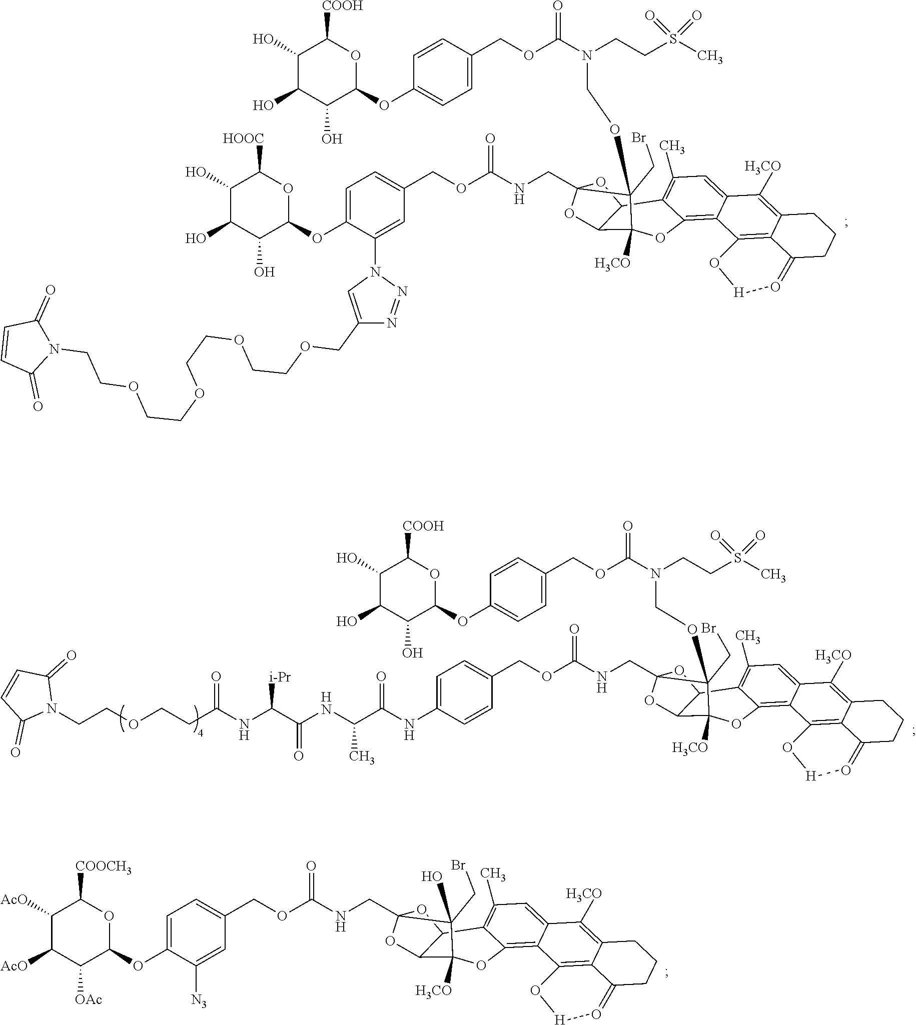

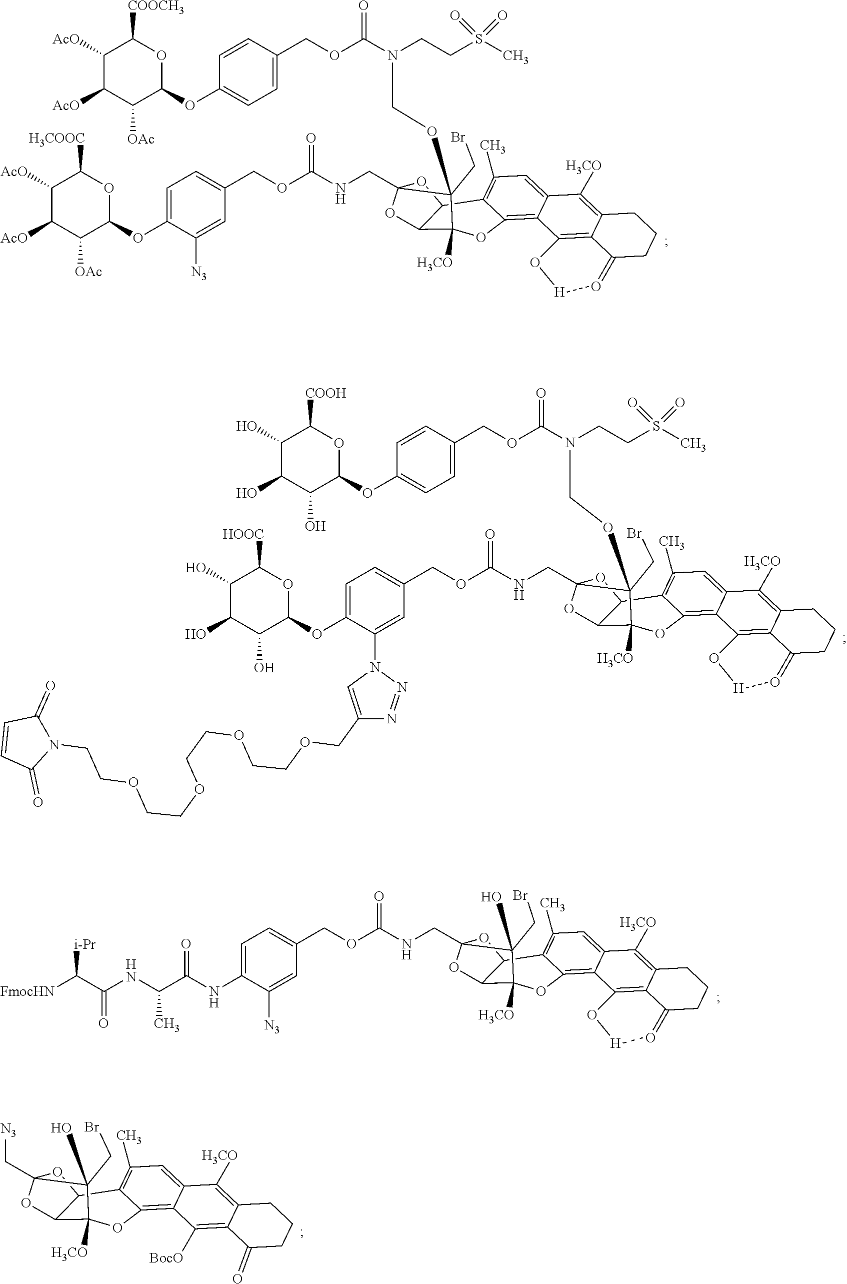

or a pharmaceutically acceptable salt thereof.

### Group B

As generally defined herein, B is a targeting moiety. In certain embodiments, a targeting moiety is an antibody. In certain embodiments, a targeting moiety is an antibody fragment.

It is generally understood that an antibody or antibody fragment is a large molecule with many possible sites of attachment of the trioxacarcin-linker moiety, i.e., a [trioxacarcin-L-A-] moiety. In certain embodiments, the antibody or antibody fragment comprises 1 to 200 independent instances of a trioxacarcin-linker moiety attached thereto, inclusive, e.g., 1 to 150, 1 to 100, 1 to 75, 1 to 50, 1 to 25, 1 to 15, 1 to 10, inclusive, e.g., 1, 2, 3, 4, 5, 6, 7, 8, 9, or 10 independent instances.

An antibody, as described herein, refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. An antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three subdomains, C_{H1}, C_{H2} and C_{H3}. Each light chain is comprised of a light chain variable region (abbreviated herein as V_{L}) and a light chain constant region. The light chain constant region is comprised of one subdomain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

As used herein, the term “monoclonal antibody” may refer to an antibody obtained from a single clonal population of immunoglobulins that bind to the same epitope of an antigen. Monoclonal antibodies have the same Ig gene rearrangement and thus demonstrate identical binding specificity. Methods for preparing monoclonal antibodies, as described herein, are known in the art. Monoclonal antibodies can be prepared by a variety of methods. For example, monoclonal antibodies may be made by a hybridoma method (see, e.g., Kohler et al., Nature, 1975, 256:495), or may be made by recombinant DNA methods (see, e.g., U.S. Pat. No. 4,816,567). The monoclonal antibodies may also be isolated from phage antibody libraries. (See e.g., Clarkson et al., Nature, 1991, 352:624-628 and Marks et al., J. Mol. Biol., 1991, 222:581-597).

Human monoclonal antibodies may be made by any of the methods known in the art, including those disclosed in U.S. Pat. Nos. 5,567,610, 5,565,354, 5,571,893, Kozber, J. Immunol., 1984, 133:3001, Brodeur, et al., Monoclonal Antibody Production Techniques and Applications, p. 51-63 (Marcel Dekker, Inc., new York, 1987), and Boerner et al., J. Immunol., 1991, 147:86-95. Human antibodies may be obtained by recovering antibody-producing lymphocytes from the blood or other tissues of humans producing antibody to an antigen of interest (e.g., CD20 or EGFR). These lymphocytes can be treated to produce cells that grow independently in the laboratory under appropriate culture conditions. The cell cultures can be screened for production of antibodies to the antigen of interest and then cloned. Clonal cultures can be used to produce human monoclonal antibodies to CD20 and/or EGFR, or the genetic elements encoding the variable portions of the heavy and light chain of the antibodies can be cloned and inserted into nucleic acid vectors for production of antibodies of different types. In addition to the conventional methods for preparing human monoclonal antibodies, such antibodies may also be prepared by immunizing transgenic animals that are capable of producing human antibodies (e.g., Jakobovits et al., PNAS USA, 1993, 90:2551, Jakobovits et al., Nature, 1993, 362:255-258, Bruggermann et al., Year in Immunol., 1993, 7:33 and U.S. Pat. No. 5,569,825).

As used herein, “humanized monoclonal antibody” may refer to monoclonal antibodies having at least human constant regions and an antigen-binding region, such as one, two, or three CDRs, from a non-human species. Humanized antibodies specifically recognize antigens of interest, but will not evoke an immune response in humans against the antibody itself. As an example, murine CDRs may be grafted into the framework region of a human antibody to prepare the humanized antibody (e.g., Riechmann et al., Nature, 1988, 332, 323, and Neuberger et al., Nature, 1985, 314, 268). Alternatively, humanized monoclonal antibodies may be constructed by replacing the non-CDR regions of non-human antibodies with similar regions of human antibodies while retaining the epitopic specificity of the original antibodies. For example, non-human CDRs and optionally some of the framework regions may be covalently joined to human FR and/or Fc/pFc′ regions to produce functional antibodies.

As used herein, the term “chimeric antibody” may refer to a monoclonal antibody comprising a variable region from one source (e.g., species) and at least a portion of a constant region derived from a different source. In some embodiments, chimeric antibodies are prepared by recombinant DNA techniques. In some embodiments, the chimeric antibodies comprise a murine variable region and a human constant region. Such chimeric antibodies may, in some embodiments, be the product of expressed immunoglobulin genes comprising DNA segments encoding murine immunoglobulin variable regions and DNA segments encoding human immunoglobulin constant regions. Methods for producing chimeric antibodies involve conventional recombinant DNA and gene transfection techniques (see, e.g., Morrison et al., Proc. Natl. Acad. Sci. USA, 1984, 81:6851-6855; U.S. Pat. Nos. 5,202,238; and 5,204,244).

An antibody fragment is a portion of an antibody such as F(ab′).sub.2, F(ab).sub.2, Fab′, Fab, Fv, scFv (single chain Fv) and the like. Such fragments may be prepared by standard methods. See, e.g., Coligan et al. Current Protocols in Immunology, John Wiley & Sons, 1991-1997. Regardless of structure, an antibody fragment binds with the same antigen that is recognized by the intact antibody. An antibody fragment may comprise one or more proteolytic fragments (i.e., fragments produced by cleavage with papain), e.g., a Fab fragment, each containing a light chain domain and a heavy chain domain (designated herein as a “Fab heavy chain domain”), and/or Fc fragment containing two Fc domains. Each light chain domain contains a V_{L} and a C_{L} subdomain, each Fab heavy chain domain contains a V_{H} and a C_{H1} subdomain, and each Fc domain contains a C_{H2} and C_{H3} subdomain.

In certain embodiments, antigen-binding antibody fragments is only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. An isolated F(ab′)₂ fragment is referred to as a bivalent monoclonal fragment because of its two antigen binding sites. Similarly, an antibody from which the Fc region has been enzymatically cleaved, or which has been produced without the Fc region, designated an Fab fragment, retains one of the antigen binding sites of an intact antibody molecule. Further, Fab fragments consist of a covalently bound antibody light chain and a portion of the antibody heavy chain denoted Fd (heavy chain variable region, referred to herein as Fab heavy chain domain). The Fd fragments are the major determinant of antibody specificity (a single Fd fragment may be associated with up to ten different light chains without altering antibody specificity) and Fd fragments retain epitope-binding ability in isolation.

The terms Fab, Fc, pFc′, F(ab′)₂ and Fv are employed with either standard immunological meanings (Klein, Immunology (John Wiley, New York, NY, 1982); Clark, W.R. (1986) The Experimental Foundations of Modern Immunology (Wiley & Sons, Inc., New York); Roitt, I. (1991) Essential Immunology, 7th Ed., (Blackwell Scientific Publications, Oxford)). Well-known functionally active antibody fragments include but are not limited to F(ab′)₂, Fab, Fv and Fd fragments of antibodies. These fragments, which lack the Fc fragment of intact antibody, clear more rapidly from the circulation, and may have less non-specific tissue binding than an intact antibody (Wahl et al., J. Nucl. Med. 24:316-325 (1983)). For example, single-chain antibodies may be constructed in accordance with the methods described in U.S. Pat. No. 4,946,778. Such single-chain antibodies include the variable regions of the light and heavy chains joined by a flexible linker moiety. Methods for obtaining a single domain antibody (“Fd”) which comprises an isolated variable heavy chain single domain, also have been reported (see, e.g., Ward et al., Nature, 1989, 341:644-646, disclosing a method of screening to identify an antibody heavy chain variable region (VH single domain antibody) with sufficient affinity for its target epitope to bind thereto in isolated form). Methods for making recombinant Fv fragments based on known antibody heavy chain and light chain variable region sequences are known in the art and have been described, (see, e.g., Moore et al., U.S. Pat. No. 4,462,334). Other references describing the use and generation of antibody fragments include, e.g., Fab fragments (Tijssen, Practice and Theory of Enzyme Immunoassays (Elsevieer, Amsterdam, 1985)), Fv fragments (Hochman et al., Biochemistry, 1973, 12:1130; Sharon et al., Biochemistry, 1976, 15:1591; Ehrilch et al., U.S. Pat. No. 4,355,023) and portions of antibody molecules (Audilore-Hargreaves, U.S. Pat. No. 4,470,925). Thus, antibody fragments may be constructed from intact antibodies without destroying the specificity of the antibodies for the CD20 or EGFR epitope.

In certain embodiments, the antibody fragment is a camelid antibody; e.g., a functional antibody devoid of light chains of which the single N-terminal domain is fully capable of antigen binding; i.e., a single-domain antibody fragment.

Exemplary antibodies and their cell markers (targets) contemplated for use include, but are not limited to, antibodies listed in Table A, and antibody fragments thereof.

TABLE A

| Antibody | Target |
|---|---|
| adecatumumab (MT201) | EpCAM - CD326 |
| afutuzumab | CD20 |
| alemtuzumab (CAMPATH) | CD52 |
| bavituximab | phosphatidylserine |
| belimumab | BAFF, BLyS |
| bevacizumab (AVASTIN) | VEGF-A |
| brentuximab (ADCETRIS) | CD30 |
| cantuzumab | MUC1 |
| cetuximab (ERBITUX) | EGF receptor |
| citatuzumab | TACSTD1 |
| cixutumumab | IGF-1 receptor |
| conatumumab | TRAIL-R2 (CD262) |
| dacetuzumab | CD40 |
| elotuzumab | SLAMF7 (CD319) |
| etaracizumab | alpha-v beta-3 integrin |
| farletuzumab | FR-alpha |
| figitumumab | IGF-1 receptor |
| gemtuzumab | CD33 |
| ibritumomab | CD20 |
| inotuzumab (CMC-533) | CD22 |
| ipilimumab (YERVOY) | CTLA-4 |
| iratumumab | CD30 |
| labetuzumab | carcinoembryonic antigen |
| lexatumumab | TRAIL-R2 |
| lintuzumab | CD33 |
| lucatumumab | CD40 |
| mapatumumab | TRAIL-receptor (death receptor 4) |
| matuzumab | EGFR |
| milatuzumab | CD74 |
| necitumumab | EGFR |
| nimotuzumab | EGFR |
| ofatumumab (ARZERRA) | CD20 |
| olaratumab | PDGF-R α |
| oportuzumab | EpCAM |
| panitumumab (VECTIBIX) | EGFR |
| pertuzumab (PERJETA) | HER2 |
| pritumumab | vimentin |
| rituximab (RITUXAN) | CD20 |
| robatumumab | CD221 |
| sibrotuzumab | FAP |
| siltuximab | IL-6 |
| tacatuzumab | α-fetoprotein |
| tigatuzumab | TNFRSF10B (TRAIL-R2) |
| tositumomab (BEXXAR) | CD20 |
| trastuzumab (HERCEPTIN) | HER2/neu |
| tucotuzumab | EpCAM |
| veltuzumab | CD20 |
| votumumab | mor antigen CTAA16.88 |
| zalutumumab | EGFr |

In certain embodiments, the antibody is any antibody directed to any of the targets listed in table A.

Additional antibodies include, but are not limited to, pembrolizumab, nivolumab, pidilizumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, varilumab, ulocuplumab, monalizumab, MEDI0562, MEDI0680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, Chi Lob 7/4, R^{G6058}, R^{G7686}, R^{G7876}, R^{G7888}, TR^{X518}, MK-4166, MGA271, IMC-CS4, emactuzumab, obinutuzumab, cabiralizumab, margetuximab, enoblituzumab, mogamulizumab, carlumab, fresolimumab, FAZ053, TSR022, MBG453, REGN2810, REGN3767, MOXR0916, PF-04518600, RO7009789, BMS986156, GWN323, JTX-2011, NKTR-214, GSK3174998, DS-8273a, NIS793, or BGB-A317

In certain embodiments, the antibody is trastuzumab (HERCEPTIN) or an antibody fragment thereof.

In certain embodiments, the antibody is rituximab (RITUXAN) or an antibody fragment thereof.

### Methods of Preparation

In certain embodiments, the method of preparing a compound of Formula (I) comprises deprotecting a cycloadduct of Formula (A):

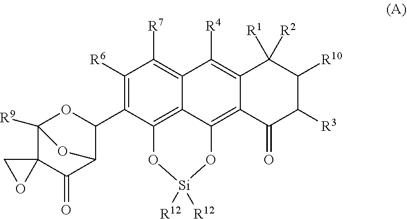

or salt thereof, wherein R^{I2} is a cyclic or acyclic, branched or unbranched, substituted or unsubstituted aliphatic, substituted or unsubstituted aryl, or substituted or unsubstituted heteroaryl.

In certain embodiments, the method of preparing a compound of Formula (I) comprises reacting a silacycle of Formula (B):

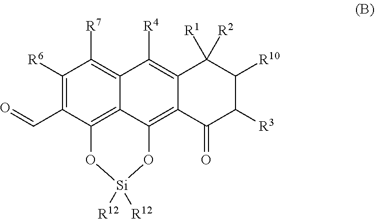

or a salt thereof, with an α-epoxy-β-oxodiazoketone of formula:

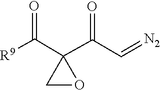

or a salt thereof.

In certain embodiments, the method of preparing a compound of Formula (I) comprises alkylating a compound of Formula (I):

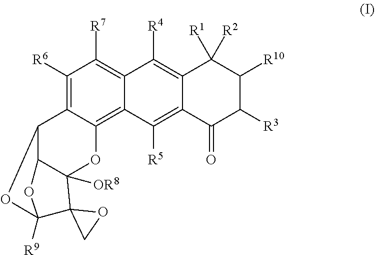

or salt thereof, wherein R⁸ is hydrogen; to provide a compound of Formula (I), or a salt thereof, wherein R⁸ is alkyl.

In certain embodiments, the method of preparing a compound of Formula (I) comprises providing a compound of Formula (I-f):

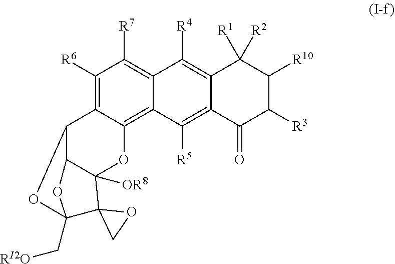

or salt thereof, wherein R⁸ is hydrogen; and R^{I2} is a protecting group; alkylating and deprotecting the compound to provide a compound of Formula (I-f) or a salt thereof, wherein R⁸ is alkyl, and R^{I2} is hydrogen.

In certain embodiments, the method of preparing a compound of Formula (II) comprises providing a compound of Formula (I):

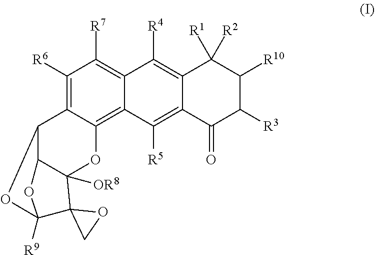

or a pharmaceutically acceptable salt thereof, and ring-opening the compound to provide a compound of Formula (II):

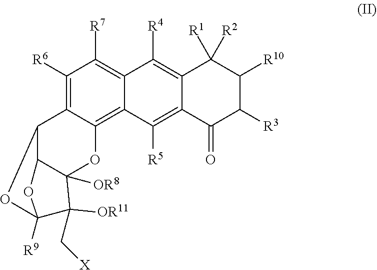

or a pharmaceutically acceptable salt thereof.

In certain embodiments, the method of preparing a compound of Formula (C):

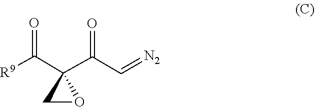

or salt thereof, comprises deprotecting and oxidizing a compound of formula (D):

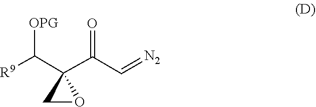

or salt thereof, wherein:
PG is a protecting group.

In certain embodiments, the method of preparing a compound of Formula (C) comprises hydrolyzing a compound of Formula (E):

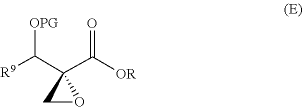

or salt thereof; wherein R is cyclic or acyclic, substituted or unsubstituted, branched or unbranched aliphatic; to provide a compound of Formula (F):

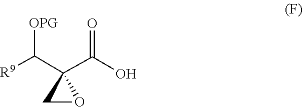

or a salt thereof; and converting the compound of Formula (F) to the compound of Formula (D):

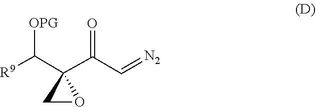

or salt thereof.

In certain embodiments, the method of preparing a compound of Formula (C) comprises epoxidizing and protecting a compound of Formula (G):

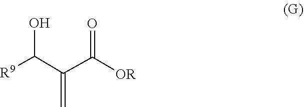

or a salt thereof; to provide the compound of Formula (E):

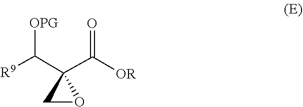

or salt thereof.

In certain embodiments, the method of preparing a compound of Formula (C) comprises reacting an aldehyde of Formula (H):

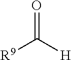

or salt thereof; with an acrylate, or salt thereof, to provide the compound of Formula (G):

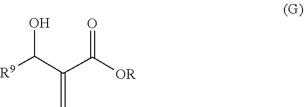

or a salt thereof.

In certain embodiments, the method of preparing a compound of Formula (C) comprises stereoselectively epoxidizing and protecting the compound of Formula (G):

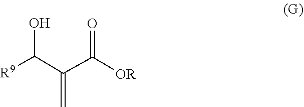

or a salt thereof; to provide the compound of Formula (E-1):

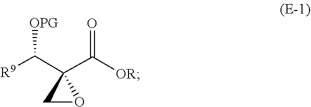

or a salt thereof.

In certain embodiments, the method of preparing a compound of Formula (C) comprises enantioenriching, protecting, and epoxidizing the compound of Formula (G):

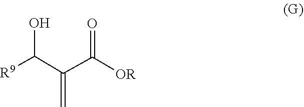

or a salt thereof; to provide the compound of Formula (E-2):

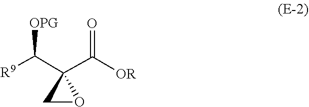

or salt thereof.

As is generally understood from the above disclosure, the compounds of Formulae (I) and (II), comprising a group T, are coupled to a targeting moiety to form an antibody-drug conjugate of Formulae (I) or (II). See, e.g., Scheme 1. In certain embodiments, the coupling takes place between a nucleophilic sidechain of an amino acid residue (e.g., cysteine, lysine, serine) of the antibody and an electrophilic T group. Exemplary coupling reactions include, but are not limited to, formation of esters, thioesters, amides (e.g., such as peptide coupling) from activated acids or acyl halides; nucleophilic displacement reactions (e.g., such as nucleophilic displacement of a halide); and Michael additions (e.g., maleimide addition).

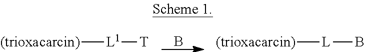

In certain embodiments, the method of preparing a compound of Formulae (I) or (II) comprises coupling a targeting moiety with a compound of Formulae (I) or (II), wherein T is

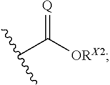

Q is —S—, or —O—; and R^{X2} is hydrogen, substituted or unsubstituted alkyl; substituted or unsubstituted heterocyclyl (e.g., succinimide); substituted or unsubstituted aryl; substituted or unsubstituted heteroaryl; or an oxygen protecting group, to provide a corresponding compound of Formulae (I) or (II). See, for example, Scheme 2.

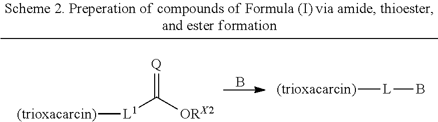

In certain embodiments, the method of preparing a compound of Formulae (I) or (II) comprises coupling a targeting moiety with a compound of Formulae (I) or (II), wherein T is

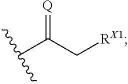

Q is —S—, or —O—; and R^{X1} is a leaving group (e.g., halogen, tosylate, mesylate, or triflate), to provide a compound of Formulae (I) or (II). See, for example, Scheme 3.

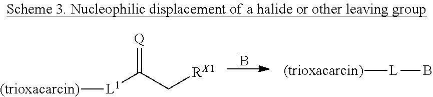

In certain embodiments, the method of preparing a compound of Formulae (I) or (II) comprises coupling a targeting moiety with a compound of Formulae (I) or (II), wherein T is —N═C═S (i.e., isothiocyanate) to provide a compound of Formulae (I) or (II). See, for example, Scheme 4.

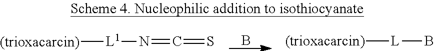

In certain embodiments, the method of preparing a compound of Formulae (I) or (II) comprises coupling a targeting moiety with a compound of Formulae (I) or (II), wherein T is a maleimide group to provide a compound of Formulae (I) or (II). See, for example, Scheme 5.

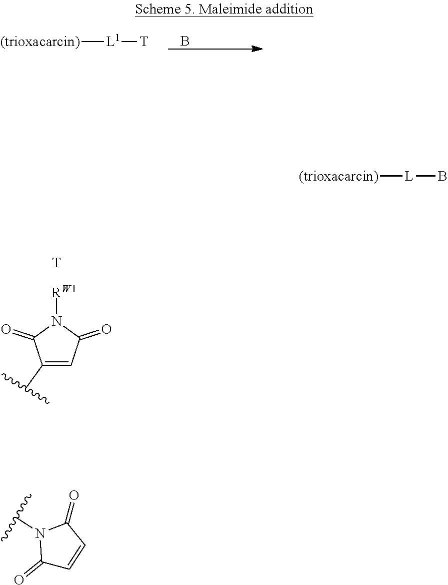

In certain embodiments, the method of preparing a compound of Formulae (I) or (II) comprises coupling a targeting moiety with a compound of Formulae (I) or (II), wherein T is 4-nitrobenzenethiol (e.g., wherein the sulfur is attached to a sulfur atom of L¹) to provide a compound of Formulae (I) or (II). See, for example, Scheme 6.

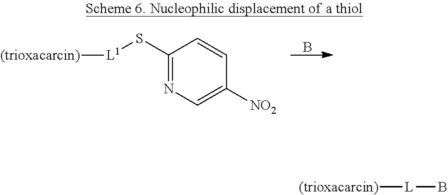

### Pharmaceutical Compositions

The present disclosure provides pharmaceutical compositions comprising an active ingredient and, optionally, a pharmaceutically acceptable carrier. In certain embodiments, the active ingredient is present in an effective amount, e.g., a therapeutically effective amount or a prophylactically effective amount.

An “active ingredient,” as used herein, refers to compounds of Formula (I) or (II), and pharmaceutically acceptable salts thereof.

A pharmaceutical composition of the present disclosure can be administered via one or more routes of administration using one or more of a variety of methods known in the art. As will be appreciated by the skilled artisan, the route and/or mode of administration will vary depending upon the desired results. Preferred routes of administration include intravenous, intramuscular, intradermal, intraperitoneal, subcutaneous, spinal, or other parenteral routes of administration, for example, by epidermal administration (e.g., by injection or infusion). The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion.

Alternatively, the pharmaceutical composition can be administered via a non-parenteral route, such as a topical, epidermal or mucosal route of administration, for example, intranasally, orally, vaginally, rectally, sublingually or topically.

Depending on the route of administration, the pharmaceutical composition or active ingredient may be coated in a material to protect the compound from the action of acids and other natural conditions that may inactivate the compound.

Pharmaceutically acceptable excipients include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

Pharmaceutical compositions described herein can be prepared by any method known in the art of pharmacology. In general, such preparatory methods include the steps of bringing the active ingredient into association with the excipient and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable carrier, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly (vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly (vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the conjugates of this invention with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

The active ingredient can be prepared with carriers that will protect the active ingredient against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Pharmaceutical compositions can be administered with medical devices known in the art. For example, in a preferred embodiment, a pharmaceutical composition of this disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21^{st} ed., Lippincott Williams & Wilkins, 2005.

The exact amount of the active ingredient required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of an active ingredient for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of the active ingredient per unit dosage form.

In certain embodiments, the active ingredient may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that the active ingredient or composition, as described herein, can be administered in combination with one or more additional therapeutically active agents. The active ingredient or compositions can be administered in combination with additional therapeutically active agents that improve their bioavailability, reduce and/or modify their metabolism, inhibit their excretion, and/or modify their distribution within the body. It will also be appreciated that the therapy employed may achieve a desired effect for the same disorder (for example, a compound can be administered in combination with an anti-cancer agent, etc.), and/or it may achieve different effects (e.g., control of adverse side-effects, e.g., emesis controlled by an anti-emetic).

The active ingredient or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the active ingredient with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, cancer therapies, antibiotics, anti-viral agents, anesthetics, anti-coagulants, inhibitors of an enzyme, steroidal agents, steroidal or non-steroidal anti-inflammatory agents, antihistamine, immunosuppressant agents, anti-neoplastic agents, antigens, vaccines, antibodies, decongestant, sedatives, opioids, pain-relieving agents, analgesics, anti-pyretics, hormones, prostaglandins, progestational agents, anti-glaucoma agents, ophthalmic agents, anti-cholinergics, anti-depressants, anti-psychotics, hypnotics, tranquilizers, anti-convulsants/anti-epileptics (e.g., Neurontin, Lyrica, valproates (e.g., Depacon), and other neurostabilizing agents), muscle relaxants, anti-spasmodics, muscle contractants, channel blockers, miotic agents, anti-secretory agents, anti-thrombotic agents, anticoagulants, anti-cholinergics, β-adrenergic blocking agents, diuretics, cardiovascular active agents, vasoactive agents, vasodilating agents, anti-hypertensive agents, angiogenic agents, modulators of cell-extracellular matrix interactions (e.g., cell growth inhibitors and anti-adhesion molecules), or inhibitors/intercalators of DNA, RNA, protein-protein interactions, protein-receptor interactions, etc. Therapeutically active agents include small organic molecules such as drug compounds (e.g., compounds approved by the Food and Drugs Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

In certain embodiments, the additional therapeutic agent is a cancer therapy. Cancer therapies include, but are not limited to, surgery and surgical treatments, radiation therapy, and administration of additional therapeutic cancer agents (e.g., biotherapeutic and chemotherapeutic cancer agents).

Exemplary biotherapeutic cancer agents include, but are not limited to, interferons, cytokines (e.g., tumor necrosis factor, interferon a, interferon y), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic cancer agents include, but are not limited to, anti-estrogens (e.g. tamoxifen, raloxifene, and megestrol), LHRH agonists (e.g. goscrclin and leuprolide), anti- androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (Abraxane), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g., 1-methyl-4-phenylpyridinium ion), cell cycle inhibitors (e.g. staurosporine), actinomycin (e.g., actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca2-ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the additional pharmaceutical agent is an immunotherapy. In certain embodiments, the immunotherapy is useful in the treatment of a cancer. Exemplary immunotherapies include, but are not limited to, T-cell therapies, interferons, cytokines (e.g., tumor necrosis factor, interferon a, interferon y), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies. In certain embodiments, the immunotherapy is a T-cell therapy. In certain embodiments, the T-cell therapy is chimeric antigen receptor T cells (CAR-T). In certain embodiments, the immunotherapy is an antibody. In certain embodiments, the antibody is an anti-PD-1 antibody, an anti-PD-L1 antibody, an anti-CTLA-4 antibody, an anti-TIM3 antibody, an anti-OX40 antibody, an anti-GITR antibody, an anti-LAG-3 antibody, an anti-CD137 antibody, an anti-CD27 antibody, an anti-CD28 antibody, an anti-CD28H antibody, an anti-CD30 antibody, an anti-CD39 antibody, an anti-CD40 antibody, an anti-CD47 antibody, an anti-CD48 antibody, an anti-CD70 antibody, an anti-CD73 antibody, an anti-CD96 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAMI antibody, an anti-BTLA antibody, an anti-LIGHT antibody, an anti-TIGIT antibody, an anti-VISTA antibody, an anti-HVEM antibody, an anti-Siglec antibody, an anti-GALI antibody, an anti-GAL3 antibody, an anti-GAL9 antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-MICA antibody, an anti-MICB antibody, an anti-NKG2D antibody, an anti-NKG2A antibody, an anti-TGFß antibody, an anti-TGFBR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER2 antibody, an anti-VEGFA antibody, an anti-VEGFR antibody, an anti-EGFR antibody, or an anti-Tie2 antibody. In certain embodiments, the antibody is pembrolizumab, nivolumab, pidilizumab, ipilimumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, varilumab, ulocuplumab, monalizumab, MEDI0562, MEDI0680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, R^{G6058}, R^{G7686}, R^{G7876}, R^{G7888}, TR^{X518}, MK-4166, MGA271, IMC-CS4, emactuzumab, trastuzumab, pertuzumab, obinutuzumab, cabiralizumab, margetuximab, enoblituzumab, mogamulizumab, panitumumab, carlumab, bevacizumab, rituximab, or cetuximab.

In certain embodiments, the compounds or pharmaceutical compositions described herein can be administered in combination with an anti-cancer therapy including, but not limited to, surgery, radiation therapy, and transplantation (e.g., stem cell transplantation, bone marrow transplantation).

In other embodiments, the additional therapeutically active agent is an anti-inflammatory agent. Exemplary anti-inflammatory agents include, but are not limited to, aspirin; ibuprofen; ketoprofen; naproxen; etodolac (LODINE®); COX-2 inhibitors such as celecoxib (CELEBREX®), rofecoxib (VIOXX®), valdecoxib (BEXTRA®), parecoxib, etoricoxib (MK663), deracoxib, 2-(4-ethoxy-phenyl)-3-(4-methanesulfonyl-phenyl)-pyrazolo[1,5-b]pyridazine, 4-(2-oxo-3-phenyl-2,3-dihydrooxazol-4-yl)benzenesulfonamide, darbufelone, flosulide, 4-(4-cyclohexyl-2-methyl-5-oxazolyl)-2-fluorobenzenesulfonamide), meloxicam, nimesulide, 1-Methylsulfonyl-4-(1,1-dimethyl-4-(4-fluorophenyl)cyclopenta-2,4-dien-3-yl)benzene, 4-(1,5-Dihydro-6-fluoro-7-methoxy-3-(trifluoromethyl)-(2)-benzothiopyrano (4,3-c)pyrazol-1-yl)benzenesulfonamide, 4,4-dimethyl-2-phenyl-3-(4-methylsulfonyl)phenyl)cyclo-butenone, 4-Amino-N-(4-(2-fluoro-5-trifluoromethyl)-thiazol-2-yl)-benzene sulfonamide, 1-(7-tert-butyl-2,3-dihydro-3,3-dimethyl-5-benzo-furanyl)-4-cyclopropyl butan-1-one, or their physiologically acceptable salts, esters or solvates; sulindac (CLINORIL®); diclofenac (VOLTAREN®); piroxicam (FELDENE®); diflunisal (DOLOBID®), nabumetone (RELAFEN®), oxaprozin (DAYPRO®), indomethacin (INDOCIN®); or steroids such as PEDIAPED® prednisolone sodium phosphate oral solution, SOLU-MEDROL® methylprednisolone sodium succinate for injection, PRELONE® brand prednisolone syrup.

Further examples of anti-inflammatory agents include naproxen, which is commercially available in the form of EC-NAPROSYN® delayed release tablets, NAPROSYN®, ANAPROX® and ANAPROX® DS tablets and NAPROSYN® suspension from Roche Labs, CELEBREX® brand of celecoxib tablets, VIOXX® brand of rofecoxib, CELESTONE® brand of betamethasone, CUPRAMINE® brand penicillamine capsules, DEPEN® brand titratable penicillamine tablets, DEPO-MEDROL brand of methylprednisolone acetate injectable suspension, ARAVA™ leflunomide tablets, AZULFIDIINE EN-tabs® brand of sulfasalazine delayed release tablets, FELDENE® brand piroxicam capsules, CATAFLAM® diclofenac potassium tablets, VOLTAREN® diclofenac sodium delayed release tablets, VOLTAREN®-XR diclofenac sodium extended release tablets, or ENBREL® etanerecept products.

In certain embodiments, the additional therapeutically active agent is a pain-relieving agent. Exemplary pain relieving agents include, but are not limited to, analgesics such as non-narcotic analgesics [e.g., salicylates such as aspirin, ibuprofen (MOTRINR, ADVIL R), ketoprofen (ORUDISR), naproxen (NAPROSYNR), acetaminophen, indomethacin] or narcotic analgesics [e.g., opioid analgesics such as tramadol, fentenyl, sufentanil, morphine, hydromorphone, codeine, oxycodone, and buprenorphine]; non-steroidal anti-inflammatory agents (NSAIDs) [e.g., aspirin, acetaminophen, COX-2 inhibitors]; steroids or anti-rheumatic agents; migraine preparations such as beta adrenergic blocking agents, ergot derivatives; tricyclic antidepressants (e.g., amitryptyline, desipramine, imipramine); anti-epileptics (e.g., clonaxepam, valproic acid, phenobarbital, phenytoin, tiagaine, gabapentin, carbamazepine, topiramate, sodium valproate); a2 agonists; selective serotonin reuptake inhibitors (SSRIs), selective norepinepherine uptake inhibitors; benzodiazepines; mexiletine (MEXITIL); flecainide (TAMBOCOR); NMDA receptor antagonists (e.g., ketamine, detromethorphan, methadone); and topical agents (e.g., capsaicin (Zostrix), EMLA cream, lidocaine, prilocaine).

### Kits

Still further contemplated herein are pharmaceutical packs and/or kits. Pharmaceutical packs and/or kits provided may comprise a provided composition and a container (e.g., a vial, ampoule, bottle, syringe, and/or dispenser package, or other suitable container). In some embodiments, provided kits may optionally further include a second container comprising a suitable aqueous carrier for dilution or suspension of the provided composition for preparation of administration to a subject. In some embodiments, contents of provided formulation container and solvent container combine to form at least one unit dosage form.

Optionally, a single container may comprise one or more compartments for containing a provided composition, and/or appropriate aqueous carrier for suspension or dilution. In some embodiments, a single container can be appropriate for modification such that the container may receive a physical modification so as to allow combination of compartments and/or components of individual compartments. For example, a foil or plastic bag may comprise two or more compartments separated by a perforated seal which can be broken so as to allow combination of contents of two individual compartments once the signal to break the seal is generated. A pharmaceutical pack or kit may thus comprise such multi-compartment containers including a provided composition and appropriate solvent and/or appropriate aqueous carrier for suspension.

Optionally, instructions for use are additionally provided in such kits of the invention. Such instructions may provide, generally, for example, instructions for dosage and administration. In other embodiments, instructions may further provide additional detail relating to specialized instructions for particular containers and/or systems for administration. Still further, instructions may provide specialized instructions for use in conjunction and/or in combination with additional therapy.

### Methods of Use and Treatment

Further provided are methods of using compounds as described herein (e.g., compounds of Formulae (I) and (II), and pharmaceutically acceptable salts thereof).

For example, in one aspect, provided is a method of treating a disease, disorder, or condition selected from the group consisting of cardiovascular disease, proliferative disease (e.g., cancer, benign tumors), diabetic retinopathy, inflammatory disease, autoimmune disease, and infectious disease (e.g., bacterial infections, fungal infections, parasitic infections) comprising administering an effective amount of a compound of the present disclosure to a subject in need thereof.

In certain embodiments, the compound of the present disclosure is useful in the treatment of cardiovascular disease. Exemplary cardiovascular diseases include, but are not limited to, coronary heart disease, cardiomyopathy, hypertensive heart disease, heart failure, inflammatory heart disease, valvular heart disease, stroke, cerebrovascular disease, and peripheral arterial disease.

In certain embodiments, the compound of the present disclosure is useful in the treatment of a proliferative disease. Exemplary proliferative diseases include, but are not limited to, cancers and benign neoplasms. In certain embodiments, the proliferative disease is cancer. Exemplary cancers include, but are not limited to, acoustic neuroma, adenocarcinoma, adrenal gland cancer, anal cancer, angiosarcoma (e.g., lymphangiosarcoma, lymphangio-endotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast, HER2+ breast cancer, TROP2+ breast cancer), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL), CD19+diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis -like T-cell lymphoma, anaplastic large cell lymphoma); a mixture of one or more leukemia/lymphoma as described above, e.g., mixed leukemia lymphoma (MLL); multiple myeloma (MM), BCMA+multiple myeloma), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), TROP2+ non-small cell lung cancer, EGFR+ non-small cell lung canceradenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma, FRa+ ovarian cancer), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

Trioxacarcins are known to be useful in the treatment of various cancers, such as ovarian, colorectal, hepatocellular, pancreatic cancer, and andenocarcinomas. See, e.g., Cassidy et al., Cancer Chemother. Pharmacol. (1993)31:395-400; Tomita et al., J. Antibiot. (1981)34:1519-1524. It is contemplated that various compounds of Formula (I) and (II) conjugated to an antibody, will have even higher efficacy against these and other cancers as described herein.

In certain embodiments, the compound of the present disclosure is useful in the treatment of diabetic retinopathy.

In certain embodiments, the compound of the present invention is useful in the treatment of an inflammatory disease. Exemplary inflammatory diseases include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, type 2 diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenia purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis.

In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds may also be useful in treating inflammation associated with cancer.

In certain embodiments, the compound of the present disclosure is useful in the treatment of an autoimmune disease. Exemplary autoimmune diseases include, but are not limited to, arthritis (e.g., including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo chondritis)).

In certain embodiments, the inflammatory disorder and/or the immune disorder is a gastrointestinal disorder. In some embodiments, the gastrointestinal disorder is selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS).

In certain embodiments, the compound of the present disclosure is useful in the treatment of an infectious disease (e.g., bacterial infection, fungal infection, and/or parasitic infection). In certain embodiments, the compound is useful in treating a parasitic infection (e.g., malaria). In certain embodiments, the compound is useful in treating a bacterial infection. In certain embodiments, the compound is useful in treating a fungal infection.

Trioxacarcins are known to have antibiotic and antiparasitic (e.g., anti-malarial) activity. See, e.g., U.S. Pat. Nos. 4,459,291; 4,511,560; Fujimoto et al., J. Antibiot. (1983)36:1216-1221; Maiese et al. J. Antibiot. (1990) 43:253-258; Tomita et al., J. Antibiot. (1981)34:1519-1524; and Maskey et al. J. Antibiot. (2004) 57:771-779 (antibacterial and antimalarial activity). It is contemplated that various compounds of Formula (I) and (II) conjugated to an antibody, will have even higher efficacy against an infectious disease, such as a bacterial infection, and other infectious diseases as described herein.

### EXAMPLES

In order that the invention described herein may be more fully understood, the following examples are set forth. It should be understood that these examples are for illustrative purposes only and are not to be construed as limiting this invention in any manner.

General Experimental Procedures. All reactions were performed in flame-dried round-bottom flasks fitted with rubber septa under positive nitrogen pressure, unless otherwise noted. Air- and moisture-sensitive liquids were transferred via syringe or stainless-steel cannula. Organic solutions were concentrated by rotary evaporation (house vacuum, ca. 25-40 Torr) at ambient temperature, unless otherwise noted. Analytical thin-layer chromatography (TLC) was performed using glass plates precoated with silica gel (0.25 mm, 60 Å pore-size, 230-400 mesh, Merck KGA) impregnated with a fluorescent indicator (254 nm). TLC plates were visualized by exposure to ultraviolet light, then were stained with either an aqueous sulfuric acid solution of ceric ammonium molybdate (CAM), an ethanol-aqueous sulfuric acid solution of 2,4-dinitrophenylhydrazine (DNP), or an aqueous sodium hydroxide-potassium carbonate solution of potassium permanganate (KMnO₄) then briefly heated using a heat gun. Flash-column chromatography was performed as described by Still et al., employing silica gel (60 Å, 15-40 μM, EMD Millipore Corp.)

Materials. Commercial solvents and reagents were used as received.

Instrumentation. Proton magnetic resonance (¹H NMR) spectra were recorded on Bruker 400 (400 MHZ) NMR spectrometers at 23° C. Proton chemical shifts are expressed in parts per million (ppm, δ scale) and are referenced to residual protium in the NMR solvent (CHCl₃, δ 7.26). Data are represented as follows: chemical shift, multiplicity (s=singlet, d=doublet, t=triplet, q=quartet, m=multiplet and/or multiple resonances, br=broad, app=apparent), integration, and coupling constant (J) in Hertz. Carbon nuclear magnetic resonance spectra (13C NMR) were recorded on Bruker 400 (100 MHz) NMR spectrometers at 23° C. Carbon chemical shifts are expressed in parts per million (ppm, 8 scale) and are referenced to the carbon resonances of the NMR solvent (CDCl₃, δ 77.0). Infrared (IR) spectra were obtained using a Bruker ALPHA FT-IR spectrometer. Data are represented as follows: frequency of absorption (cm⁻¹), intensity of absorption (s=strong, m=medium, w=weak, br=broad). High resolution mass spectra were obtained at the Harvard University Mass Spectrometry Facility. High performance liquid chromatography purifications were performed using an Agilent Technologies 1200 series preparative HPLC system.

### Synthesis of C16-Modified Trioxacarcins

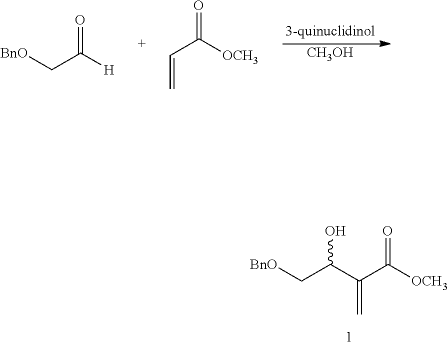

Methyl 4-(benzyloxy)-3-hydroxy-2-methylenebutanoate (1). 3-quinuclidinol (0.847 g, 6.66 mmol, 0.2 equiv.) was added to a solution of 2-(benzyloxy) acetaldehyde (5.00 g, 33.3 mmol, 1.0 equiv), methyl acrylate (9.05 mL, 100 mmol, 3.0 equiv.), and anhydrous methanol (1.00 mL, 25.0 mmol, 0.75 equiv) at 23° C. After stirring at 23° C. overnight (˜18 h), the reaction mixture was partitioned between sat. aq. NH₄Cl solution and CH₂Cl₂. The aqueous layer was then extracted with CH₂Cl₂. The organic layers were combined, dried over anhydrous sodium sulfate, filtered, and concentrated under reduced pressure. The residue was purified by flash-column chromatography (10% EtOAc in Hexanes grading to 30% EtOAc in Hexanes) to afford 5.81 g (74% yield) of 1 as a colorless oil. Spectroscopic data was identical to previously reported data.

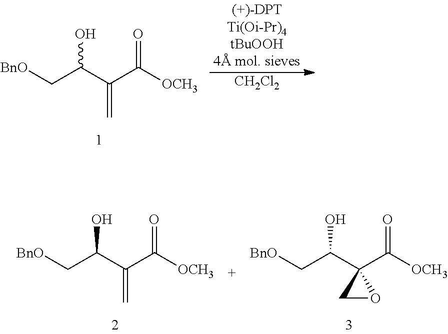

Methyl (R)-2-((S)-2-(benzyloxy)-1-hydroxyethyl)oxirane-2-carboxylate (3). A flask charged with anhydrous CH₂Cl₂ (215 mL) and activated 4 Å molecular sieves (700 mg) was cooled to −20° C. before the sequential dropwise addition of Ti(Oi-Pr)₄ (6.65 mL, 22.5 mmol, 1.0 equiv.) and (+)-DIPT (5.65 mL, 27.0 mmol, 1.2 equiv.). The resultant solution was then stirred at −20° C. for 30 minutes and a/BuOOH solution (4.49 mL, 5 M in decane, 22.5 mmol, 1.0 equiv.) was added dropwise and the temperature of the solution was allowed to warm to −10° C. over 30 minutes. Next, 1 (5.31 g, 22.5 mmol, 1.0 equiv.) was added dropwise as a solution in anhydrous CH₂Cl₂ (10 mL, resultant solution 0.1 M). The reaction was then stirred at −20° C. for 20 h before it was filtered through celite and partitioned between 1 N HCl solution and CH₂Cl₂. The aqueous layer was extracted with CH₂Cl₂ and the organic layers were combined, dried over anhydrous sodium sulfate, filtered, and concentrated under reduced pressure. The residue was purified by flash-column chromatography (10% EtOAc in Hexanes grading to 30% EtOAc in Hexanes) to afford 2.75 g (49% yield. 96% ee) of 2 as a colorless oil and 3 contaminated with (+)-DIPT. 2 contaminated with (+)-DIPT was then taken up in CH₂Cl₂ (100 mL). To this solution was added distilled water (80 mL) and brine (20 mL). To this biphasic mixture was then added solid NaOH pellets (6.00 g). The resultant biphasic mixture was vigorously stirred for 30 minutes at 23° C. After 30 minutes, the biphasic mixture was partitioned between sat. aq. sodium chloride and CH₂Cl₂. The aqueous layer was extracted with CH₂Cl₂. The organic layers were combined, dried over anhydrous sodium sulfate, filtered, and concentrated under reduced pressure to afford 2.47 g (47% recovery, 96% ee) of 3 as a colorless oil. ¹H NMR (400 MHZ, CDCl₃) δ 7.53-7.25 (m, 5H), 4.56 (s, 2H), 4.15 (dd, J=5.2, 4.0 Hz, 1H), 3.78-3.64 (m, 4H), 3.13 (d, J=5.8 Hz, 1H), 3.07 (d, J=5.8 Hz, 1H), 2.74 (s, 1H). ¹³C NMR (101 MHz, CDCl₃) δ 169.73, 137.78, 128.43, 127.79, 127.71, 73.55, 70.75, 69.35, 56.28, 52.65, 50.06. FTIR (neat), cm⁻¹:3476 (br), 3030 (s), 2866 (s), 1736 (s). HRMS (ESI): Calcd for (C₁₃H₁₆O₅+H)⁺ 253.1071, found 253.1073.

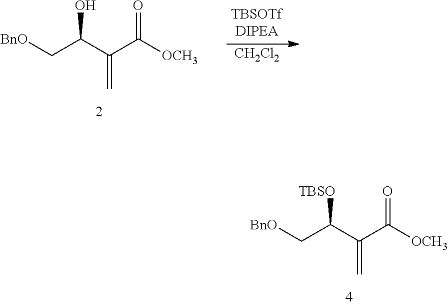

Methyl(S)-4-(benzyloxy)-3-((tert-butyldimethylsilyl)oxy)-2-methylenebutanoate (4). TBSOTf (3.65 mL, 20.9 mmol, 2.0 equiv.) was added dropwise to an ice-cooled solution 2 (2.47 g, 10.4 mmol, 1.0 equiv.) and DIPEA (2.64 mL, 11.5 mmol, 1.1 equiv.) in anhydrous CH₂Cl₂ (33.0 mL, 0.32 M). The solution was then stirred at 0° C. for 1 h. After 1 h, the solution was partitioned between sat. aq. NH₄Cl and CH₂Cl₂. The aqueous layer was then extracted with CH₂Cl₂. The organic layers were combined, dried over anhydrous sodium sulfate, filtered, and concentrated under reduced pressure. The residue was purified by flash-column chromatography (5% EtOAc in Hexanes grading to 15% EtOAc in Hexanes) to afford 3.66 g (quant. yield) of 4 as a colorless oil. Spectroscopic data was identical to previously reported data.

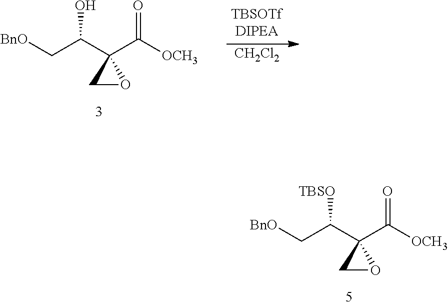

Methyl (R)-2-((S)-2-(benzyloxy)-1-((tert-butyldimethylsilyl)oxy)ethyl)oxirane-2-carboxylate (5). TBSOTf (3.81 mL, 21.8 mmol, 2.0 equiv.) was added dropwise to an ice-cooled solution 3 (2.47 g, 10.4 mmol, 1.0 equiv.) and DIPEA (2.76 mL, 12.0 mmol, 1.1 equiv.) in anhydrous CH₂Cl₂ (33.0 mL, 0.32 M). The solution was then stirred at 0° C. for 1 h. After 1 h, the solution was partitioned between sat. aq. NH₄Cl and CH₂Cl₂. The aqueous layer was then extracted with CH₂Cl₂. The organic layers were combined, dried over anhydrous sodium sulfate, filtered, and concentrated under reduced pressure. The residue was purified by flash-column chromatography (5% EtOAc in Hexanes grading to 15% EtOAc in Hexanes) to afford 3.78 g (95% yield) of 5 as a colorless oil. ¹H NMR (400 MHZ, CDCl₃) δ 7.38-7.25 (m, 5H), 4.51 (s, 2H), 4.47 (t, J=5.0 Hz, 1H), 3.70 (s, 3H), 3.59 (d, J=5.1 Hz, 2H), 3.04 (q, J=6.2 Hz, 2H), 0.86 (s, 9H), 0.07 (d, J=1.6 Hz, 6H). ¹³C NMR (101 MHz, CDCl₃) δ 169.67, 138.08, 128.35, 127.59, 73.41, 71.89, 68.69, 57.59, 52.40, 48.10, 25.71, 18.20,−4.87, −4.98. FTIR (neat), cm⁻¹:2952 (s), 2928 (s), 2886 (s), 2856 (s), 1737 (s). HRMS (ESI): Calcd for (C₁₉H₃₀O₅Si+H)⁺367.1935, found 367.1934.

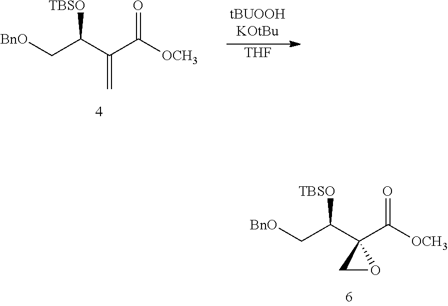 [SMILES: C=C(C(=O)OC)[C@@H](COCc1ccccc1)O[Si](C)(C)C(C)(C)C ; COC(=O)[C@]1([C@@H](COCc2ccccc2)O[Si](C)(C)C(C)(C)C)CO1 ; "THF"]

Methyl (R)-2-((R)-2-(benzyloxy)-1-((tert-butyldimethylsilyl)oxy)ethyl)oxirane-2-carboxylate (6). A solution of KOtBu in THF (1.04 mL, 1 M, 1.09 mmol, 0.1 equiv.) was added dropwise to a solution of 4 (3.66 g, 10.4 mmol) and/BuOOH (4.20 mL, 5 M, 20.9 mmol, 2.0 equiv.) at 0° C. After 5 h, a second portion of/BuOOH (4.20 mL, 5 M, 20.9 mmol, 2.0 equiv.) was added to the solution and a second portion of KOtBu in THF (1.04 mL, 1 M, 1.09 mmol, 0.1 equiv.) was added dropwise. The solution was warmed to 4° C. and stirred overnight. After a total reaction period of 24 h, solid Na₂SO₃ (6.58 g, 52.2 mmol, 5 equiv.) was added in one portion. The resulting mixture was warmed to 23° C. and then stirred at 23° C. for 30 minutes. The reaction mixture was filtered through a pad of Celite eluting with ether. The filtrate was then collected and concentrated under reduced pressure. The residue was purified by flash-column chromatography (5% EtOAc in Hexanes grading to 15% EtOAc in Hexanes) to afford 3.40 g (89% yield) of 6 as a colorless oil. Spectroscopic data was identical to previously reported data.

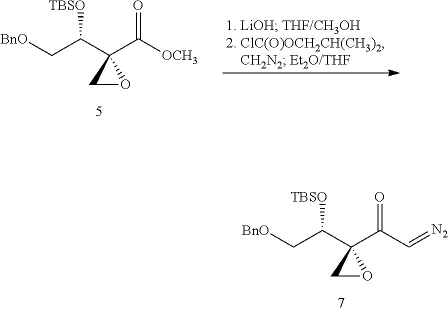

1-((R)-2-((S)-2-(Benzyloxy)-1-((tert-butyldimethylsilyl)oxy)ethyl)oxiran-2-yl)-2-diazoethan-1-one (7). Aqueous LiOH solution (20.6 mL, 1 M, 20.6 mmol, 2.0 equiv.) was added to an ice-cooled solution of 5 (3.78 g, 10.3 mmol) in THF (30 mL) and methanol (15 mL). The resultant solution was stirred overnight at 0° C. After stirring overnight (˜18 h), the solution was diluted with EtOAc (40 mL) and adjusted to pH 1 by addition of 1 N HCl (30 mL). The resultant biphasic mixture was then partitioned between EtOAc and brine. The aqueous layer was then extracted with EtOAc. The organic layers were combined, dried over anhydrous sodium sulfate, filtered, and concentrated under reduced pressure to afford crude intermediate as a pale yellow oil. ¹H NMR (400 MHZ, CDCl₃) δ 7.37-7.25 (m, 5H), 4.56 (q, J=12.1 Hz, 2H), 3.91 (t, J=4.1 Hz, 1H), 3.70-3.56 (m, 2H), 3.12 (d, J=5.9 Hz, 1H), 2.92 (d, J=5.9 Hz, 1H), 0.91 (s, 9H), 0.12 (d, J=4.4 Hz, 6H). HRMS (ESI): Calcd for (C₁₈H₂₈O₅Si+H)⁺ 353.1779, found 353.1783. **CAUTION!!** Diazomethane is an explosion hazard and is extremely toxic! The following operation was conducted behind a blast shield. The reaction was run under low lighting; the hood lights were turned off, and after addition of diazomethane the reaction flask was covered with aluminum foil to exclude light. Isobutyl chloroformate (2.81 mL, 21.7 mmol, 2.1 equiv.) was added dropwise to a solution of triethylamine (5.76 mL, 41.3 mmol, 4.0 equiv.) in anhydrous THF (54 mL) at −15° C. After a fine white suspension forms, the suspension was then stirred at −15° C. for 20 minutes. After 20 minutes, crude intermediate was added dropwise as a solution in anhydrous THF (22 mL). After stirring at −15° C., TLC (20% EtOAc in Hexanes) shows consumption of crude intermediate and a single major intermediate, the putative mixed anhydride. Next, freshly distilled diazomethane in ether (20.6 mmol, 2.0 equiv., procedure to safely distill diazomethane below) was added portion-wise using a graduated plastic pipette. The resultant suspension was bright yellow. The cooling bath was removed and the solution was allowed to warm to 23° C. After 2 h, TLC (20% EtOAc in Hexanes) shows consumption of the putative mixed anhydride and the appearance of a single, strongly-UV active spot below. To quench any unreacted diazomethane, a stream of nitrogen was passed into the headspace of the flask through a 19-gauge needle through the septum, and the flask was vented through tubing to an Erlenmeyer flask containing ˜300 mL of 50% aq. acetic solution. The mixture was vented for 30 minutes, bubbling of the acid solution indicates that the diazomethane is being quenched. The quenched mixture (still pale yellow) was then partitioned between sat. aq. NaHCO₃ solution and CH₂C₁₂. The aqueous layer was then extracted with CH₂Cl₂. The organic layers were combined, dried over anhydrous sodium sulfate, filtered, and concentrated under reduced pressure. The residue was purified by flash-column chromatography (5% EtOAc in Hexanes grading to 15% EtOAc in Hexanes) to afford 3.06 g (79% yield) of 7 as a yellow oil. ¹H NMR (400 MHZ, CDCl₃) δ 7.38-7.26 (m, 5H), 5.66 (s, 1H), 4.55 (t, J=5.5 Hz, 1H), 4.51 (s, 2H), 3.61 (dd, J=10.0, 5.8 Hz, 1H), 3.46 (dd, J=10.0, 5.2 Hz, 1H), 3.12 (d, J=5.4 Hz, 1H), 2.74 (d, J=5.4 Hz, 1H), 0.85 (s, 9H), 0.06 (d, J=1.1 Hz, 6H). ¹³C NMR (101 MHZ, CDCl₃) δ 192.44, 138.05, 128.37, 127.62, 73.26, 70.96, 68.81, 61.92, 52.62, 49.68, 25.72, 18.17,-4.72,-5.12. FTIR (neat), cm⁻¹:2954 (s), 2928 (s), 2856 (s), 2107 (s), 1740 (s), 1637 (s). HRMS (ESI): Calcd for (C₁₉H₂₈N₂O+Si+H)⁺377.1891, found 377.1894.

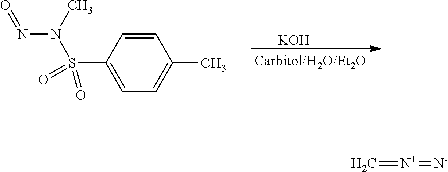

To begin, the Aldrich Mini Diazald® apparatus was charged with a stir bar, KOH (3.20 g, 57.0 mmol, 1.9 equiv.), deionized water (5 mL), and 2-(2-Ethoxyethoxy) ethanol (18 mL). The addition funnel was then charged with Diazald® (6.40 g, 29.9 mmol) in diethyl ether (25 mL) and the receiving flask was cooled to −78° C. in a dry ice/acetone bath. The round bottom flask containing KOH, water, and 2-(2-Ethoxyethoxy) ethanol was then heated to 65° C. in an oil bath. The Diazald® ethereal solution was added such that the rate of addition and diazomethane distillation was equal. After Diazald® addition was complete, ether (25 mL) was added until the resulting distillate was colorless. Distillation of diazomethane was assumed to be 70% yielding as per the manufacturer's label. Next, the receiving flask containing yellow diazomethane solution was removed and a few pellets of KOH were added. If the ethereal diazomethane solution was not used immediately, the flask was sealed with a yellow plastic cap and covered in aluminum foil to exclude light. The ethereal diazomethane solution was stored at −20° C. The reaction mixture in the distillation apparatus was carefully quenched by dropwise addition of 50% aq. acetic acid solution. The disassembled glassware was left at 23° C. overnight (˜18 h), then washed with water and acetone, and air-dried.

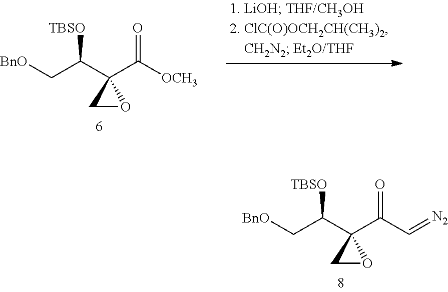

1-((R)-2-((R)-2-(Benzyloxy)-1-((tert-butyldimethylsilyl)oxy)ethyl)oxiran-2-yl)-2-diazoethan-1-one (8). 6 was transformed to 8 using an identical procedure to 7. Crude intermediate was obtained as a faint yellow oil. ¹H NMR (400 MHZ, CDCl₃) δ 7.42-7.28 (m, 5H), 4.57 (d, J=1.3 Hz, 2H), 4.21 (dd, J=6.5, 5.2 Hz, 1H), 3.64 (qd, J=9.6, 5.9 Hz, 2H), 3.09 (s, 2H), 0.95-0.84 (m, 9H), 0.15-0.05 (m, 6H). HRMS (ESI): Calcd for (C₁₈H₂₈O₅Si+H)⁺353.1779, found 353.1783. 8 was obtained as a yellow oil. ¹H NMR (400 MHz, CDCl₃) δ 7.42-7.27 (m, 5H), 5.58 (s, 1H), 4.62-4.46 (m, 2H), 3.60 (qd, J=9.6, 6.5 Hz, 2H), 3.16 (d, J=5.8 Hz, 1H), 2.75 (d, J=5.9 Hz, 1H), 0.88 (s, 9H), 0.09 (d, J=4.9 Hz, 6H). ¹³C NMR (101 MHz, CDCl₃) δ 192.35, 138.16, 128.30, 127.48, 73.34, 71.81, 68.82, 63.95, 52.03, 50.32, 25.75, 18.16-4.80,-5.02. FTIR (neat), cm⁻¹:2954 (s), 2928 (s), 2856 (s), 2107 (s), 1753 (s), 1637 (s). HRMS (ESI): Calcd for (C₁₉H₂₈N₂O₄Si+H)⁺377.1891, found 377.1895.

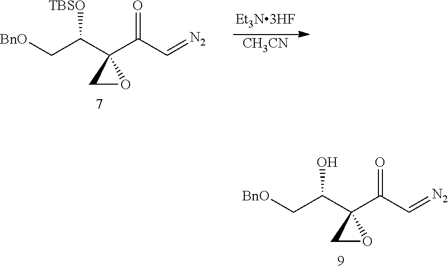

1-((R)-2-((S)-2-(Benzyloxy)-1-hydroxyethyl)oxiran-2-yl)-2-diazoethan-1-one (9). Triethylamine trihydrofluoride (5.55 mL, 34.1 mmol, 6.0 equiv.) was added dropwise to 7 (2.14 g, 5.68 mmol) in anhydrous acetonitrile (11.0 mL, 0.5 M) at 23° C. The reaction vessel was wrapped in aluminum foil to exclude light and then stirred at 23° C. for 24 h. After 24 h, the solution was diluted with CH₂Cl₂ and added slowly to a solution of sat. aq. NaHCO₃ (40 mL). The biphasic mixture was then partitioned between brine and CH₂Cl₂. The aqueous layer was then extracted with CH₂Cl₂. The organic layers were combined, dried over anhydrous sodium sulfate, filtered, and concentrated under reduced pressure. The residue was purified by flash-column chromatography (10% EtOAc in Hexanes grading to 30% EtOAc in Hexanes) to afford 1.49 g (quant. yield) of 9 as a yellow oil. ¹H NMR (400 MHZ, CDCl₃) δ 7.32-7.17 (m, 5H), 5.52 (s, 1H), 4.50 (s, 2H), 3.87 (ddd, J=7.5, 5.5, 4.3 Hz, 1H), 3.68 (dd, J=10.1, 4.3 Hz, 1H), 3.60 (dd, J=10.0, 5.6 Hz, 1H), 3.39 (dd, J=7.7, 1.7 Hz, 1H), 3.02 (d, J=5.1 Hz, 1H), 2.79 (d, J=5.1 Hz, 1H). ¹³C NMR (101 MHZ, CDCl₃) δ 193.65, 137.91, 128.41, 127.71, 127.56, 73.51, 71.50, 71.10, 59.94, 52.81, 51.58. FTIR (neat), cm⁻¹:3448 (br), 3112 (s), 2862 (s), 2105 (s), 1624 (s). HRMS (ESI): Calcd for (C₁₃H₁₄N₂O₄+H)⁺263.1026, found 263.1030.

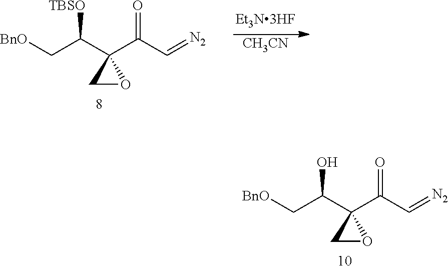

1-((R)-2-((R)-2-(Benzyloxy)-1-hydroxyethyl)oxiran-2-yl)-2-diazoethan-1-one (10). 8 was transformed to 10 using an identical procedure to 9. 10 was obtained as a yellow oil. 1H NMR (400 MHZ, CDCl₃) δ 7.32-7.17 (m, 5H), 5.54 (s, 1H), 4.64-4.31 (m, 3H), 3.60 (d, J=4.8 Hz, 2H), 3.15 (d, J=5.5 Hz, 1H), 2.66 (d, J=5.5 Hz, 1H), 2.44-2.26 (m, 1H). ¹³C NMR (101 MHz, CDCl₃) δ 192.32, 137.71, 128.46, 127.83, 127.71, 73.47, 70.75, 67.14, 61.99, 52.57 49.12. FTIR (neat), cm⁻¹:3441 (br), 3115 (s), 2862 (s), 2105 (s), 1742 (s), 1624 (s). HRMS (ESI): Calcd for (C₁₃H₁₄N₂O₄+Na)⁺285.0846, found 285.0846.

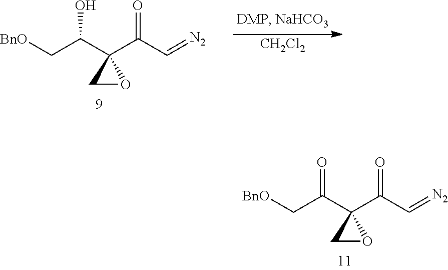

(S)-2-(Benzyloxy)-1-(2-(2-diazoacetyl)oxiran-2-yl) ethan-1-one (11). Dess-Martin periodinane (3.00 g, 7.07 mmol, 1.1 equiv.) was added to a suspension of 9 (1.68 g, 6.42 mmol) and NaHCO₃ (5.40 g, 64.2 mmol, 10 equiv.) in CH₂Cl₂ (64 mL, 0.1 M). The suspension was then stirred at 23° C. for 1 h. After 1 h the reaction mixture was diluted with diethyl ether (50 mL). The reaction mixture was then filtered through a pad of Celite. The filtrate was then concentrated under reduced pressure. The residue was purified by flash-column chromatography (20% EtOAc in Hexanes grading to 40% EtOAc in Hexanes) to afford 1.24 g (75% yield) of 11 as a yellow oil. 10 was transformed to 11 using an identical procedure to provide 11 as a yellow oil. ¹H NMR (400 MHZ, CDCl₃) δ 7.32-7.17 (m, 5H), 5.64 (s, 1H), 4.58-4.45 (m, 2H), 4.34 (s, 2H), 3.16 (d, J=6.1 Hz, 1H), 2.92 (d, J=6.1 Hz, 1H). ¹³C NMR (101 MHz, CDCl₃) δ 199.42, 187.43, 136.95, 128.53, 128.11, 128.02, 73.60, 73.35, 61.08, 53.75, 52.32. FTIR (neat), cm⁻¹:3114 (br), 2923 (br), 2110 (s), 2105 (s), 1729 (s), 1628 (s). HRMS (ESI): Calcd for (C₁₃H₁₂N₂O₄+H)⁺261.0870, found 261.0873.

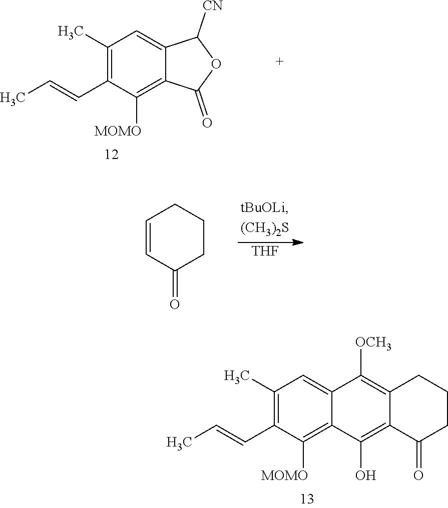

(E)-9-Hydroxy-10-methoxy-8-(methoxymethoxy)-6-methyl-7-(prop-1-en-1-yl)-3,4-dihydroanthracen-1 (2H)-one (13). Lithium tert-butoxide (1.0 M solution in THF, 193 mL, 193 mmol, 3.0 equiv.) was added to a solution of 12 (10:1 E/Z, 17.5 g, 64.2 mmol) in anhydrous THF (320 mL) at −78° C. After 5 minutes, a solution of cyclohexenone (6.21 mL, 64.2 mmol, 1.0 equiv) in anhydrous THF (320 mL) was added by cannula. The reaction flask was allowed to warm to −25° C. over 2.5 h, then dimethyl sulfate (55.2 mL, 578 mmol, 9.0 equiv.) was added. The reaction flask was allowed to warm to 23° C. over 2 h. After an additional 2.5 h, the reaction mixture was diluted with 10% aqueous ammonium hydroxide solution (500 mL) and stirred for 10 minutes to quench excess dimethyl sulfate. The layers were separated and the organic layer was concentrated. The solid residue was dissolved in ethyl acetate (1 L) and the resultant solution was washed sequentially with water (500 mL) then sat. aq. sodium chloride solution (500 mL), and the washed solution was dried over anhydrous sodium sulfate. The dried solution was filtered and the filtrate was concentrated to provide 13 (22.8 g, quant. yield) as an orange solid. This material was analytically pure and used in subsequent steps without further purification. 1H NMR (10:1 E/Z ratio of alkene isomers, major isomer reported, 500 MHz, CDCl₃) δ:15.02 (s, 1H), 7.59 (s, 1H), 6.53 (dq, J=16.1, 1.8 Hz, 1H), 6.10 (dq, J=16.1, 6.2 Hz, 1H), 5.09 (s, 2H), 3.80 (s, 3H), 3.60 (s, 3H), 3.04 (t, J=6.2 Hz, 2H), 2.74 (t, J=6.4 Hz, 2H), 2.49 (s, 3H), 2.09 (p, J=6.4 Hz, 2H), 1.97 (dd, J=6.5, 1.8 Hz, 3H). ¹³C NMR (125 MHz, CDCl₃) δ 205.0, 161.6, 153.8, 143.1, 141.3, 133.5, 132.2, 130.8, 127.9, 125.4, 118.5, 117.3, 110.9, 101.3, 61.1, 58.0, 39.1, 23.6, 22.4, 22.3, 19.5. FTIR (neat), cm-1:2944 (m), 1612 (s), 1561 (m), 1443 (m), 1396 (m), 1375 (s), 1153 (s), 1040 (s), 961 (s), 912 (s), 731 (w). HRMS (ESI): Calcd for (C₂₁H₂₄O₅+H)+357.1697, found 357.1689.

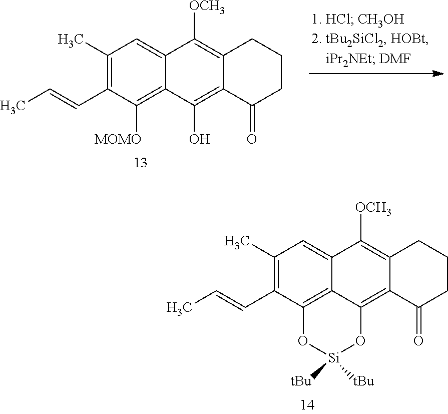

(E)-2,2-Di-tert-butyl-7-methoxy-5-methyl-4-(prop-1-en-1-yl)-9,10-dihydroanthra [1,9-de][1,3,2]dioxasilin-11 (8H)-one (14). 37% w/w Aqueous hydrochloric acid solution (11.7 mL, 142 mmol, 2.22 equiv.) was added to a suspension of 13 (10:1 E/Z, 22.9 g, 643 mmol) in methanol (640 mL, 1 M) at 0° C. After 30 minutes, the cooling bath was removed and the resulting red suspension was allowed to warm to 23° C. The reaction mixture was stirred vigorously at 23° C. for 48 h and then was carefully poured onto a mixture of ethyl acetate (1 L), saturated aqueous NaHCO₃ solution (500 mL), and deionized water (500 mL). The organic layer was separated and the aqueous layer was extracted with ethyl acetate. The combined organic layers were concentrated to approximately 1.5 L total volume and washed with sat. aq. sodium chloride solution (500 mL). The washed organic solution was dried over anhydrous sodium sulfate, filtered, and concentrated to provide the crude free phenol as a dark orange solid. The crude product was carried through to the following transformation. Di-tert-butyldichlorosilane (24.3 mL, 115 mmol, 1.8 equiv.) was added to a dark red solution of the crude product, N,N-diisopropylamine (55.8 mL, 320 mmol, 5.0 equiv.), and anhydrous HOBt (4.32 g, 32.0 mmol, 0.5 equiv.) in DMF (1.3 L) at 23° C. The reaction flask was then heated in an oil bath to 55° C. After 1 h, the heating bath was removed and the reaction flask was allowed to cool to 23° C. The reaction mixture was then partitioned between sat. aq. NaHCO₃ solution (1 L) and diethyl ether (1.5 L). The layers were separated and the aqueous layer was extracted with diethyl ether. The combined organic layers were washed successively with water and sat. aq. sodium chloride solution. The washed solution was then dried over sodium sulfate, filtered, and concentrated under reduced pressure. The residue was purified by flash-column chromatography (0% EtOAc in Hexanes grading to 5% EtOAc in Hexanes; containing 1% triethylamine) to provide 14 (20.6 g, 20:1 E/Z, 71% yield over two steps) as a flaky yellow solid. ¹H NMR (20:1 E/Z ratio of alkene isomers, major isomer reported, 400 MHz, CDCl₃) δ:7.36 (d, J=1.2 Hz, 1H), 6.46 (dd, J=15.9, 1.5 Hz, 1H), 6.36 (dq, J=15.7, 6.1 Hz, 1H), 3.82 (s, 3H), 3.03 (t, J=6.1 Hz, 2H), 2.64 (t, J=6.5 Hz, 2H), 2.49 (d, J=1.0 Hz, 3H), 2.07 (p, J=5.8 Hz, 2H), 1.97 (dd, J=6.3, 1.4 Hz, 3H), 1.12 (s, 18H). ¹³C NMR (100 MHz, CDCl₃) δ:196.8, 150.7, 150.6, 144.4, 139.8, 131.1, 130.9, 130.3, 124.3, 121.6, 116.0, 114.8, 114.6, 61.1, 41.4, 26.4, 24.2, 22.5, 22.4, 21.3, 20.0. FTIR (neat), cm⁻¹:2935 (m), 2860 (w), 1681 (s), 1606 (m), 1553 (m), 1472 (m), 1445 (m), 1402 (m), 1389 (s), 1057 (s), 1014 (m), 887 (s), 828 (s), 661 (m). HRMS (ESI): Calcd for (C₂₇H₃₆O₄Si+H)⁺453.2456, found 453.2457.

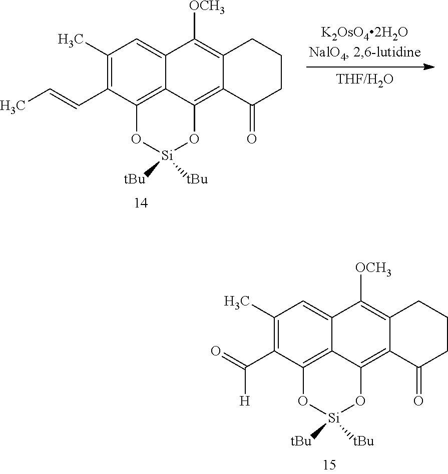

2,2-Di-tert-butyl-7-methoxy-5-methyl-11-oxo-8,9,10,11-tetrahydroanthra [1,9-de][1,3,2]dioxasiline-4-carbaldehyde (15). 2,6-Lutidine (2.56 mL, 22.1 mmol, 2.0 equiv.) was added to a vigorously stirring, ice-cooled solution of 14 (5.00 g, 11.0 mol) in a mixture of THF (150 mL) and water (75 mL). Sodium periodate (9.45 g, 44.2 mmol, 4.0 equiv.) and potassium osmate dihydrate (0.203 g, 0.552 mmol, 0.05 equiv.) were then added sequentially. After 10 minutes, the cooling bath was removed and the reaction flask was allowed to warm to 23° C. After 1.5 h, the reaction mixture was partitioned between water (600 mL), ethyl acetate (1.2 L), and hexanes (600 mL). The layers were separated and the organic layer was washed with water (600 mL) and then sat. aq. sodium chloride solution (600 mL). The washed solution was then dried over anhydrous sodium sulfate, filtered, and then concentrated under reduced pressure. The residue was purified by flash-column chromatography (0% EtOAc in Hexanes grading to 10% EtOAc in Hexanes) to provide 15 (4.06 g, 83% yield) as a yellow solid. ¹H NMR (500 MHZ, CDCl₃) δ:10.82 (s, 1H), 7.31 (s, 1H), 3.83 (s, 3H), 3.07 (t, J=6.0 Hz, 2H), 2.72 (s, 2H), 2.66 (t, J=6.5 Hz, 2H), 2.10 (p, J=6.5 Hz, 2H), 1.15 (s, 17H). ¹³C NMR (100 MHZ, CDCl₃) δ:196.2, 191.0, 160.9, 150.9, 144.8, 140.7, 136.2, 134.1, 119.0, 117.1, 116.0, 114.0, 61.2, 41.2, 26.2, 24.5, 22.7, 22.3, 21.4. FTIR (neat), cm-1:2936 (m), 2862 (w), 1681 (s), 1606 (s), 1558 (w), 1327 (s), 1245 (m), 876 (m), 827 (m). HRMS (ESI): Calcd for (C₂₅H₃₂O₅+H)⁺441.2092, found 441.1980.

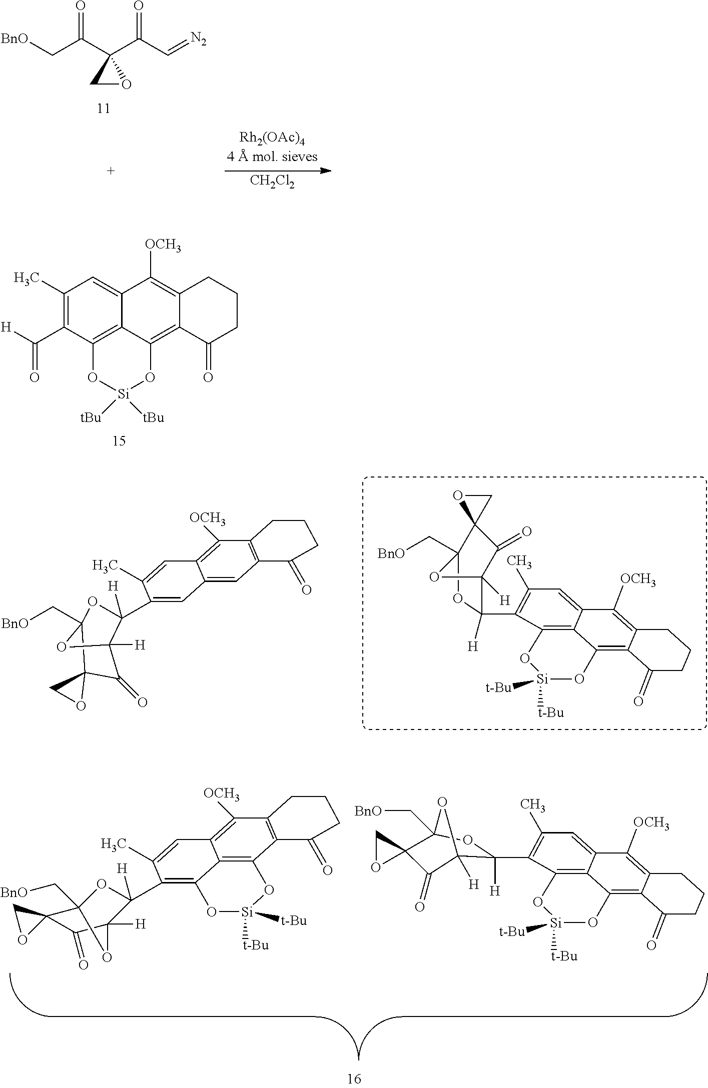

4-((IS, 2S,4S, 5R)-1-((Benzyloxy) methyl)-3-oxo-6,7-dioxaspiro [bicyclo[2.2.1]heptane-2,2′-oxiran]-5-yl)-2,2-di-tert-butyl-7-methoxy-5-methyl-9,10-dihydroanthra [1,9-de][1,3,2]dioxasilin-11 (8H)-one (16). 11 (0.673 g, 2.59 mmol, 2.0 equiv.) in anhydrous CH₂Cl₂ (5.75 mL) was added dropwise via syringe pump at a rate of 0.4 equiv. per hour to a suspension of 15 (0.57 g, 1.29 mmol), diacetoxyrhodium dimer (0.029 g, 0.065 mmol, 0.05 equiv.), activated 4 Å mol. sieves in anhydrous CH₂Cl₂ (5.75 mL, 0.255 M). After 5 h, all of 11 was dispensed and the reaction mixture was allowed to stir at 23° C. for 10 minutes. After 10 minutes, the reaction mixture was filtered through a plug of silica eluting with CH₂Cl₂ (50 mL) and 50% EtOAc in Hexanes (50 mL) to remove the mol. sieves and diacetoxyrhodium dimer. The filtrate was concentrated under reduced pressure and the residue was purified by flash column chromatography (0% EtOAc in Hexanes grading to 20% EtOAc in Hexanes) to afford a mixture of the cycloadduct products (0.587 g, 67% combined yield) and unreacted 15 (0.120 g, 21% recovery). The cycloadduct products were not separated and carried forward together to the next transformation.

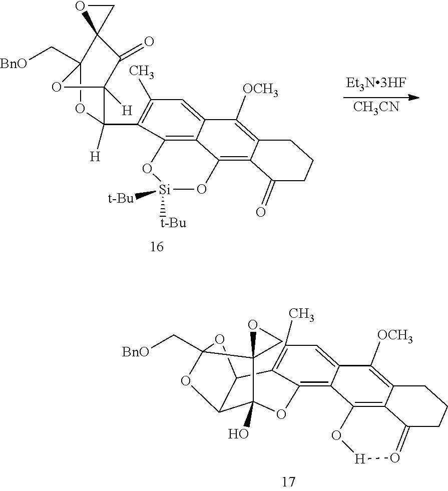

(2S,2′R,3a′R, 13a′R)-2′-((Benzyloxy) methyl)-12′, 13a′-dihydroxy-7′-methoxy-5′-methyl-3a′ 4′,8′,9′,10′,13a′-hexahydro-2′H,11′H-spiro [oxirane-2,1′-[2,4]epoxyfuro[3,2-b]naphtho[2,3-h]chromen]-11′-one (17). To a solution of the mixture of cycloadducts (16, 0.855 g, 1.27 mmol) in anhydrous acetonitrile (25.4 mL, 0.05 M) at 23° C. was added dropwise triethylamine trihydrofluoride (0.828 mL, 5.08 mmol, 4.0 equiv.). The yellow solution darkened to a light orange color. After 30 minutes, the solution was diluted with CH₂Cl₂ (25 mL) and added dropwise to a separatory funnel containing sat. aq. NaHCO₃ solution (50 mL). The aqueous layer was then extracted with CH₂Cl₂. The organic layers were combined, dried over anhydrous sodium sulfate, filtered, and concentrated under reduced pressure. The residue was purified by flash-column chromatography (40% EtOAc in Hexanes grading to 60% EtOAc in Hexanes) to afford 0.205 g (quant. yield) of 17 as a yellow solid. ¹H NMR (400 MHZ, CDCl₃) δ 14.86 (d, J=1.7 Hz, 1H), 7.42-7.36 (s, 1H), 7.35-7.14 (m, 5H), 5.22-5.16 (d, 1H), 4.77 (d, J=4.0 Hz, 1H), 4.55 (q, J=12.2 Hz, 2H), 4.39 (s, 1H), 3.76 (s, 2H), 3.73 (s, 3H), 3.16-2.91 (m, 4H), 2.68 (t, J=6.1, 2.9 Hz, 2H), 2.54 (d, J=0.9 Hz, 3H), 2.09-2.00 (m, 2H). ¹³C NMR (101 MHz, CDCl₃) δ 204.48, 162.87, 151.49, 142.43, 141.57, 137.23, 135.22, 130.25, 128.45, 127.89, 115.80, 113.76, 113.26, 111.02, 104.31, 98.53, 74.13, 73.54, 69.92, 69.59, 65.21, 60.90, 50.57, 38.78, 23.57, 22.08, 20.32. FTIR (neat), cm⁻¹:3350 (br. s) 2930 (s), 1730 (s), 1620 (s), 1569 (w). HRMS (ESI): Calcd for (C₃₀H₂₈O₉+H)⁺ 533.1806, found 533.1809.

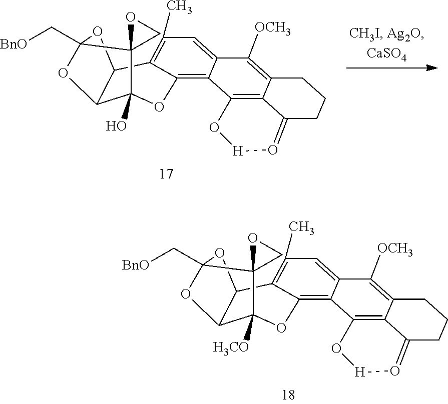

(2R,2′R,3a′R, 13a′R)-2′-((Benzyloxy) methyl)-12′-hydroxy-7′,13a′-dimethoxy-5′-methyl-3a′,4′,8′,9′,10′,13a′-hexahydro-2′H, 11′H-spiro [oxirane-2,1′-[2,4]epoxyfuro[3,2-b]naphtho[2,3-h]chromen]-11′-one (18). To a mixture of 17 (0.218 g, 0.409 mmol) and CaSO₄ (0.223 g, 1.64 mmol, 4.0 equiv.) in iodomethane (7.63 mL, 123 mmol, 300 equiv.) at 23° C. was added Ag20 (0.104 g, 0.450 mmol, 1.1 equiv.) as one portion. The reaction vessel was covered with aluminum foil to exclude light and stirred at 23° C. for 1.5 h. After 1.5 h, the reaction mixture was diluted with CH₂Cl₂ (10 mL) and filtered through a pad of Celite. The pad of Celite was washed with CH₂Cl₂ (25 mL) and filtrate was concentrated under reduced pressure to afford 0.223 g (quant. yield) of 18 as a yellow-green oil. The crude material was used in subsequent transformations without the need for purification. ¹H NMR (400 MHZ, CDCl₃) δ 14.74 (s, 1H), 7.40 (s, 1H), 7.32-7.17 (m, 5H), 5.16 (d, J=4.1 Hz, 1H), 4.75 (d, J=4.2 Hz, 1H), 4.63-4.48 (q, 2H), 3.71 (m, 5H), 3.69 (s, 3H), 2.99 (td, J=5.8, 2.5 Hz, 2H), 2.83 (d, J=5.2 Hz, 1H), 2.75 (d, J=5.3 Hz, 1H), 2.71-2.64 (m, 2H), 2.55 (s, 3H), 2.04 (p, J=6.3 Hz, 2H). 13C NMR (101 MHZ, CDCl₃) δ 204.46, 163.01, 151.43, 142.43, 141.67, 137.39, 135.24, 130.28, 128.42, 127.85, 115.65, 113.58, 113.29, 111.05, 105.11, 102.10, 74.15, 71.67, 69.30, 69.20, 64.96, 60.92, 52.85, 48.15, 38.84, 23.59, 22.13, 20.30. FTIR (neat), cm⁻¹: 2951 (s) 2855 (s), 1715 (s), 1618 (s), 1570 (s), 1495 (s). HRMS (ESI): Calcd for (C₃₁H₃₀O₉+H)⁺547.1963, found 547.1966.

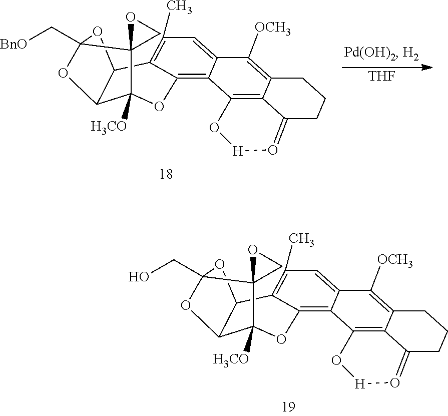

(2R.2′R,3a′R, 13a′R)-12′-Hydroxy-2′-(hydroxymethyl)-7′,13a′-dimethoxy-5′-methyl-3a′, 4′,8′,9′,10′,13a′-hexahydro-2′H, 11′H-spiro [oxirane-2,1′-[2,4]epoxyfuro[3,2-b]naphtho[2,3-h]chromen]-11′-one (19). To a solution of 18 (0.223 g, 0.408 mmol) in anhydrous THF (8.16 mL, 0.05 M) was added Pd(OH)₂ (0.143 g, 0.204 mmol, 0.5 equiv.). A hydrogen balloon was placed on the flask and the solution was degassed for 5 minutes. After 5 minutes, the outlet was removed and the reaction was allowed to stir at 23° C. for 1 h. After 1 h, the reaction mixture was diluted with EtOAc (20 mL) and filtered through a pad of Celite. The pad of Celite was washed with EtOAc (20 mL) and the filtrate was concentrated under reduced pressure. The residue was purified by flash-column chromatography (40% EtOAc in Hexanes grading to 60% EtOAc in Hexanes) to afford 0.186 g (quant. yield) of 19 as a yellow solid. ¹H NMR (400 MHZ, CDCl₃) δ 14.80 (s, 1H), 7.47 (s, 1H), 5.20 (d, J=4.1 Hz, 1H), 4.85 (d, J=4.2 Hz, 1H), 3.95 (d, J=13.0 Hz, 1H), 3.80 (m, 4H), 3.76 (s, 3H), 3.05 (dd, J=7.1, 5.1 Hz, 2H), 2.95 (d, J=4.8 Hz, 1H), 2.79-2.67 (m, 3H), 2.58 (s, 3H), 2.16-2.06 (m, 2H). ¹³C NMR (101 MHz, CDCl₃) δ 204.52, 162.95, 151.40, 142.44, 141.38, 135.30, 130.47, 115.76, 113.31, 111.13, 105.11, 102.09, 71.80, 69.29, 69.14, 60.94, 58.18, 52.89, 47.88, 38.84, 29.71, 23.61, 22.12, 20.14. FTIR (neat), cm⁻¹: 3448 (br) 2930 (br s), 2853 (s), 1715 (s), 1618 (s), 1570 (s). HRMS (ESI): Calcd for (C₂₄H₂₄O₉+H)⁺457.1493, found 457.1510.

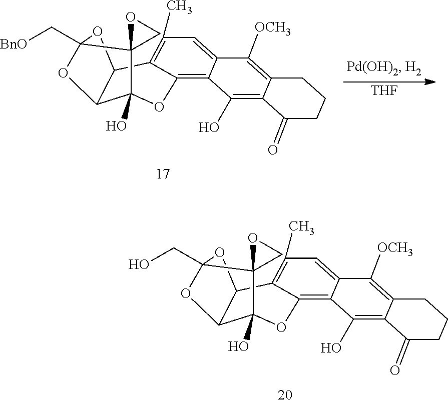

(25,2′R,3a′R, 13a′R)-12′, 13a′-dihydroxy-2′-(hydroxymethyl)-7′-methoxy-5′-methyl-3a′, 4′,8′,9′,10′,13a′-hexahydro-2′H,11′H-spiro[oxirane-2,1′-[2.4]epoxyfuro[3,2-b]naphtho[2,3-h]chromen]-11′-one (20). To a solution of 17 (5.00 mg, 9.39 μmol) in anhydrous THF (0.671 mL, 0.014 M) was added Pd(OH)₂ (6.59 mg, 9.39 μmol, 1.0 equiv.). A hydrogen balloon was placed on the flask and the solution was degassed for 5 minutes. After 5 minutes, the outlet was removed and the reaction was allowed to stir at 23° C. for 1 h. After 1 h, the reaction mixture was diluted with EtOAc (5 mL) and filtered through a pad of Celite. The pad of Celite was washed with EtOAc (10 mL) and the filtrate was concentrated under reduced pressure. The residue was purified by flash-column chromatography (40% EtOAc in Hexanes grading to 60% EtOAc in Hexanes) to afford 4.15 mg (99% yield) of 20 as a yellow solid. ¹H NMR (400 MHZ, CDCl₃) δ 14.93 (s, 1H), 7.47 (s, 1H), 5.24 (d, J=4.0 Hz, 1H), 4.85 (d, J=4.1 Hz, 1H), 4.34 (s, 1H), 3.97 (d, J=12.8 Hz, 1H), 3.85 (d, J=12.9 Hz, 1H), 3.79 (s, 3H), 3.13 (d, J=4.4 Hz, 1H), 3.09-3.00 (m, 3H), 2.73 (td, J=6.1, 1.4 Hz, 2H), 2.59 (s, 3H), 2.16-2.04 (m, 2H), 1.89 (br s, 1H). ¹³C NMR (101 MHz, CDCl₃) δ 204.52, 162.84, 151.46, 142.47, 141.30, 135.31, 130.47, 128.60, 125.39, 115.94, 113.50, 113.30, 111.11, 104.40, 98.52, 73.77, 69.95, 69.40, 60.93, 58.22, 50.20, 38.79, 29.71. FTIR (neat), cm⁻¹:3433 (br) 2924 (br s), 2853 (s), 1618 (s), 1570 (s), 1523 (s). HRMS (ESI): Calcd for (C₂₃H₂₃O₉+H)⁺ 443.1337, found 443.1340.

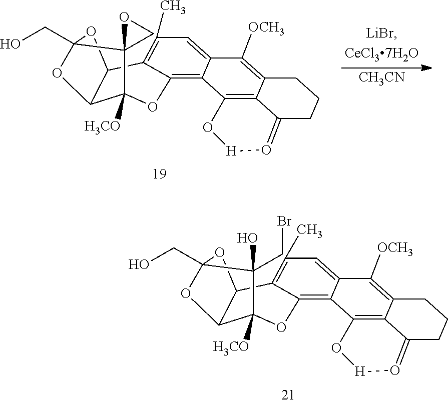

(1R,2R,3aR,13aR)-1-(bromomethyl)-1,12-dihydroxy-2-(hydroxymethyl)-7,13a-dimethoxy-5-methyl-1,2,3a,4,8,9,10,13a-octahydro-11H-2,4-epoxyfuro[3,2-b]naphtho[2,3-h]chromen-11-one (21). To a solution of 19 (5.00 mg, 11.0 μmol) in anhydrous acetonitrile (0.440 mL, 0.025 M) was added lithium bromide (0.029 g, 0.329 mmol, 30 equiv.) followed by cerium trichloride heptahydrate (0.012 g, 0.033 mmol, 3.0 equiv.). The reaction mixture was then wrapped in aluminum foil to exclude light and stirred at 23° C. overnight. After stirring overnight (˜18 h), the reaction mixture was partitioned between sat. aq. sodium sulfite solution (5 mL) and EtOAc (5 mL). The aqueous layer was then extracted with EtOAc. The organic layers were combined, dried over anhydrous sodium sulfate, filtered, and concentrated under reduced pressure. The residue was purified by reverse phase preparatory LCMS (10% acetonitrile in water grading to 90% acetonitrile in water over 30 minutes) to afford 5.89 mg (quant. yield) of 21 as a yellow solid. ¹H NMR (400 MHZ, CDCl₃) δ 14.87 (s, 1H), 7.48 (s, 1H), 5.11 (d, J=3.8 Hz, 1H), 4.69 (d, J=3.8 Hz, 1H), 4.27 (d, J=13.0 Hz, 1H), 4.19 (d, J=13.0 Hz, 1H), 3.86 (s, 1H), 3.81 (s, 3H), 3.79 (s, 3H), 3.45 (d, J=11.4 Hz, 1H), 3.36 (d, J=11.3 Hz, 1H), 3.13-3.02 (m, 2H), 2.75 (dd, J=7.2, 5.3 Hz, 2H), 2.57 (s, 3H), 2.19-2.03 (m, 2H), 2.01 (s, 1H). ¹³C NMR (101 MHz, CDCl₃) δ 204.59, 162.87, 152.18, 142.45, 141.02, 135.44, 130.75, 115.91, 112.59, 111.52, 111.25, 109.35, 104.51, 82.94, 70.66, 69.45, 60.97, 59.78, 53.17, 38.84, 32.59, 23.63, 22.09, 20.01. FTIR (neat), cm⁻¹:3467 (br) 2954 (s), 2928 (s), 2855 (s), 1729 (s), 1620 (s), 1570 (s). HRMS (ESI): Calcd for (C₂₄H₂₅BrO₉+H)⁺ 537.0755, found 537.0759.

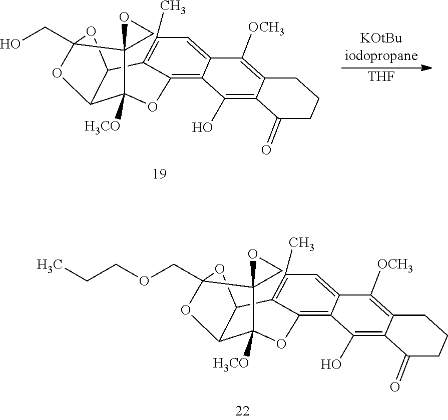

(2R,2′R,3a′R,13a′R)-12′-Hydroxy-7′,13a′-dimethoxy-5′-methyl-2′-(propoxymethyl)-3a′, 4′,8′,9′,10′,13a′-hexahydro-2′H,11′H-spiro[oxirane-2,1′-[2.4]epoxyfuro[3,2-b]naphtho[2,3-h]chromen]-11′-one (22). To a solution of 19 (5.00 mg, 11.0 μmol) in anhydrous THF (0.440 mL, 0.025 M) was added KOtBu (0.022 mL, 1M in THF, 0.022 mmol, 2 equiv.). After the addition was complete, the mixture was allowed to stir at 23° C. for 30 minutes. After 30 minutes, iodopropane (0.011 mL, 0.110 mmol, 10 equiv.) was added in one portion. The next day (˜18 h), the reaction mixture was partitioned between EtOAc (5 mL) and sat aq. NH₄Cl solution (5 mL). The aqueous layer was then extracted with EtOAc. The organic layers were combined, dried over anhydrous sodium sulfate, filtered, and concentrated under reduced pressure. The residue was purified by reverse phase preparatory LCMS (10% acetonitrile in water grading to 90% acetonitrile in water over 30 minutes) to afford 1.60 mg (30% yield) of 22 as a yellow solid. ¹H NMR (400 MHZ, CDCl₃) δ 14.80 (s, 1H), 7.45 (s, 1H), 5.20 (d, J=4.1 Hz, 1H), 4.80 (d, J=4.2 Hz, 1H), 3.85-3.70 (m, 8H), 3.52 (dt, J=9.7, 6.7 Hz, 1H), 3.43 (dt, J=9.6, 6.9 Hz, 1H), 3.05 (dd, J=7.3, 4.4 Hz, 2H), 2.90 (d, J=5.4 Hz, 1H), 2.83 (d, J=5.4 Hz, 1H), 2.73 (t, J=6.4 Hz, 2H), 2.60 (s, 3H), 2.08 (dq, J=12.4, 6.2 Hz, 2H), 0.88 (t, J=7.4 Hz, 3H). ¹³C NMR (101 MHz, CDCl₃) δ 204.48, 163.04, 151.47, 142.41, 141.73, 130.91, 128.82, 115.61, 113.57, 111.03, 105.01, 102.12, 74.25, 71.62, 69.30, 69.16, 68.17, 65.72, 60.93, 52.85, 48.19, 23.74, 23.01, 20.32, 14.08, 10.98, 10.33. FTIR (neat), cm⁻¹: 2928 (br) 2858 (s), 1726 (s), 1621 (s), 1572 (s), 1446 (s), 1389 (s).

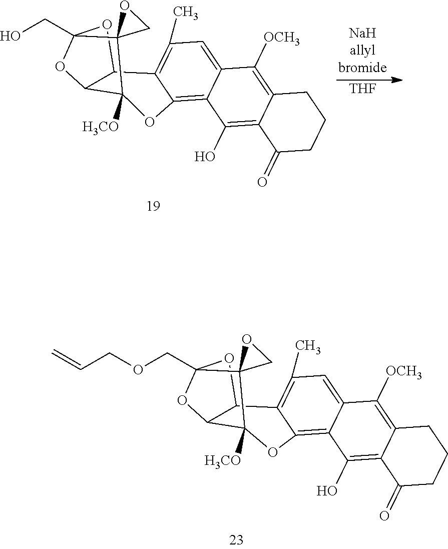

(2R,2′R,3a′R,13a′R)-2′-((Allyloxy)methyl)-12′-hydroxy-7′,13a′-dimethoxy-5′-methyl-3a′,4′,8′,9′,10′,13a′-hexahydro-2′H, 11′H-spiro[oxirane-2,1′-[2,4]epoxyfuro[3,2-b]naphtho[2.3-h]chromen]-11′-one (23). To a suspension of sodium hydride (3.42 mg, 0.085 mmol, 5.0 equiv., 60% weight suspension) in anhydrous THF (0.342 mL) was added a solution of 19 (7.80 mg, 0.017 mmol) in anhydrous THF (0.342 mL, 0.025 M). After the addition was complete, the mixture was allowed to stir at 23° C. for 30 minutes. After 30 minutes, allyl bromide (0.015 mL, 0.171 mmol, 10 equiv.) was added in one portion. The next day (˜18 h), the reaction mixture was partitioned between EtOAc (5 mL) and sat aq. NH₄Cl solution (5 mL). The aqueous layer was then extracted with EtOAc. The organic layers were combined, dried over anhydrous sodium sulfate, filtered, and concentrated under reduced pressure. The residue was purified by flash-column chromatography (10% EtOAc in Hexanes) to afford 5.60 mg of 23 (66% yield) as a yellow solid. ¹H NMR (400 MHZ, CDCl₃) δ 14.80 (s, 1H), 7.45 (s, 1H), 5.86 (ddt, J=17.3, 10.4, 5.8 Hz, 1H), 5.27 (dq, J=17.3, 1.6 Hz, 1H), 5.23-5.15 (m, 2H), 4.81 (d, J=4.1 Hz, 1H), 4.14-3.99 (m, 2H), 3.91-3.69 (m, 8H), 3.05 (td, J=5.8, 2.3 Hz, 2H), 2.90 (d, J=5.3 Hz, 1H), 2.81 (d, J=5.2 Hz, 1H), 2.73 (dd, J=7.0, 5.8 Hz, 2H), 2.60 (s, 3H), 2.09 (p, J=6.3 Hz, 2H). ¹³C NMR (101 MHZ, CDCl₃) δ 204.46, 163.02, 151.45, 142.43, 141.69, 135.24, 134.01, 130.27, 118.00, 115.63, 113.55, 113.29, 111.05, 105.03, 102.09, 73.14, 71.67, 69.19, 64.79, 60.92, 52.85, 48.16, 38.84, 29.71, 23.60, 22.13, 20.30. FTIR (neat), cm⁻¹: 2927 (br s) 2853 (br s), 2108 (s), 1621 (s), 1572 (s), 1444 (s). HRMS (ESI): Calcd for (C₂₇H₂₈O₉+H)⁺ 497.1806, found 497.1811.

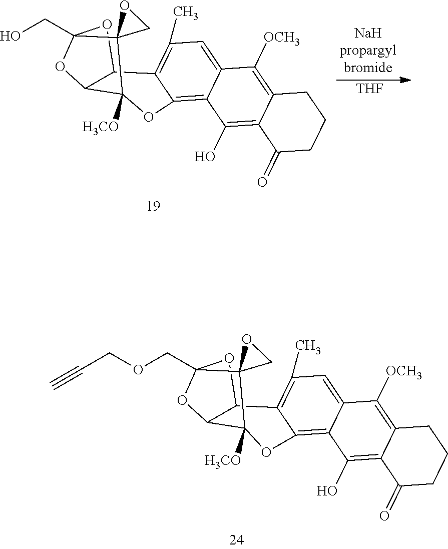

(2R,2′R,3a′R,13a′R)-12′-Hydroxy-7′,13a′-dimethoxy-5′-methyl-2′-((prop-2-yn-1-yloxy)methyl)-3a′,4′,8′,9′,10′,13a′-hexahydro-2′H,11′H-spiro[oxirane-2,1′-[2,4]epoxyfuro[3,2-b]naphtho[2.3-h]chromen]-11′-one (24). To a suspension of sodium hydride (2.19 mg, 0.055 mmol, 5.0 equiv., 60% weight suspension) in anhydrous THF (0.220 mL) was added a solution of 19 (5.00 mg, 11.0 μmol) in anhydrous THF (0.220 mL, 0.025 M). After the addition was complete, the mixture was allowed to stir at 23° C. for 30 minutes. After 30 minutes, propargyl bromide (0.012 mL, 0.171 mmol, 10 equiv., 80% w/w in toluene) was added in one portion. The next day (˜18 h), the reaction mixture was partitioned between EtOAc (5 mL) and sat aq. NH₄Cl solution (5 mL). The aqueous layer was then extracted with EtOAc. The organic layers were combined, dried over anhydrous sodium sulfate, filtered, and concentrated under reduced pressure. The residue was purified by flash-column chromatography (10% EtOAc in Hexanes) to afford 2.50 mg of 24 (46% yield) as a yellow solid. ¹H NMR (400 MHZ, CDCl₃) δ 14.80 (s, 1H), 7.46 (s, 1H), 5.22 (d, J=4.2 Hz, 1H), 4.82 (d, J=4.2 Hz, 1H), 4.26 (d, J=2.4 Hz, 2H), 3.95 (d, J=11.4 Hz, 1H), 3.86 (d, J=11.4 Hz, 1H), 3.79 (s, 3H), 3.75 (s, 3H), 3.05 (td, J=5.8, 2.4 Hz, 2H), 2.91 (d, J=5.1 Hz, 1H), 2.80 (d, J=5.1 Hz, 1H), 2.73 (t, J=6.4 Hz, 2H), 2.60 (s, 3H), 2.45 (t, J=2.4 Hz, 1H), 2.15-1.99 (m, 2H). ¹³C NMR (101 MHz, CDCl₃) δ 204.50, 162.99, 151.42, 142.42, 141.63, 135.26, 130.35, 115.70, 113.41, 113.23, 111.07, 104.90, 102.01, 78.72, 75.44, 71.70, 69.25, 69.24, 64.34, 60.94, 59.31, 52.89, 48.09, 38.85, 23.60, 22.13, 20.33. FTIR (neat), cm⁻¹:3272 (br s) 2923 (s), 2853 (s), 2107 (s), 1710 (s), 1620 (s). HRMS (ESI): Calcd for (C₂₇H₂₆O₉+H)⁺ 495.1650, found 495.1653.

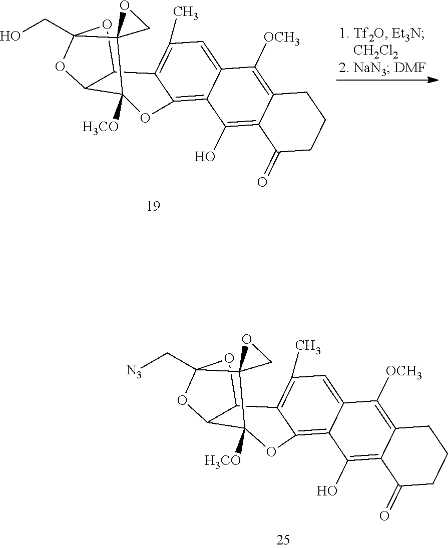

(2R,2′R,3a′R,13a′R)-2′-(Azidomethyl)-12′-hydroxy-7′,13a′-dimethoxy-5′-methyl-3a′, 4′,8′,9′,10′,13a′-hexahydro-2′H,11′H-spiro[oxirane-2,1′-[2,4]epoxyfuro[3,2-b]naphtho[2,3-h]chromen]-11′-one (25). To a solution of 19 (5.00 mg, 11.0 μmol) in anhydrous CH₂Cl₂ (0.220 mL, 0.05 M) was added triethylamine (0.015 mL, 0.110 mmol, 10 equiv.). The reaction mixture was cooled to −20° C. and triflic anhydride (10.0 μL, 0.055 mmol, 5.0 equiv.) was added in one portion. The reaction mixture was then stirred at 0° C. for 1 h. After 1 h, the reaction mixture was warmed to 23° C. and partitioned between brine (5 mL) and CH₂Cl₂ (5 mL). The aqueous layer was then extracted with CH₂Cl₂. The organic layers were combined, dried over anhydrous sodium sulfate, filtered, and concentrated under reduced pressure. The crude residue was then dissolved in anhydrous DMF (0.220 mL, 0.05 M) and sodium azide (7.13 mg, 0.110 mmol, 10 equiv.) was added in one portion. The reaction mixture was then let to stir at 23° C. overnight. After stirring at 23° C. overnight (˜18 h), the reaction mixture was partitioned between sat. aq. NaHCO₃ (5 mL) and CH₂Cl₂ (5 mL). The aqueous layer was then extracted with CH₂Cl₂. The organic layers were combined, dried over anhydrous sodium sulfate, filtered, and concentrated under reduced pressure. The crude residue was purified by reverse phase preparatory LCMS (10% acetonitrile in water grading to 90% acetonitrile in water over 30 minutes) to afford 3.90 mg of 25 (74% yield) as a yellow solid. ¹H NMR (400 MHz, CDCl₃) δ 14.80 (s, 1H), 7.48 (s, 1H), 5.25 (d, J=4.2 Hz, 1H), 4.86 (d, J=4.1 Hz, 1H), 3.80 (s, 3H), 3.75 (s, 3H), 3.66 (d, J=13.9 Hz, 1H), 3.38 (d, J=14.0 Hz, 1H), 3.05 (dd, J=7.3, 5.1 Hz, 2H), 2.93 (d, J=4.7 Hz, 1H), 2.74 (dt, J=5.6, 4.1 Hz, 3H), 2.60 (d, J=1.0 Hz, 3H), 2.10 (p, J=6.3 Hz, 2H). ¹³C NMR (101 MHZ, CDCl₃) δ 204.53, 162.91, 151.31, 142.47, 141.47, 135.34, 130.50, 115.84, 113.28, 113.22, 111.15, 105.32, 101.90, 72.00, 69.44, 69.15, 60.95, 52.91, 47.77, 46.52, 38.84, 23.61, 22.12, 20.12. FTIR (neat), cm⁻¹: 2951 (br s) 2104 (s), 1621 (s), 1572 (s), 1444 (s), 1399 (s). HRMS (ESI): Calcd for (C₂₄H₂₃N₃O₈+H)⁺ 482.1558, found 482.1560.

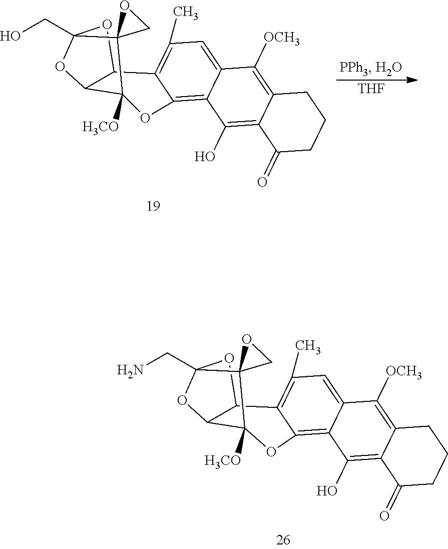

(2R,2′R,3a′R,13a′R)-2′-(Aminomethyl)-12′-hydroxy-7′,13a′-dimethoxy-5′-methyl-3a′, 4′,8′,9′,10′, 13a′-hexahydro-2′H, 11′H-spiro[oxirane-2,1′-[2,4]epoxyfuro[3,2-b]naphtho[2,3-h]chromen]-11′-one (26). To a solution of 25 (1.05 mg, 2.18 μmol) in anhydrous THF (0.220 mL, 0.01 M) was added sequentially PPh₃ (1.72 mg, 6.54 μmol, 3.0 equiv.) and distilled water (0.393 μL, 0.022 mmol, 10 equiv.). The resulting solution was stirred at 23° C. overnight. After stirring overnight (˜18 h), the reaction mixture was concentrated under reduced pressure. The crude residue was purified by reverse phase preparatory LCMS (10% acetonitrile in water grading to 90% acetonitrile in water over 30 minutes) to afford 0.70 mg of the 26 (71% yield, 2 steps) as a yellow solid. ¹H NMR (400 MHZ, CDCl₃) δ 14.80 (s, 1H), 7.47 (s, 1H), 5.17 (d, J=4.2 Hz, 1H), 4.82 (d, J=4.1 Hz, 1H), 3.78 (m, 8H), 3.05 (dd, J=7.2, 5.1 Hz, 2H), 2.91 (d, J=4.6 Hz, 1H), 2.74 (t, J=6.4 Hz, 2H), 2.65 (d, J=4.7 Hz, 1H), 2.61-2.56 (s, 3H), 2.10 (p, J=6.3 Hz, 2H). ¹³C NMR (101 MHz, CDCl₃) δ 204.53, 162.96, 151.34, 142.43, 141.36, 135.27, 130.42, 115.74, 113.50, 113.31, 111.11, 102.20, 71.59, 69.44, 69.03, 60.95, 52.91, 47.49, 38.85, 23.60, 22.12, 20.15. FTIR (neat), cm⁻¹: 2951 (br s) 2855 (s), 1713 (s), 1570 (s), 1506 (s), 1444 (s). HRMS (ESI): Calcd for (C₂₄H₂₅NO₈+H)⁺ 456.1653, found 456.1649.

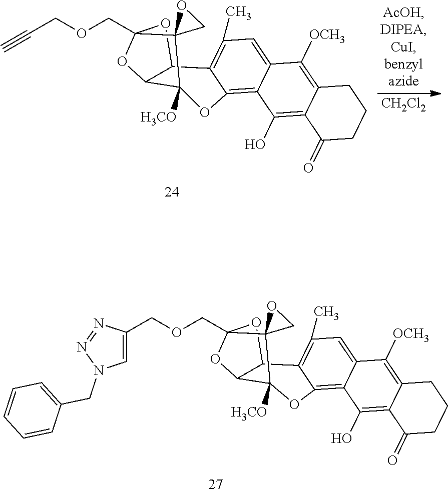

(2R,2′R,3a′R,13a′R)-2′-(((1-Benzyl-1H-1,2,3-triazol-4-yl)methoxy)methyl)-12′-hydroxy-7′,13a′-dimethoxy-5′-methyl-3a′,4′,8′,9′, 10′, 13a′-hexahydro-2′H,11′H-spiro [oxirane-2, 1′-[2,4]epoxyfuro[3,2-b]naphtho[2.3-h]chromen]-11′-one (27). To a solution of benzyl azide (0.051 mL, 0.404 mmol, 200 equiv.) and 24 (1.00 mg, 2.02 μmol) in anhydrous CH₂Cl₂ (0.200 mL) was added an anhydrous CH₂Cl₂ solution (0.200 mL, 0.005 M resultant solution) containing CuI (0.015 g, 0.081 mmol, 40 equiv.), DIPEA (0.028 mL, 0.162 mmol, 80 equiv.), and AcOH (9.26 μL, 0.162 mmol, 80 equiv.). The resulting mixture was stirred at 23° C. overnight. After stirring at 23° C. overnight (˜18 h), the reaction mixture was partitioned between sat. aq. NaHCO₃ (5 mL) and CH₂Cl₂ (5 mL). The aqueous layer was then extracted with CH₂Cl₂. The organic layers were combined, dried over anhydrous sodium sulfate, filtered, and concentrated under reduced pressure. The crude residue was purified by reverse phase preparatory LCMS (10% acetonitrile in water grading to 90% acetonitrile in water over 30 minutes) to afford 1.27 mg of 27 (quant. yield) as a yellow solid. ¹H NMR (400 MHZ, CDCl₃) δ 14.80 (s, 1H), 7.58-7.31 (m, 7H), 5.51 (s, 2H), 5.18 (d, J=4.2 Hz, 1H), 4.82-4.63 (m, 3H), 3.90-3.77 (m, 5H), 3.73 (s, 3H), 3.05 (td, J=5.8, 2.6 Hz, 2H), 2.86 (d, J=5.2 Hz, 1H), 2.74 (dd, J=11.3, 5.8 Hz, 3H), 2.57 (s, 3H), 2.09 (p, J=6.3 Hz, 2H). ¹³C NMR (101 MHz, CDCl₃) δ 204.50, 162.98, 151.41, 144.99, 142.41, 141.61, 135.25, 134.53, 130.33, 129.13, 128.78, 128.13, 122.61, 115.68, 113.43, 111.06, 104.93, 102.03, 71.58, 69.24, 65.69, 65.17, 60.94, 54.19, 52.86, 48.08, 38.85, 31.84, 29.21, 23.60, 22.67, 22.13. FTIR (neat), cm-1:2927 (br s) 1749 (s), 1699 (s), 1621 (s), 1399 (s), 1208 (s).

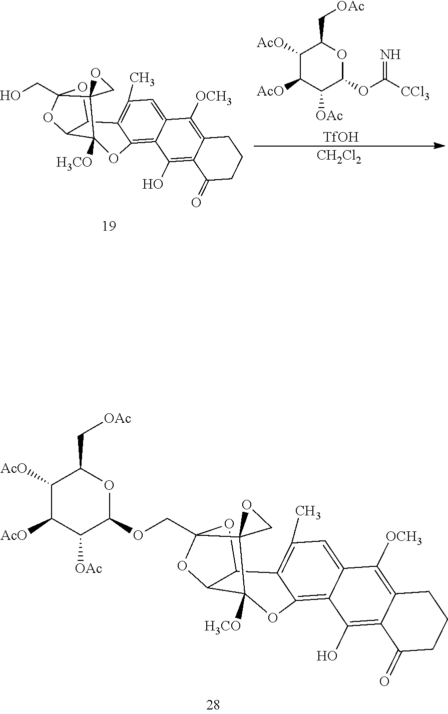

(2R,3R,4S,5R,6R)-2-(Acetoxymethyl)-6-(((2R,13a′R)-12′-hydroxy-7′,13a′-dimethoxy-5′-methyl-11′-oxo-3a′,8′,9′,10′,11′,13a′-hexahydro-2′H,4′H-spiro [oxirane-2,1′-[2,4]epoxyfuro[3,2-b]naphtho[2,3-h]chromen]-2′-yl)methoxy) tetrahydro-2H-pyran-3,4,5-triyl triacetate (28). To a solution of 19 (5.00 mg, 11.0 μmol) and the trichloracetimidate sugar (0.054 g, 0.110 mmol, 10 equiv.) in anhydrous CH₂Cl₂ (0.220 mL, 0.05 M) at −78° C. was added TfOH (0.10 μL, 1.10 μmol, 0.1 equiv.). The reaction mixture was maintained at −78° C. for 1 h. After 1 h, the reaction mixture was warmed to 23° C. and was then partitioned between sat. aq. NaHCO₃ (5 mL) and CH₂Cl₂ (5 mL). The aqueous layer was then extracted with CH₂Cl₂. The organic layers were combined, dried over anhydrous sodium sulfate, filtered, and concentrated under reduced pressure. The crude residue was purified by reverse phase preparatory LCMS (10% acetonitrile in water grading to 90% acetonitrile in water over 30 minutes) to afford 3.30 mg of 28 (38% yield) as a yellow solid. 1H NMR (400 MHZ, CDCl₃) δ 14.79 (s, 1H), 7.47 (s 1H), 5.23-5.11 (m, 2H), 5.06 (t, J=9.7 Hz, 1H), 4.96 (dd, J=9.5, 8.0 Hz, 1H), 4.80 (d, J=4.1 Hz, 1H), 4.58 (d, J=7.9 Hz, 1H), 4.36-4.02 (m, 3H), 3.93 (d, J=11.0 Hz, 1H), 3.80 (s, 3H), 3.74 (s, 3H), 3.05 (q, J=5.5 Hz, 2H), 2.82 (d, J=5.4 Hz, 1H), 2.73 (q, J=6.0, 5.5 Hz, 3H), 2.59 (s, 3H), 2.13-2.07 (m, 5H), 2.03 (s, 3H), 2.01 (s, 3H), 1.98 (s, 3H). ¹³C NMR (101 MHZ, CDCl₃) δ 204.53, 170.72, 170.28, 169.52, 169.41, 162.95, 151.35, 142.45, 141.57, 135.27, 130.37, 115.76, 113.40, 113.27, 111.08, 104.24, 102.13, 100.87, 72.79, 71.96, 71.47, 71.01, 69.44, 69.22, 68.22, 65.37, 61.86, 60.96, 52.84, 47.95, 38.85, 23.60, 22.13, 20.78, 20.67, 20.62, 20.26. FTIR (neat), cm-1:2952 (s) 1756 (s), 1621 (s), 1572 (s), 1444 (s), 1387 (s). HRMS (ESI): Calcd for (C₃₈H₄₂O₁₈+H)⁺787.2444, found 787.2446.

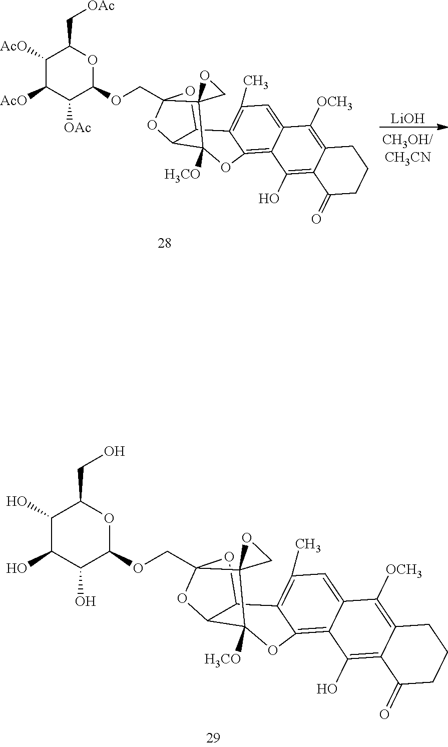

(2R,13a′R)-12′-Hydroxy-7′,13a′-dimethoxy-5′-methyl-2′-((((2R,3R,4S,5S,6R)-3,4,5-trihydroxy-6-(hydroxymethyl)tetrahydro-2H-pyran-2-yl)oxy) methyl)-3a′ 4′,8′,9′, 10′,13a′-hexahydro-2′H. 11′H-spiro [oxirane-2,1′-[2,4]epoxyfuro[3,2-b]naphtho[2,3-h]chromen]-11′-one (29). To a 0° C. solution of 28 (3.00 mg, 3.81 μmol) in anhydrous methanol (0.953 mL) and acetonitrile (0.318 mL, resultant 0.003 M solution) was added in one portion LiOH (0.381 mL, 1M, 0.381 mmol, 100 equiv.). The reaction mixture was then vigorously stirred for 2 h at 0° C. After 2 h, the reaction mixture was partitioned between 2:1 CH₂Cl₂: methanol (5 mL) and brine (5 mL). The residual LiOH was then quenched via addition of formic acid (0.022 mL, 0.572 mmol, 150 equiv.). The aqueous layer was then extracted with 2:1 CH₂Cl₂: methanol. The organic layers were combined, dried over anhydrous sodium sulfate, filtered, and concentrated under reduced pressure. The crude residue was purified by reverse phase preparatory LCMS (10% acetonitrile in water grading to 90% acetonitrile in water over 30 minutes) to afford 1.40 mg of 28 (60% yield) as a yellow solid. ¹H NMR (400 MHZ, MeOD) δ 7.50 (s, 1H), 5.26 (dd, J=4.4, 2.0 Hz, 1H), 5.02 (dd, J=4.2, 1.3 Hz, 1H), 4.27 (d, J=7.8 Hz, 1H), 4.17 (d, J=11.5 Hz, 1H), 3.85 (d, J=11.4 Hz, 2H), 3.79 (s, 3H), 3.71 (s, 3H), 3.69-3.61 (m, 1H), 3.29-3.20 (m, 2H), 3.19-3.11 (m, 1H), 3.07 (dt, J=5.1, 3.5 Hz, 3H), 2.82-2.70 (m, 3H), 2.58 (d, J=1.1 Hz, 3H), 2.08 (p, J=6.4 Hz, 2H). ¹³C NMR (101 MHz, CDCl₃) δ 209.03, 166.03, 154.95, 146.48, 145.68, 138.98, 134.56, 119.10, 117.86, 116.81, 114.67, 108.59, 107.29, 106.09, 80.63, 80.40, 77.49, 75.50, 74.02, 73.09, 72.95, 67.88, 65.17, 63.84, 55.51, 42.31, 27.06, 25.80, 22.62. FTIR (neat), cm-1:3390 (br s) 2924 (s), 1730 (s), 1642 (s), 1623 (s), 1572 (s). HRMS (ESI): Calcd for (C₃₀H₃₄O₁₄+H)⁺ 619.2021, found 619.2023.

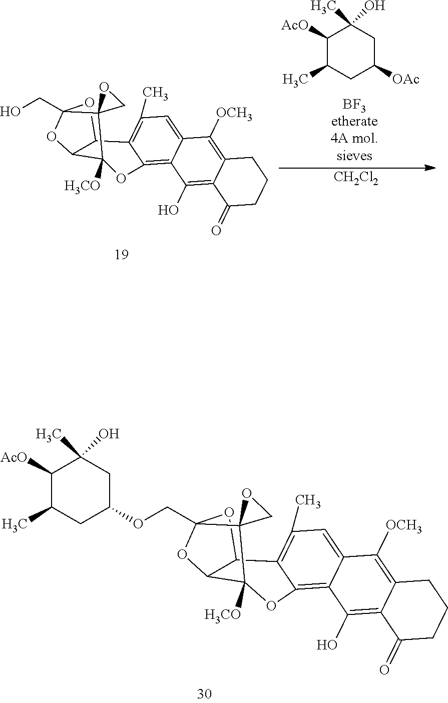

(25,3S,4R,6S)-4-Hydroxy-6-(((2R,13a′R)-12′-hydroxy-7′,13a′-dimethoxy-5′-methyl-11′-oxo-3a′,8′,9′,10′,11′,13a′-hexahydro-2′H,4′H-spiro [oxirane-2,1′-[2,4]epoxyfuro[3,2-b]naphtho[2,3-h]chromen]-2′-yl)methoxy)-2,4-dimethyltetrahydro-2H-pyran-3-yl acetate (30). To a suspension of 1-O-Acetyl Trioxacarcinose A (Prepared according to the procedures of Smaltz et. al., Organic Letters. 2012; 14 (7): 1812-1815) (22.0 mg, 0.088 mmol, 2.0 equiv.), 19 (20.0 mg, 44.0 μmol), and crushed 4 Å mol. sieves (1 mg per micromole of 1-0)-Acetyl Trioxacarcinose A) in anhydrous CH₂Cl₂ (0.876 mL, 0.05 M) at −40° C. was added BF₃·OEt₂ (5.55 μL, 44.0 μmol, 1.0 equiv.) in one portion. The solution changed color from green to red and was stirred at −40° C. for 5 minutes, at which point sat. aq. NaHCO₃ (1.0 mL) was rapidly added, restoring the solution's original green color, and the reaction vessel was warmed to 23° C. The reaction mixture was then partitioned between brine (5 mL) and CH₂Cl₂ (5 mL). The aqueous layer was then extracted with CH₂Cl₂. The organic layers were combined, dried over anhydrous sodium sulfate, filtered, and concentrated under reduced pressure. The crude residue was purified by flash-column chromatography (40% EtOAc in Hexanes) to afford 23.0 mg of 30 (82% yield) as a yellow solid. ¹H NMR (400 MHZ, CDCl₃) δ 14.80 (s, 1H), 7.46 (s, 1H), 5.16 (d, J=4.2 Hz, 1H), 4.99 (d, J=3.5 Hz, 1H), 4.83 (d, J=4.1 Hz, 1H), 4.72 (s, 1H), 4.46-4.37 (m, 1H), 4.32 (s, 1H), 4.01 (d, J=11.1 Hz, 1H), 3.80 (s, 3H), 3.75 (s, 3H), 3.68 (d, J=11.1 Hz, 1H), 3.05 (q, J=5.7 Hz, 2H), 2.89 (d, J=5.0 Hz, 1H), 2.78-2.69 (m, 3H), 2.57 (d, J=1.0 Hz, 3H), 2.13-2.05 (m, 5H), 1.91 (dd, J=14.5, 3.7 Hz, 1H), 1.72 (dt, J=14.6, 1.4 Hz, 1H), 1.14 (d, J=6.5 Hz, 3H), 1.08 (s, 3H). ¹³C NMR (101 MHz, CDCl₃) δ 204.55, 170.41, 162.92, 151.23, 142.46, 141.56, 135.30, 130.40, 115.83, 113.36, 113.21, 111.10, 104.53, 101.98, 98.00, 74.53, 71.93, 69.11, 68.60, 62.23, 61.62, 60.96, 52.92, 48.06, 38.85, 35.82, 30.97, 25.57, 23.60, 22.13, 20.92, 20.16, 16.66. FTIR (neat), cm-1:3503 (s) 2935 (s), 1734 (s), 1621 (s), 1572 (s), 1446 (s). HRMS (ESI): Calcd for (C₃₃H₃₈O₁₃+H)⁺ 665.2205, found 665.2190.

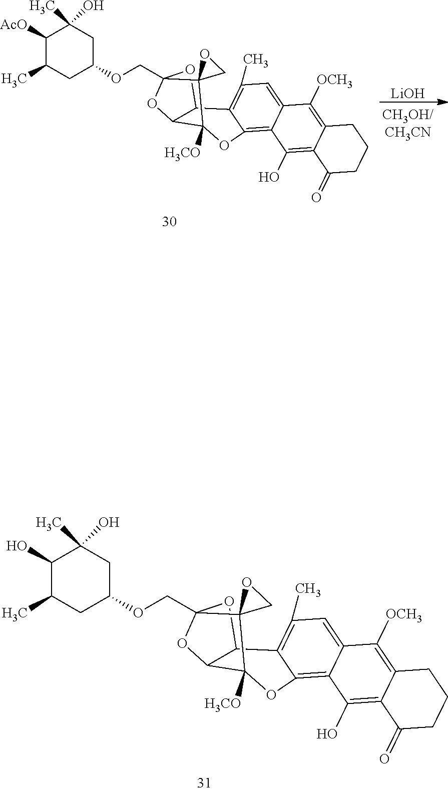

(2R,13a′R)-2′-((((25,4R,5S,6S)-4,5-dihydroxy-4,6-dimethyltetrahydro-2H-pyran-2-yl)oxy)methyl)-12′-hydroxy-7′,13a′-dimethoxy-5′-methyl-3a′,4′,8′,9′,10′,13a′-hexahydro-2′H,11′H-spiro[oxirane-2, 1′-[2.4]epoxyfuro[3.2-b]naphtho[2,3-h]chromen]-11′-one (31). To a 0° C. solution of 30 (23.0 mg, 0.036 mmol, 1.0 equiv) in 3:1 MeOH/MeCN (0.70 mL, 0.05 M) was added LiOH (86 mg, 3.6 mmol, 100 equiv). The reaction was stirred vigorously for 2 hours at 0° C. before it was partitioned between 2:1 CH₂Cl₂/MeOH (5 mL) and brine (5 mL). The residual LiOH was quenched with the addition of formic acid (0.206 mL, 5.37 mmol, 150 equiv) and the aqueous layer was extracted with 2:1 CH₂Cl₂/MeOH. The organic layers were combined, dried over Na₂SO₄, and concentrated in vacuo. The residue was purified by reverse-phase preparative HPLC-MS to afford 31 (21.0 mg, 23% yield) as a yellow solid. ¹H NMR (400 MHZ, CDCl₃) δ 14.80 (s, 1H), 7.45 (s, 1H), 5.16 (d, J=4.1 Hz, 1H), 4.93 (d, J=3.6 Hz, 1H), 4.82 (d, J=4.2 Hz, 1H), 4.38 (q, J=6.6 Hz, 1H), 4.00 (d, J=11.1 Hz, 1H), 3.80 (s, 3H), 3.75 (s, 3H), 3.67 (d, J=11.1 Hz, 1H), 3.16 (s, 1H), 3.05 (q, J=5.7 Hz, 2H), 2.89 (d, J=5.0 Hz, 1H), 2.78-2.68 (m, 3H), 2.56 (s, 3H), 2.11 (q, J=6.7 Hz, 2H), 1.91 (dd, J=14.7, 3.8 Hz, 1H), 1.28 (s, 1H), 1.26 (s, 1H), 1.23 (s, 3H). ¹³C NMR (101 MHz, CDCl₃) δ 204.54, 162.93, 151.24, 142.45, 141.54, 135.29, 130.40, 115.80, 113.39, 113.22, 111.10, 104.55, 101.99, 98.04, 74.51, 71.94, 69.92, 69.12, 62.93, 61.66, 60.95, 53.45, 52.92, 48.06, 38.85, 35.03, 25.91, 23.60, 22.13, 20.18, 16.72. FTIR (neat), cm-1:3498 (s), 2933 (s), 1621 (s), 1399 (s), 1234 (s), 1094 (s). HRMS (ESI): Calcd for (C₃₁H₃₆O₁₂+H)⁺ 623.2099, found 623.2085.

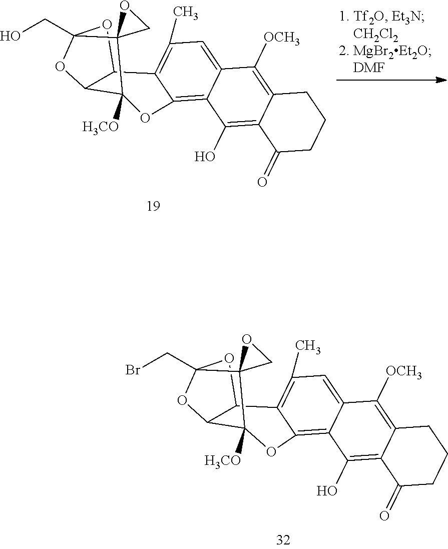

(2S,2′S,3a′R,13a′R)-2′-(Bromomethyl)-12′-hydroxy-7′,13a′-dimethoxy-5′-methyl-3a′,4′,8′9′,10′,13a′-hexahydro-2′H,11′H-spiro [oxirane-2,1′-[2,4]epoxyfuro[3,2-b]naphtho[2,3-h]chromen]-11′-one (32). To a solution of 19 (5.00 mg, 11.0 μmol) in anhydrous CH₂Cl₂ (0.220 mL, 0.05 M) was added triethylamine (0.015 mL, 0.110 mmol, 10 equiv.). The reaction mixture was cooled to −20° C. and triflic anhydride (10.0 μL, 0.055 mmol, 5.0 equiv.) was added in one portion. The reaction mixture was then stirred at 0° C. for 1 h. After 1 h, the reaction mixture was warmed to 23° C. and partitioned between brine (5 mL) and CH₂Cl₂ (5 mL). The aqueous layer was then extracted with CH₂Cl₂. The organic layers were combined, dried over anhydrous sodium sulfate, filtered, and concentrated under reduced pressure. The crude residue was then dissolved in anhydrous DMF (0.220 mL, 0.05 M) and magnesium bromide etherate (28.0 mg, 0.110 mmol, 10 equiv.) was added in one portion. The reaction mixture was then let to stir at 23° C. overnight. After stirring at 23° C. overnight (˜18 h), the reaction mixture was partitioned between sat. aq. NaHCO₃ (5 mL) and CH₂Cl₂ (5 mL). The aqueous layer was then extracted with CH₂Cl₂. The organic layers were combined, dried over anhydrous sodium sulfate, filtered, and concentrated under reduced pressure. The crude residue was purified by reverse phase preparatory LCMS (10% acetonitrile in water grading to 90% acetonitrile in water over 30 minutes) to afford 1.10 mg of 32 (20% yield, 2 steps) as a yellow solid. ¹H NMR (400 MHZ, CDCl₃) δ 14.80 (s, 1H), 7.48 (s, 1H), 5.24 (d, J=4.2 Hz, 1H), 4.86 (d, J=4.2 Hz, 1H), 3.80 (s, 3H), 3.75 (s, 3H), 3.69 (d, J=12.2 Hz, 1H), 3.37 (d, J=12.2 Hz, 1H), 3.06 (q, J=6.4 Hz, 3H), 2.94 (d, J=4.6 Hz, 1H), 2.79-2.69 (m, 3H), 2.60 (d, J=0.9 Hz, 3H), 2.10 (p, J=6.6 Hz, 2H). ¹³C NMR (101 MHz, CDCl₃) δ 204.54, 190.95, 162.93, 151.37, 142.44, 141.50, 140.55, 135.32, 130.50, 115.86, 113.25, 111.13, 103.95, 102.30, 71.76, 69.67, 69.28, 60.96, 52.89, 47.92, 41.06, 38.85, 26.08, 24.35. FTIR (neat), cm-1:2955 (s) 1681 (s), 1621 (s), 1572 (s), 1444 (s), 1389 (s). HRMS (ESI): Calcd for (C₂₄H₂₃BrO₈+H)⁺ 519.0649, found 519.0654.

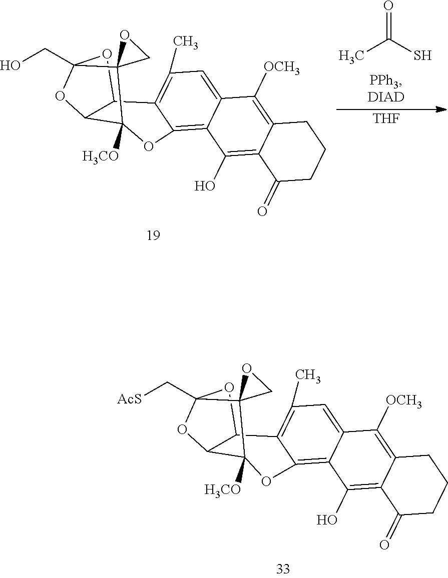

S-(((25,2'S,3a′R,13a′R)-12′-hydroxy-7′,13a′-dimethoxy-5′-methyl-11′-oxo-3a′,8′,9′,10′,11′,13a′-hexahydro-2′H,4′H-spiro [oxirane-2,1′-[2,4]epoxyfuro[3,2-b]naphtho[2,3-h]chromen]-2′-yl)methyl)ethanethioate (33). To begin, DIAD (8.63 μL, 0.044 mmol, 4 equiv.) was added in one portion to a 0° C. solution of PPh₃ (0.011 g, 0.044 mmol, 4 equiv.) in anhydrous THF (0.220 mL, 0.05 M). The resulting solution was stirred for 10 minutes at 0° C. after which a yellow precipitate formed. To a 23° C. solution of 19 (5.00 mg, 11.0 μmol) and thioacetic acid (4.63 μL, 0.066 mmol, 6 equiv.) in anhydrous THF (0.220 mL, 0.05 M) was then added the PPh₃/DIAD solution in one portion. The yellow precipitate clarified and the resulting homogenous solution was stirred at 23° C. for 72 h. After stirring at 23° C. for 72 h, the reaction mixture was partitioned between sat. aq. NaHCO₃ (5 mL) and CH₂Cl₂ (5 mL). The aqueous layer was then extracted with CH₂Cl₂. The organic layers were combined, dried over anhydrous sodium sulfate, filtered, and concentrated under reduced pressure. The crude residue was purified by reverse phase preparatory LCMS (10% acetonitrile in water grading to 90% acetonitrile in water over 30 minutes) to afford the desired compound alongside the thioacetate opened-epoxide and residual triphenylphosphine oxide. This combination of material was then purified by flash-column chromatography (10% EtOAc in Hexanes eluting to 30% EtOAc in Hexanes) to afford 2.60 mg of 33 (46% yield) as a yellow solid. 1H NMR (400 MHz, CDCl₃) δ 14.79 (s, 1H), 7.46 (s, 1H), 5.15 (d, J=4.2 Hz, 1H), 4.79 (d, J=4.1 Hz, 1H), 3.79 (s, 3H), 3.75 (s, 3H), 3.42-3.33 (d, J=14.8 Hz, 1H), 3.21 (d, J=14.8 Hz, 1H), 3.05 (dd, J=7.3, 4.9 Hz, 2H), 2.92 (d, J=4.6 Hz, 1H), 2.77-2.67 (m, 3H), 2.56 (s, 3H), 2.38 (s, 3H), 2.18-2.02 (m, J=6.8 Hz, 2H). ¹³C NMR (101 MHZ, CDCl₃) δ 204.51, 193.85, 162.95, 151.34, 142.42, 141.47, 135.26, 130.41, 115.71, 113.38, 111.10, 105.37, 102.03, 71.71, 69.51, 69.36, 60.94, 52.89, 47.30, 38.85, 30.34, 29.73, 25.11, 23.60, 22.12, 20.20. FTIR (neat), cm-1:2923 (s) 2852 (s), 1698 (s), 1698 (s), 1623 (s), 1446 (s).

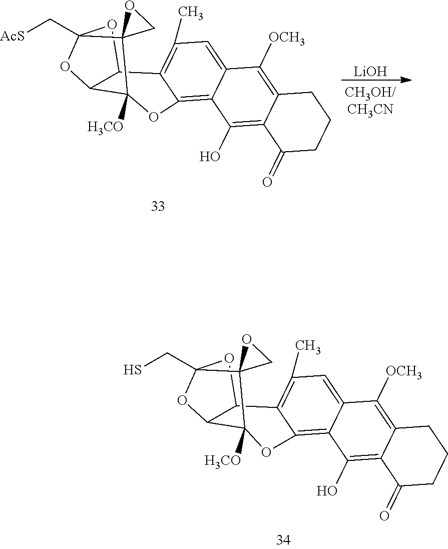

(25,2'S,3a′R,13a′R)-12′-hydroxy-2′-(mercaptomethyl)-7′,13a′-dimethoxy-5′-methyl-3a′, 4′,8′,9′,10′,13a′-hexahydro-2′H,11′H-spiro [oxirane-2,1′-[2,4]epoxyfuro[3,2-b]naphtho[2,3-h]chromen]-11′-one (34). To a 0° C. solution of 33 (2.60 mg, 5.05 μmol) in anhydrous methanol (1.263 mL) and acetonitrile (0.421 mL, resultant 0.003 M solution) was added in one portion LiOH (0.505 mL, 1M, 0.505 mmol, 100 equiv.). The reaction mixture was then vigorously stirred for 2 h at 0° C. After 2 h, the reaction mixture was partitioned between CH₂Cl₂ (5 mL) and brine (5 mL). The residual LiOH was then quenched via addition of formic acid (0.029 mL, 0.758 mmol, 150 equiv.). The aqueous layer was then extracted with CH₂Cl₂. The organic layers were combined, dried over anhydrous sodium sulfate, filtered, and concentrated under reduced pressure. The crude residue was purified by reverse phase preparatory LCMS (10% acetonitrile in water grading to 90% acetonitrile in water over 30 minutes) to afford 0.90 mg of 34 (38% yield) as a yellow solid. ¹H NMR (400 MHZ, CDCl₃) δ 14.80 (s, 1H), 7.47 (s, 1H), 5.21 (d, J=4.2 Hz, 1H), 4.83 (d, J=4.2 Hz, 1H), 3.80 (s, 3H), 3.75 (s, 3H), 3.09-2.98 (m, 3H), 2.92 (d, J=4.8 Hz, 1H), 2.79 (d, J=4.8 Hz, 1H), 2.75-2.66 (m, 3H), 2.60 (d, J=0.9 Hz, 3H), 2.10 (t, J=6.2 Hz, 2H).

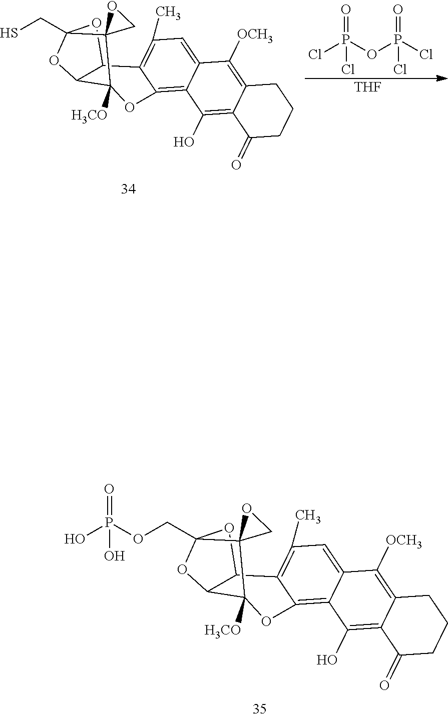

((2R,2′R,3a'S,4'S,13a′R)-12′-hydroxy-7′,13a′-dimethoxy-5′-methyl-11′-oxo-3a′,8′,9′,10′,11′,13a′-hexahydro-2′H,4′H-spiro [oxirane-2,1′-[2,4]epoxyfuro[3,2-b]naphtho[2,3-h]chromen]-2′-yl)methyl dihydrogen phosphate (35). To a flame-dried vial charged with 19 and a stir bar was added anhydrous THF (0.4 mL). The reaction mixture was cooled to −40° C. and the phosphoryl dichloride was added in one portion. The reaction was allowed to warm to 0° C. over 4 hours, then partitioned between 2:1 CH₂Cl₂: methanol (5 mL) and brine (5 mL). The aqueous layer was extracted with further 2:1 CH₂Cl₂: methanol (5 mL) and the combined organic layers were concentrated under reduced pressure. The crude residue was purified by reverse phase preparatory LCMS (10% acetonitrile in water grading to 90% acetonitrile in water over 30 minutes) to afford 1.0 mg of 35 (17% yield) as an orange oil. ¹H NMR (400 MHz, CDCl₃) δ 14.80 (s, 1H), 7.47 (s, 1H), 5.21 (d, J=4.1 Hz, 1H), 4.84 (d, J=4.1 Hz, 1H), 4.37 (dd, J=11.9, 7.1 Hz, 1H), 4.24 (dd, J=11.9, 6.6 Hz, 1H), 3.80 (s, 3H), 3.75 (s, 3H), 3.05 (dd, J=7.3, 5.1 Hz, 2H), 2.91 (d, J=4.8 Hz, 1H), 2.80-2.68 (m, 3H), 2.58 (s, 3H), 2.10 (p, J=6.2 Hz, 2H). ¹³C NMR (101 MHz, CDCl₃) δ 204.69, 163.05, 151.45, 142.58, 141.53, 135.44, 130.64, 115.92, 113.37, 111.28, 103.95, 102.08, 72.10, 69.49, 69.31, 62.16, 61.09, 54.72, 53.05, 48.02, 38.98, 23.74, 22.25, 20.25. ³¹P NMR (162 MHZ, CDCl₃) δ 0.95. FTIR (neat), cm-1:2956 (s) 1621 (s), 1572 (s), 1446 (s), 1389 (s), 1349 (s). HRMS (ESI): Calcd for (C₂₄H₂₅PO₁₂+H)⁺ 537.1156, found 537.1172.

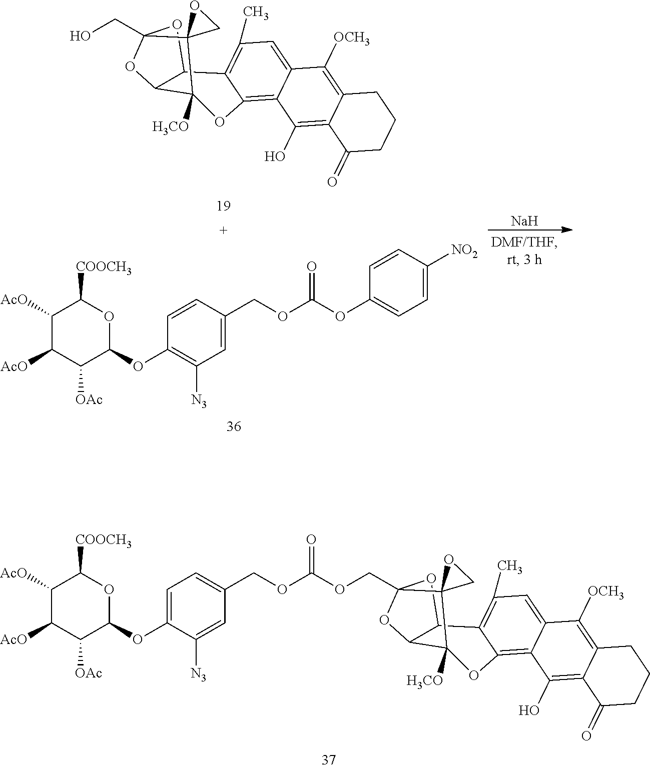

(2S,3R,4S,5S,6S)-2-(2-Azido-4-((((((2R,2′R,3a′R,4'S,13a′R)-12′-hydroxy-7′,13a′-dimethoxy-5′-methyl-11′-oxo-3a′,8′,9′,10′,11′,13a′-hexahydro-2′H,4′H-spiro [oxirane-2,1′-[2,4]epoxyfuro[3,2-b]naphtho[2,3-h]chromen]-2′-yl)methoxy)carbonyl)oxy)methyl)phenoxy)-6-(methoxycarbonyl) tetrahydro-2H-pyran-3,4,5-triyl triacetate (37). To a solution of 36 (21.2 mg, 33.0 μmol, 1.0 equiv.) and 19 (5.00 mg, 11.0 μmol, 1.0 equiv.) in dimethylformamide (60.9 μL) and tetrahydrofuran (60.9 μL) was added sodium hydride (0.394 mg, 16.0 μmol, 1.5 equiv.) at 23° C. The reaction mixture was stirred for 3 h, until LCMS analysis indicated full consumption of the starting material. The crude residue was purified by reverse phase preparatory LCMS (10% acetonitrile in water grading to 90% acetonitrile in water over 30 minutes) to afford 5.20 mg of 37 (49% yield) as a yellow solid. ¹H NMR (400 MHZ, CDCl₃) δ 14.79 (s, 1H), 7.46 (s, 1H), 7.09 (d, J=1.3 Hz, 2H), 7.05 (s, 1H), 5.37-5.25 (m, 3H), 5.19 (d, J=4.1 Hz, 1H), 5.10-5.08 (m, 3H), 4.83 (d, J=4.1 Hz, 1H), 4.53 (d, J=12.4 Hz, 1H), 4.35 (d, J=12.4 Hz, 1H), 4.19-4.07 (m, 1H), 3.79 (s, 3H), 3.74 (s, 3H), 3.74 (s, 3H), 3.05 (t, J=5.7 Hz, 2H), 2.91 (d, J=4.8 Hz, 1H), 2.75-2.73 (m, 3H), 2.55 (s, 3H), 2.14-2.10 (m, 2H), 2.09 (s, 3H), 2.05 (s, 3H), 2.04 (s, 3H). ¹³C NMR (101 MHz, CDCl₃) δ 204.66, 170.28, 169.48, 169.35, 166.85, 163.05, 154.55, 151.47, 148.70, 142.56, 141.61, 135.43, 131.84, 130.82, 130.61, 125.96, 121.01, 118.92, 115.91, 111.38, 113.30, 111.26, 103.87, 102.03, 100.05, 72.79, 72.01, 71.86, 70.94, 69.49, 69.32, 69.10, 62.07, 61.08, 53.19, 53.04, 47.92, 38.98, 29.85, 23.73, 22.25, 20.78, 20.67, 20.29, 1.17. FTIR (neat), cm-1:2955.74 (w), 2120.14 (m), 1757.58 (vs), 1621.62 (m), 1572.05 (w), 1508.32 (w), 1443.17 (w), 1355.36 (m), 1374.32 (m), 1226.48 (vs), 1181.16 (w), 1093.63 (m), 1069.28 (m), 1038.12 (m), 982.89 (m), 953.14 (w), 913.49 (w), 787.44 (w). HRMS (ESI): Calcd for (C₄₅H₄₅N₃O₂₁+H)⁺964.2618, found 964.2607.

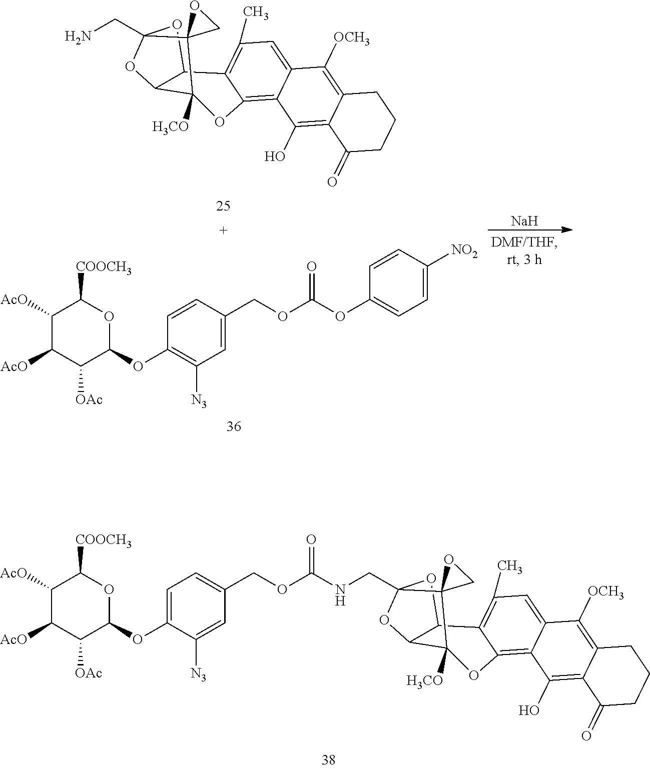

(2S,3R,4S,5S,6S)-2-(2-Azido-4-((((((2R,2′R,3a′R.4'S,13a′R)-12′-hydroxy-7′,13a′-dimethoxy-5′-methyl-11′-oxo-3a′,8′,9′,10′,11′,13a′-hexahydro-2′H,4′H-spiro [oxirane-2,1′-[2.4]epoxyfuro[3,2-b]naphtho[2,3-h]chromen]-2′-yl)methyl)carbamoyl)oxy)methyl)phenoxy)-6-(methoxycarbonyl)tetrahydro-2H-pyran-3,4,5-triyl triacetate (38). To a solution of PNP-carbonate 36 (21.3 mg, 0.033 mmol, 3.0 equiv.) in dimethylformamide (61.0 μL) was added N,N-diisopropylethylamine (19.1 μL, 0.110 mmol, 10 equiv.) at 23° C. Amine 26 (5.00 mg, 11.0 μmol, 1.0 equiv.) in dimethylformamide (61.0 μL) was added dropwise to the solution and stirred for 30 min. The reaction solution was diluted with dry dimethylformamide to a total volume of 900 μL and purified directly by preparatory HPLC (10% acetonitrile in water grading to 90% acetonitrile in water over 30 minutes) to provide drug-linker 38 as a yellow solid (2.10 mg, 20%). ¹H NMR (400 MHZ, CDCl3) δ 14.80 (s, 1H), 7.47 (s, 1H), 7.10 (d, J=2.0 Hz, 2H), 7.06 (s, 1H), 5.36-5.23 (m, 3H), 5.15 (d, J=4.1 Hz, 1H), 5.08 (d, J=7.1 Hz, 1H), 5.05 (s, 2H), 4.81 (d, J=4.2 Hz, 1H), 4.18-4.09 (m, 1H), 3.79 (s, 3H), 3.74 (s, 6H), 3.56 (d, J=5.7 Hz, 2H), 3.05 (t, J=6.1 Hz, 2H), 2.92 (d, J=4.6 Hz, 1H), 2.74 (t, J=6.3 Hz, 2H), 2.67-2.61 (m, 1H), 2.55 (s, 3H), 2.12-2.08 (m, 5H), 2.05 (s, 3H), 2.04 (s, 3H).

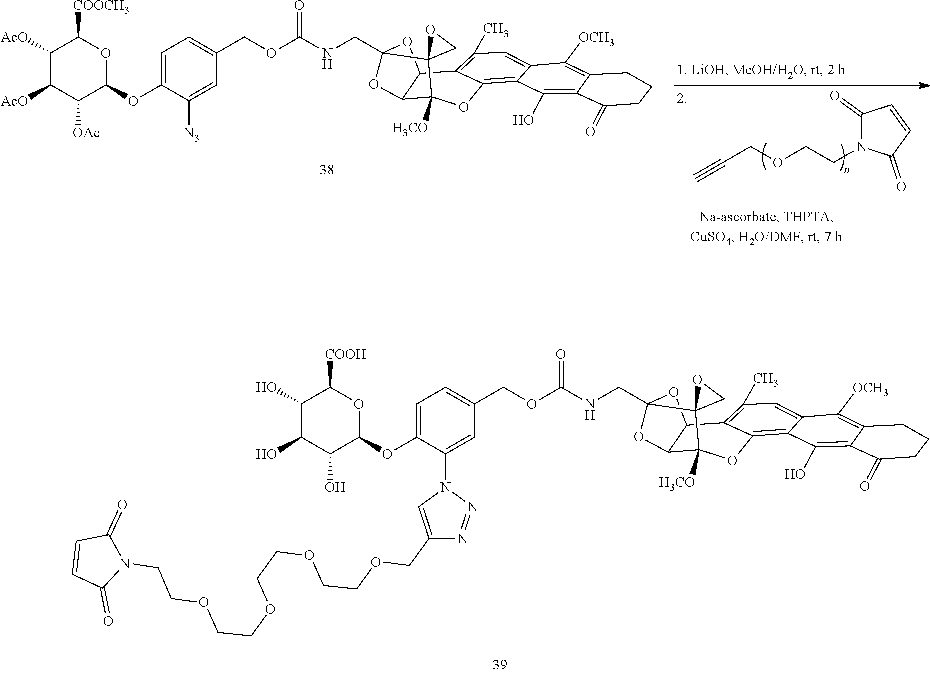

(2S,3S,4S,5R,6S)-6-(2-(4-(13-(2,5-Dioxo-2,5-dihydro-1H-pyrrol-1-yl)-2,5,8,11-tetraoxatridecyl)-1H-1,2,3-triazol-1-yl)-4-((((((2R,2′R,3a′R,4'S,13a′R)-12′-hydroxy-7′,13a′-dimethoxy-5′-methyl-11′-oxo-3a′,8′,9′,10′,11′,13a′-hexahydro-2′H, 4′H-spiro [oxirane-2,1′-[2.4]epoxyfuro[3,2-b]naphtho[2,3-h]chromen]-2′-yl)methyl)carbamoyl)oxy)methyl)phenoxy)-3,4,5-trihydroxytetrahydro-2H-pyran-2-carboxylic acid (39). Protected glucuronic ester linker 38 (2.00 mg, 2.08 μmol, 1.0 equiv.) was dissolved in methanol (519 μL) and acetonitrile (173 μL) at 23° C. and the solution was purged with nitrogen for 5 min. The solution was cooled to 0° C. in an ice bath and a 0.4 M aqueous lithium hydroxide solution (519 μL, 0.208 mmol, 100 equiv.) was added dropwise. The yellow reaction mixture was stirred vigorously at 0° C. for 2 h. Upon complete saponification of the glucuronide esters, the reaction was diluted with 2:1 dichloromethane-methanol (1.5 mL) and saturated aqueous sodium chloride solution (1 mL) then quenched by the addition of formic acid (11.8 μL, 0.312 mmol, 150 equiv.) at 0° C. The mixture was stirred for 5 min, then transferred to a separatory funnel. The funnel was shaken vigorously and the layers were separated. The aqueous layer (now pH ˜4.5-5 or adjusted to this pH by further addition of formic acid) was extracted with 2:1 dichloromethane-methanol (3×1 mL). The combined organic layers were dried by filtration through a plug of sodium sulfate and the filtrate was concentrated to provide glucuronic acid intermediate as a yellow solid. The crude material was taken forward without further purification assuming quantitative conversion. The intermediate (crude from previous reaction, assuming 1.71 mg, 2.08 μmol, 1.00 equiv.) and Mal-PEG4-alkyne (1.26 μL, 6.24 μmol, 3.00 equiv.) were dissolved in 4:1 water/DMSO (692 μL) at 23° C. A pre-mixed solution of 0.1 M aqueous cupric sulfate (5.20 μL, 0.520 μmol, 0.25 equiv.) and 0.05 M THPTA (52.0 μL, 2.60 μmol, 1.25 equiv.) was added, followed by a 2.0 M aqueous sodium ascorbate solution (5.20 μL, 10.4 μmol, 5.00 equiv.). The reaction mixture was stirred vigorously for 7 h, at which point LCMS analysis indicated complete conversion to product. The reaction mixture was diluted with 2:1 dichloromethane-methanol (2 mL) and saturated aqueous sodium chloride solution (1.5 mL). The pH of the aqueous layer was adjusted to ˜4.5-5 by addition of formic acid, then the layers were shaken and separated. The aqueous layer was extracted with 2:1 dichloromethane-methanol (3×1.5 mL) and the combined organic layers were dried over sodium sulfate. The dried solution was filtered and the filtrate concentrated. The residue was purified by preparatory HPLC (10% acetonitrile in water grading to 90% acetonitrile in water over 30 minutes) to provide drug-linker 39 as a yellow solid. HRMS (ESI): Calcd for (C₅₃H₅₉N₅O₂₃+H)⁺1134.3674, found 1134.3668.

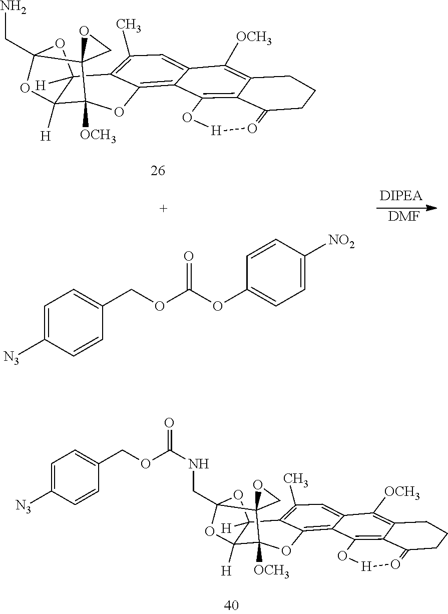

4-azidobenzyl (((2R,2′R,3a'S,4′R)-12′-hydroxy-7′,13a′-dimethoxy-5′-methyl-11′-oxo-3a′,8′,9′,10′,11′,13a′-hexahydro-2′H,4′H-spiro [oxirane-2,l′-[2,4]epoxyfuro[3,2-b]naphtho[2,3-h]chromen]-2′-yl)methyl)carbamate (40). To a solution of 26 (22.5 mg, 0.049 mmol) in anhydrous DMF (1.5 mL, 0.033 M) was added DIPEA (0.086 mL, 0.494 mmol, 10 equiv.) followed by the mixed carbonate (78.0 mg, 0.247 mmol, 5 equiv.). The reaction mixture was stirred at 23° C. overnight. The next day (˜18 h), the reaction mixture was diluted with DMF to a total volume of 1.8 mL and purified directly by preparatory HPLC (10% acetonitrile in water grading to 90% acetonitrile in water over 30 minutes) to provide carbamate 40 as a yellow solid (12.9 mg, 41% yield). ¹H NMR (400 MHZ, CDCl₃) δ 14.80 (s, 1H), 7.46 (s, 1H), 7.37 (d, J=8.5 Hz, 2H), 7.02 (d, J=8.5 Hz, 2H), 5.14 (d, J=4.2 Hz, 1H), 5.09 (s, 2H), 4.81 (d, J=4.2 Hz, 1H), 3.79 (s, 3H), 3.74 (s, 3H), 3.56 (d, J=5.8 Hz, 2H), 3.09-3.01 (m, 2H), 2.92 (d, J=4.6 Hz, 1H), 2.74 (t, J=6.4 Hz, 2H), 2.65 (d, J=4.5 Hz, 1H), 2.55 (s, 3H), 2.09 (p, J=6.2 Hz, 2H). ¹³C NMR (101 MHz, CDCl₃) δ 204.54, 162.92, 154.94, 151.32, 142.44, 141.32, 140.47, 135.29, 131.76, 130.15, 129.91, 119.19, 119.11, 115.76, 113.27, 113.23, 111.14, 104.87, 101.94, 71.73, 69.39, 69.17, 68.99, 66.40, 60.95, 52.90, 47.36, 38.84, 37.58, 23.61, 22.11, 20.15. FTIR (neat), cm-1:2958 (s) 2110 (s), 1725 (s), 1618 (s), 1570 (s), 1508 (s). HRMS (ESI): Calcd for (C₃₂H₃₀N₄O₁₀+Na)⁺653.1854, found 653.1836.

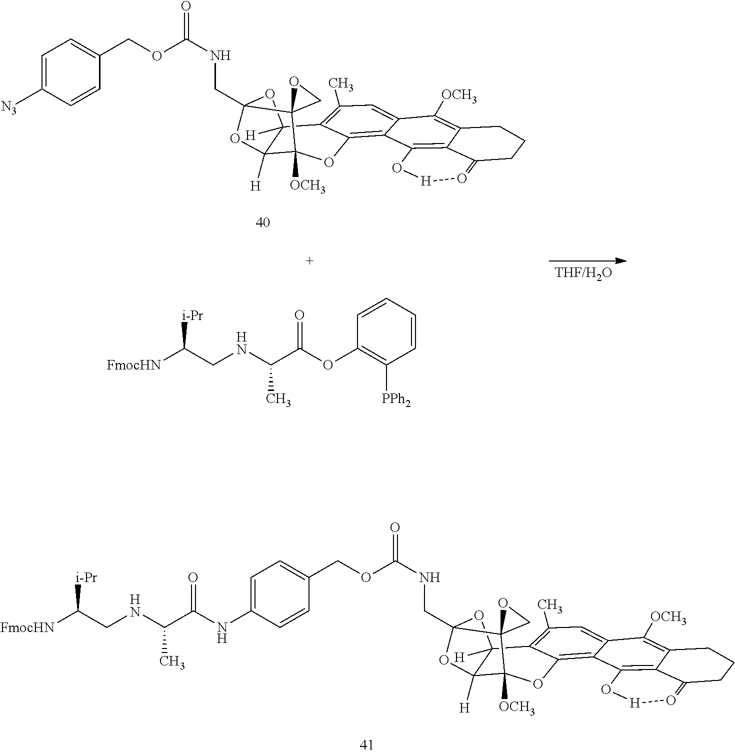

4-((S)-2-((S)-2-((((9H-fluoren-9-yl)methoxy)carbonyl)amino)-3-methylbutanamido)propanamido)benzyl (((2R,2′R,3a'S,4′R)-12′-hydroxy-7′,13a′-dimethoxy-5′-methyl-11′-oxo-3a′,8′,9′,10′,11′,13a′-hexahydro-2′H,4′H-spiro[oxirane-2,1′-[2.4]epoxyfuro[3,2-b]naphtho[2,3-h]chromen]-2′-yl)methyl) carbamate (41). To a flame-dried round bottom flask charged with a magnetic stir bar was added carbamate 40 (12.9 mg, 0.020 mmol) as a solution in THF (0.818 mL) and water (0.205 mL, resultant soln. 0.02 M). The dipeptide was added in one portion (0.027 g, 0.041 mmol, 2 equiv.) and the reaction mixture was stirred at 23° C. overnight. After 24 h, the reaction mixture was diluted with ethyl acetate and partitioned between sat. aq. brine and ethyl acetate. The combined organic layers were dried over anhydrous sodium sulfate, filtered, and concentrated under reduced pressure. The residue by preparatory HPLC (10% acetonitrile in water grading to 90% acetonitrile in water over 30 minutes) to provide 41 as a yellow solid (9.5 mg, 47% yield). ¹H NMR (400 MHZ, CDCl₃) δ 14.79 (s, 1H), 8.38 (s, 1H), 7.76 (d, J=7.6 Hz, 2H), 7.54 (q, J=6.9, 5.3 Hz, 4H), 7.46 (s, 1H), 7.40 (t, J=7.5 Hz, 2H), 7.34-7.28 (m, 4H), 6.33 (s, 1H), 5.23 (s, 1H), 5.16-5.05 (m, 4H), 4.80 (d, J=4.1 Hz, 1H), 4.70-4.60 (m, 1H), 4.48 (d, J=6.6 Hz, 2H), 4.20 (t, J=6.8 Hz, 1H), 3.97 (d, J=6.9 Hz, 1H), 3.79 (s, 3H), 3.74 (s, 3H), 3.56 (t, J=5.1 Hz, 1H), 3.05 (t, J=6.1 Hz, 2H), 2.91 (dd, J=5.2, 3.2 Hz, 1H), 2.73 (t, J=6.4 Hz, 2H), 2.64 (d, J=4.6 Hz, 1H), 2.55 (s, 3H), 2.13-2.06 (m, 2H), 1.56 (s, 1H), 1.45 (t, J=6.4 Hz, 3H), 0.91 (s, 6H), 0.12 (d, J=4.7 Hz, 1H). ¹³C NMR (101 MHz, CDCl₃) δ 204.52, 171.61, 171.20, 169.66, 162.94, 156.74, 156.36, 151.32, 143.63, 143.57, 142.43, 141.41, 141.36, 135.28, 132.34, 130.45, 129.12, 128.48, 127.83, 127.70, 127.12, 124.91, 120.07, 119.80, 115.75, 113.28 (d, J=2.3 Hz), 111.12, 104.89, 101.97, 71.70, 70.92, 69.36, 68.98, 67.03, 66.63, 60.95, 60.78, 60.43, 52.89, 49.57, 47.37, 47.20, 38.84, 37.53, 30.75, 29.72, 25.58, 23.60, 22.12, 21.09, 20.16, 19.21, 17.82, 17.14, 14.22. FTIR (neat), cm-1:3309 (br. s) 2931 (s), 1702 (s), 1648 (s), 1621 (s), 1545 (s). HRMS (ESI): Calcd for (C₅₅H₅₆N₄O₁₄+NH₄)⁺ 1014.4131, found 1014.4104

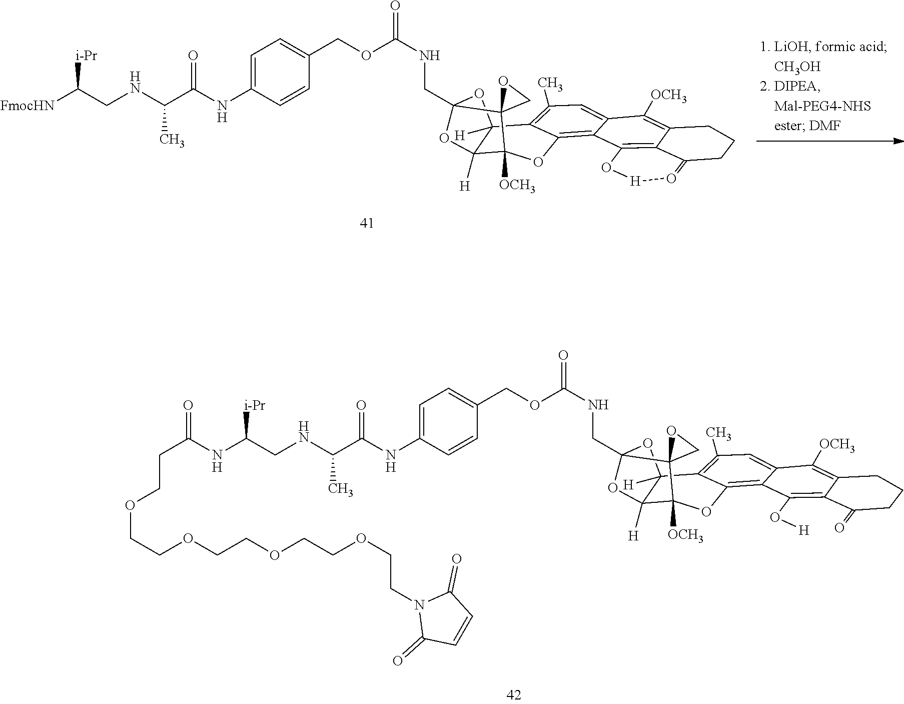

4-((17S,20S)-1-(2,5-dioxo-2,5-dihydro-1H-pyrrol-1-yl)-17-isopropyl-20-methyl-15,18-dioxo-3,6,9,12-tetraoxa-16,19-diazahenicosan-21-amido)benzyl (((2R,2′R,3a'S,4′R)-12′-hydroxy-7′,13a′-dimethoxy-5′-methyl-11′-oxo-3a′,8′,9′,10′, 11′, 13a′-hexahydro-2′H,4′H-spiro [oxirane-2,1′-[2,4]epoxyfuro[3,2-b]naphtho[2,3-h]chromen]-2′-yl)methyl)carbamate (42). Carbamate 41 (9.50 mg, 9.53 μmol) was dissolved in methanol (3.18 mL) at 23° C. 1 M aqueous lithium hydroxide solution (0.953 mL, 0.953 mmol, 100 equiv.) was added dropwise. The yellow reaction mixture was stirred vigorously at 23° C. for 2 h. After 2 h, the reaction was diluted with dichloromethane and saturated aqueous sodium chloride solution and then quenched by the addition of formic acid (0.036 mL, 0.953 mmol, 100 equiv.). The mixture was then transferred to a separatory funnel. The funnel was shaken vigorously and the layers were separated. The aqueous layer was extracted with 2:1 dichloromethane-methanol (3×1 mL). The combined organic layers were dried by filtration through a plug of sodium sulfate and the filtrate was concentrated to provide the free amine as a yellow solid. The crude material was taken forward without further purification assuming quantitative conversion. HRMS (ESI): Calcd for (C₄₀H₄₆N₄O₁₂+NH₄)⁺ 1014.4131, found 1014.4104. To a scintillation vial containing the crude free amine and a magnetic stir bar was added anhydrous DMF (0.476 mL, 0.02 M). To the solution was added DIPEA (10 μL, 0.057 mmol, 6 equiv.) and Mal-PEG4—NHS ester (7.4 mg, 0.057 mmol, 6 equiv.). The reaction mixture was allowed to stir at 23° C. overnight. After stirring overnight (˜18 h), the reaction mixture was diluted to a total volume of 900 μL and purified directly by preparatory HPLC (10% acetonitrile in water grading to 90% acetonitrile in water over 30 minutes) to provide drug-linker 42 as a yellow solid (4.7 mg, 45% yield). ¹H NMR (400 MHZ, CDCl₃) δ 14.79 (s, 1H), 8.57 (s, 1H), 7.70 (d, J=8.3 Hz, 2H), 7.46 (s, 1H), 7.31 (d, J=8.1 Hz, 2H), 6.98 (d, J=16.6 Hz, 1H), 6.84 (s, 1H), 6.70 (s, 1H), 5.20-4.97 (m, 4H), 4.81 (d, J=4.2 Hz, 1H), 4.66 (p, J=7.2 Hz, 1H), 4.20 (t, J=6.1 Hz, 1H), 3.79 (s, 3H), 3.74 (s, 3H), 3.65-3.52 (m, 18H), 3.04 (dt, J=5.8, 3.5 Hz, 2H), 2.91 (d, J=4.6 Hz, 1H), 2.73 (t, J=6.3 Hz, 2H), 2.70-2.60 (m, 2H), 2.55 (s, 3H), 2.48 (ddd, J=15.0, 5.8, 3.5 Hz, 1H), 2.14-2.06 (m, 2H), 1.63 (s, 3H), 1.45 (d, J=7.1 Hz, 3H), 0.99 (dd, J=12.2, 6.8 Hz, 6H), 0.07 (s, 1H). ¹³C NMR (101 MHZ, CDCl₃) δ 204.51, 188.27, 173.43, 171.52, 170.70, 162.94, 156.40, 151.32, 142.43, 141.46, 135.28, 134.20, 130.42, 129.00, 119.84, 115.74, 114.69, 114.67, 113.32, 113.26, 111.11, 104.90, 101.99, 71.68, 70.61, 70.48, 70.46, 70.41, 70.19, 69.98, 69.35, 68.98, 67.81, 66.75, 61.24, 60.95, 52.89, 49.40, 47.38, 41.05, 38.85, 37.49, 37.21, 37.19, 37.10, 29.50, 26.07, 23.60, 22.12, 21.25, 20.18, 19.43, 17.89, 17.26, 1.04. FTIR (neat), cm-1:3306 (br. s) 2930 (s), 1708 (s), 1621 (s), 1535 (s), 1444 (s). HRMS (ESI): Calcd for (C₅₅H₆₇N₅O₁₉+Na)⁺ 1124.4322, found 1124.4297.

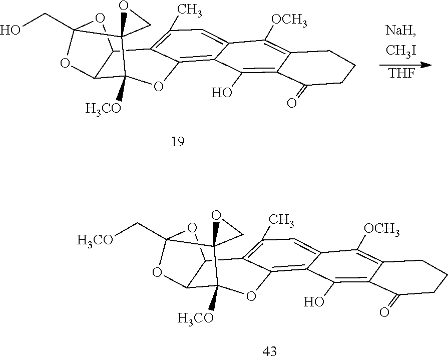

(2R,2′R,3a′R,4'S, 13a′R)-12′-hydroxy-7′, 13a′-dimethoxy-2′-(methoxymethyl)-5′-methyl-3a′ 4′,8′,9′,10′,13a′-hexahydro-2′H, 11′H-spiro[oxirane-2,1′-[2,4]epoxyfuro[3,2-b]naphtho[2,3-h]chromen]-11′-one (43). In a flame-dried flask, to a 0° C. suspension of sodium hydride (5 eq., 0.110 mmol, 2.6 mg) in anhydrous THF (0.22 mL) was slowly added a solution of 19 (0.022 mmol, 10.0 mg) in anhydrous THF (0.22 mL). After the addition was complete, the mixture was allowed to stir at rt for 2 h before the dropwise addition of methyl iodide (10 eq., 0.220 mmol, 31.0 mg). After stirring overnight, the suspension was partitioned between EtOAc and sat. aq. NH₄Cl solution. The organic layer was washed with brine, dried over anhydrous sodium sulfate, filtered, and concentrated in vacuo. The crude material was purified via HPLC to afford 43 (8.0 mg, 78% yield) as a yellow-green solid. ¹H NMR (400 MHz, CDCl₃) δ 14.80 (s, 1H), 7.45 (s, J=1.0 Hz, 1H), 5.22 (d, J=4.2 Hz, 1H), 4.81 (d, J=4.2 Hz, 1H), 3.79 (s, 3H), 3.76 (s, 1H), 3.75 (s, 3H), 3.72 (s, 1H), 3.43 (s, 3H), 3.05 (ddd, J=2.9 Hz, 2H), 2.90 (d, J=5.2 Hz, 1H), 2.79 (d, J=5.2 Hz, 1H), 2.73 (dd, J=7.1, 5.8 Hz, 2H), 2.60 (s, J=0.9 Hz, 3H), 2.13-2.06 (m, 2H). ¹³C NMR (101 MHz, CDCl₃) δ 204.47, 163.02, 151.44, 142.43, 141.68, 135.25, 130.30, 115.66, 113.50, 113.29, 111.06, 104.89, 102.05, 71.69, 69.26, 69.22, 67.49, 60.92, 60.31, 52.86, 48.13, 38.84, 23.60, 22.12, 20.31. HRMS (ESI): Calcd for (C₂₅H₂₆O₉+H)⁺471.1650, found 471.1651.

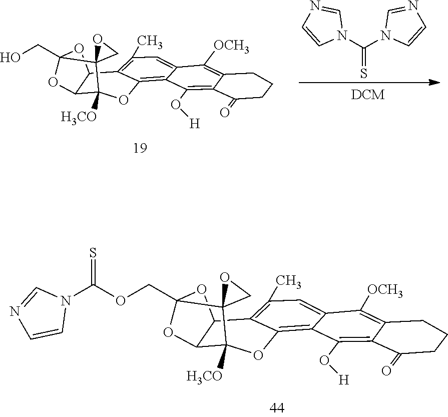

O-(((2R,2′R,3a'S,4'S)-12′-hydroxy-7′,13a′-dimethoxy-5′-methyl-11′-oxo-3a′,8′,9′,10′,11′,13a′-hexahydro-2′H,4′H-spiro[oxirane-2,1′-[2,4]epoxyfuro[3,2-b]naphtho[2,3-h]chromen]-2′-yl)methyl) 1H-imidazole-1-carbothioate (44). 19 (35 mg, 1 Eq, 77 μmol) was added to a flame-dried vial equipped with a stir bar. DCM (0.38 mL) was added, followed by Di-1H-imidazol-1-ylmethanethione (16 mg, 1.2 Eq, 92 μmol). The vial was wrapped in aluminum foil and stirred at RT for 16 hours. The reaction was then quenched with sat. aq ammonium chloride (0.5 mL), extracted with DCM (3×0.5 mL), washed with brine (0.5 mL), dried over sodium sulfate and concentrated in vacuo. The crude product was purified by FCC (25 to 45% EtOAc in Hexanes) to afford 44 (24 mg, 42 μmol, 55%) as a bright yellow solid. ¹H NMR (400 MHZ, Chloroform-d) δ 14.81 (s, 1H), 8.36 (s, 1H), 7.66 (t, J=1.5 Hz, 1H), 7.49 (s, 1H), 7.05 (s, 1H), 5.23 (d, J=4.1 Hz, 1H), 4.95-4.86 (m, 2H), 4.83 (d, J=12.7 Hz, 1H), 3.81 (s, 3H), 3.77 (s, 3H), 3.10-3.02 (m, 2H), 2.98 (d, J=4.5 Hz, 1H), 2.78-2.69 (m, 3H), 2.58 (d, J=0.9 Hz, 3H), 2.14-2.02 (m, 2H). ¹³C NMR (101 MHz, CDCl₃) δ 204.6, 183.4, 162.9, 151.3, 142.5, 141.3, 136.9, 135.4, 131.0, 130.6, 118.3, 115.9, 113.3, 113.0, 111.2, 103.8, 101.8, 72.1, 69.6, 69.2, 65.9, 61.0, 53.0, 47.6, 38.8, 23.6, 22.1, 20.1. HRMS (ESI): Calcd for (C₂₈H₂₆N₂O₉S+H)⁺ 567.1432, found 567.1432.

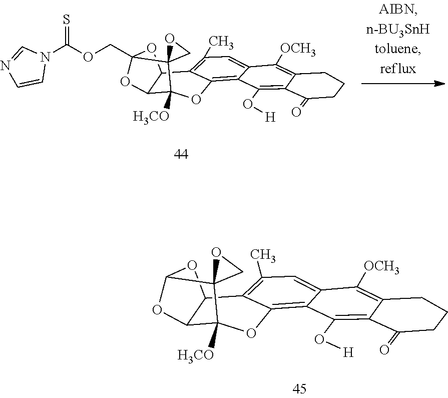

(2R,2′R,3a'S,4'S)-12′-hydroxy-7′,13a′-dimethoxy-2′,5′-dimethyl-3a′ 4′,8′,9′,10′,13a′-hexahydro-2′H,11′H-spiro [oxirane-2,1′-[2,4]epoxyfuro[3,2-b]naphtho[2,3-h]chromen]-11′-one (45). 44 (4.00 mg, 1 Eq, 7.06 μmol) was added to a microwave vial. Tributyltin hydride (4.11 mg, 3.82 μL, 2 Eq, 14.1 μmol) and 2,2′-Azobisisobutyronitrile (174 μg, 215 nL, 0.15 Eq, 1.06 μmol) were dissolved in Toluene (200 μL) and added to the vial. It was then heated to 110° C. for 3 hours, after which the reaction was cooled down to RT and the solvent removed under reduced pressure. The crude residue was purified by reverse phase preparatory LCMS (10% acetonitrile in water grading to 90% acetonitrile in water over 30 minutes) to afford 45 (0.300 mg, 0.681 μmol, 9.65%) as a yellow solid. ¹H NMR (400 MHZ, Chloroform-d) δ 14.80 (s, 1H), 7.46 (d, J=1.0 Hz, 1H), 5.13 (d, J=4.2 Hz, 1H), 4.76 (d, J=4.2 Hz, 1H), 3.79 (s, 3H), 3.76 (s, 3H), 3.05 (dd, J=7.2, 5.1 Hz, 2H), 2.91 (d, J=4.8 Hz, 1H), 2.73 (t, J=6.4 Hz, 2H), 2.60-2.55 (m, 4H), 2.14-2.05 (m, 2H), 1.42 (s, 3H). HRMS (ESI): Calcd for (C₂₄H₂₄O₈+H)⁺ 441.1544, found 441.1544.

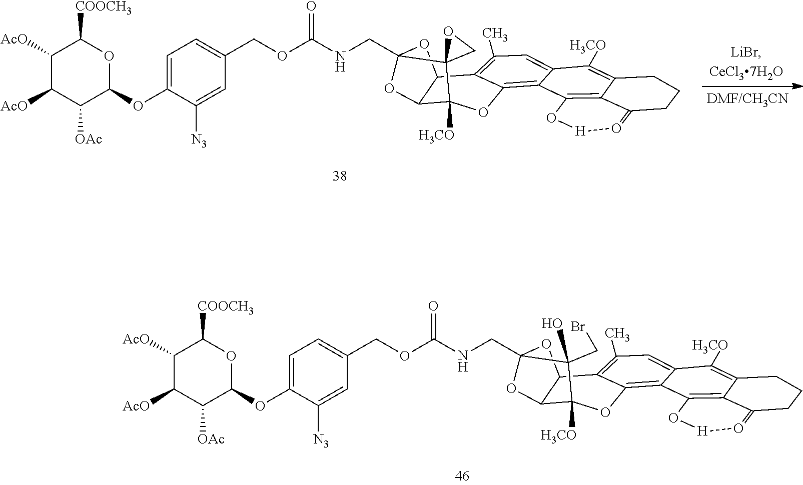

(2S,3R.4S,5S,6S)-2-(2-azido-4-((((((1R,13aR)-1-(bromomethyl)-1.12-dihydroxy-7,13a-dimethoxy-5-methyl-11-oxo-1,3a,4,8,9,10,11,13a-octahydro-2H-2,4-epoxyfuro [3,2-b]naphtho[2,3-h]chromen-2-yl)methyl)carbamoyl)oxy)methyl)phenoxy)-6-(methoxycarbonyl) tetrahydro-2H-pyran-3,4,5-triyl triacetate (46). To a solution of 38 (8.1 mg, 8.41 μmol) in anhydrous acetonitrile (0.841 mL, 0.01 M) was added lithium bromide (0.022 g, 0.252 mmol, 30 equiv.) followed by cerium trichloride heptahydrate (9.40 mg, 0.025 mmol, 3.0 equiv.). The reaction mixture was then wrapped in aluminum foil to exclude light and stirred at 23° C. overnight. After stirring overnight (˜18 h), the reaction mixture was partitioned between sat. aq. sodium sulfite solution (5 mL) and EtOAc (5 mL). The aqueous layer was then extracted with EtOAc. The organic layers were combined, dried over anhydrous sodium sulfate, filtered, and concentrated under reduced pressure. The residue was purified by reverse phase preparatory LCMS (10% acetonitrile in water grading to 90% acetonitrile in water over 30 minutes) to afford 7.9 mg (90% yield) of 46 as a yellow solid.

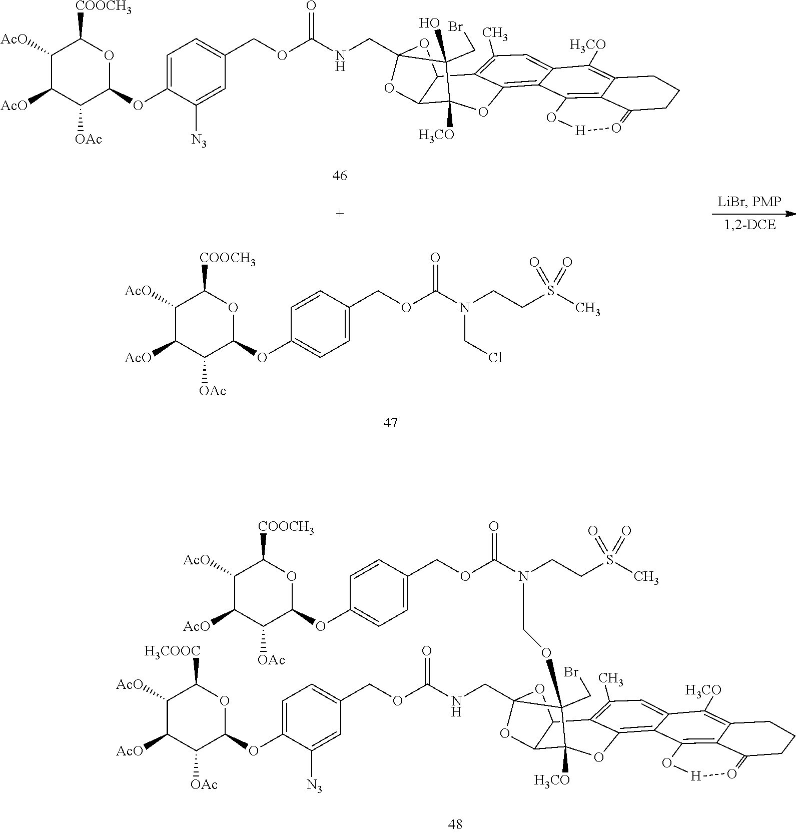

(2S,3R,4S,5S,6S)-2-(2-azido-4-((((((1R,13aR)-1-(bromomethyl)-12-hydroxy-7,13a-dimethoxy-5-methyl-1-(((2-(methylsulfonyl)ethyl)(((4-(((2S,3R,4S,5S,6S)-3,4,5-triacetoxy-6-(methoxycarbonyl)tetrahydro-2H-pyran-2-yl)oxy)benzyl)oxy)carbonyl)amino)methoxy)-11-oxo-1,3a,4,8,9,10,11,13a-octahydro-2H-2,4-epoxyfuro[3,2-b]naphtho[2,3-h]chromen-2-yl)methyl)carbamoyl)oxy)methyl)phenoxy)-6-(methoxycarbonyl)tetrahydro-2H-pyran-3,4,5-triyl triacetate (48). To begin, lithium bromide (3.94 mg, 0.045 mmol, 6 equiv.) was added to a flame-dried reaction vessel. Carbamate 46 was dissolved in anhydrous 1,2-dichloroethane (0.378 mL) and then transferred to the reaction vessel. Next, methyl sulfone (0.024 g, 0.151 mmol, 20 equiv.) 47 as a solution in anhydrous 1,2-dichloroethane (0.378 mL) and 1,2,2,6,6-pentamethylpiperidine were added simultaneously to the reaction mixture. The reaction mixture was then heated to 40° C. and maintained at that temperature for 2 h. After 2 h, the reaction was cooled to room temperature and allowed to stir at rt overnight. After stirring overnight (˜18 h), the reaction mixture was concentrated and the residue was resuspended in 900 μL of DMF and purified directly by preparatory HPLC (10% acetonitrile in water grading to 90% acetonitrile in water over 30 minutes) to provide protected glucuronic ester linker 48 as a yellow solid (1.2 mg, 5% yield).

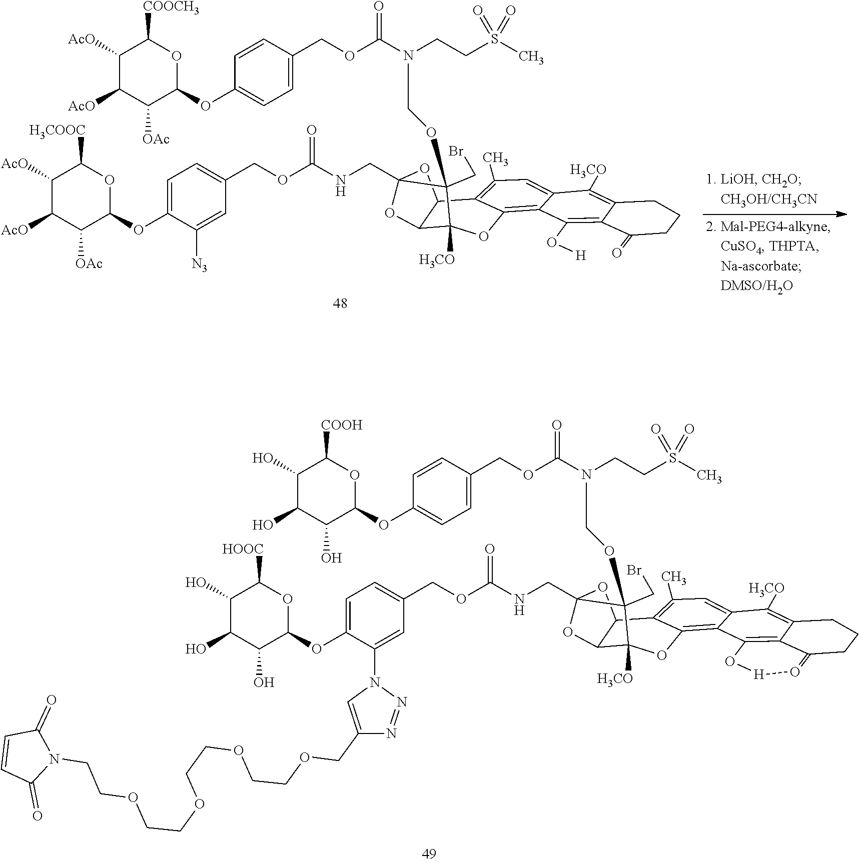

(2S,3S,4S,5R,6S)-6-(4-((((((1R,13aR)-1-(bromomethyl)-1-(((((4-(((25,3R,4S,5S,6S)-6-carboxy-3,4,5-trihydroxytetrahydro-2H-pyran-2-yl)oxy)benzyl)oxy)carbonyl) (2-(methylsulfonyl)ethyl)amino)methoxy)-12-hydroxy-7,13a-dimethoxy-5-methyl-11-oxo-1,3a,4,8,9,10,11,13a-octahydro-2H-2,4-epoxyfuro[3,2-b]naphtho[2,3-h]chromen-2-yl)methyl)carbamoyl)oxy)methyl)-2-(4-(13-(2,5-dioxo-2,5-dihydro-1H-pyrrol-1-yl)-2,5,8,11-tetraoxatridecyl)-1H-1,2,3-triazol-1-yl)phenoxy)-3,4,5-trihydroxytetrahydro-2H-pyran-2-carboxylic acid (49). Protected glucuronic ester linker 48 (0.6 mg, 0.365 μmol) was dissolved in methanol (0.912 mL) and acetonitrile (0.304 mL, 0.0003 M) at room temperature and the solution was purged with nitrogen for 5 minutes. After 5 minutes, the reaction mixture was cooled to 0° C. in an ice bath and lithium hydroxide (0.365 mL, 1 M, 1000 equiv.) was added dropwise. The yellow reaction mixture was stirred vigorously at 0° C. for 2 h. After 2 h, formic acid (0.014 mL, 0.383 mmol, 1050 equiv.) was added and the reaction mixture was filtered through celite, washing with additional methanol. The filtrate was then concentrated to afford a thin off-white/yellow film which was taken forward without further purification assuming quantitative conversion.

Next, the crude product was transferred to a flame-dried vial charged with a magnetic stir bar. To the vial was added DMSO (0.203 mL), water (1.00 mL, 0.3 mM), and Mal-PEG4-alkyne (6.81 mg, 21.9 μmol, 60 equiv.) in that order. To the reaction flask was then added aqueous copper (II) sulfate (18.2 μL, 0.1 M, 1.82 μmol, 5 equiv.), THPTA (0.498 mg, 0.365 μmol, 1 equiv.), and aqueous sodium ascorbate (18.2 μL, 2 M, 36.5 μmol, 100 equiv.) in that order. The reaction mixture was then stirred vigorously at rt for 7 h. After 7 h, the reaction mixture was concentrated under reduced pressure and the residue was resuspended in 900 μL of DMF and purified directly by preparatory HPLC (10% acetonitrile in water grading to 90% acetonitrile in water over 30 minutes) to provide drug-linker 49 as a yellow solid (0.6 mg, quant. yield).

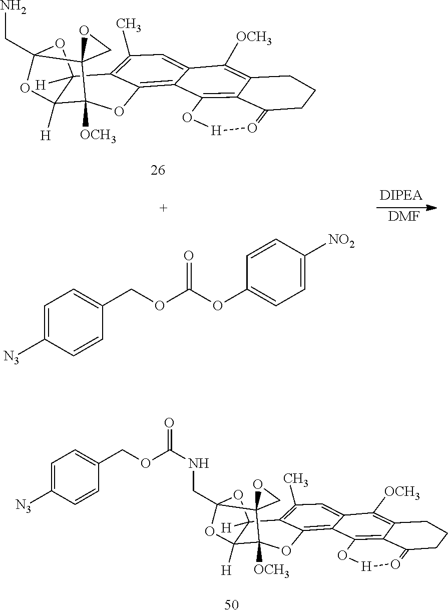

4-azidobenzyl (((2R,2′R,3a'S,4′R)-12′-hydroxy-7′,13a′-dimethoxy-5′-methyl-11′-oxo-3a′,8′,9′,10′,11′,13a′-hexahydro-2′H,4′H-spiro[oxirane-2,1′-[2,4]epoxyfuro[3,2-b]naphtho[2,3-h]chromen]-2′-yl)methyl)carbamate (50). To a solution of 26 (22.5 mg, 0.049 mmol) in anhydrous DMF (1.5 mL, 0.033 M) was added DIPEA (0.086 mL, 0.494 mmol, 10 equiv.) followed by the mixed carbonate (78.0 mg, 0.247 mmol, 5 equiv.). The reaction mixture was stirred at 23° C. overnight. The next day (˜18 h), the reaction mixture was diluted with DMF to a total volume of 1.8 mL and purified directly by preparatory HPLC (10% acetonitrile in water grading to 90% acetonitrile in water over 30 minutes) to provide carbamate 50 as a yellow solid (12.9 mg, 41% yield).

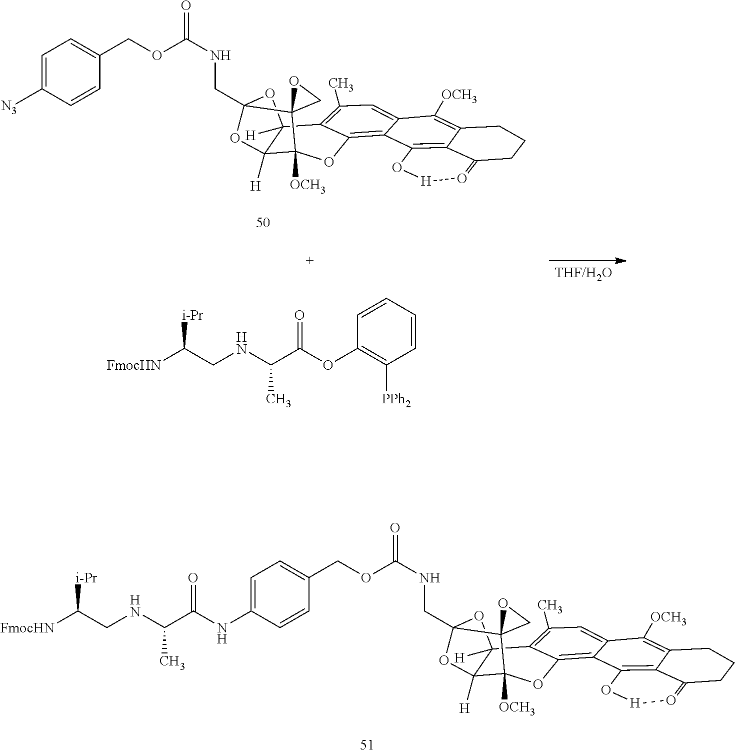

4-((S)-2-((S)-2-((((9H-fluoren-9-yl)methoxy) carbonyl)amino)-3-methylbutanamido)propanamido)benzyl (((2R,2′R,3a'S,4′R)-12′-hydroxy-7′,13a′-dimethoxy-5′-methyl-11′-oxo-3a′,8′,9′,10′,11′,13a′-hexahydro-2′H,4′H-spiro [oxirane-2,1′-[2,4]epoxyfuro[3,2-b]naphtho[2,3-h]chromen]-2′-yl)methyl)carbamate (51). To a flame-dried round bottom flask charged with a magnetic stir bar was added carbamate 50 (12.9 mg, 0.020 mmol) as a solution in THF (0.818 mL) and water (0.205 mL, resultant soln. 0.02 M). The dipeptide was added in one portion (0.027 g, 0.041 mmol, 2 equiv.) and the reaction mixture was stirred at 23° C. overnight. After 24 h, the reaction mixture was diluted with ethyl acetate and partitioned between sat. aq. brine and ethyl acetate. The combined organic layers were dried over anhydrous sodium sulfate, filtered, and concentrated under reduced pressure. The residue by preparatory HPLC (10% acetonitrile in water grading to 90% acetonitrile in water over 30 minutes) to provide 51 as a yellow solid (9.5 mg, 47% yield).

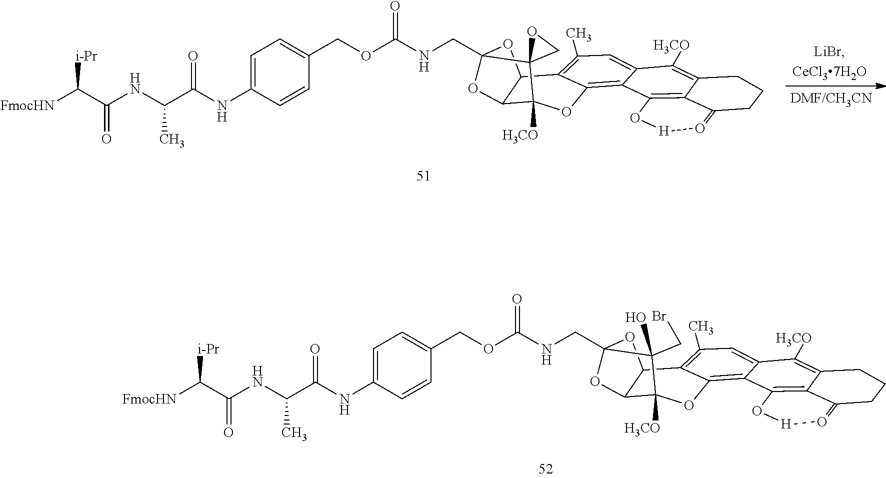

4-((S)-2-((S)-2-((((9H-fluoren-9-yl)methoxy)carbonyl)amino)-3-methylbutanamido)propanamido)benzyl (((1R,2R.3aR)-1-(bromomethyl)-1,12-dihydroxy-7,13a-dimethoxy-5-methyl-11-oxo-1,3a,4,8,9,10,11,13a-octahydro-2H-2,4-epoxyfuro [3,2-b]naphtho[2,3-h]chromen-2-yl)methyl)carbamate (52). To a solution of 51 (11.5 mg, 0.012 mmol) in anhydrous acetonitrile (1.326 mL) and DMF (0.663 mL, 0.0058 M) was added lithium bromide (0.030 g, 0.346 mmol, 30 equiv.) followed by cerium trichloride heptahydrate (0.013 g, 0.035 mmol, 3.0 equiv.). The reaction mixture was then wrapped in aluminum foil to exclude light and stirred at 23° C. overnight. After stirring overnight (˜18 h), the reaction mixture was partitioned between sat. aq. sodium sulfite solution (5 mL) and EtOAc (5 mL). The aqueous layer was then extracted with EtOAc. The organic layers were combined, dried over anhydrous sodium sulfate, filtered, and concentrated under reduced pressure. The residue was purified by reverse phase preparatory LCMS (10% acetonitrile in water grading to 90% acetonitrile in water over 30 minutes) to afford 7.00 mg (56% yield) of 52 as a yellow solid.

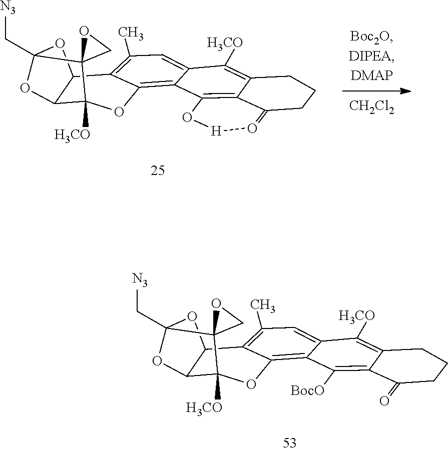

(2R,2′R,3a'S)-2′-(azidomethyl)-7′,13a′-dimethoxy-5′-methyl-11′-oxo-3a′, 8′,9′,10′,11′,13a′-hexahydro-2′H,4′H-spiro[oxirane-2,1′-[2,4]epoxyfuro[3,2-b]naphtho[2,3-h]chromen]-12′-yl tert-butyl carbonate (53). To a solution of azide 25 (26.6 mg, 55.2 μmol) in anhydrous dichloromethane (1.10 mL, 0.05 M) was added Hunig's base (0.043 mL, 0.249 mmol, 4.5 equiv.), boc-anhydride (0.064 mL, 0.276 mmol, 5 equiv.), and DMAP (3.37 mg, 27.6 μmol, 0.5 equiv.) in that order. After stirring at rt for 3 h, the reaction mixture was diluted with dichloromethane (1 mL) and quenched with sat. aq. ammonium chloride (2 mL). The layers were separated and the aqueous layer was extracted with dichloromethane (2 mL). The combined organic phases were dried over sodium sulfate, filtered, and concentrated under reduced pressure. The crude material was purified via silica gel flash chromatography to afford 53 (26.4 mg, 82% yield) as an off-white solid.

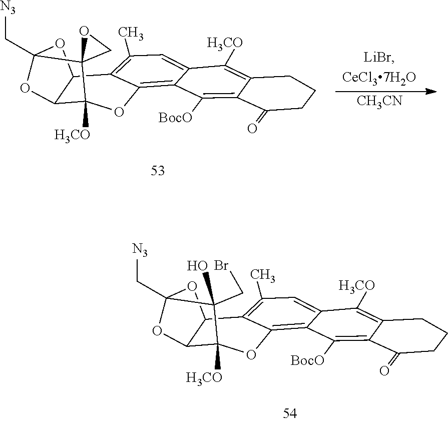

(1R,2R,3aS)-2-(azidomethyl)-1-(bromomethyl)-1-hydroxy-7.13a-dimethoxy-5-methyl-11-oxo-1,3a,4,8,9,10,11,13a-octahydro-2H-2,4-epoxyfuro[3,2-b]naphtho[2,3-h]chromen-12-yltert-butyl carbonate (54). To a solution of boc-protected azide 53 (40.0 mg, 68.8 μmol) dissolved in acetonitrile (2.30 mL, 0.03M) was added lithium bromide (179 mg, 2.06 mmol, 30 equiv.) and cerium (III) chloride heptahydrate (76.9 mg, 206 μmol, 3 equiv.). After stirring overnight (˜18 h), the reaction mixture was partitioned between sat. aq. sodium sulfite solution and ethyl acetate. The organic phase was separate, washed with brine, dried over anhydrous sodium sulfate, filtered, and concentrated under reduced pressure to afford a crude residue. The crude residue was purified via silica gel flash chromatography to afford 54 (39.0 mg, 86% yield) as an off-white solid.

### Antiproliferative Assays

Cell Culture: All cell-culture work was conducted in a class II biological safety cabinet. H460 cells (P2-P4, American Type Culture Collection) were maintained in RPMI-1640 medium supplemented with 10% fetal bovine serum (FBS). HL-60 cells (P2-P4, American Type Cuture Collection) were maintained in RPMI-1640 medium supplemented with 10% FBS. MES-SA/Dx-5 cells (P2-P4, European Collection of Authenticated Cell Cultures) were maintained in McCoy's 5A medium supplemented with 10% FBS.

Cell Proliferation Assay: H460 and MES-SA/Dx-5 cells were grown to approximately 80% confluence, and then were trpysinized, collected, and pelleted by centrifugation (10 min at 183 x g, 24° C.), and HL-60 cells were grown to confluence and directly pelleted by centrifugation (10 min at 183 × g, 24° C.). The supernatant was discarded and the cell pellet was resuspended in 10 mL of fresh medium. A sample was diluted 10-fold in fresh medium, and the concentration of cells was determined using a hemacytometer. The cell suspension was diluted to a concentration of 3000 cells/100 μL. The wells of a pre-sterilized 96-well plate were charged with 100 μL per well of the diluted cellular suspension. The plate was incubated for 24 h at 37° C. (5% CO₂).

Stock solutions of each compound in DMSO were diluted with the appropriate media to 100 nM solutions (1% DMSO), which were then serially diluted with the appropriate media, and 100-uL aliquots of the resulting solutions were added to the wells containing adhered cells to achieve final concentrations of 0.39 nM to 50 nM. After incubating at 37° C. for 72 h (5% CO₂), 20 μL of resazurin solution (Promega CellTiter-Blue® Cell Viability Assay) was added to each well. After incubating at 37° C. for 2.0 h (5% CO₂), the fluorescence (544 nm excitation/590 nm emission) was recorded using a microplate reader (SpectraMax PLUS384) as a measure of viable cells.

Percent growth inhibition was calculated for each well, based upon the following formula: Percent growth inhibition=100×(S−B0)/(Bt−B0) where S is the sample fluorescence, Bt is the average fluorescence of an untreated population of cells at the completion of the assay, and B0 is the average fluorescence of an untreated population of cells at the beginning of the assay.

Each compound was assayed at eight separate concentrations per experiment. The percent inhibition at each concentration was plotted against log (concentration), and a curve fit was generated using the XLfit4 plugin (IDBS Software) running in Excel (Microsoft). GI50 values were computed to reflect the concentrations at which the resulting curves pass through 50% inhibition. GI50 values for each compound are reported as the average of at least six experiments, with standard deviation.

Compounds of the disclosure are potent inhibitors of the growth of MES-SA/Dx-5 cells as shown in Table 1.

TABLE 1

| Compound | GI₅₀ (nM) |
|---|---|
| 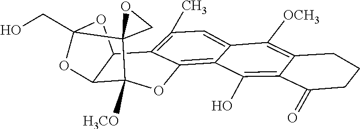 | 8 |
| TP-1 | |
| 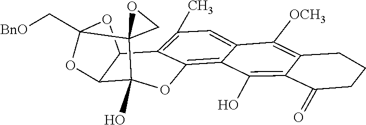 | >100 |
| TP-4 | |
| 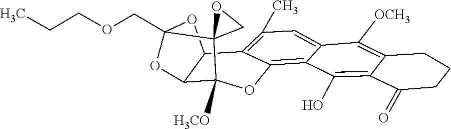 | 25 |
| TP-5 | |
| 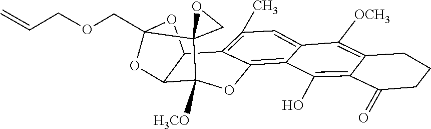 | 17 |
| TP-6 | |
| 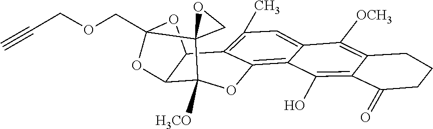 | 15 |
| TP-7 | |
| 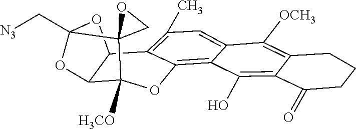 | 33 |
| TP-9 | |
| 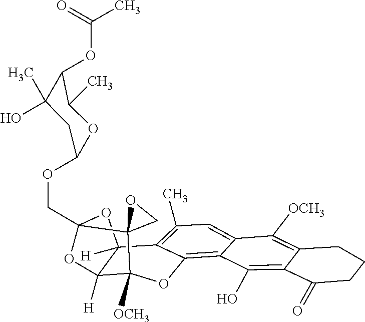 | 50 |
| TP-12 | |
| 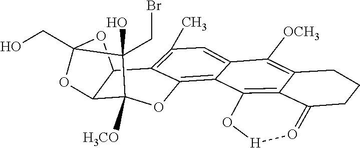 | 8 |
| TP-14 | |

Compounds of the disclosure are potent inhibitors of the growth of H460 cells as shown in Table 2.

TABLE 2

| Compound | GI₅₀ (nM) |
|---|---|
| 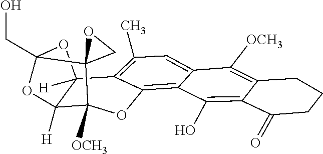 | 5.5 |
| 19 | |
| 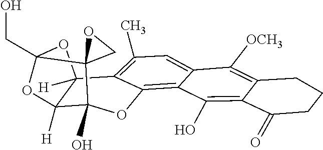 | 15 |
| 20 | |
| 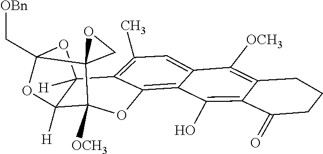 | >100 |
| 18 | |
| 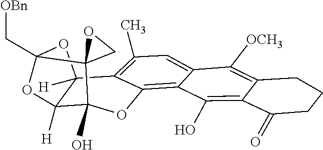 | >100 |
| 17 | |
| 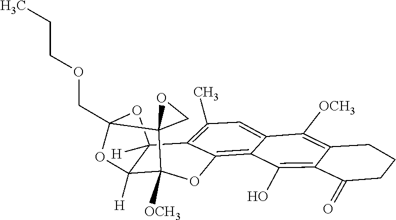 | 22 |
| 22 | |
| 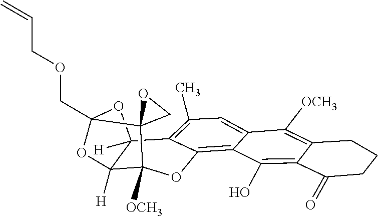 | 14 |
| 23 | |
| 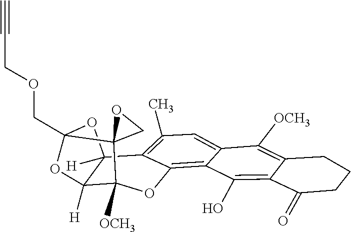 | 14 |
| 24 | |
| 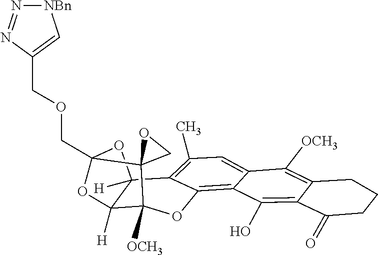 | >100 |
| 27 | |
| 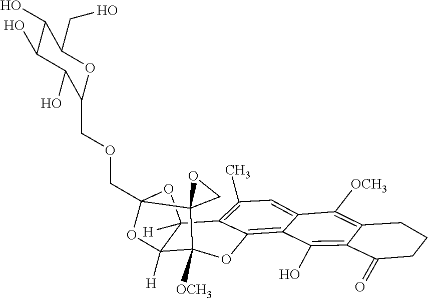 | >100 |
| 30 | |
| 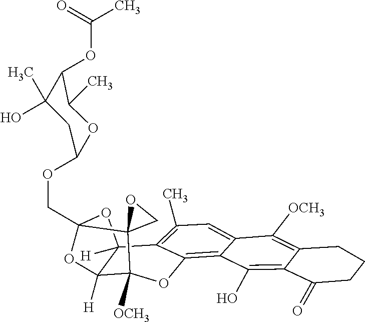 | 20 |
| 30 | |
| 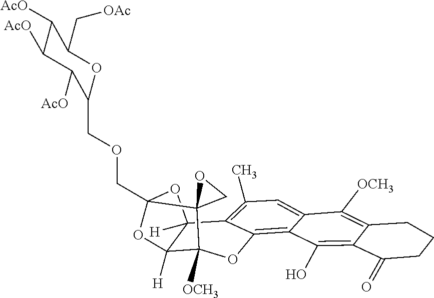 | >100 |
| 28 | |
| 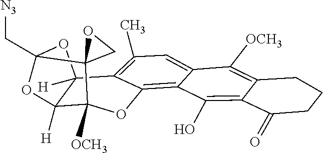 | 33 |
| 25 | |
| 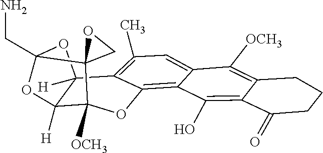 | 10 |
| 26 | |
| 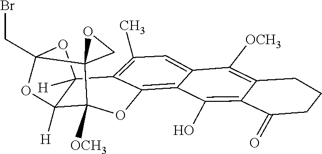 | 20 |
| 32 | |
| 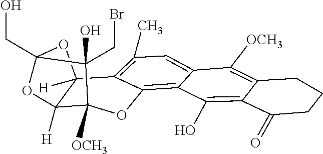 | 8 |
| 21 | |
| 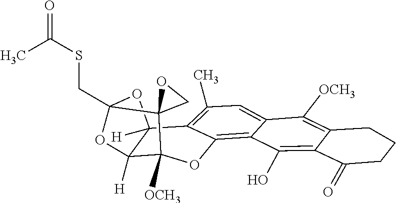 | 100 |
| 33 | |
| 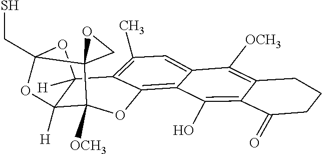 | >100 |
| 34 | |
| 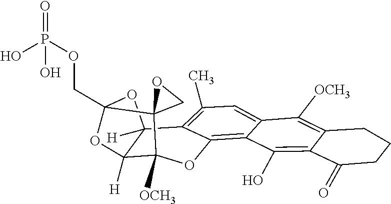 | 35 |
| 35 | |

These data indicate that small permutations to the chemical moiety present at C16 do not drastically perturb the potency of the resulting compound. Increasing the oxygenation of the propyl chain (22) to the propargyl alkyne (14) results in a corresponding increase in potency which is consistent with a reduction in rotatable bonds. Addition of a triazole-benzyl projection via ‘click’ chemistry (27) ablates potency, likely through a steric clash that prevents the compound from properly intercalating dsDNA. The trioxacarcinose A residue is well tolerated (30) while the non-native glucose residue (28, 29) is poorly tolerated, likely through increased uptake in efflux pathways. Substitution of the C16 alcohol with a halogen (32), azide (25), amine (26), or phosphate (35) all showed good tolerance. The surprising activity of the primary amine suggests that its pKa is high enough to not be protonated under physiologically relevant conditions. Sulfur based substitutions were not well tolerated at C16. ¹H NMR studies pointed to a rapid, non-productive, decomposition pathway that occurred on an hour time scale when the thiol (34) was not protected as the thioacetate (33). Thioesterase activity on the thioacetate likely forms the unstable thiol in situ which likely explains the similar lack of activity in both sulfur-containing compounds.

Compounds of the disclosure are potent inhibitors of the growth of Ramos cells (B lymphocyte cells) as shown in Table 3.

TABLE 3

| | GI₅₀ |
|---|---|
| Compound | (nM) |
| 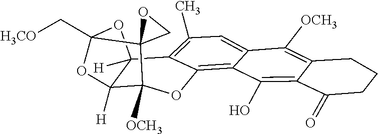 | 2.6 ± 0.8 |
| 43 | |
| 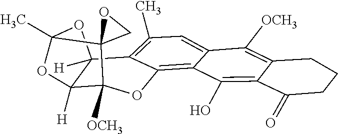 | 20 ± 5 |
| 45 | |

### Stability and Release Assays

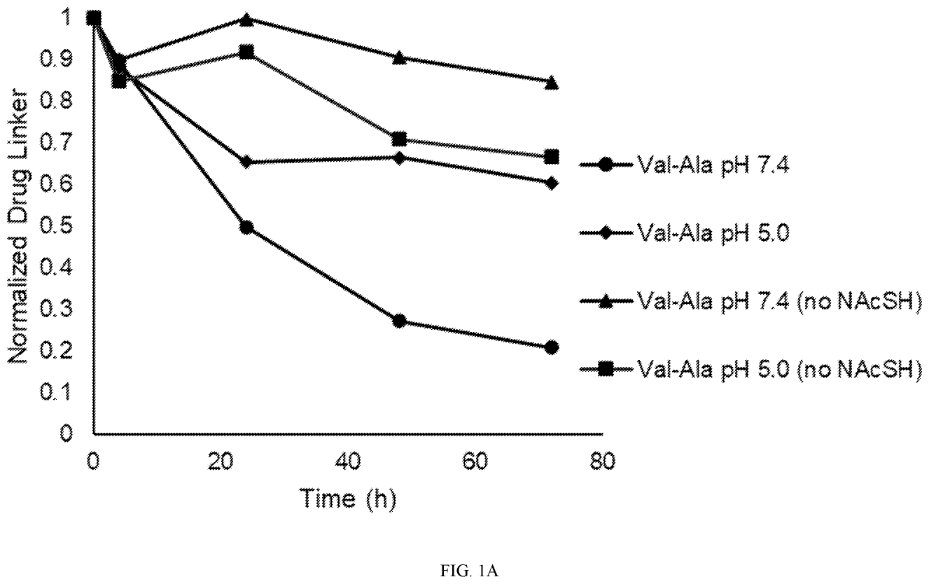

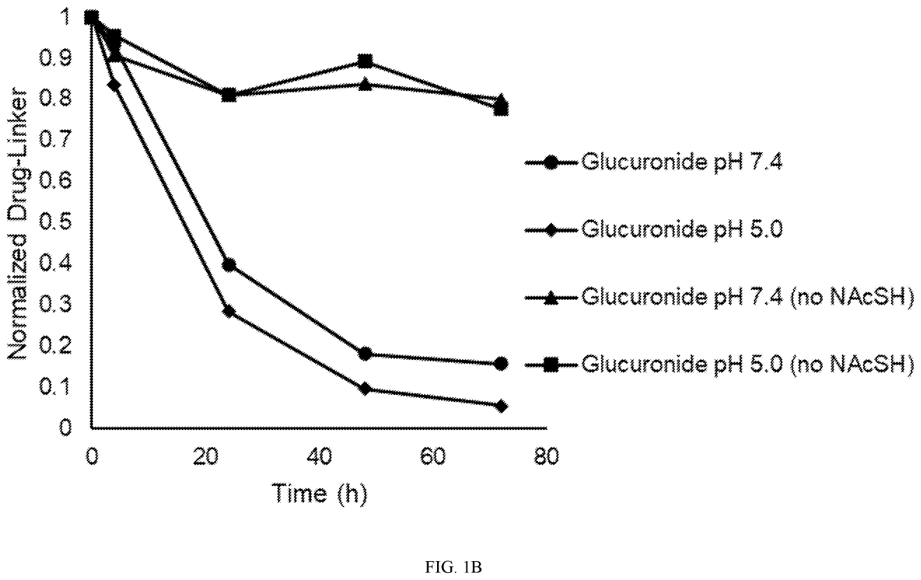

Stability Assay: In a 1.5 mL screw-top conical plastic vial, 42 (FIG. 1A) or 39 (FIG. 1B) was dissolved in 270 μL of DMSO (5 mM). 12 μL of this DMSO drug-linker solution was mixed with 576 μL pH 5.0 sodium acetate buffer (50 mM sodium acetate, 100 mM NaCl, 4 mM EDTA). 1-naphthalene acetic acid in DMSO (12 μL, 5 mM, 1 equiv.) was added as an internal standard. The vial was then placed in a heating block at 37 C and protected from direct light. 25 μL aliquots of the reaction mixture were removed at 0 h, 4 h, 24 h, 48 h, 72 h, and 1 week and analyzed by LC-MS. LC-MS peaks were integrated using Agilent OpenLab ChemStation and the areas of each peak were normalized to 1-napthalene acetic acid at 280 nM. Percent drug-linker remaining is defined as the normalized peak area at a given time point divided by the normalized peak area at 0 hours x 100%. Each assay was also performed at pH 7.4 (phosphate-buffered solution, Corning), and assays at each pH value were performed with and without the addition of 8 mM N-acetylcysteine.

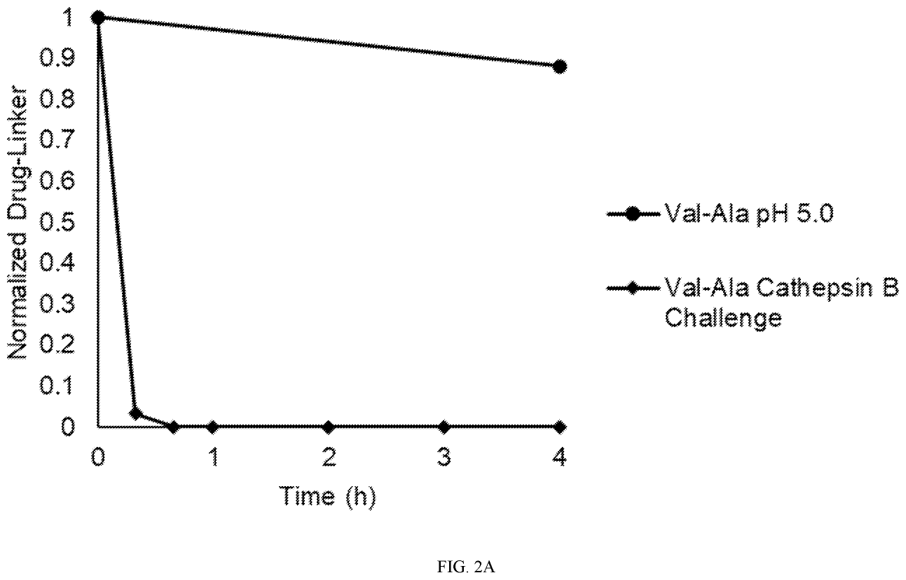

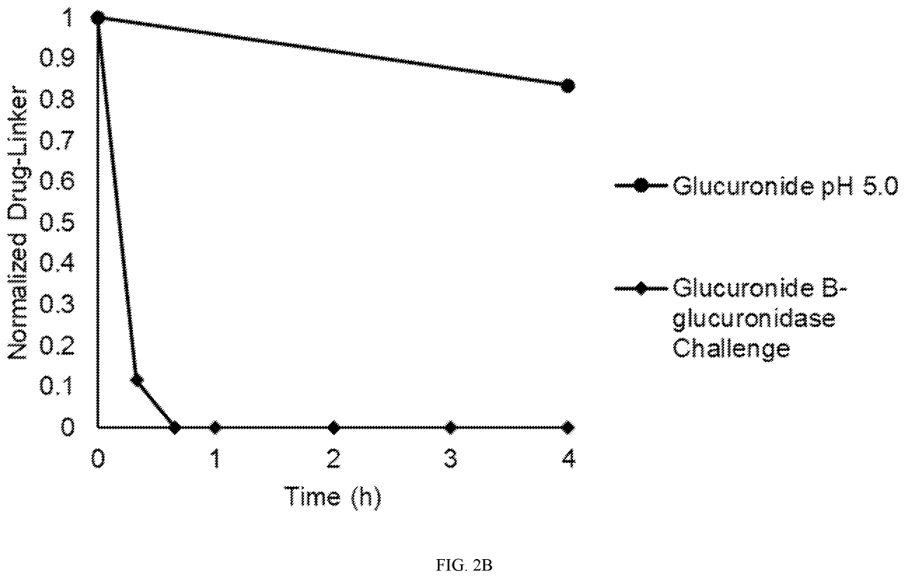

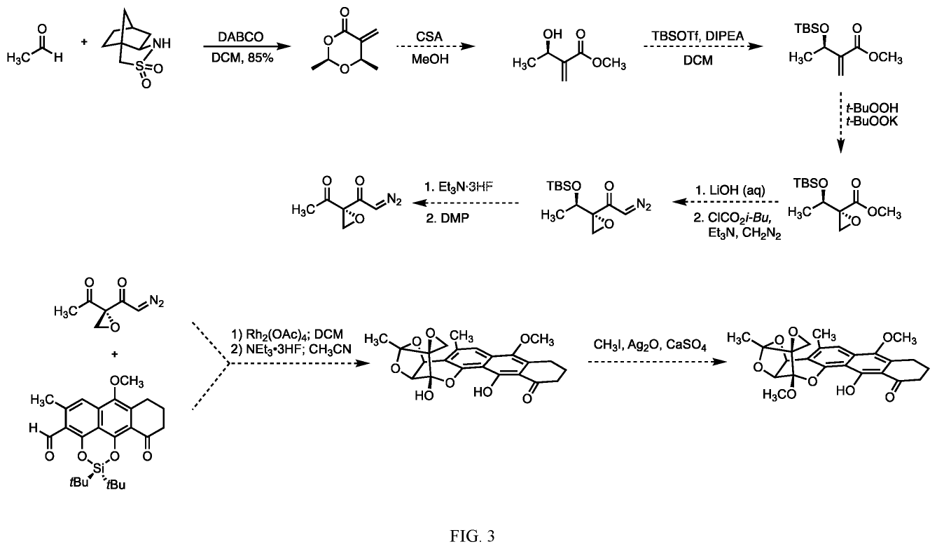

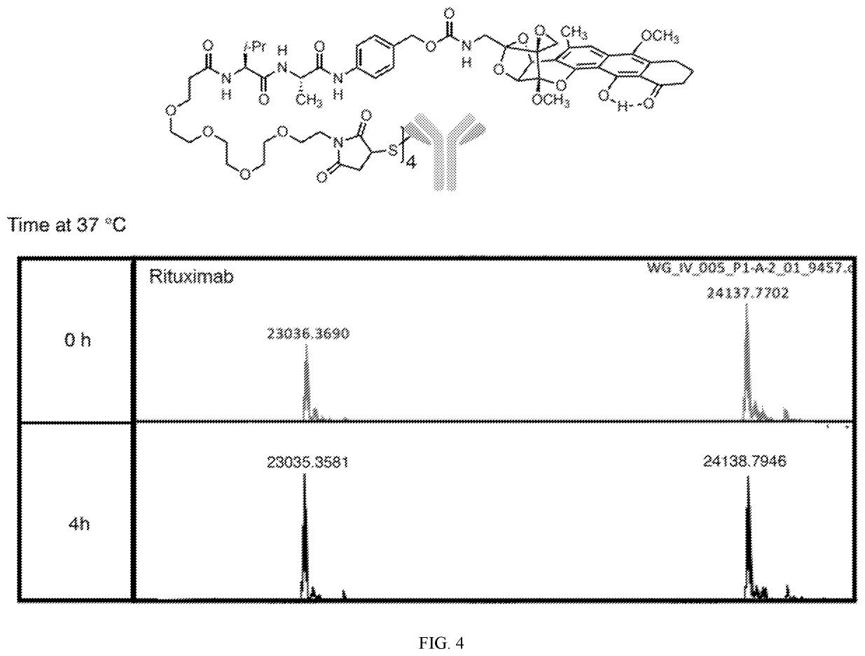

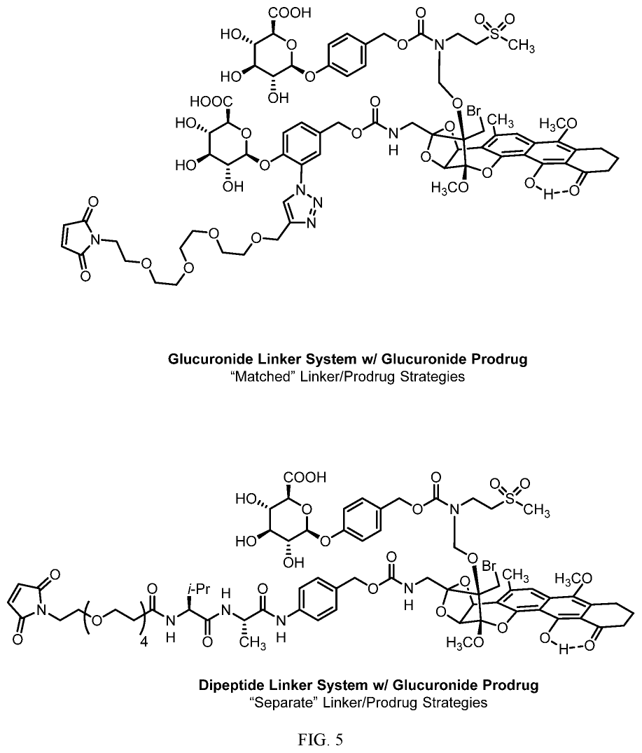

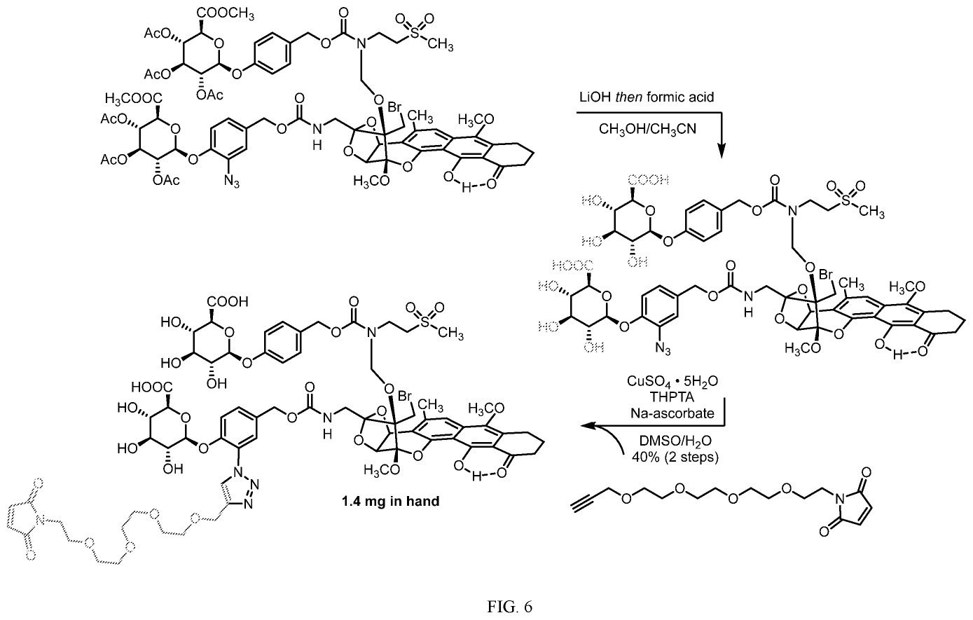

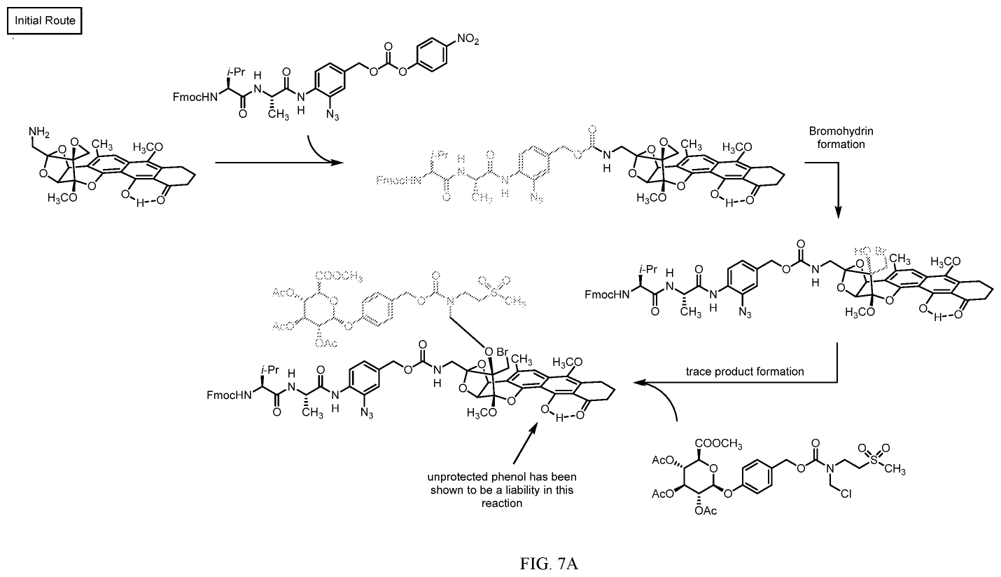

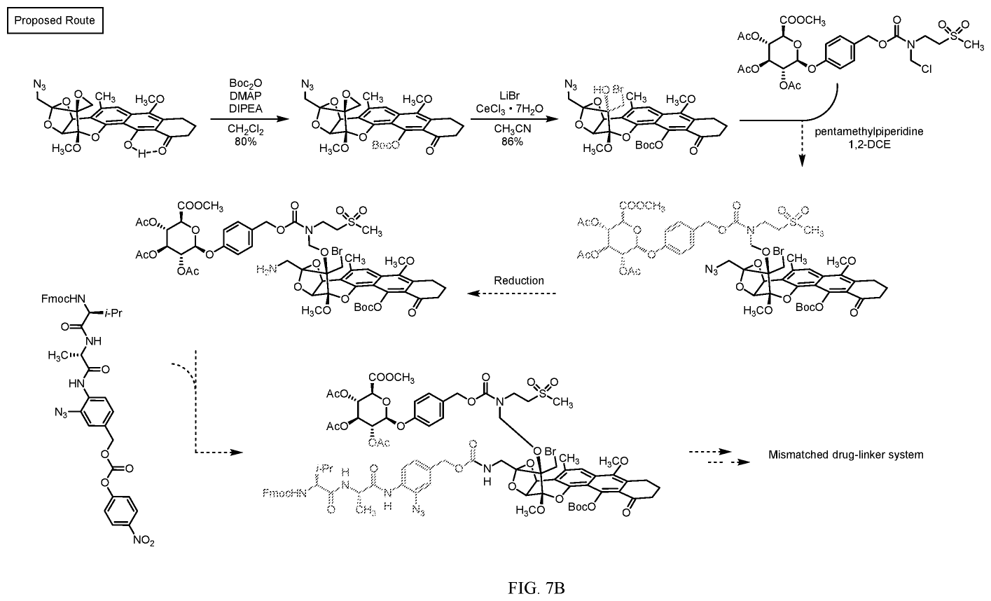

Release Assay: In a 1.5 mL screw-top conical plastic vial, 42 was dissolved in 270 μL of DMSO (5 mM). 12 μL of this DMSO drug-linker solution was mixed with a DMSO solution of N-acetylcysteine (10 mM, 12 μL, 2 equiv.). To the solution was then added 510 μL pH 5.0 sodium acetate buffer (50 mM sodium acetate, 100 mM NaCl, 4 mM EDTA, 8 mM N-acetylcysteine). 1—Naphthalene acetic acid in DMSO (12 μL, 5 mM, 1 equiv.) was added as an internal standard. The vial was then placed in a heating block at 37 C for 30 minutes. After 30 minutes, 54 μL of cathepsin B solution (379 U/mL, Enzo Life Sciences) was added. The vial was returned to the heating block at 37 C and protected from direct light. 25 μL aliquots of the reaction mixture were removed at 0 min, 20 min, 40 min, 60 min, 120 min, and 180 min and analyzed by LC-MS. LC-MS peaks were integrated using Agilent OpenLab ChemStation and the areas of each peak were normalized to 1-napthalene acetic acid at 280 nM. Percent drug-linker remaining is defined as the normalized peak area at a given time point divided by the normalized peak area at 0 hours×100% (FIG. 2A). A similar procedure was used for the release assay of 39, using glucuronidase instead of cathepsin B (FIG. 2B).

Both drug-linker systems (i.e., 42 and 39) were stable at physiologically relevant pH and temperature for over 1 week. After 1 week, 75% of the glucuronide- and dipeptide drug-linker system remained. Then, when the appropriate enzyme was added, both drug-linker systems rapidly liberated the C16 primary amine warhead with a half-life of approximately 15 minutes.

### Preparation of Trioxacarcin-Antiboduy Conjugates

The methods for preparing and characterizing the antibody-drug conjugates are described below.

Partial reduction of interchain disulfides: To a solution of rituximab (500 μg, 25 μL of 20 mg/mL in PBS) was added a solution of tris(2-carboxyethyl)phosphine hydrochloride (1.7 μL, 5 mM in water, 2.5 equiv) and diluted to 15 mg/mL final antibody concentration using PBS pH 7.4 containing ethylenediaminetetraacetic acid (5 mM, 8.3 μL). The reaction was incubated at 37° C. for 2 h. The resulting antibodies contained on average 4 free thiols per antibody.

Conjugation of trioxacarcin-linker: To a solution of partially reduced rituximab was added drug-linker (6.9 μL, 5 mM in DMSO, 11.5 equiv). DPBS pH 7.4 containing ethylenediaminetetraacetic acid (5 mM, 8.3 μL) was added to achieve 10 mg/mL final antibody concentration. The reaction was incubated for 2 h at 37° C. with agitation at 500 rpm. After conjugation, the antibody solution was diluted to 100 μL with PBS (47.5 μL) and loaded onto a PD SpinTrap G-25 Column (GE Healthcare, Chicago, IL, USA) pre-equilibrated with PBS according to the manufacturer's protocol. An additional 40 μL of PBS was added to the column once the ADC solution had fully entered the media. The column was centrifuged at 800 Å˜ g for 2 min and the filtrate was collected (140 μL total). The filtrate was diluted to 200 μL with PBS (60 μL), generating an ADC concentration of ˜2.5 mg/mL. The resulting antibody-drug conjugate was analyzed by HRMS and stored at −80° C.

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 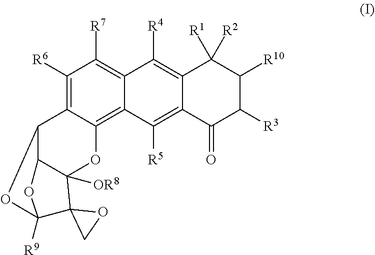
or a pharmaceutically acceptable salt, solvate, hydrate, polymorph, co-crystal, tautomer, stereoisomer, isotopically labeled derivative, or prodrug thereof;
wherein:
R¹ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{A1}; —C(═O)R^{A2}; —CO₂R^{A2}; —CN; —SCN; —SR^{A1}; —SOR^{A1}; —SO₂R^{A1}; —NO₂; —N₃; —N(R^{A2})₂; —NR^{A2}C(═O)R^{A2}; —NR^{A2}C(═O)N(R^{A2})₂; —OC(═O)OR^{A1}; —OC(═O)R^{A2}; —OC(═O)N(R^{A2})₂; —NR^{A}C(═O)OR^{A1}; or —C(R^{A2})₃; wherein each occurrence of R^{A1} is independently hydrogen; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{A1} groups are optionally joined to form a substituted or unsubstituted heterocyclyl or substituted or unsubstituted heteroaryl ring; and wherein each occurrence of R^{A2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{A1}; —SR^{A1}; or —N(R^{A1})₂, or two R^{A2} groups are optionally joined to form a substituted or unsubstituted heterocyclyl or substituted or unsubstituted heteroaryl ring; with the proviso that if R¹ is —OR^{A1}, R^{A1} is not 4-methoxybenzyl or carbohydrate.
R² is hydrogen; or R¹ and R² are joined to form ═O; ═N(R^{A2}); or ═S;
R³ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{C1}; —C(═O)R^{C2}; —CO₂R^{C1}; —CN; —SCN; —SR^{C1}; —SOR^{C1}; —SO₂R^{C2}; —NO₂; —N₃; —N(R^{C2})₂; —NHC(═O)R^{C2}; —NR^{C2}C(═O)N(R^{C2})₂; —OC(═O)OR^{C1}; —OC(═O)R^{C2}; —OC(═O)N(R^{C2})₂; —NR^{C2}C(═O)OR^{C1}; or —C(R^{C2})₃; wherein each occurrence of R^{C1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{C1} groups are optionally joined to form a substituted or unsubstituted heterocyclyl or substituted or unsubstituted heteroaryl ring; and wherein each occurrence of R^{C2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{C1}; —SR^{C1}; or —N(R^{C1})₂; or two R^{C2} groups are optionally joined to form a substituted or unsubstituted heterocyclyl or substituted or unsubstituted heteroaryl ring;
R⁴ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{D1}; —C(═O)R^{D2}; —CO₂R^{D2}; —CN; —SCN; —SR^{D1}; —SOR^{D1}; —SO₂R^{D2}; —NO₂; —N₃; —N(R^{D2})₂; —NR^{D2}C(═O)R^{D2}; —NR^{D2}C(═O)N(R^{D2})₂; —OC(═O)OR^{D1}; —OC(═O)R^{D2}; —OC(═O)N(R^{D2})₂; —NR^{D2}C(═O)OR^{D1}; or —C(R^{D2})₃; wherein each occurrence of R^{D1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{D1} groups are optionally joined to form a substituted or unsubstituted heterocyclyl or substituted or unsubstituted heteroaryl ring; and wherein each occurrence of R^{D2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{D1}; —SR^{D1}; or —N(R^{D1})₂; or two R^{D2} groups are optionally joined to form a substituted or unsubstituted heterocyclyl or substituted or unsubstituted heteroaryl ring;
R⁵ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{E1}; —C(═O)R^{E2}; —CO₂R^{E1}; —CN; —SCN; —SR^{E1}; —SOREL; —SO₂R^{E2}; —NO₂; —N₃; —N(R^{E2})₂; —NR^{E2}C(═O)R^{E2}; —NR^{E2}C(═O)N(R^{E2})₂; —OC(═O)OR^{E1}; —OC(═O)R^{E2}; —OC(═O)N(R^{E2})₂; —NR^{E2}C(═O)OR^{E1}; or —C(R^{E2})₃; wherein each occurrence of R^{E1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{E1} groups are optionally joined to form a substituted or unsubstituted heterocyclyl or substituted or unsubstituted heteroaryl ring; and wherein each occurrence of R^{E2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{E1}; —SR^{E1}; or —N(R^{E1})₂; or two R^{E2} groups are optionally joined to form a substituted or unsubstituted heterocyclyl or substituted or unsubstituted heteroaryl ring;
R⁶ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{F1}; —C(═O)R^{F2}; —CO₂R^{F1}; —CN; —SCN; —SR^{F1}; —SOR^{F1}; —SO₂R^{F2}; —NO₂; —N₃; —N(R^{F2})₂; —NR^{F2}C(═O)R^{F2}; —NR^{F2}C(═O)N(R^{F2})₂; —OC(═O)OR^{F1}; —OC(═O)R^{F2}; —OC(═O)N(R^{F2})₂; —NR^{F2}C(═O)OR^{F1}; or —C(R^{F2})₃; wherein each occurrence of R^{F1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{F1} groups are optionally joined to form a substituted or unsubstituted heterocyclyl or substituted or unsubstituted heteroaryl ring; and wherein each occurrence of R^{F2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{F1}; —SR^{F1}; or —N(R^{F1})₂; or two R^{F2} groups are optionally joined to form a substituted or unsubstituted heterocyclyl or substituted or unsubstituted heteroaryl ring;
R⁷ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{G1}; —C(═O)R^{G2}; —CO₂R^{G1}; —CN; —SCN; —SR^{G1}; —SOR^{G1}; —SO₂R^{G2}; —NO₂; —N₃; —N(R^{G})₂; —NR^{G2}C(═O)R^{G2}; —NR^{G2}C(═O)N(R^{G2})₂; —OC(═O)OR^{G1}; —OC(═O)R^{G2}; —OC(═O)N(R^{G2})₂; —NR^{G2}C(═O)OR^{G1}; or —C(R^{G2})₃; wherein each occurrence of R^{G1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{G1} groups are optionally joined to form a substituted or unsubstituted heterocyclyl or substituted or unsubstituted heteroaryl ring; and wherein each occurrence of R^{G2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{G1}; —SR^{G1}; or —N(R^{G1})₂; or two R^{G2} groups are optionally joined to form a substituted or unsubstituted heterocyclyl or substituted or unsubstituted heteroaryl ring;
R⁸ is hydrogen; an oxygen protecting group; a carbohydrate; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; substituted or unsubstituted heteroaliphatic; acyl; —OR^{H9}; —OC(═O)R^{H9}; —N(R^{H9})₂; or —NHC(═O)R^{H9}; wherein each occurrence of R^{H9} is independently hydrogen; substituted or unsubstituted alkyl; an oxygen protecting group when attached to an oxygen atom; or a nitrogen protecting group when attached to a nitrogen atom; or two R^{H9} groups are joined to form a substituted or unsubstituted heterocyclic or substituted or unsubstituted heteroaryl ring;
R⁹ is hydrogen or —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring;
R¹⁰ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{B1}; —C(═O)R^{B2}; —CO₂R^{B2}; —CN; —SCN; —SR^{B1}; —SOR^{B1}; —SO₂R^{B2}; —NO₂; —N₃; —N(R^{B2})₂; —NR^{B2}C(═O)R^{B2}; —NR^{B2}C(═O)N(R^{B2})₂; —OC(═O)OR^{B1}; —OC(═O)R^{B2}; —OC(═O)N(R^{B2})₂; —NR^{B2}C(═O)OR^{B1}; or —C(R^{B2})₃; wherein each occurrence of R^{B1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{B1} groups are optionally joined to form a substituted or unsubstituted heterocyclyl or substituted or unsubstituted heteroaryl ring; and wherein each occurrence of R^{B2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{B1}; —SR^{B1}; or —N(R^{B1})₂; or two R^{B2} groups are optionally joined to form a substituted or unsubstituted heterocyclyl or substituted or unsubstituted heteroaryl ring;
or R¹ and R¹⁰ are optionally taken together with the intervening carbon atoms to form an optionally substituted cyclic moiety;
or R¹⁰ and R³ are optionally taken together with the intervening carbon atoms to form an optionally substituted cyclic moiety;
or R¹ and R⁴ are optionally taken together with the intervening carbon atoms to form an optionally substituted cyclic moiety;
or R⁴ and R⁷ are optionally taken together with the intervening carbon atoms to form an optionally substituted cyclic moiety;
or R⁶ and R⁷ are optionally taken together with the intervening carbon atoms to form an optionally substituted cyclic moiety;
L is a linker;
A is a bond or a group of formula: 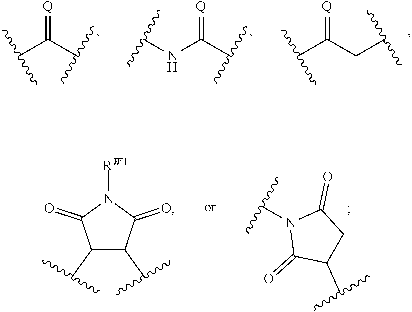
Q is —S— or —O—;
R^{W1} is independently hydrogen, substituted or unsubstituted alkyl; or a nitrogen protecting group;
B is a targeting moiety; and
T is hydrogen, —N═C═S, 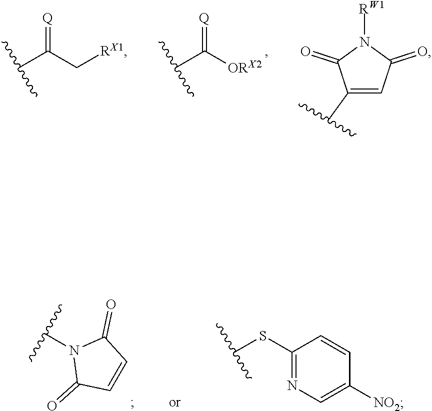
R^{X1} is a leaving group; and
R^{X2} is hydrogen; substituted or unsubstituted alkyl; substituted or unsubstituted heterocyclyl; substituted or unsubstituted aryl; substituted or unsubstituted heteroaryl; or an oxygen protecting group.

2. The compound of claim 1, wherein the compound is of Formula (I-a): 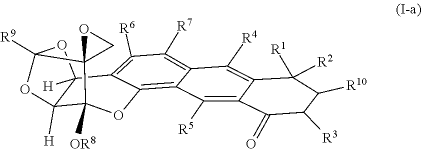
or a pharmaceutically acceptable salt thereof.

3. The compound of any of claims 1-2, wherein the compound is of Formula (I-b): 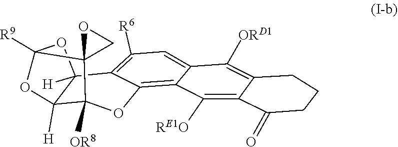
or a pharmaceutically acceptable salt thereof.

4. The compound of any of claims 1-3, wherein the compound is of Formula (I-c): 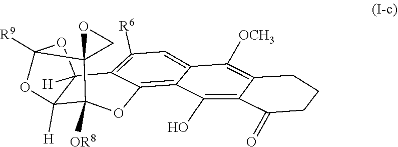
or a pharmaceutically acceptable salt thereof.

5. The compound of any of claims 1-4, wherein R¹ is hydrogen.

6. The compound of any of claims 1-4, wherein R¹ and R² together form ═O.

7. The compound of any of claims 1-6, wherein R⁴ is —OR^{D1}.

8. The compound of any of claims 1-7, wherein R^{D1} is C₁₋₆ alkyl.

9. The compound of any of claims 1-8, wherein R⁵ is —OR^{E1}.

10. The compound of claim 9, wherein R^{E1} is hydrogen.

11. The compound of any of claims 1-10, wherein R⁶ is C₁₋₆ alkyl.

12. The compound of any of claims 1-11, wherein R⁶ is —CH₃.

13. The compound of any of claims 1-12, wherein R⁷ is hydrogen.

14. The compound of any of claims 1-13, wherein R⁸ is hydrogen, a carbohydrate, or C₁₋₆ alkyl.

15. The compound of claim 14, wherein the carbohydrate of R⁸ is: 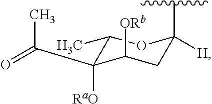
wherein R^{a} and R^{b} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl.

16. The compound of any of claims 1-14, wherein R⁸ is C₁₋₆ alkyl.

17. The compound of any of claim 1-14 or 16, wherein R⁸ is —CH₃.

18. The compound of any of claims 1-14, wherein R⁸ is hydrogen.

19. The compound of any of claims 1-18, wherein the compound is of Formula (I-d): 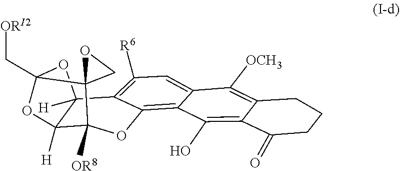
or a pharmaceutically acceptable salt thereof, wherein R^{I2} is hydrogen, carbohydrate, protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted phosphono; substituted or unsubstituted aryl; or substituted or unsubstituted heteroaryl.

20. The compound of claim 19, wherein R^{I2} is hydrogen; oxygen protecting group; carbohydrate; substituted or unsubstituted alkyl; substituted or unsubstituted alkenyl; substituted or unsubstituted alkynyl; substituted or unsubstituted phosphono; or substituted or unsubstituted heteroarylalkyl.

21. The compound of any of claims 19-20, wherein R^{I2} is hydrogen, oxygen protecting group, substituted or unsubstituted alkyl, substituted or unsubstituted alkenyl; substituted or unsubstituted alkynyl; or substituted or unsubstituted phosphono.

22. The compound of any of claims 19-21, wherein R^{I2} is hydrogen, protecting group, substituted or unsubstituted C₁₋₆ alkyl, substituted or unsubstituted C₁₋₆ alkenyl, substituted or unsubstituted C₁₋₆ alkynyl, or substituted or unsubstituted phosphono.

23. The compound of any of claims 19-22, wherein R^{I2} is hydrogen; benzyl; n-propyl; 2-propenyl; 2-propynyl; or —P(═O)(OH)₂.

24. The compound of any of claims 19-20, wherein R^{I2} is substituted or unsubstituted heteroarylalkyl.

25. The compound of any of claims 19-20, wherein R^{I2} is a carbohydrate.

26. The compound of any of claim 19-20 or 25, wherein R^{I2} is: 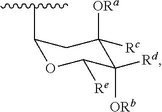
wherein R^{a} and R^{b} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; or acyl; and
R^{c}, R^{d} and R^{e} are independently hydrogen; cyclic or acyclic, substituted or unsubstituted, branched or unbranched aliphatic; cyclic or acyclic, substituted or unsubstituted, branched or unbranched heteroaliphatic; or acyl.

27. The compound of claim 26, wherein R^{a} and R^{b} are independently hydrogen; carbohydrate; oxygen protecting group; or acyl.

28. The compound of any of claim 26 or 27, wherein R^{c}, R^{d}, and R^{e} are independently hydrogen; substituted or unsubstituted alkyl; or acyl.

29. The compound of any of claims 26-28, wherein R^{c}, R^{d}, and R^{e} are independently hydrogen; substituted or unsubstituted C₁₋₆ alkyl; or acyl.

30. The compound of any of claim 1-20 or 25-29, wherein R^{I2} is: 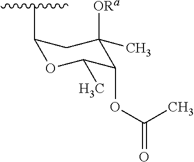
wherein R^{a} is hydrogen; carbohydrate; or oxygen protecting group.

31. The compound of any of claim 1-20 or 25-30, wherein R^{I2} is:

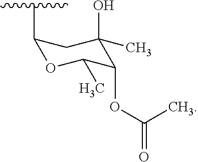 [SMILES: *C1CC(C)(O)C(OC(C)=O)C(C)O1]

32. The compound of any of claim 1-20 or 25-29, wherein R^{I2} is 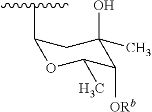
wherein R^{b} is hydrogen; carbohydrate; or oxygen protecting group.

33. The compound of any of claim 1-20, 25-29, or 32, wherein R^{I2} is

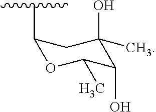 [SMILES: *C1CC(C)(O)C(O)C(C)O1]

34. The compound of any of claim 1-20, 25-29, or 32, wherein R^{I2} is

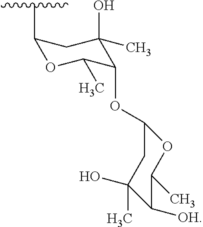 [SMILES: *C1CC(C)(O)C(OC2CC(C)(O)C(O)C(C)O2)C(C)O1]

35. The compound of any of claim 1-20, 25-29, or 32, wherein R^{I2} is

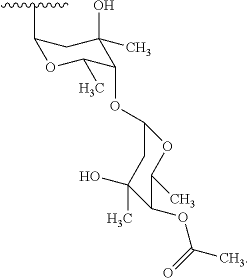 [SMILES: *C1CC(C)(O)C(OC2CC(C)(O)C(OC(C)=O)C(C)O2)C(C)O1]

36. The compound of any of claim 1-20 or 25-29, wherein R^{I2} is: 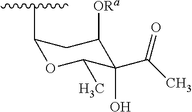
wherein R^{a} is hydrogen; carbohydrate; or oxygen protecting group.

37. The compound of any of claim 1-20, 25-29, or 36, wherein R^{I2} is:

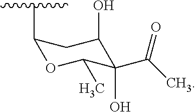 [SMILES: *C1CC(O)C(O)(C(C)=O)C(C)O1]

38. The compound of any of claim 1-20 or 25, wherein R^{I2} is: 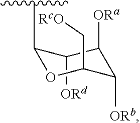
wherein R^{a}, R^{b}, R^{c}, and R^{d} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl.

39. The compound of any of claim 1-20, 25, or 38, wherein R^{a}, R^{b}, R^{c}, and R^{d} are independently hydrogen; carbohydrate; or oxygen protecting group.

40. The compound of any of claim 1-20, 25, or 38-39, wherein R^{I2} is:

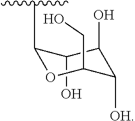 [SMILES: *C1OC(CO)C(O)C(O)C1O]

41. The compound of any of claims 1-18, wherein the compound is of Formula (I-e): 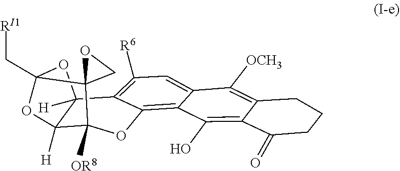
or a pharmaceutically acceptable salt thereof.

42. The compound of claim 41, wherein R^{I1} is hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂.

43. The compound of any of claim 41 or 42, wherein R^{I1} is azido; —N(R^{I2})₂; —SR^{I2}; or halogen.

44. The compound of claim 43, wherein each occurrence of R^{I2} is independently hydrogen; protecting group; cyclic or acyclic, substituted or unsubstituted, branched or unbranched aliphatic; or acyl; or two R^{I2} groups are optionally joined to form a substituted or unsubstituted heterocyclyl or substituted or unsubstituted heteroaryl ring.

45. The compound of any of claims 41-44, wherein R^{I1} is azido; —NH₂; —SH; —S(C═O)CH₃; chloro; bromo; or iodo.

46. The compound of claim 1, wherein the compound is: 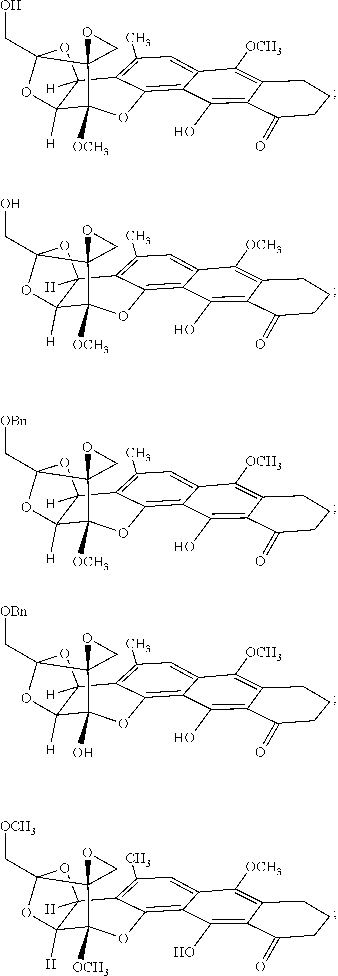 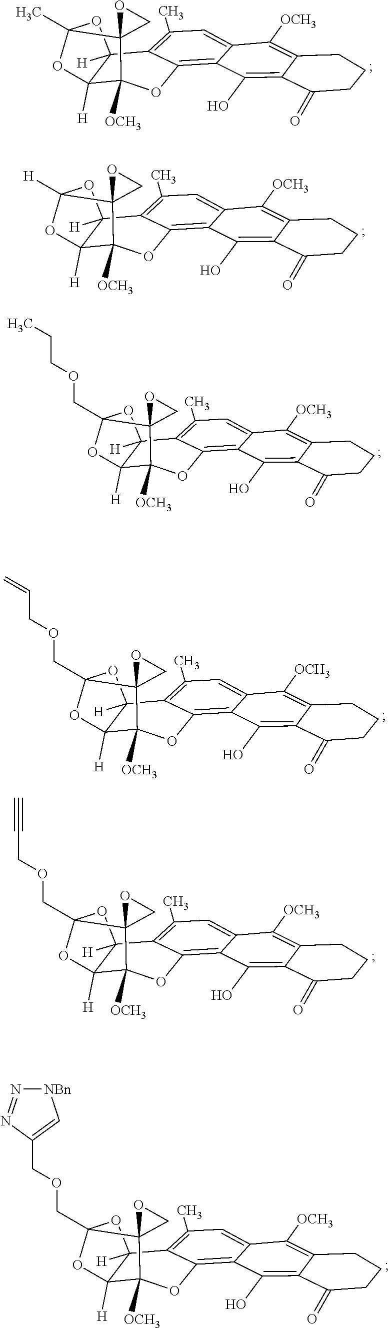 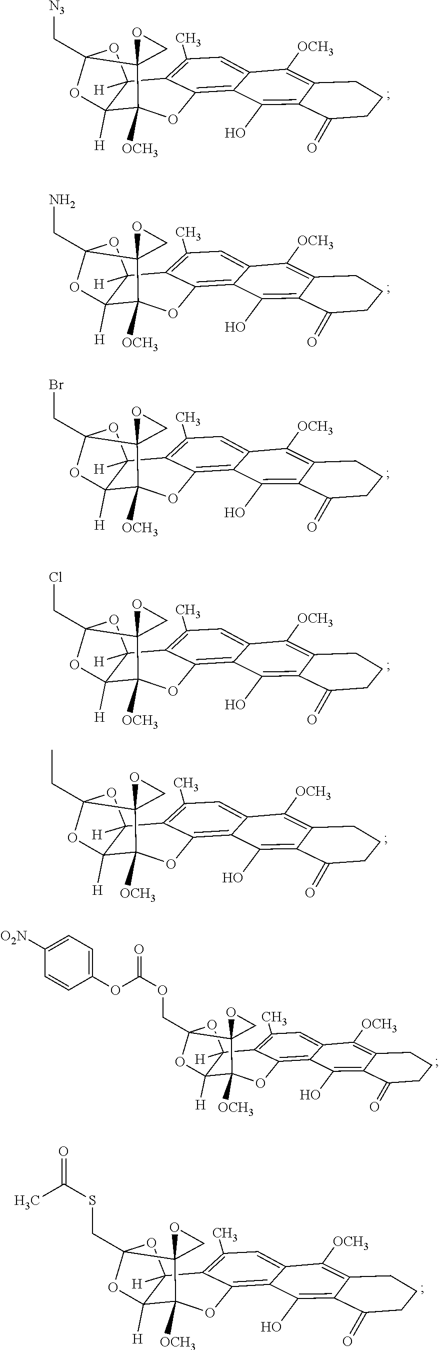 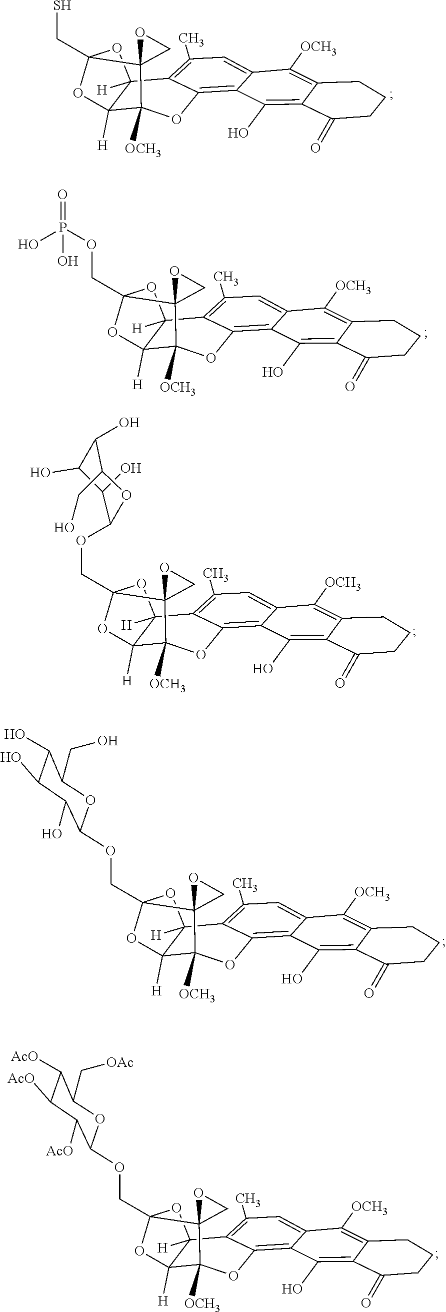 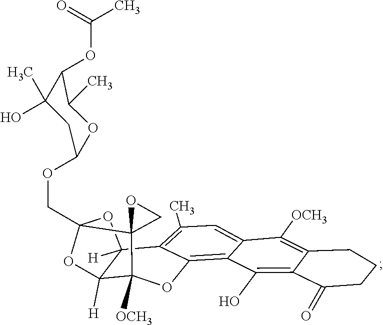 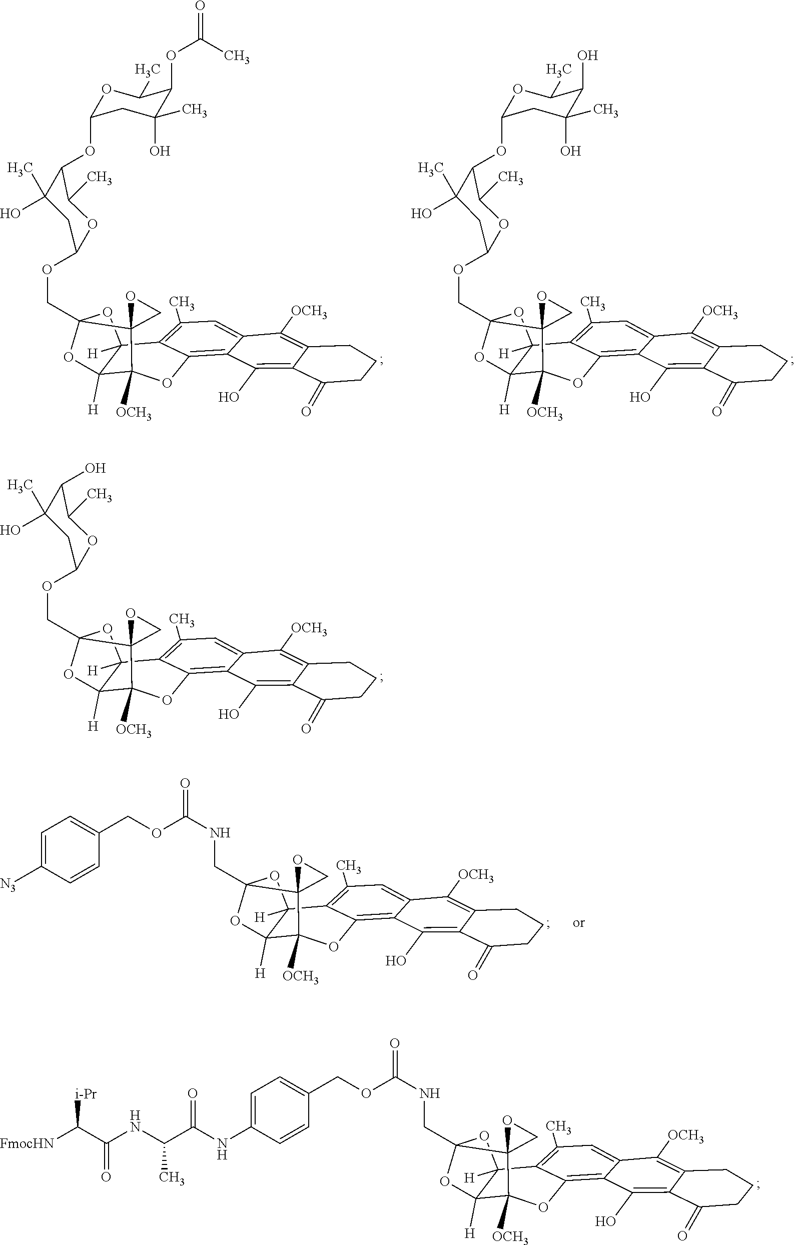
or a pharmaceutically acceptable salt thereof.

47. The compound of any of claims 1-18, wherein at least one occurrence of R^{I1} is —OR^{I2}, —N(R^{I2})₂, or —SR^{I2}.

48. The compound of claim 47, wherein one occurrence of R^{I2} is -L-A-B.

49. The compound of any of claim 1, 47, or 48, wherein the compound is: 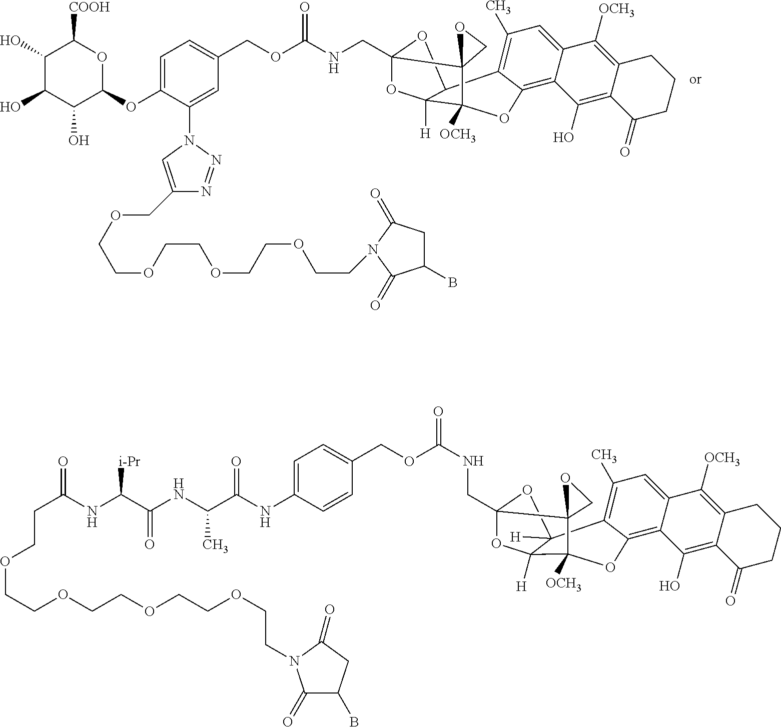
or a pharmaceutically acceptable salt thereof.

50. The compound of claim 47, wherein one occurrence of R^{I2} is -L-T.

51. The compound of any of claim 1, 47, or 50, wherein the compound is: 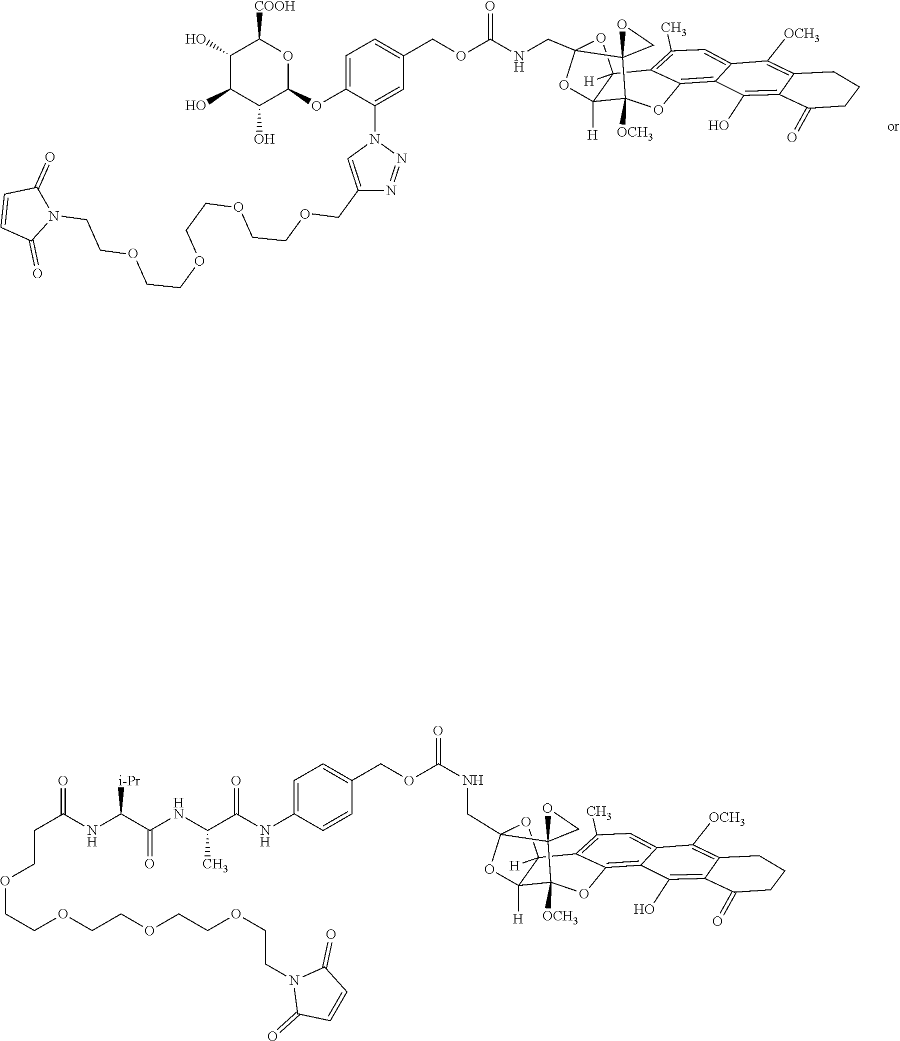
or a pharmaceutically acceptable salt thereof.

52. A compound of Formula (II): 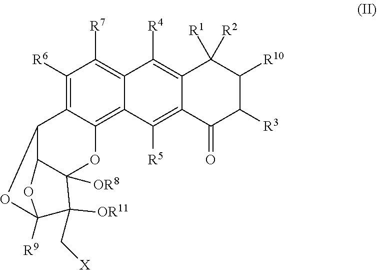
or a pharmaceutically acceptable salt, solvate, hydrate, polymorph, co-crystal, tautomer, stereoisomer, isotopically labeled derivative, or prodrug thereof;
wherein:
R¹ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{A1}; —C(═O)R^{A2}; —CO₂R^{A2}; —CN; —SCN; —SR^{A1}; —SOR^{A1}; —SO₂R^{A1}; —NO₂; —N₃; —N(R^{A2})₂; —NR^{A2}C(═O)R^{A2}; —NR^{A2}C(═O)N(R^{A2})₂; —OC(═O)OR^{A1}; —OC(═O)R^{A2}; —OC(═O)N(R^{A2})₂; —NR^{A}C(═O)OR^{A1}; or —C(R^{A2})₃; wherein each occurrence of R^{A1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{A1} groups are optionally joined to form a substituted or unsubstituted heterocyclyl or substituted or unsubstituted heteroaryl ring; and wherein each occurrence of R^{A2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{A1}; —SR^{A1}; or —N(R^{A1})₂; or two R^{A2} groups are optionally joined to form a substituted or unsubstituted heterocyclyl or substituted or unsubstituted heteroaryl ring; with the proviso that if R¹ is —OR^{A1}, R^{A1} is not 4-methoxybenzyl or carbohydrate.
R² is hydrogen; or R¹ and R² are joined to form-O; =N(R^{A2}); or ═S;
R³ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{C1}; —C(═O)R^{C2}; —CO₂R^{C1}; —CN; —SCN; —SR^{C1}; —SOR^{C1}; —SO₂R^{C2}; —NO₂; —N₃; —N(R^{C2})₂; —NHC(═O)R^{C2}; —NR^{C2}C(═O)N(R^{C2})₂; —OC(═O)OR^{C1}; —OC(═O)R^{C2}; —OC(═O)N(R^{C2})₂; —NR^{C2}C(═O)OR^{C1}; or —C(R^{C2})₃; wherein each occurrence of R^{C1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{C1} groups are optionally joined to form a substituted or unsubstituted heterocyclyl or substituted or unsubstituted heteroaryl ring; and wherein each occurrence of R^{C2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{C1}; —SR^{C1}; or —N(R^{C1})₂; or two R^{C2} groups are optionally joined to form a substituted or unsubstituted heterocyclyl or substituted or unsubstituted heteroaryl ring;
R⁴ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{D1}; —C(═O)R^{D2}; —CO₂R^{D2}; —CN; —SCN; —SR^{D1}; —SOR^{D1}; —SO₂R^{D2}; —NO₂; —N₃; —N(R^{D2})₂; —NR^{D2}C(═O)R^{D2}; —NR^{D2}C(═O)N(R^{D2})₂; —OC(═O)OR^{D1}; —OC(═O)R^{D2}; —OC(═O)N(R^{D2})₂; —NR^{D2}C(═O)OR^{D1}; or —C(R^{D2})₃; wherein each occurrence of R^{D1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{D1} groups are optionally joined to form a substituted or unsubstituted heterocyclyl or substituted or unsubstituted heteroaryl ring; and wherein each occurrence of R^{D2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{D1}; —SR^{D1}; or —N(R^{D1})₂; or two R^{D2} groups are optionally joined to form a substituted or unsubstituted heterocyclyl or substituted or unsubstituted heteroaryl ring;
R⁵ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{E1}; —C(═O)R^{E2}; —CO₂R^{E1}; —CN; —SCN; —SR^{E1}; —SOR^{E1}; —SO₂R^{E2}; —NO₂; —N₃; —N(R^{E2})₂; —NR^{E2}C(═O)R^{E2}; —NR^{E2}C(═O)N(R^{E2})₂; —OC(═O)OR^{E1}; —OC(═O)R^{E2}; —OC(═O)N(R^{E2})₂; —NR^{E2}C(═O)OR^{E1}; or —C(R^{E2})₃; wherein each occurrence of R^{E1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{E1} groups are optionally joined to form a substituted or unsubstituted heterocyclyl or substituted or unsubstituted heteroaryl ring; and wherein each occurrence of R^{E2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{E1}; —SR^{E1}; or —N(R^{E1})₂; or two R^{E2} groups are optionally joined to form a substituted or unsubstituted heterocyclyl or substituted or unsubstituted heteroaryl ring;
R⁶ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{F1}; —C(═O)R^{F2}; —CO₂R^{F1}; —CN; —SCN; —SR^{F1}; —SOR^{F1}; —SO₂R^{F2}; —NO₂; —N₃; —N(R^{F2})₂; —NR^{F2}C(═O)R^{F2}; —NR^{F2}C(═O)N(R^{F2})₂; —OC(═O)OR^{F1}; —OC(═O)R^{F2}; —OC(═O)N(R^{F2})₂; —NR^{F2}C(═O)OR^{F1}; or —C(R^{F2})₃; wherein each occurrence of R^{F1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{F1} groups are optionally joined to form a substituted or unsubstituted heterocyclyl or substituted or unsubstituted heteroaryl ring; and wherein each occurrence of R^{F2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{F1}; —SR^{F1}; or —N(R^{F1})₂; or two R^{F2} groups are optionally joined to form a substituted or unsubstituted heterocyclyl or substituted or unsubstituted heteroaryl ring;
R⁷ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{G1}; —C(═O)R^{G2}; —CO₂R^{G1}; —CN; —SCN; —SR^{G1}; —SOR^{G1}; —SO₂R^{G2}; —NO₂; —N₃; —N(R^{G})₂; —NR^{G2}C(═O)R^{G2}; —NR^{G2}C(═O)N(R^{G2})₂; —OC(═O)OR^{G1}; —OC(═O)R^{G2}; —OC(═O)N(R^{G2})₂; —NR^{G2}C(═O)OR^{G1}; or —C(R^{G2})₃; wherein each occurrence of R^{G1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{G1} groups are optionally joined to form a substituted or unsubstituted heterocyclyl or substituted or unsubstituted heteroaryl ring; and wherein each occurrence of R^{G2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{G1}; —SR^{G1}; or —N(R^{G1})₂; or two R^{G2} groups are optionally joined to form a substituted or unsubstituted heterocyclyl or substituted or unsubstituted heteroaryl ring;
R⁸ is hydrogen; an oxygen protecting group; a carbohydrate; substituted or unsubstituted alkyl; substituted or unsubstituted alkenyl; substituted or unsubstituted alkynyl; substituted or unsubstituted carbocyclyl; substituted or unsubstituted heterocyclyl; substituted or unsubstituted aryl; substituted or unsubstituted heteroaryl; substituted or unsubstituted heteroaliphatic; acyl; —OR^{H9}; —OC(═O)R^{H9}; —N(R^{H9})₂; or —NHC(═O)R^{H9}; wherein each occurrence of R^{H9} is independently hydrogen; substituted or unsubstituted alkyl; an oxygen protecting group when attached to an oxygen atom; or a nitrogen protecting group when attached to a nitrogen atom; or two R^{H9} groups are joined to form a substituted or unsubstituted heterocyclic or substituted or unsubstituted heteroaryl ring;
R⁹ is hydrogen or —C(R^{I1})₃; wherein each occurrence of R^{I1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{I2}; —SR^{I2}; —N(R^{I2})₂; azido; or halogen; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; substituted or unsubstituted phosphono; -L-A-B; or -L-T; or two R^{I2} groups are optionally joined to form a substituted or unsubstituted heterocyclyl or substituted or unsubstituted heteroaryl ring;
R¹⁰ is hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted aryl; substituted or unsubstituted heteroaryl; —OR^{B1}; —C(═O)R^{B2}; —CO₂R^{B2}; —CN; —SCN; —SR^{B1}; —SOR^{B1}; —SO₂R^{B2}; —NO₂; —N₃; —N(R^{B2})₂; —NR^{B2}C(═O)R^{B2}; —NR^{B2}C(═O)N(R^{B2})₂; —OC(═O)OR^{B1}; —OC(═O)R^{B2}; —OC(═O)N(R^{B2})₂; —NR^{B2}C(═O)OR^{B1}; or —C(R^{B2})₃; wherein each occurrence of R^{B1} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or two R^{B1} groups are optionally joined to form a substituted or unsubstituted heterocyclyl or substituted or unsubstituted heteroaryl ring; and wherein each occurrence of R^{B2} is independently hydrogen; carbohydrate; a protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; —OR^{B1}; —SR^{B1}; or —N(R^{B1})₂; or two R^{B2} groups are optionally joined to form a substituted or unsubstituted heterocyclyl or substituted or unsubstituted heteroaryl ring;
R^{I1} is hydrogen; substituted or unsubstituted aliphatic; acyl; -L-A-B; or -L-T;
X is a halogen;
or R¹ and R¹⁰ are optionally taken together with the intervening carbon atoms to form an optionally substituted cyclic moiety;
or R¹⁰ and R³ are optionally taken together with the intervening carbon atoms to form an optionally substituted cyclic moiety;
or R¹ and R⁴ are optionally taken together with the intervening carbon atoms to form an optionally substituted cyclic moiety;
or R⁴ and R⁷ are optionally taken together with the intervening carbon atoms to form an optionally substituted cyclic moiety;
or R⁶ and R⁷ are optionally taken together with the intervening carbon atoms to form an optionally substituted cyclic moiety;
L is a linker;
A is a bond or a group of formula: 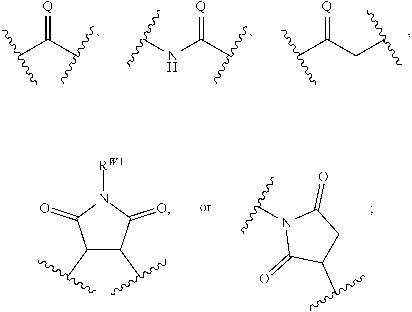
Q is —S— or —O—;
R^{W1} is independently hydrogen, substituted or unsubstituted alkyl; or a nitrogen protecting group;
B is a targeting moiety; and
T is hydrogen, —N═C═S, 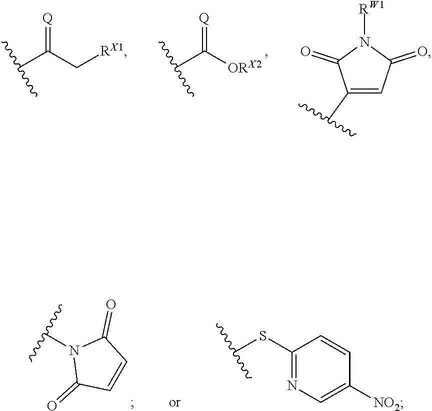
R^{X1} is a leaving group; and
R^{X2} is hydrogen; substituted or unsubstituted alkyl; substituted or unsubstituted heterocyclyl; substituted or unsubstituted aryl; substituted or unsubstituted heteroaryl; or an oxygen protecting group.

53. The compound of claim 52, wherein the compound is of Formula (II-a): 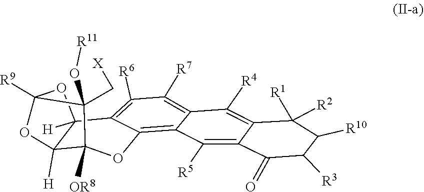
or a pharmaceutically acceptable salt thereof.

54. The compound of any of claims 52-53, wherein the compound is of Formula (II-b): 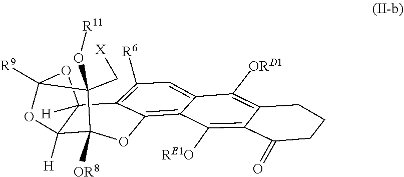
or a pharmaceutically acceptable salt thereof.

55. The compound of any of claims 52-54, wherein the compound is of Formula (II-c): 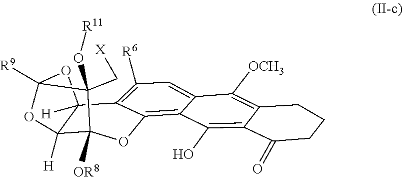
or a pharmaceutically acceptable salt thereof.

56. The compound of any of claims 52-55, wherein R¹ is hydrogen.

57. The compound of any of claims 52-55, wherein R¹ and R² together form ═O.

58. The compound of any of claims 52-57, wherein R⁴ is —OR^{D1}.

59. The compound of any of claims 52-58, wherein R^{D1} is C₁₋₆alkyl.

60. The compound of any of claims 52-59, wherein R⁵ is —OR^{E1}.

61. The compound of claim 60, wherein R^{E1} is hydrogen.

62. The compound of any of claims 52-61, wherein R⁶ is C₁₋₆ alkyl.

63. The compound of any of claims 52-62, wherein R⁶ is —CH₃.

64. The compound of any of claims 52-63, wherein R⁷ is hydrogen.

65. The compound of any of claims 52-64, wherein R⁸ is hydrogen, a carbohydrate, or C₁₋₆ alkyl.

66. The compound of claim 65, wherein R⁸ is a carbohydrate of formula: 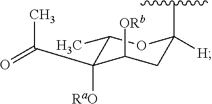
wherein R^{a} and R^{b} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl.

67. The compound of any of claims 52-65, wherein R⁸ is C₁₋₆ alkyl.

68. The compound of any of claim 52-65 or 67, wherein R⁸ is —CH₃.

69. The compound of any of claims 52-65, wherein R⁸ is hydrogen.

70. The compound of any of claims 52-69, wherein X is —C₁, —Br, or —I.

71. The compound of any of claims 52-70, wherein X is —Br.

72. The compound of any of claims 52-71, wherein the compound is of Formula (II-d): 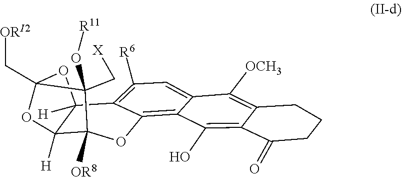
or a pharmaceutically acceptable form thereof, wherein R^{I2} is hydrogen, carbohydrate, protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted phosphono; or substituted or unsubstituted heteroarylalkyl.

73. The compound of claim 72, wherein R^{I2} is hydrogen; oxygen protecting group; substituted or unsubstituted phosphono; carbohydrate; substituted or unsubstituted alkyl; substituted or unsubstituted alkenyl; substituted or unsubstituted alkynyl; or substituted or unsubstituted heteroarylalkyl.

74. The compound of claim 72 or 73, wherein R^{I2} is hydrogen, oxygen protecting group, substituted or unsubstituted phosphono, substituted or unsubstituted alkyl, substituted or unsubstituted alkenyl, or substituted or unsubstituted alkynyl.

75. The compound of any of claims 72-74, wherein R^{I2} is hydrogen, protecting group, substituted or unsubstituted phosphono, substituted or unsubstituted C₁₋₆ alkyl, substituted or unsubstituted C₁₋₆ alkenyl, or substituted or unsubstituted C₁₋₆ alkynyl.

76. The compound of any of claims 72-75, wherein R^{I2} is hydrogen; benzyl; n-propyl; 2-propenyl; 2-propynyl; or —P(═O)(OH)₂.

77. The compound of any of claim 72 or 73, wherein R^{I2} is substituted or unsubstituted heteroarylalkyl.

78. The compound of any of claim 72 or 73, wherein R^{I2} is a carbohydrate.

79. The compound of any of claim 72, 73, or 78 wherein R^{I2} is: 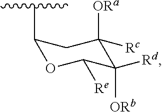
wherein R^{a} and R^{b} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl; or acyl; and
R^{c}, R^{d}, and R^{e} are independently hydrogen; cyclic or acyclic, substituted or unsubstituted, branched or unbranched aliphatic; cyclic or acyclic, substituted or unsubstituted, branched or unbranched heteroaliphatic; substituted or unsubstituted alkenyl; substituted or unsubstituted alkynyl; or acyl.

80. The compound of any of claim 72, 73, 78, or 79, wherein R^{a} and R^{b} are independently hydrogen; carbohydrate; oxygen protecting group; or acyl.

81. The compound of any of claim 72, 73, or 78-80, wherein R^{c}, R^{d}, and R^{e} are independently hydrogen; substituted or unsubstituted alkyl; or acyl.

82. The compound of any of claim 72, 73, or 78-81, wherein R^{c}, R^{d}, and R^{e} are independently hydrogen; substituted or unsubstituted C₁₋₆ alkyl; or acyl.

83. The compound of any of claim 72, 73, or 78-82, wherein R^{I2} is: 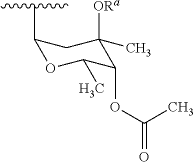
wherein R^{a} is hydrogen; carbohydrate; or oxygen protecting group.

84. The compound of any of claim 72, 73, or 78-83, wherein R^{I2} is:

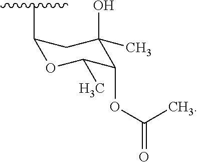 [SMILES: *C1CC(C)(O)C(OC(C)=O)C(C)O1]

85. The compound of any of claim 72, 73, or 78-82, wherein R^{I2} is: 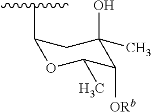
wherein R^{b} is hydrogen; carbohydrate; or oxygen protecting group.

86. The compound of any of claim 72, 73, 78-82, or 85, wherein R^{I2} is:

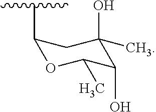 [SMILES: *C1CC(C)(O)C(O)C(C)O1]

87. The compound of any of claim 72, 73, 78-82, or 85, wherein R^{I2} is:

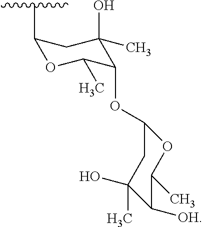 [SMILES: *C1CC(C)(O)C(OC2CC(C)(O)C(O)C(C)O2)C(C)O1]

88. The compound of any of claim 72, 73, 78-82, or 85, wherein R^{I2} is:

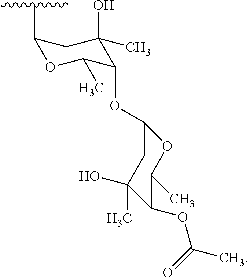 [SMILES: *C1CC(C)(O)C(OC2CC(C)(O)C(OC(C)=O)C(C)O2)C(C)O1]

89. The compound of any of claim 72, 73, or 78-82, wherein R^{I2} is: 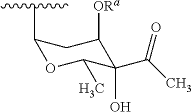
wherein R^{a} is hydrogen; carbohydrate; or oxygen protecting group.

90. The compound of any of claim 72, 73, 78-82, or 89, wherein R^{I2} is:

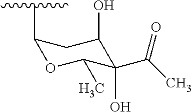 [SMILES: *C1CC(O)C(O)(C(C)=O)C(C)O1]

91. The compound of any of claim 72, 73, or 78, wherein R^{I2} is: 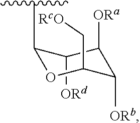
wherein R^{a}, R^{b}, R^{c}, and R^{d} are independently hydrogen; carbohydrate; an oxygen protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; or substituted or unsubstituted heteroaryl.

92. The compound of any of claim 72, 73, 78, or 91, wherein R^{a}, R^{b}, R^{c}, and R^{d} are independently hydrogen; carbohydrate; or oxygen protecting group.

93. The compound of any of claim 72, 73, 78, 91, or 92, wherein R^{I2} is:

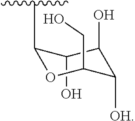 [SMILES: *C1OC(CO)C(O)C(O)C1O]

94. The compound of any of claims 52-71, wherein the compound is of Formula (II-e): 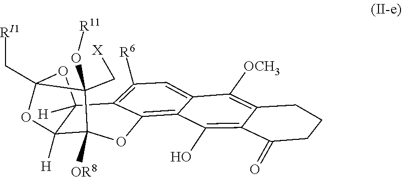
or a pharmaceutically acceptable salt thereof.

95. The compound of claim 94, wherein R^{I1} is hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂.

96. The compound of any of claim 94 or 95, wherein R^{I1} is azido; —N(R^{I2})₂; —SR^{I2}; or halogen.

97. The compound of claim 96, wherein each occurrence of R^{I2} is independently hydrogen; protecting group; cyclic or acyclic, substituted or unsubstituted, branched or unbranched aliphatic; or acyl; or two R^{I2} groups are optionally joined to form a substituted or unsubstituted heterocyclyl or substituted or unsubstituted heteroaryl ring.

98. The compound of any of claims 94-97, wherein R^{I1} is azido; —NH₂; —SH; —SAc; chloro; bromo; or iodo.

99. The compound of any of claims 52-98, wherein R^{I1} is of formula: 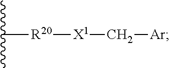
R²⁰ is substituted or unsubstituted alkylene; or substituted or unsubstituted heteroalkylene;
X¹ is 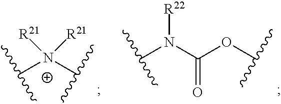
heterocyclylene; or heteroarylene;
R²¹ is independently substituted or unsubstituted alkyl; or substituted or unsubstituted carbocyclyl; or two R²¹ groups are joined to form an optionally substituted heterocyclyl ring;
R²² is hydrogen; substituted or unsubstituted alkyl; substituted or unsubstituted carbocyclyl; or 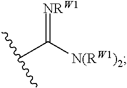
Ar is substituted or unsubstituted aryl.

100. The compound of claim 52, wherein the compound is: 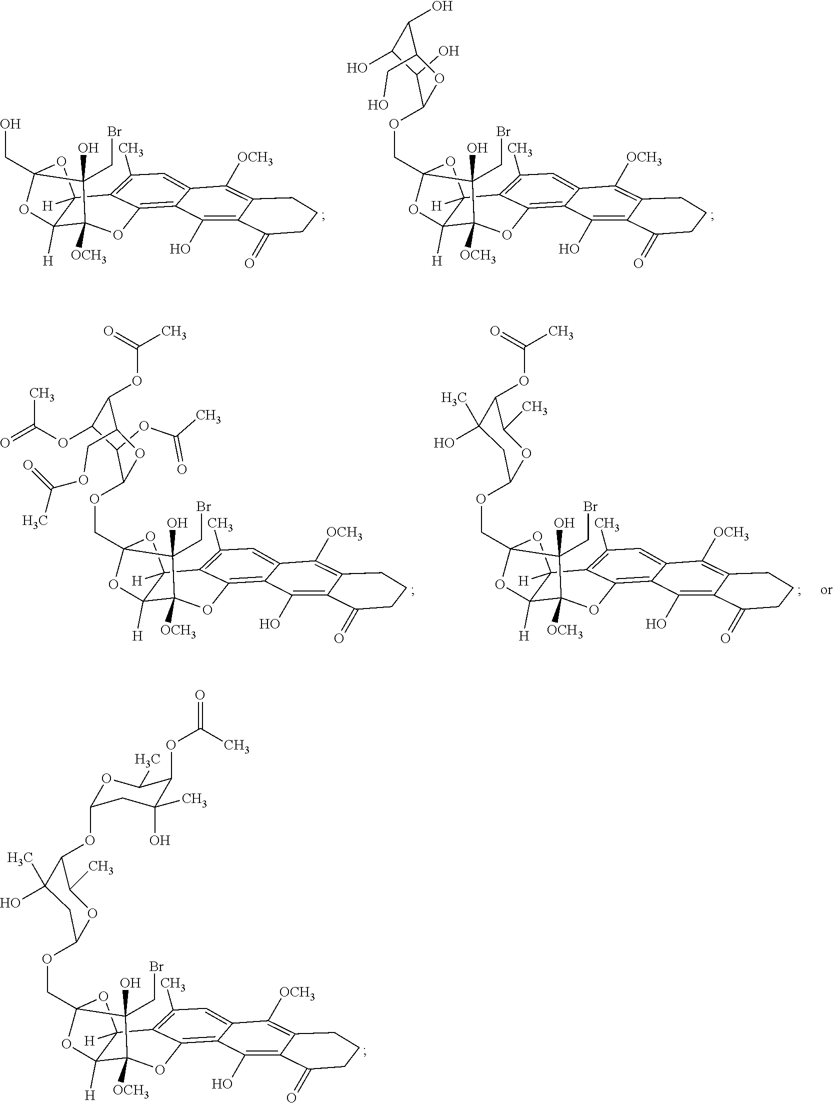 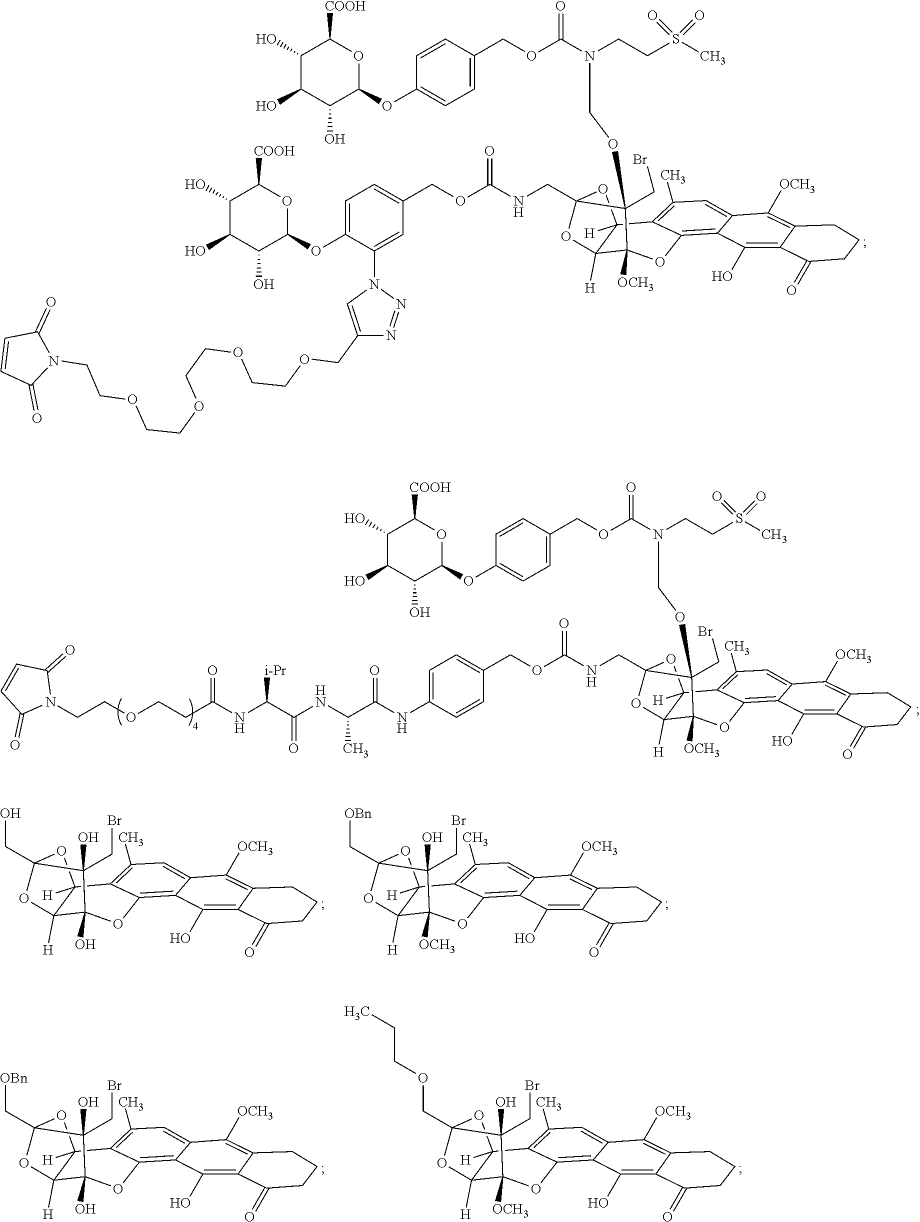 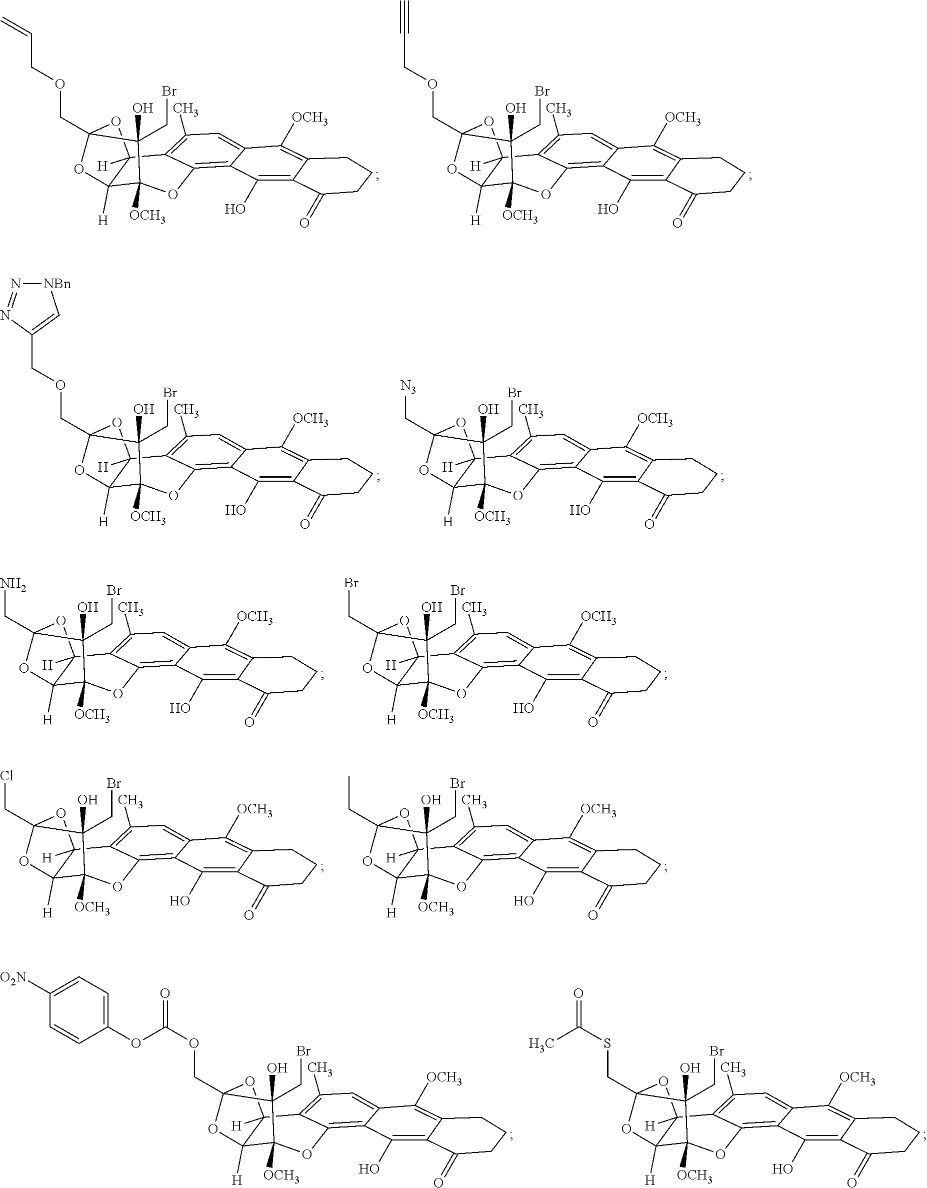 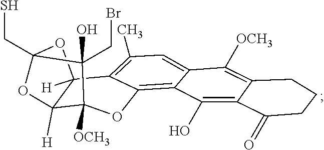 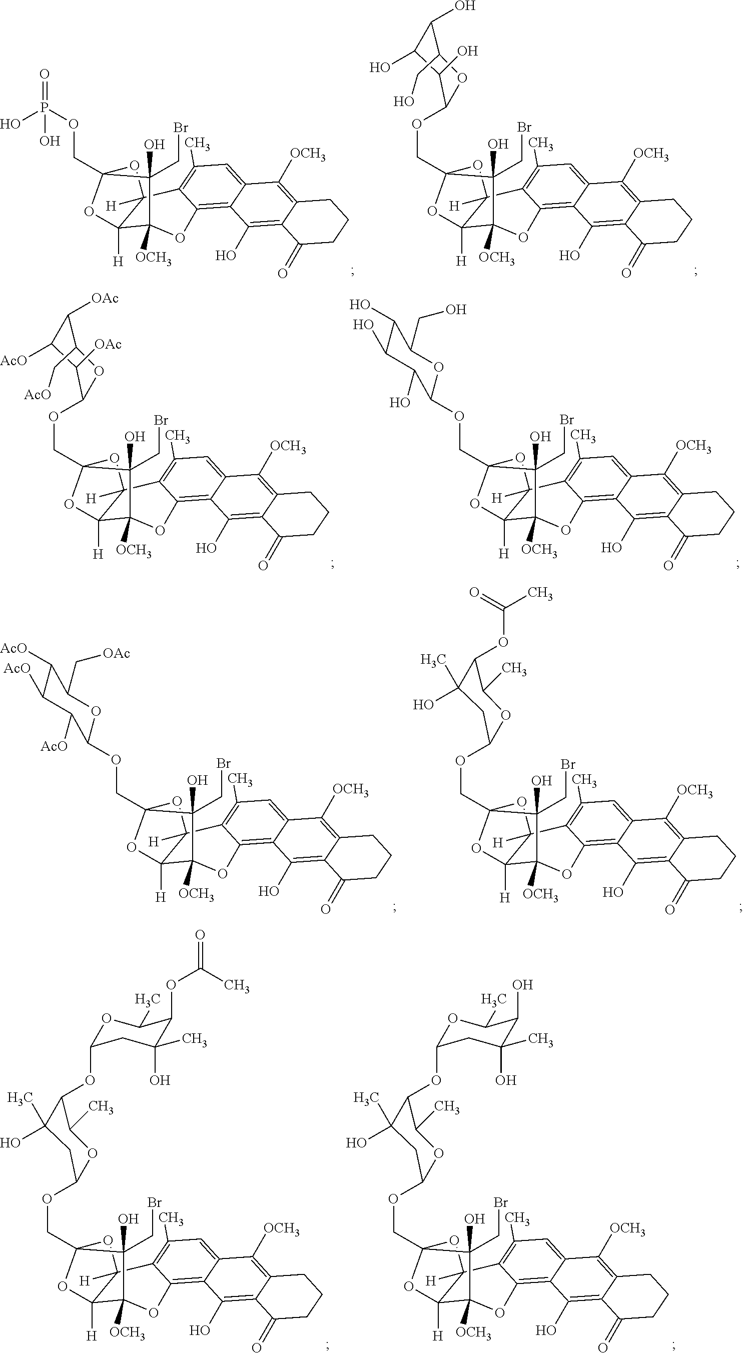 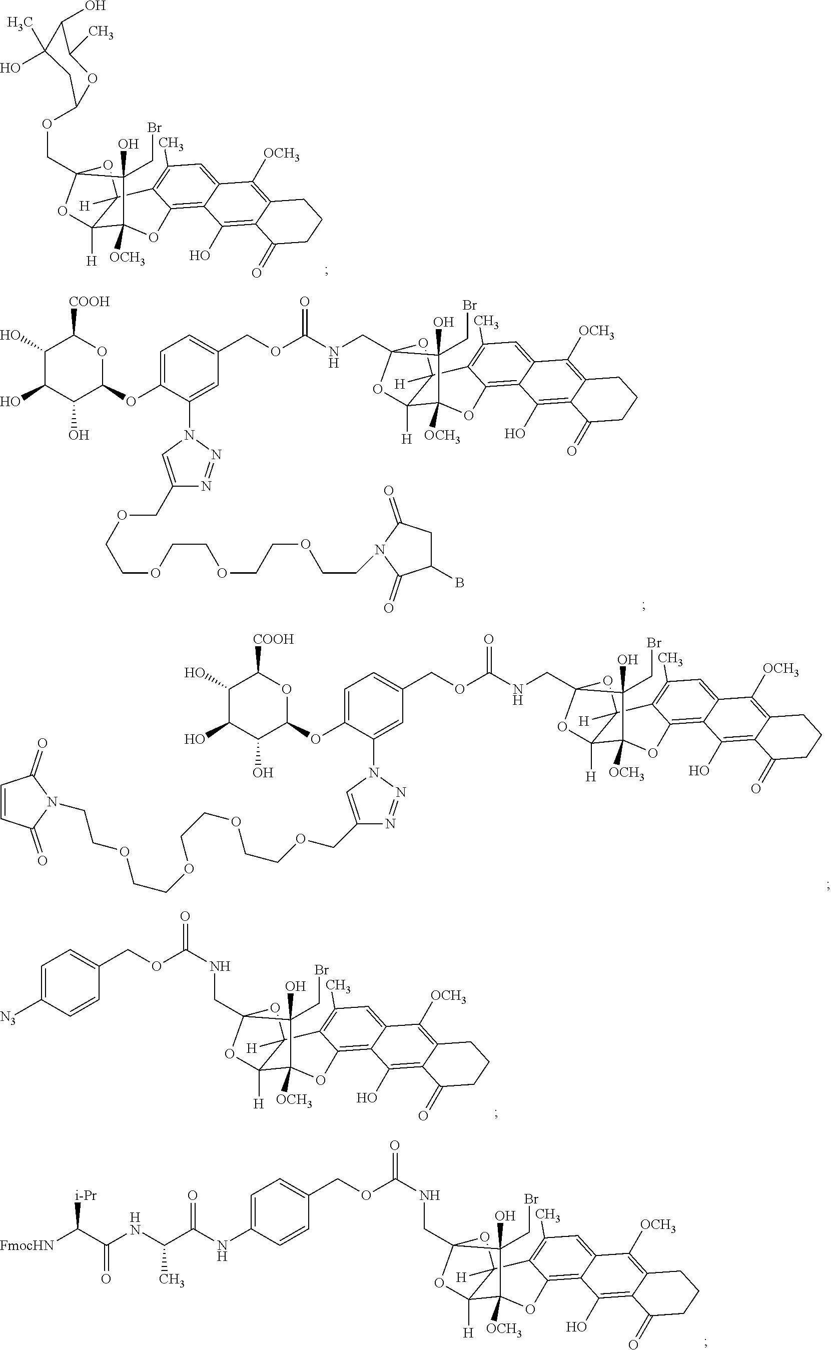 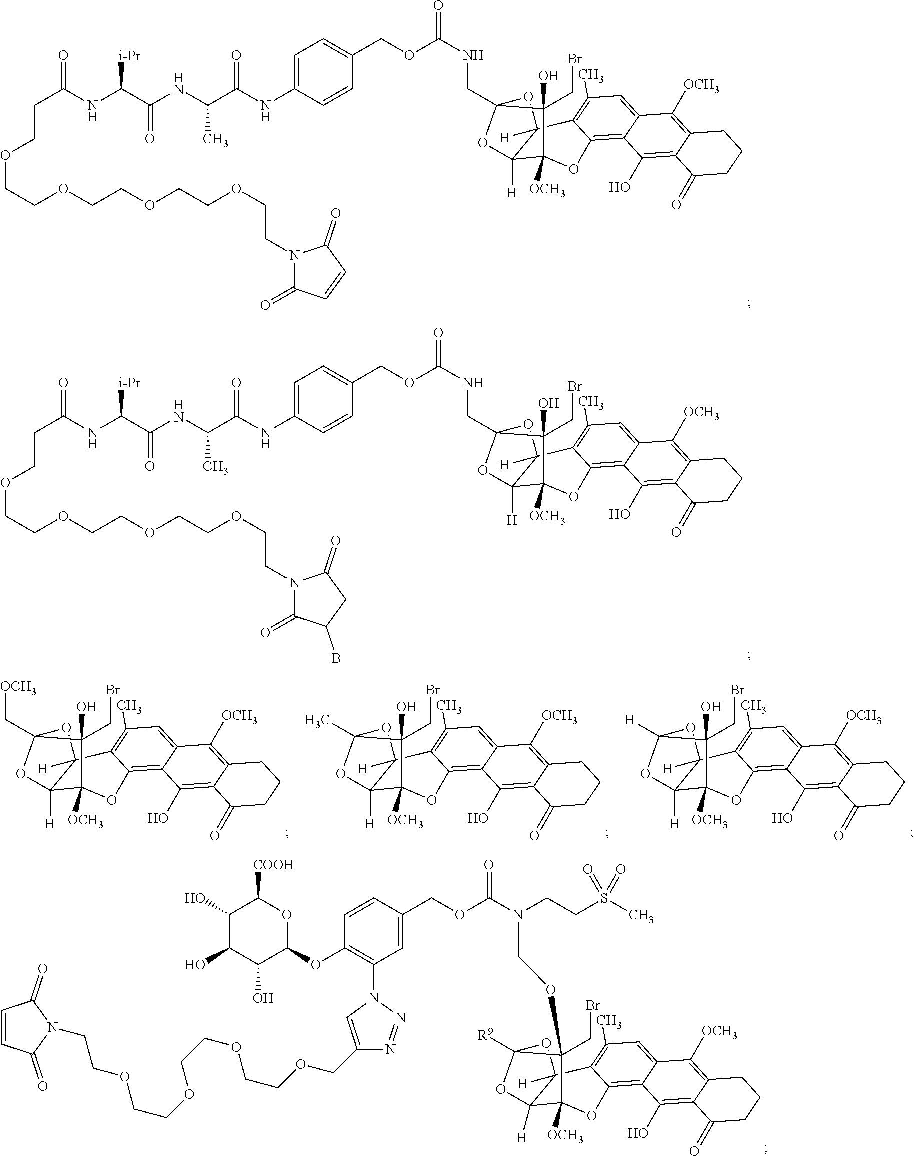 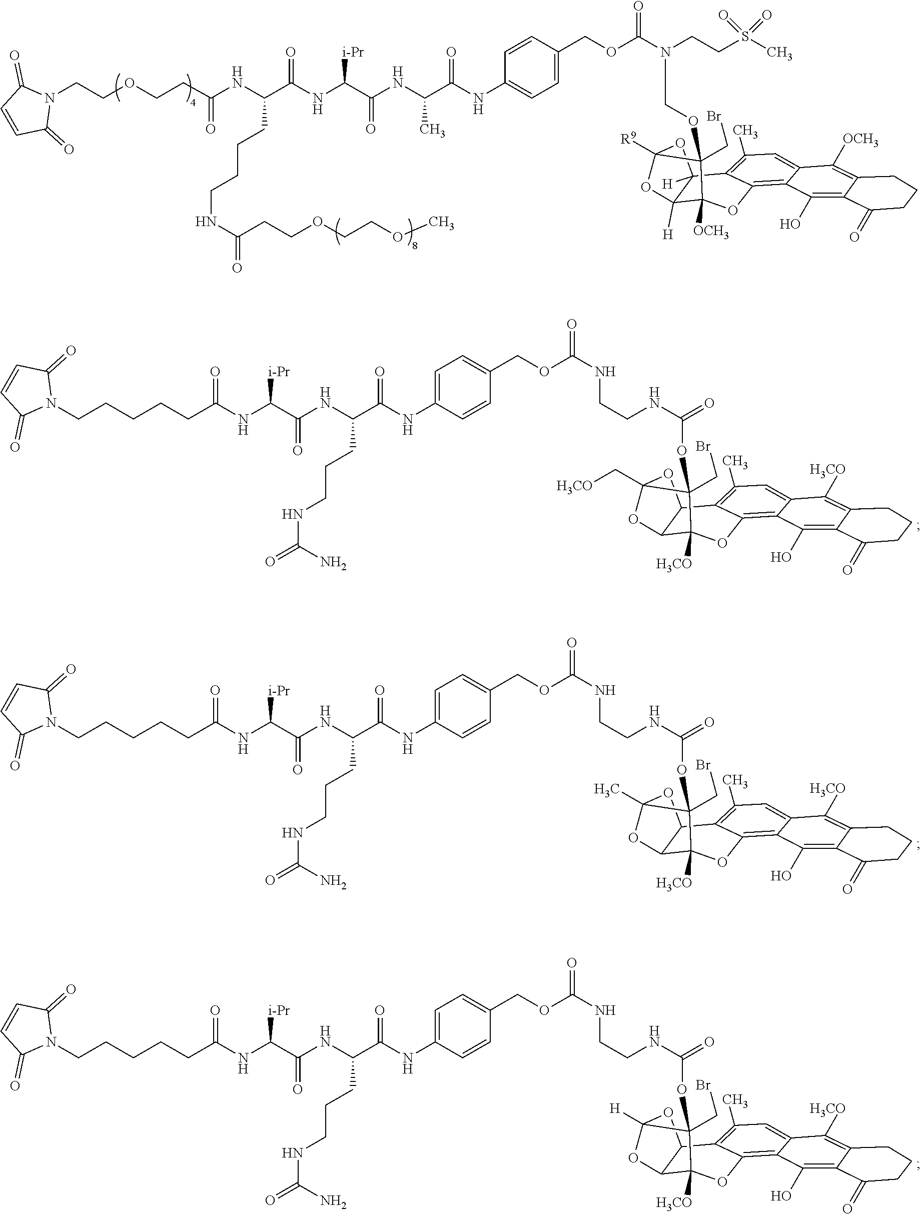 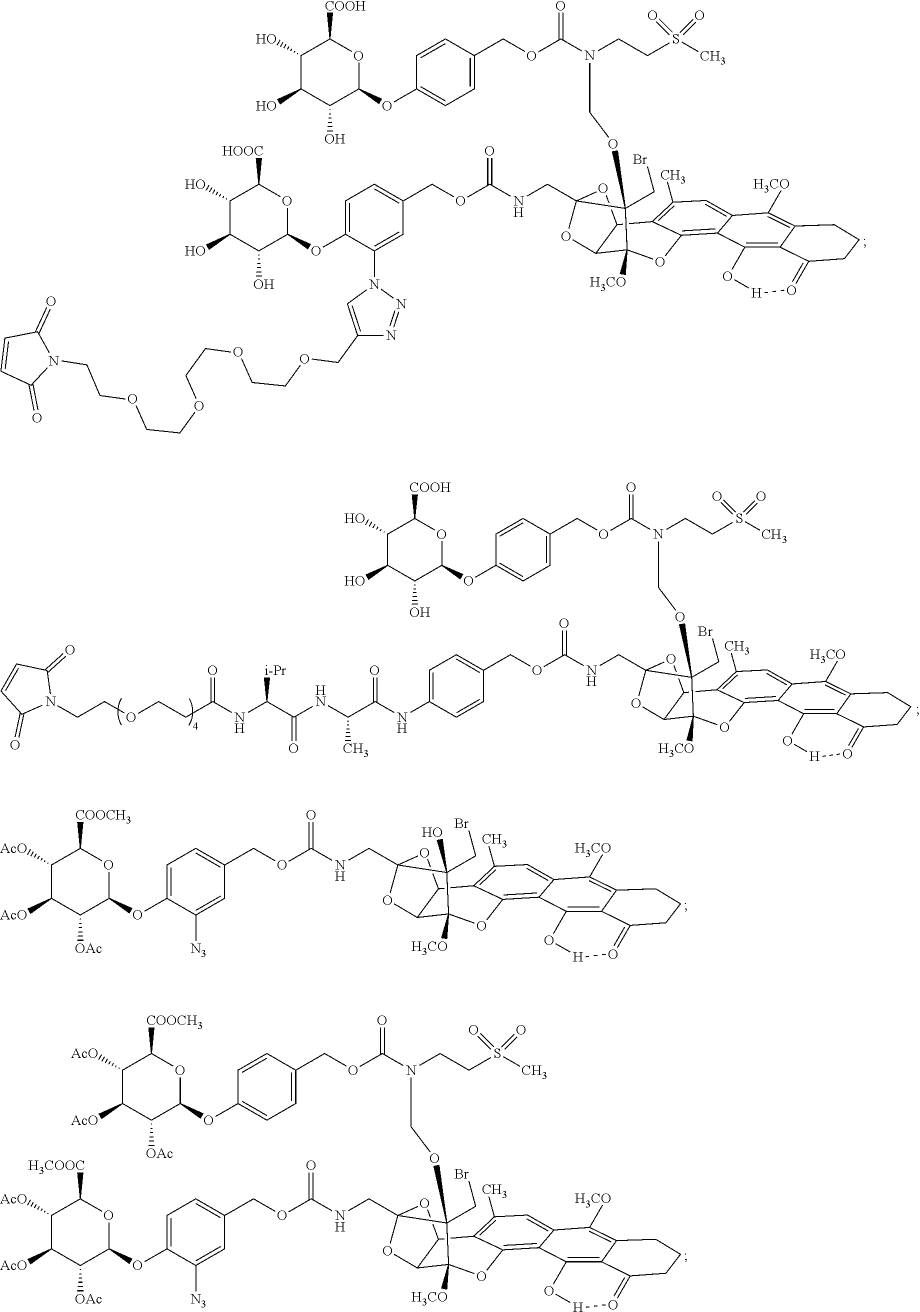 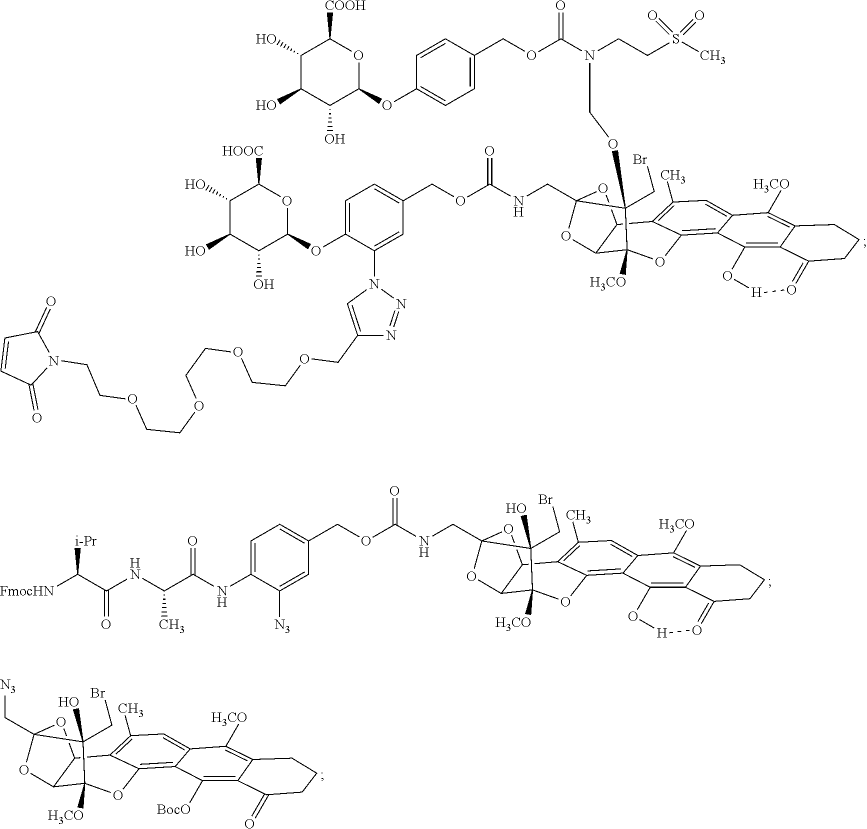
or a pharmaceutically acceptable salt thereof.

101. The compound of any of claims 52-71, wherein at least one occurrence of R^{I1} is —OR^{I2}, —N(R^{I2})₂, or-SR^{I2}.

102. The compound of claim 101, wherein R^{I2} is -L-A-B, provided that R^{I1} is not -L-A-B or -L-T.

103. The compound of claim 101, wherein R^{I2} is -L-T, provided that R^{I1} is not -L-A-B or -L-T.

104. The compound of any of claim 1-18, 51-71, 102, or 103, wherein L is of formula: 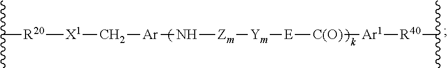
R²⁰ is substituted or unsubstituted alkylene; or substituted or unsubstituted heteroalkylene;
X¹ is 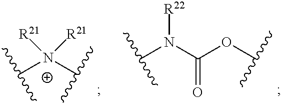
heterocyclylene; or heteroarylene;
R²¹ is independently substituted or unsubstituted alkyl; or substituted or unsubstituted carbocyclyl; or two R²¹ groups are joined to form an optionally substituted heterocyclyl ring;
R²² is hydrogen; substituted or unsubstituted alkyl; substituted or unsubstituted carbocyclyl; or 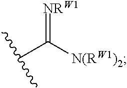
Ar is substituted or unsubstituted arylene;
each occurrence of Z is independently an amino acid;
each occurrence of Y is independently an amino acid;
E is a bond or an amino acid;
m is independently 1, 2, or 3;
k is 0 or 1;
Ar¹ is a bond or substituted or unsubstituted heteroarylene;
R⁴⁰ is substituted or unsubstituted alkylene; or substituted or unsubstituted heteroalkylene.

105. The compound of claim 104, or a pharmaceutically acceptable salt thereof, wherein R²⁰ is substituted or unsubstituted alkylene.

106. The compound of any of claim 104 or 105, or a pharmaceutically acceptable salt thereof, wherein R²⁰ is unsubstituted alkylene.

107. The compound of any of claims 104-106, or a pharmaceutically acceptable salt thereof, wherein:
X¹ is 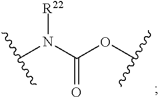
and
R²² is hydrogen; substituted or unsubstituted alkyl; or substituted or unsubstituted carbocyclyl.

108. The compound of any of claims 104-107, or a pharmaceutically acceptable salt thereof, wherein:
X¹ is 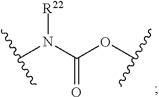
and R²² is substituted alkyl.

109. The compound of any of claims 104-108, or a pharmaceutically acceptable salt thereof, wherein Ar is substituted or unsubstituted phenylene.

110. The compound of any of claims 104-109, or a pharmaceutically acceptable salt thereof, wherein Z is alanine, lysine, arginine, histidine, omnithine, or citrulline.

111. The compound of any of claims 104-110, or a pharmaceutically acceptable salt thereof, wherein Z is alanine or citrulline.

112. The compound of any of claims 104-111, or a pharmaceutically acceptable salt thereof, wherein Z is alanine.

113. The compound of any of claims 104-112, or a pharmaceutically acceptable salt thereof, wherein Y is alanine, valine, leucine, isoleucine, methionine, phenylalanine, or tryptophan.

114. The compound of any of claims 104-113, or a pharmaceutically acceptable salt thereof, wherein Y is valine.

115. The compound of any of claims 104-114, or a pharmaceutically acceptable salt thereof, wherein each occurrence of m is 1.

116. The compound of any of claims 104-115, or a pharmaceutically acceptable salt thereof, wherein —Zₘ—Yₘ— is -alanine-valine-.

117. The compound of any of claims 104-116, or a pharmaceutically acceptable salt thereof, wherein Ar¹ is a bond.

118. The compound of any of claims 104-116, or a pharmaceutically acceptable salt thereof, wherein Ar¹ is substituted or unsubstituted heteroarylene.

119. The compound of any of claims 104-118, or a pharmaceutically acceptable salt thereof, wherein k is 0.

120. The compound of any of claims 104-118, or a pharmaceutically acceptable salt thereof, wherein k is 1.

121. The compound of any of claims 104-120, or a pharmaceutically acceptable salt thereof, wherein E is a bond.

122. The compound of any of claims 104-120, or a pharmaceutically acceptable salt thereof, wherein E is of formula: 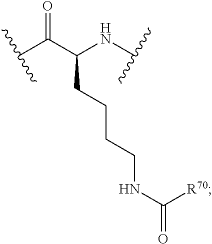
wherein R⁷⁰ is substituted or unsubstituted alkyl, or substituted or unsubstituted heteroalkyl.

123. The compound of any of claims 104-122, or a pharmaceutically acceptable salt thereof, wherein R⁴⁰ is substituted or unsubstituted alkylene.

124. The compound of any of claims 104-122, or a pharmaceutically acceptable salt thereof, wherein R⁴⁰ is substituted or unsubstituted heteroalkylene.

125. The compound of any of claims 104-124, or a pharmaceutically acceptable salt thereof, wherein A is a group of formula:

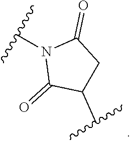 [SMILES: *C1CC(=O)N(*)C1=O]

126. The compound of any of claims 104-125, or a pharmaceutically acceptable salt thereof, wherein L is of formula: 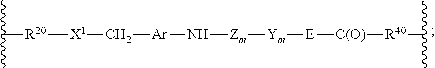
R²⁰ is substituted or unsubstituted alkylene; or substituted or unsubstituted heteroalkylene;
X¹ is 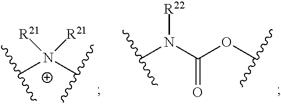
heterocyclylene; or heteroarylene;
R²¹ is independently substituted or unsubstituted alkyl; or substituted or unsubstituted carbocyclyl; or two R²¹ groups are joined to form an optionally substituted heterocyclyl ring;
R²² is hydrogen; substituted or unsubstituted alkyl; substituted or unsubstituted carbocyclyl; or 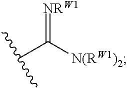
Ar is substituted or unsubstituted arylene;
each occurrence of Z is independently an amino acid;
each occurrence of Y is independently an amino acid;
E is a bond or an amino acid;
m is independently 1, 2, or 3;
R⁴⁰ is substituted or unsubstituted alkylene; or substituted or unsubstituted heteroalkylene;
A is a group of formula: 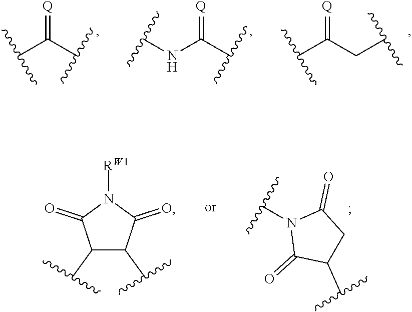
Q is —S— or —O—; and
R^{W1} is independently hydrogen, substituted or unsubstituted alkyl; or a nitrogen protecting group.

127. The compound of any of claims 104-126, or a pharmaceutically acceptable salt thereof, wherein -L- is a group of formula: 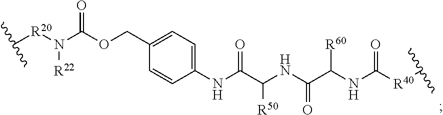
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene;
R⁵⁰ is the sidechain of alanine, lysine, arginine, histidine, ornithine, or citrulline; and
R⁶⁰ is the sidechain of alanine, valine, leucine, isoleucine, methionine, phenylalanine, or tryptophan.

128. The compound of any of claims 104-126, or a pharmaceutically acceptable salt thereof, wherein -L- is a group of formula: 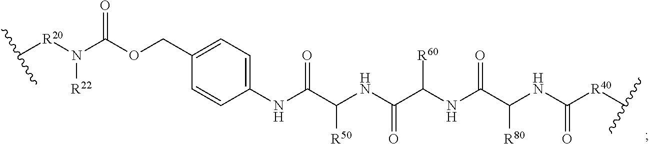
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene;
R⁵⁰ is the sidechain of alanine, lysine, arginine, histidine, ornithine, or citrulline; and
R⁶⁰ is the sidechain of alanine, valine, leucine, isoleucine, methionine, phenylalanine, or tryptophan; and
R⁸⁰ is a substituted sidechain of a lysine, arginine, histidine, ornithine, or citrulline.

129. The compound of any of claims 104-125, or a pharmaceutically acceptable salt thereof, wherein -L- is a group of formula: 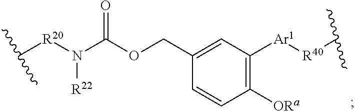
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene;
Ar¹ is substituted or unsubstituted heteroarylene; and
R^{a} is a substituted heterocycle.

130. The compound of any of claims 104-124, or a pharmaceutically acceptable salt thereof, wherein -L-A- is a group of formula: 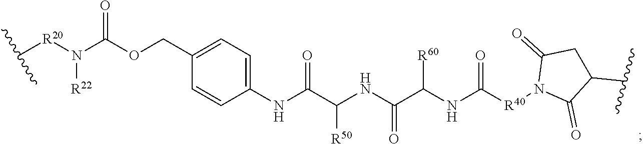
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene;
R⁵⁰ is the sidechain of alanine, lysine, arginine, histidine, ornithine, or citrulline; and
R⁶⁰ is the sidechain of alanine, valine, leucine, isoleucine, methionine, phenylalanine, or tryptophan.

131. The compound of any of claims 104-126, or a pharmaceutically acceptable salt thereof, wherein -L-A- is a group of formula: 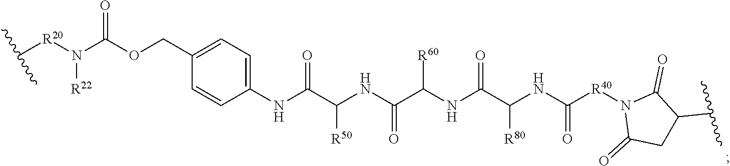
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene;
R⁵⁰ is the sidechain of alanine, lysine, arginine, histidine, ornithine, or citrulline; and
R⁶⁰ is the sidechain of alanine, valine, leucine, isoleucine, methionine, phenylalanine, or tryptophan; and
R⁸⁰ is an optionally substituted sidechain of a lysine, arginine, histidine, ornithine, or citrulline.

132. The compound of any of claims 104-124, or a pharmaceutically acceptable salt thereof, wherein -L-A- is a group of formula: 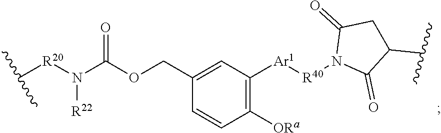
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene;
Ar¹ is substituted or unsubstituted heteroarylene; and
R^{a} is a substituted heterocycle.

133. The compound of any of claims 104-126, or a pharmaceutically acceptable salt thereof, wherein -L-A- is a group of formula: 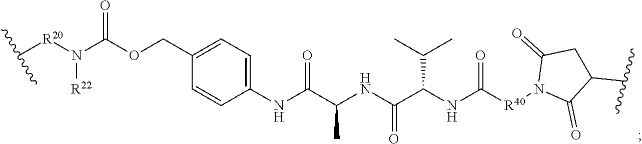
wherein:
R²⁰ is substituted or unsubstituted C₁₋₆ alkylene;
R²² is hydrogen, or substituted or unsubstituted C₁₋₆ alkyl; and
R⁴⁰ is substituted or unsubstituted C₁₋₆ alkylene, or substituted or unsubstituted C₁₋₄₀ heteroalkylene.

134. The compound of any of claims 104-126, or a pharmaceutically acceptable salt thereof, wherein -L-A- is a group of formula: 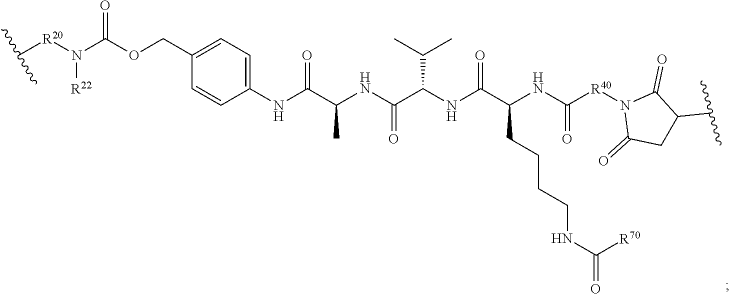
wherein:
R²⁰ is substituted or unsubstituted C₁₄ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene; and
R⁷⁰ is substituted or unsubstituted heteroalkyl.

135. The compound of any of claims 104-124, or a pharmaceutically acceptable salt thereof, wherein -L-A- is a group of formula: 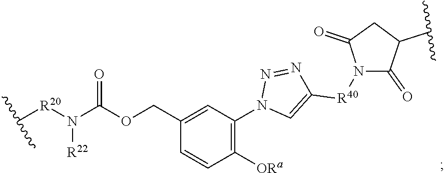
wherein:
R²⁰ is substituted or unsubstituted C₁₋₆ alkylene;
R²² is hydrogen, or substituted or unsubstituted C₁₋₆ alkyl; and
R⁴⁰ is substituted or unsubstituted C₁₋₆ alkylene, or substituted or unsubstituted C₁₋₄₀ heteroalkylene; and
R^{a} is a substituted heterocycle.

136. The compound of any of claims 104-126, or a pharmaceutically acceptable salt thereof, wherein -L-A- is a group of formula: 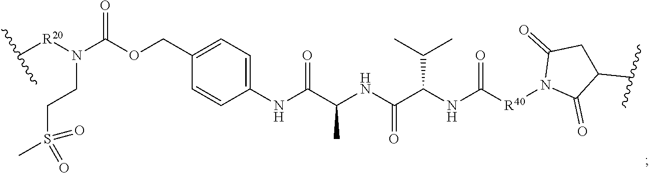
wherein:
R²⁰ is substituted or unsubstituted C₁₋₆ alkylene; and
R⁴⁰ is substituted or unsubstituted C₁₋₆ alkylene, or substituted or unsubstituted C₁₋₄₀ heteroalkylene.

137. The compound of any of claims 104-126, or a pharmaceutically acceptable salt thereof, wherein -L-A- is a group of formula: 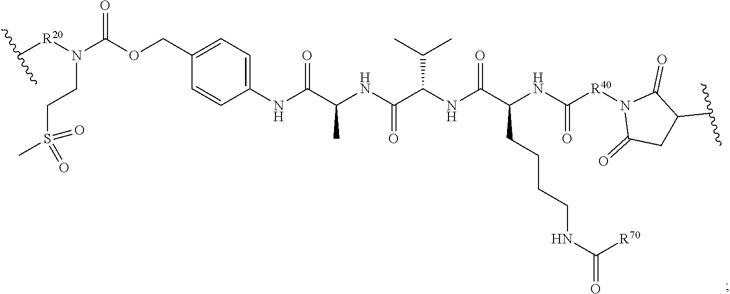
wherein:
R²⁰ is substituted or unsubstituted C₁₋₆ alkylene;
R⁴⁰ is substituted or unsubstituted C₁₋₆ alkylene, or substituted or unsubstituted C₁₋₄₀ heteroalkylene; and
R⁷⁰ is substituted or unsubstituted heteroalkyl.

138. The compound of any of claims 104-124, or a pharmaceutically acceptable salt thereof, wherein -L-A- is a group of formula: 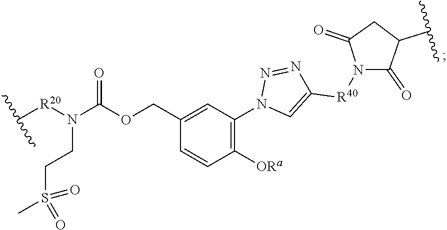
wherein:
R²⁰ is substituted or unsubstituted C₁₋₆ alkylene;
R⁴⁰ is substituted or unsubstituted C₁₋₆ alkylene, or substituted or unsubstituted C₁₋₄₀ heteroalkylene; and
R^{a} is substituted or unsubstituted heterocycle.

139. The compound of any of claims 104-126, or a pharmaceutically acceptable salt thereof, wherein -L-A- is a group of formula: 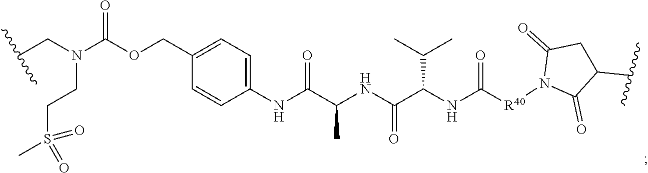
wherein:
R⁴⁰ is substituted or unsubstituted C₁₋₆ alkylene, or substituted or unsubstituted C₁₋₄₀ heteroalkylene.

140. The compound of any of claims 104-126, or a pharmaceutically acceptable salt thereof, wherein -L-A- is a group of formula: 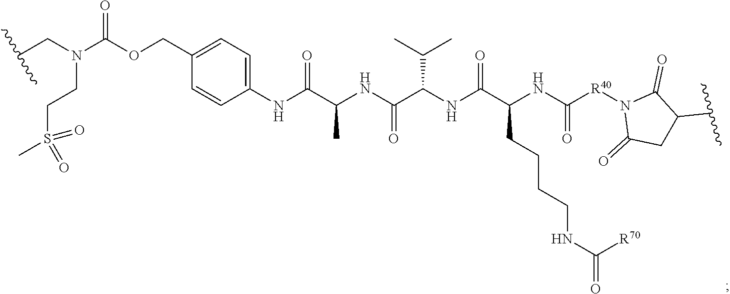
wherein:
R⁴⁰ is substituted or unsubstituted C₁₋₆ alkylene, or substituted or unsubstituted C₁₋₄₀ heteroalkylene; and
R⁷⁰ is substituted or unsubstituted heteroalkyl.

141. The compound of any of claims 104-124, or a pharmaceutically acceptable salt thereof, wherein -L-A- is a group of formula: 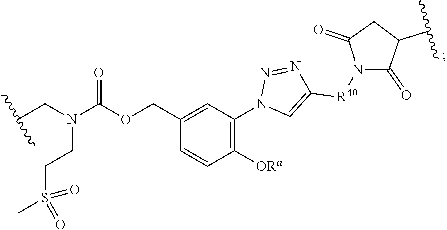
wherein:
R⁴⁰ is substituted or unsubstituted C₁₋₆ alkylene, or substituted or unsubstituted C₁₋₄₀ heteroalkylene; and
R^{a} is substituted or unsubstituted heterocycle.

142. The compound of any of claims 104-124, or a pharmaceutically acceptable salt thereof, wherein -L-A- is a group of formula:

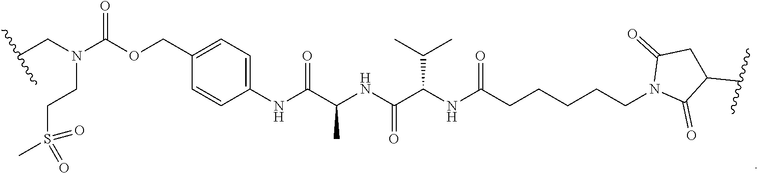 [SMILES: *CN(CCS(C)(=O)=O)C(=O)OCc1ccc(NC(=O)[C@H](C)NC(=O)[C@@H](NC(=O)CCCCCN2C(=O)CC(*)C2=O)C(C)C)cc1]

143. The compound of any of claims 104-124, or a pharmaceutically acceptable salt thereof, wherein -L-A- is a group of formula:

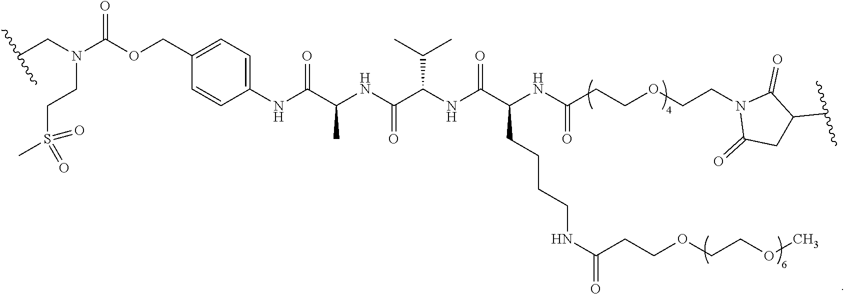

144. The compound of any of claims 104-124, or a pharmaceutically acceptable salt thereof, wherein -L-A- is a group of formula:

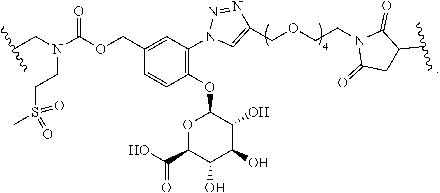

145. The compound of any of claims 104-124, or a pharmaceutically acceptable salt thereof, wherein -L-A- is a group of formula:

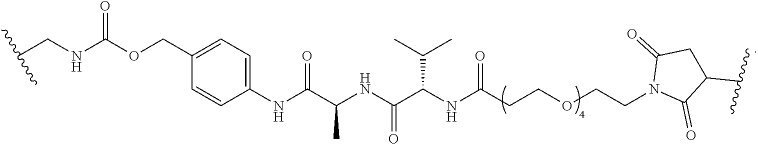

146. The compound of any of claims 104-124, or a pharmaceutically acceptable salt thereof, wherein T is a group of formula:

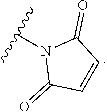 [SMILES: *N1C(=O)C=CC1=O]

147. The compound of any of claims 104-124, or a pharmaceutically acceptable salt thereof, wherein -L-T is a group of formula: 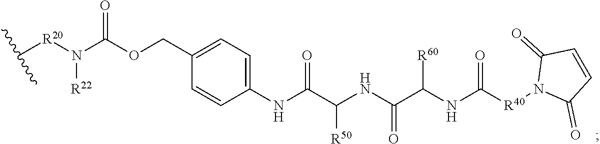
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene;
R⁵⁰ is the sidechain of alanine, lysine, arginine, histidine, ornithine, or citrulline; and
R⁶⁰ is the sidechain of alanine, valine, leucine, isoleucine, methionine, phenylalanine, or tryptophan.

148. The compound of any of claims 104-124, or a pharmaceutically acceptable salt thereof, wherein -L-T is a group of formula: 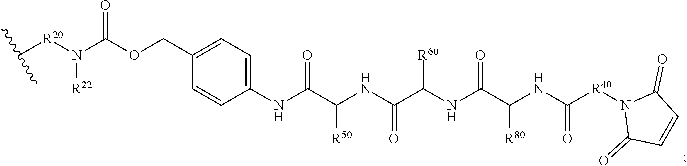
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene;
R⁵⁰ is the sidechain of alanine, lysine, arginine, histidine, ornithine, or citrulline;
R⁶⁰ is the sidechain of alanine, valine, leucine, isoleucine, methionine, phenylalanine, or tryptophan; and
and R⁸⁰ is a substituted sidechain of a lysine, arginine, histidine, ornithine, or citrulline.

149. The compound of any of claims 104-124, or a pharmaceutically acceptable salt thereof, wherein -L-T is a group of formula: 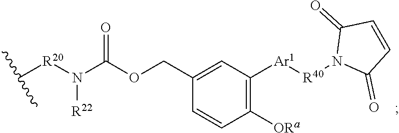
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene;
R^{a} is a substituted heterocycle; and
Ar¹ is substituted or unsubstituted heteroarylene.

150. The compound of any of claims 104-124, or a pharmaceutically acceptable salt thereof, wherein -L-T is a group of formula: 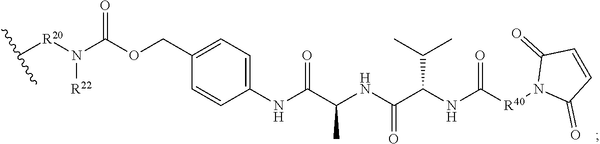
wherein:
R²⁰ is substituted or unsubstituted C₁₋₆ alkylene;
R²² is hydrogen, or substituted or unsubstituted C₁₋₆ alkyl; and
R⁴⁰ is substituted or unsubstituted C₁₋₆ alkylene, or substituted or unsubstituted C₁₋₄₀ heteroalkylene.

151. The compound of any of claims 104-124, or a pharmaceutically acceptable salt thereof, wherein -L-T is a group of formula: 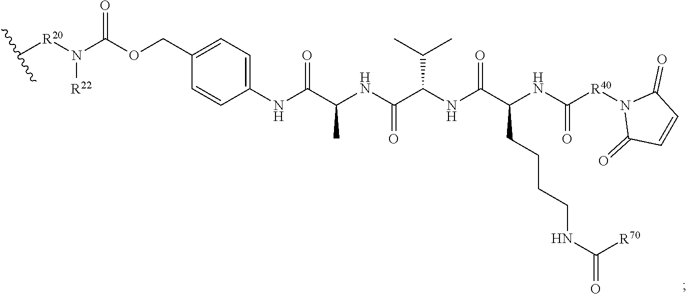
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene; and
and R⁷⁰ is substituted or unsubstituted heteroalkyl.

152. The compound of any of claims 104-124, or a pharmaceutically acceptable salt thereof, wherein -L-T is a group of formula: 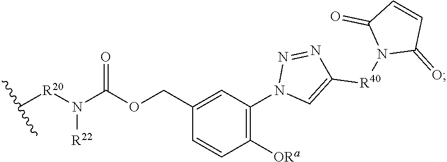
wherein:
R²⁰ is substituted or unsubstituted C₁₋₆ alkylene;
R²² is hydrogen, or substituted or unsubstituted C₁₋₆ alkyl;
R⁴⁰ is substituted or unsubstituted C₁₋₆ alkylene, or substituted or unsubstituted C₁₋₄₀ heteroalkylene; and
R^{a} is substituted or unsubstituted heterocycle.

153. The compound of any of claims 104-124, or a pharmaceutically acceptable salt thereof, wherein -L-T is a group of formula: 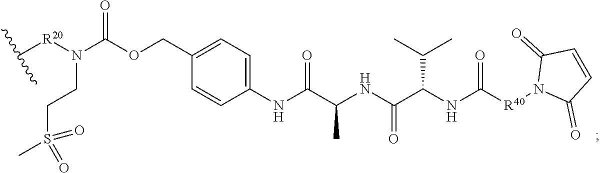
wherein:
R²⁰ is substituted or unsubstituted C₁₋₆ alkylene; and
R⁴⁰ is substituted or unsubstituted C₁₋₆ alkylene, or substituted or unsubstituted C₁₋₄₀ heteroalkylene.

154. The compound of any of claims 104-124, or a pharmaceutically acceptable salt thereof, wherein -L-T is a group of formula: 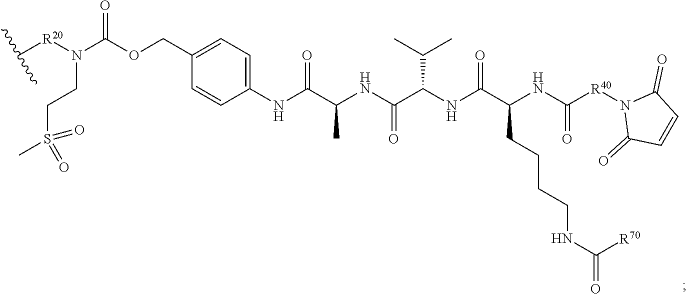
wherein:
R²⁰ is substituted or unsubstituted C₁₋₆ alkylene;
R⁴⁰ is substituted or unsubstituted C₁₋₆ alkylene, or substituted or unsubstituted C₁₋₄₀ heteroalkylene; and
R⁷⁰ is substituted or unsubstituted heteroalkyl.

155. The compound of any of claims 104-124, or a pharmaceutically acceptable salt thereof, wherein -L-T is a group of formula: 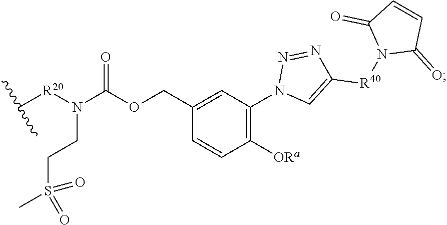
wherein:
R²⁰ is substituted or unsubstituted C₁₋₆ alkylene;
R⁴⁰ is substituted or unsubstituted C₁₋₆ alkylene, or substituted or unsubstituted C₁₋₄₀ heteroalkylene; and
R^{a} is substituted or unsubstituted heterocycle.

156. The compound of any of claims 104-124, or a pharmaceutically acceptable salt thereof, wherein -L-T is a group of formula: 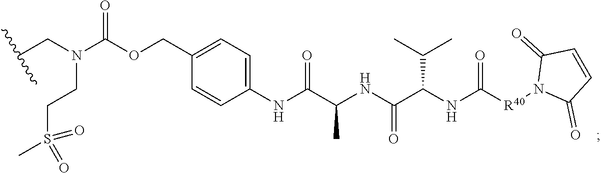
wherein:
R⁴⁰ is substituted or unsubstituted C₁₄ alkylene, or substituted or unsubstituted C₁₋₄₀ heteroalkylene.

157. The compound of any of claims 104-124, or a pharmaceutically acceptable salt thereof, wherein -L-T is a group of formula: 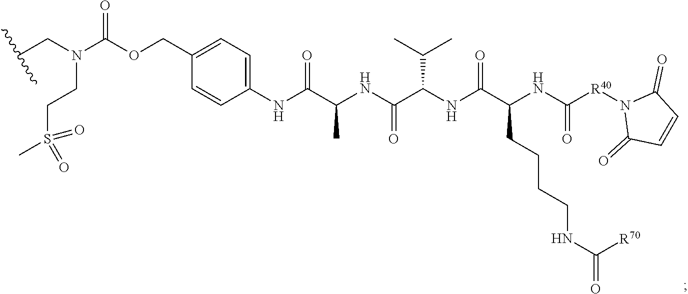
wherein:
R⁴⁰ is substituted or unsubstituted C₁₋₆ alkylene, or substituted or unsubstituted C₁₋₄₀ heteroalkylene; and
R⁷⁰ is substituted or unsubstituted heteroalkyl.

158. The compound of any of claims 104-124, or a pharmaceutically acceptable salt thereof, wherein -L-T is a group of formula: 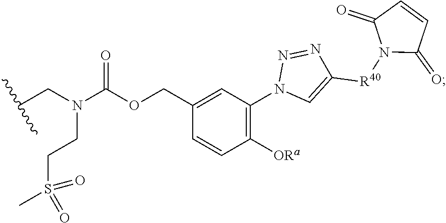
wherein:
R⁴⁰ is substituted or unsubstituted C₁₋₆ alkylene, or substituted or unsubstituted C₁₋₄₀ heteroalkylene; and
R^{a} is substituted or unsubstituted heterocycle.

159. The compound of any of claims 104-124, or a pharmaceutically acceptable salt thereof, wherein -L-T is a group of formula:

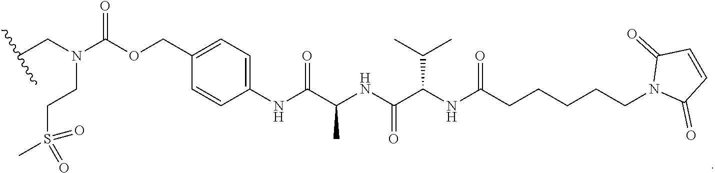 [SMILES: *CN(CCS(C)(=O)=O)C(=O)OCc1ccc(NC(=O)[C@H](C)NC(=O)[C@@H](NC(=O)CCCCCN2C(=O)C=CC2=O)C(C)C)cc1]

160. The compound of any of claims 104-124, or a pharmaceutically acceptable salt thereof, wherein -L-T is a group of formula:

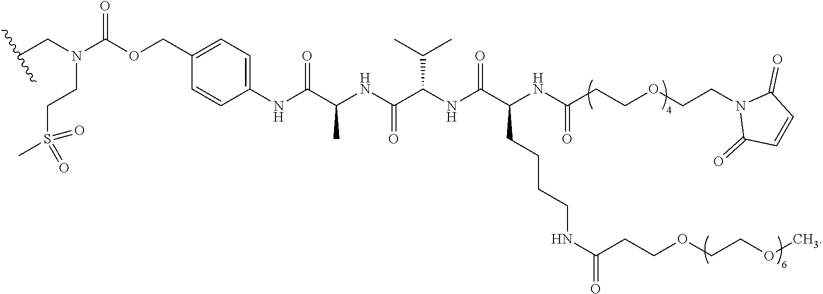

161. The compound of any of claims 104-124, or a pharmaceutically acceptable salt thereof, wherein -L-T is a group of formula:

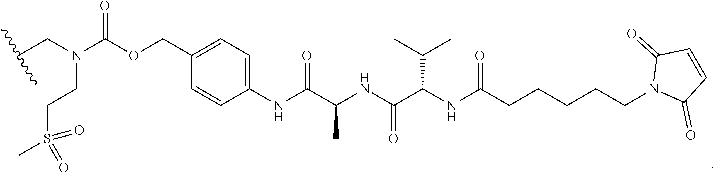 [SMILES: *CN(CCS(C)(=O)=O)C(=O)OCc1ccc(NC(=O)[C@H](C)NC(=O)[C@@H](NC(=O)CCCCCN2C(=O)C=CC2=O)C(C)C)cc1]

162. The compound of any of claims 104-124, or a pharmaceutically acceptable salt thereof, wherein -L-T is a group of formula:

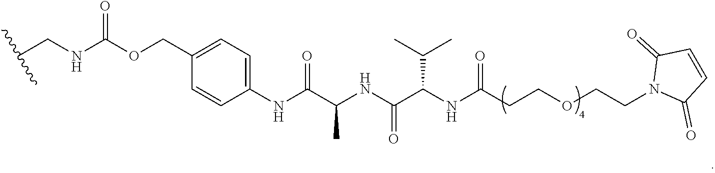

163. The compound of any of claims 104-124, or a pharmaceutically acceptable salt thereof, wherein L is a bond; and T is hydrogen.

164. The compound of any of claim 52-99 or 146-163, wherein the compound is: 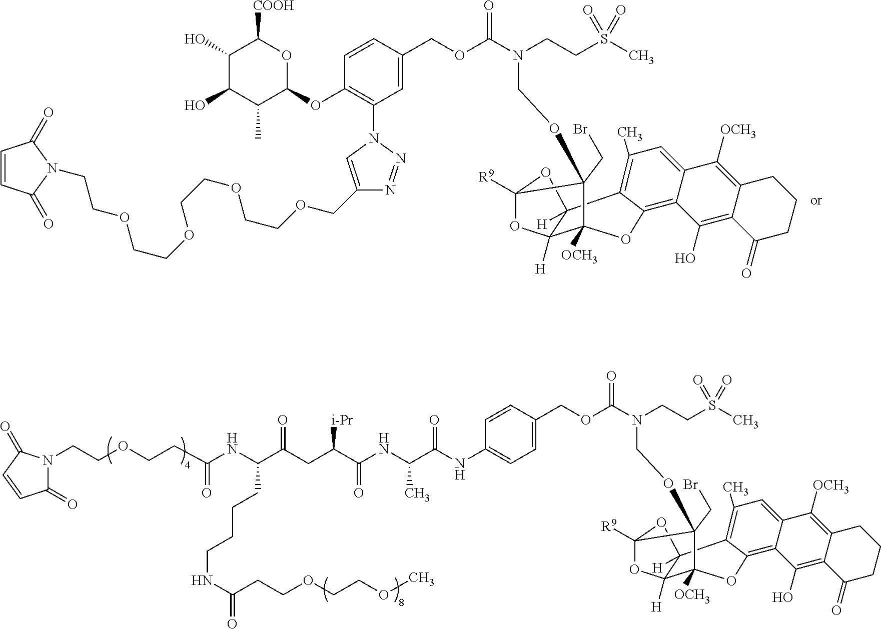
or a pharmaceutically acceptable salt thereof.

165. The compound of claim 52, wherein the compound is: 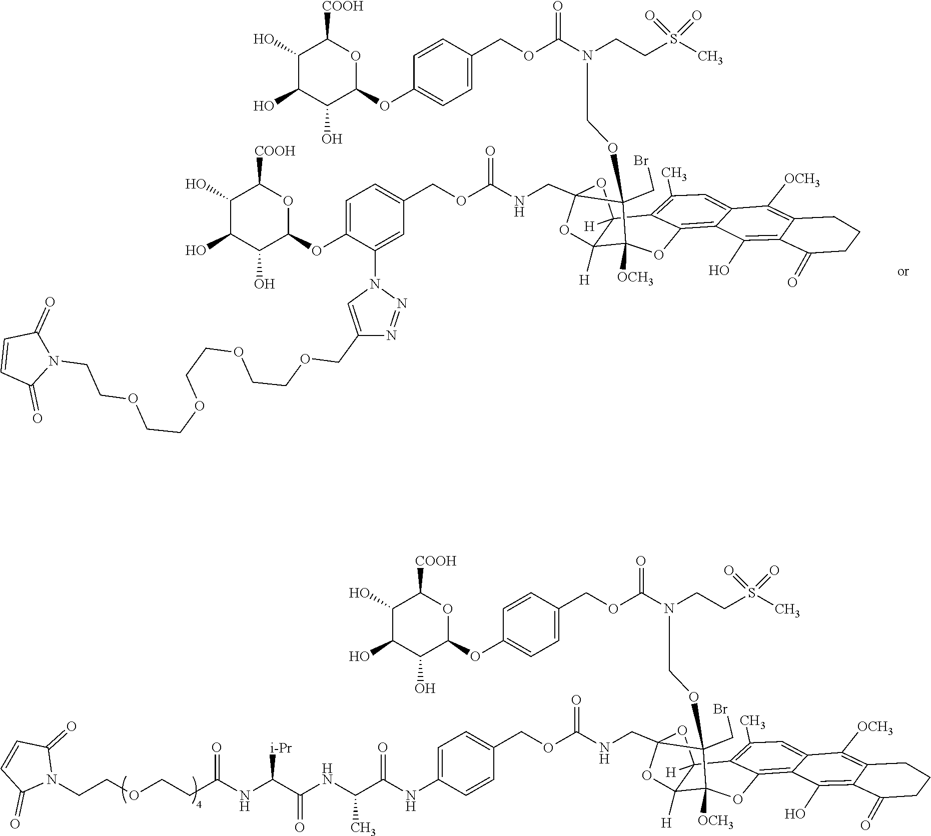
or a pharmaceutically acceptable salt thereof.

166. The compound of any preceding claim, wherein the compound is: 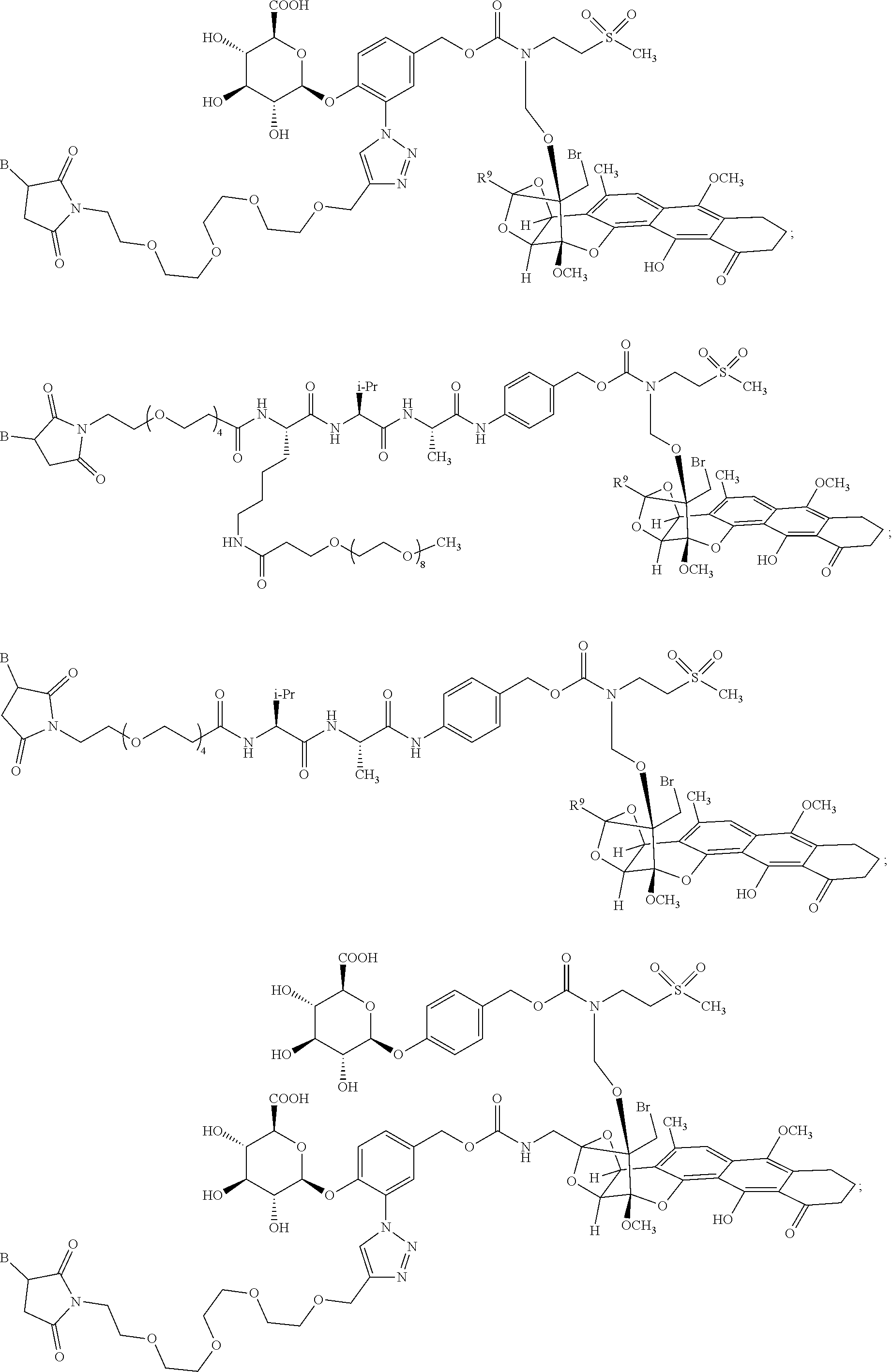 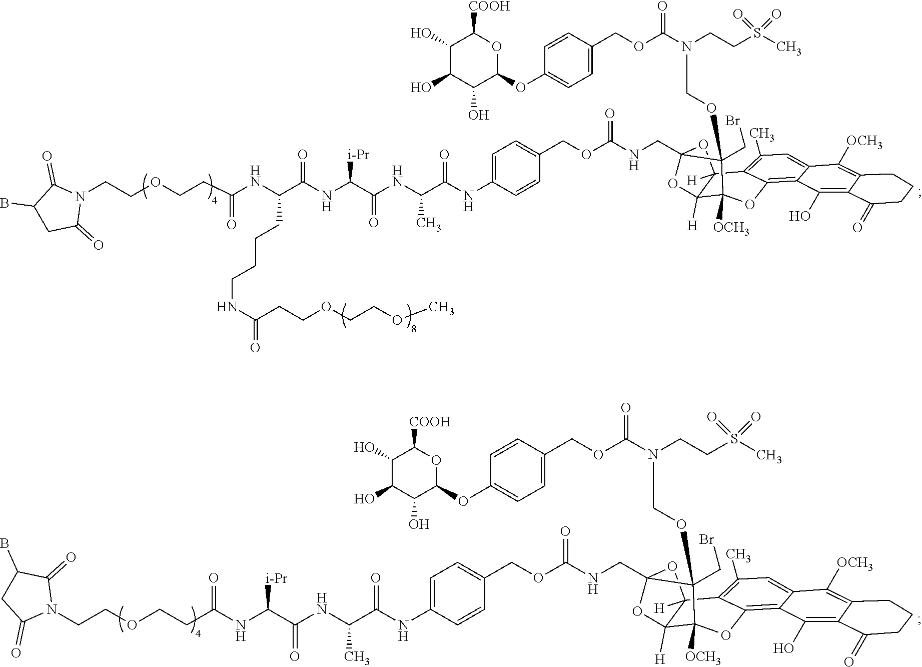
or a pharmaceutically acceptable salt thereof.

167. The compound of any of claims 1-166, or a pharmaceutically acceptable salt thereof, wherein B is an antibody or an antibody fragment.

168. The compound of any of claims 1-167, or a pharmaceutically acceptable salt thereof, wherein B is an antibody selected from Adecatumumab, Afutuzumab, Alemtuzumab (CAMPATH), Bavituximab, Belantamab, Belimumab, Bevacizumab (AVASTIN), Brentuximab, Cantuzumab, Cetuximab (ERBITUX), Citatuzumab, Cixutumumab, Conatumumab, Dacetuzumab, Elotuzumab, Enfortumab, Etaracizumab, Farletuzumab, Figitumumab, Gemtuzumab, Ibritumomab, Inotuzumab, Ipilimumab (YERVOY), Iratumumab, Labetuzumab, Lexatumumab, Lintuzumab, Loncastuximab, Lucatumumab, Mapatumumab, Matuzumab, Milatuzumab, Moxetumomab, Necitumumab, Nimotuzumab, Ofatumumab (ARZERRA), Olaratumab, Oportuzumab, Panitumumab (VECTIBIX), Pertuzumab (PERJETA), Polatuzumab, Pritumumab, Rituximab (RITUXAN), Robatumumab, Sacituzumab, Sibrotuzumab, Siltuximab, Tacatuzumab, Tigatuzumab, Tositumomab (BEXXAR), Trastuzumab (HERCEPTIN), Tucotuzumab, Veltuzumab, Votumumab, Zalutumumab, and fragments thereof.

169. A method of preparing a compound of any of claims 1-51, the method comprising deprotecting a cycloadduct of Formula (A): 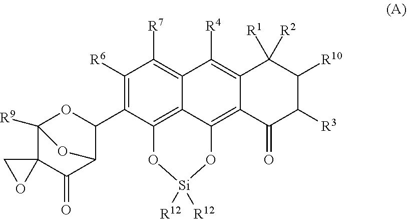
or salt thereof, wherein R^{I2} is a cyclic or acyclic, branched or unbranched, substituted or unsubstituted aliphatic, substituted or unsubstituted aryl, or substituted or unsubstituted heteroaryl.

170. The method of claim 169 further comprising reacting a silacycle of Formula (B): 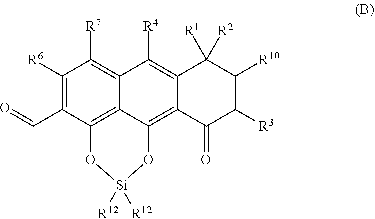
or a salt thereof, with an α-epoxy-β-oxodiazoketone of formula: 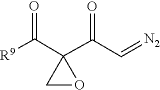
or a salt thereof, to provide the cycloadduct of Formula (A): 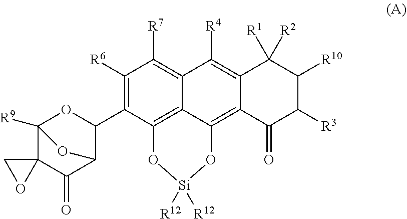
or salt thereof.

171. A method of preparing a compound of any of claims 1-51, the method comprising alkylating a compound of Formula (I): 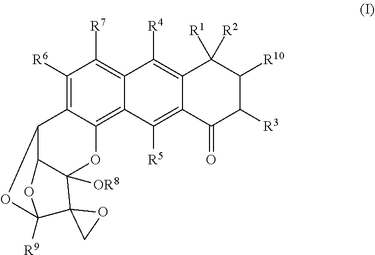
or salt thereof, wherein R⁸ is hydrogen; to provide a compound of Formula (I), or a salt thereof, wherein R⁸ is alkyl.

172. A method of preparing a compound of any of claims 1-51, the method comprising providing a compound of Formula (I-f): 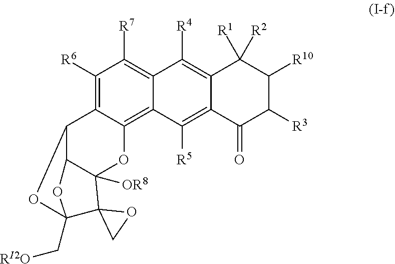
or salt thereof, wherein R⁸ is hydrogen; and R^{I2} is a protecting group; alkylating and deprotecting the compound to provide a compound of Formula (I-f): 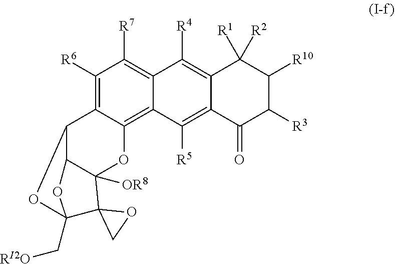
or a salt thereof, wherein R⁸ is alkyl, and R^{I2} is hydrogen.

173. A method of preparing a compound of any of claims 52-100, the method comprising providing a compound of Formula (I): 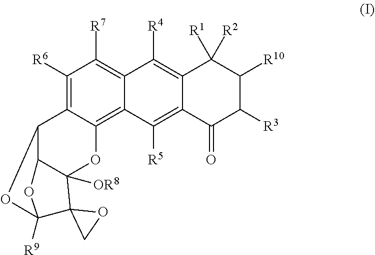
or a pharmaceutically acceptable salt thereof, and ring-opening the compound to provide a compound of Formula (II): 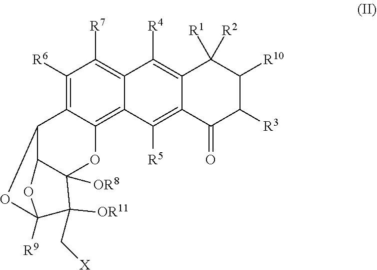
or a pharmaceutically acceptable salt thereof.

174. A method of preparing a compound of Formula (C): 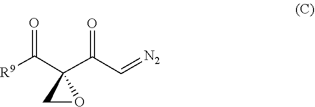
or salt thereof, the method comprising deprotecting and oxidizing a compound of formula (D): 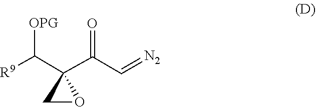
or salt thereof, wherein:
PG is a protecting group; and
R⁹ is hydrogen or —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; C(═O)R^{I1}; -L-A-B; or -L-T; or two R^{I2} groups are optionally joined to form a substituted or unsubstituted heterocyclyl or substituted or unsubstituted heteroaryl ring.

175. The method of claim 174 further comprising hydrolyzing a compound of Formula (E): 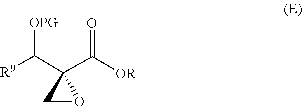
or salt thereof; wherein R is cyclic or acyclic, substituted or unsubstituted, branched or unbranched aliphatic; to provide a compound of Formula (F): 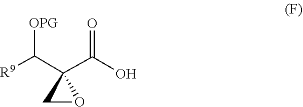
or a salt thereof; and converting the compound of Formula (F) to the compound of Formula (D): 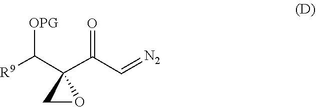
or salt thereof.

176. The method of claim 175 further comprising epoxidizing and protecting a compound of Formula (G): 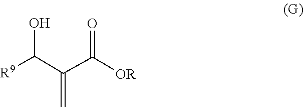
or a salt thereof; to provide the compound of Formula (E): 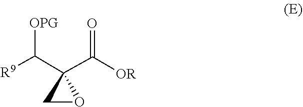
or salt thereof.

177. The method of claim 176, further comprising reacting an aldehyde of Formula (H): 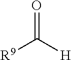
or salt thereof; with an acrylate, or salt thereof, to provide the compound of Formula (G): 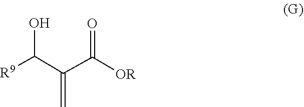
or a salt thereof.

178. The method of claim 175, wherein the compound of Formula (E) is the compound of Formula (E-1): 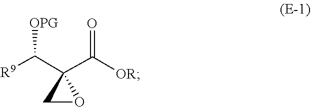
the method further comprising stereoselectively epoxidizing and protecting the compound of Formula (G): 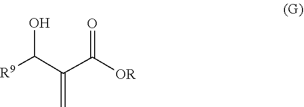
or a salt thereof; to provide the compound of Formula (E-1), or a salt thereof.

179. The method of claim 175, wherein the compound of Formula (E) is the compound of Formula (E-2): 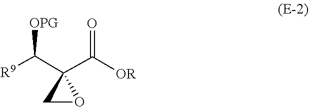
the method further comprising enantioenriching, protecting, and epoxidizing the compound of Formula (G): 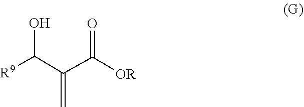
or a salt thereof; to provide the compound of Formula (E-2), or salt thereof.

180. A compound of Formula: 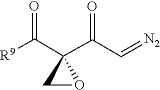
or a salt thereof; wherein:
R⁹ is hydrogen or —C(R^{I1})₃; wherein each occurrence of R^{I1} is independently hydrogen; carbohydrate; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; —OR^{I2}; —SR^{I2}; azido; halogen; or —N(R^{I2})₂; with the proviso that not more than one occurrence of R^{I1} is —OR^{I2}; wherein each occurrence of R^{I2} is independently hydrogen; carbohydrate; protecting group; cyclic or acyclic, substituted or unsubstituted, branched or unbranched aliphatic; cyclic or acyclic, substituted or unsubstituted, branched or unbranched heteroaliphatic; acyl; substituted or unsubstituted aryl; substituted or unsubstituted heteroaryl; substituted or unsubstituted heteroarylalkyl; C(═O)R^{I1}; -L-A-B; or -L-T; or two R^{I2} groups are optionally joined to form a substituted or unsubstituted heterocyclyl or substituted or unsubstituted heteroaryl ring.

181. The compound of claim 180, wherein the compound is 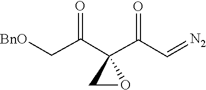
or a salt thereof.

182. The compound of any one of claim 1-22 or 52-75, wherein R⁹ is CH₂OCH₃.

183. The compound of any one of claims 1-18, 41, 42, 52-71, 94, 95, wherein R⁹ is methyl.

184. The compound of any one of claims 1-18; 52-71, wherein R⁹ is hydrogen.

185. A pharmaceutical composition comprising a compound of any of claim 1-168 or 182-184, or a pharmaceutically acceptable salt thereof, and a pharmaceutically acceptable carrier.

186. A method of treating a cardiovascular disease, a proliferative disease, diabetic retinopathy, an inflammatory disease, an autoimmune disease, or an infectious disease in a subject in need thereof, the method comprising administering to the subject an effective amount of the compound of any of claim 1-168 or 182-184, or a pharmaceutically acceptable salt thereof, or the pharmaceutical composition of claim 185.

187. The method of claim 186, wherein the proliferative disease is a cancer.

188. The method of any of claim 186 or 187, wherein the cancer is breast cancer, non-small cell lung cancer, ovarian cancer, colorectal cancer, diffuse large B-cell lymphoma, or multiple myeloma.

189. The method of any of claims 186-188, wherein the cancer is Her2+ breast cancer, TROP2+ breast cancer, TROP2+ non-small cell lung cancer, EGFR+ non-small cell lung cancer, or FRa+ ovarian cancer.

190. The method of any of claims 186-188, wherein the cancer is CD19+ diffuse large B-cell lymphoma or BCMA+ Multiple Myeloma.

191. The method of claim 186, wherein the infectious disease is a bacterial, fungal, or parasitic infection.

192. The method of claim 191, wherein the parasitic infection is malaria.

193. A kit comprising the compound of any of claim 1-168 or 182-184, or the pharmaceutical composition of claim 185, and instructions for its use.
